(19) United States
(12) Patent Application Publication
(10) Pub. No.: US 2025/0163162 A1
(43) Pub. Date: May 22, 2025

(54) COMPOSITIONS AND METHODS RELATED TO RECEPTOR PAIRING

(71) Applicant: Synthekine, Inc., Menlo Park, CA (US)

(72) Inventors: Robert Kastelein, Menlo Park, CA (US); Sandro Vivona, Menlo Park, CA (US); Deepti Rokkam, Menlo Park, CA (US); Patrick Lupardus, Menlo Park, CA (US); Mahalakshmi Ramadass, Menlo Park, CA (US)

(21) Appl. No.: 18/260,688

(22) PCT Filed: Jan. 11, 2022
(86) PCT No.: PCT/US2022/012049

§ 371 Date: Jul. 7, 2023

Related U.S. Application Data
(63) Continuation-in-part of application No. PCT/US21/44858, filed on Aug. 6, 2021.
(60) Provisional application No. 63/136,098, filed on Jan. 11, 2021.
(60) Provisional application No. 63/135,884, filed on Jan. 11, 2021.

(51) Int. Cl. C07K 16/28; A61P 37/04

(52) U.S. Cl. CPC C07K 16/2866; A61P 37/04; C07K 2317/31; C07K 2317/524; C07K 2317/526; C07K 2317/53; C07K 2317/565; C07K 2317/569; C07K 2317/75; C07K 2319/00

(57) ABSTRACT

Provided herein are IL10Rα/IL2Rγ binding proteins that bind to IL10Rα and IL2Rγ and comprise an anti-IL10Rα V_{H}H antibody and an anti-IL2Rγ V_{H}H antibody.

### CROSS-REFERENCE TO RELATED PATENT APPLICATIONS

The present patent application claims benefit of priority to U.S. Provisional Patent Application No. 63/136,098, filed Jan. 11, 2021; U.S. Provisional Patent Application No. 63/135,884, filed Jan. 11, 2021 and PCT Patent Application No. PCT/US2021/044858, filed Aug. 6, 2021, each of which is incorporated by references.

### BACKGROUND OF THE DISCLOSURE

The binding of IL10 to the IL10 receptor (IL10R) can trigger both immunosuppressive and immunostimulatory effects on various cell types. IL10 can cause a number of adverse and undesirable effects by a variety of mechanisms resulting from, among other factors, the presence of IL10R on different cell types.

The anti-inflammatory cytokine interleukin-10 (IL-10), also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, -β, -γ, -δ, -ε, -κ, -Ω, and -τ) and interferon-like molecules (limitin, IL-28A, IL-28B, and IL-29). Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds. The IL-10 receptor, a type II cytokine receptor, consists of alpha (IL10Ra) and beta (IL10Rb) subunits, which are also referred to as R1 and R2, respectively. Receptor activation requires binding to both alpha and beta. One homodimer of an IL-10 polypeptide binds to alpha and the other homodimer of the same IL-10 polypeptide binds to beta.

IL-10 exhibits pleiotropic effects in immunoregulation and inflammation through actions on T cells, B cells, macrophages, and antigen presenting cells (APC). IL-10 is produced by mast cells, counteracting the inflammatory effect that these cells have at the site of an allergic reaction. Although IL-10 is predominantly expressed in macrophages, expression has also been detected in activated T cells, B cells, mast cells, and monocytes. IL-10 can suppress immune responses by inhibiting expression of IL-1α, IL-1β, IL-6, IL8, TNFα, GM-CSF and G-CSF in activated monocytes and activated macrophages, and it also suppresses IFN-γ production by NK cells. IL10 can block NF-κB activity and is involved in the regulation of the JAK-STAT signaling pathway.

IL2 is a pluripotent cytokine which is produced by antigen activated T cells. IL2 exerts a wide spectrum of effects on the immune system and plays important roles in regulating both immune activation, suppression and homeostasis. IL2 promotes the proliferation and expansion of activated T lymphocytes, induces proliferation and activation of naïve T cells, potentiates B cell growth, and promotes the proliferation and expansion of NK cells. Human interleukin 2 (IL2) is a 4 alpha-helix bundle cytokine of 133 amino acids. IL2 is a member of the IL2 family of cytokines which includes IL2, IL-4, IL-7, IL 9, IL-15 and IL21.

IL2 exerts its effect on mammalian immune cells through interaction with three different cell surface proteins: (1) CD25 (also referred to as the IL2 receptor alpha, IL2Rα, p55), CD122 (also referred to as the interleukin-2 receptor beta, IL2Rβ, IL15Rβ and p70-75), and CD132 (also referred to as the interleukin 2 receptor gamma, IL2Rγ; or common gamma chain as it is a component of other multimeric receptors in the IL2 receptor family). In addition to the “low affinity” CD25 IL2 receptor, two additional IL2 receptor complexes have been characterized: (a) an “intermediate affinity” dimeric IL2 receptor comprising CD122 and CD132 (also referred to as “IL2Rβγ”), and (b) a “high affinity” trimeric IL2 receptor complex comprising the CD25, CD122 and CD132 proteins (also referred to as “IL2Rαβγ”). hIL2 possesses a Kd of approximately 10⁻⁹M with respect to the intermediate affinity CD122/CD132 (IL2βγ) receptor complex. hIL2 possesses a Kd of approximately 10⁻¹¹M with respect to the high IL2 affinity receptor complex.

In addition to forming a subunit of the high affinity IL2 receptor, CD132 is a type 1 cytokine receptor and is shared by the receptor complexes for IL-4, IL-7, IL-9, IL-15, and IL21, hence it being referred to in the literature as the “common” gamma chain. Human CD132 (hCD132) is expressed as a 369 amino acid pre-protein comprising a 22 amino acid N-terminal signal sequence. Amino acids 23-262 (amino acids 1-240 of the mature protein) correspond to the extracellular domain, amino acids 263-283 (amino acids 241-262 of the mature protein) correspond to the 21 amino acid transmembrane domain, and amino acids 284-369 (amino acids 262-347 of the mature protein) correspond to the intracellular domain. hCD132 is referenced at UniProtKB database as entry P31785. Human CD132 nucleic acid and protein sequences may be found as Genbank accession numbers: NM_000206 and NP_000197 respectively.

### SUMMARY OF THE DISCLOSURE

In one aspect, provided herein is an IL10Rα/IL2Rγ binding protein that specifically binds to IL10Rα and IL2Rγ, comprising an anti-IL10Rα V_{H}H antibody and an anti-IL2Rγ V_{H}H antibody.

In some embodiments, the IL10Rα/IL2Rγ binding protein that specifically binds to IL10Rα and IL2Rγ comprises an anti-IL10Rα VHH antibody and an anti-IL2Rγ VHH antibody, wherein,
(A) the anti-IL10Rα VHH antibody comprises a complementarity determining region 1 (CDR1) comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(B) the anti-IL10Rα VHH antibody comprises a complementarity determining region 1 (CDR1) comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(C) the anti-IL10Rα VHH antibody comprises a complementarity determining region 1 (CDR1) comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO: 11; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(D) the anti-IL10Rα VHH antibody comprises a complementarity determining region 1 (CDR1) comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(E) the anti-IL10Rα VHH antibody comprises a complementarity determining region 1 (CDR1) comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(F) the anti-IL10Rα VHH antibody comprises a complementarity determining region 1 (CDR1) comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23; and
wherein the anti-IL2Rγ VHH antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47.

In some embodiments, the anti-IL10Rα V_{H}H antibody comprises: (1) a complementarity determining region 1 (CDR1) having a sequence of any one of SEQ ID NOS:1, 5, 9, 13, 1, and 21; (2) a CDR2 having a sequence of any one of SEQ ID NOS:2, 6, 10, 14, 18, and 22; and (3) a CDR3 having a sequence of any one of SEQ ID NOS:3, 7, 11, 15, 19, and 23.

In some embodiments, the anti-IL10Rα V_{H}H antibody comprises CDR1, CDR2, and CDR3 sequences of an anti-IL10Rα V_{H}H antibody selected from the group consisting of DR235, DR236, DR237, DR239, DR240, and DR241. In some embodiments, the anti-IL10Rα V_{H}H antibody comprises a sequence having at least 90% (e.g., 90%, 91%, 92%, 93%, 94%, 95%, 96%, 97%, 98%, 99%, or 100%) identity to a sequence of any one of DR235 (SEQ ID NO:4), DR236 (SEQ ID NO:8), DR237 (SEQ ID NO:12), DR239 (SEQ ID NO:16), DR240 (SEQ ID NO:20), and DR241 (SEQ ID NO:24).

In some embodiments, the anti-IL10Rα V_{H}H antibody comprises: (1) a complementarity determining region 1 (CDR1) having a sequence of any one of SEQ ID NOS: 25, 29, 33, 37, 41, and 45; (2) a CDR2 having a sequence of any one of SEQ ID NOS: 26, 30, 34, 38, 42, and 46; and (3) a CDR3 having a sequence of any one of SEQ ID NOS: 27, 31, 35, 39, 43, and 47.

In some embodiments, the anti-IL2Rγ V_{H}H antibody comprises CDR1, CDR2, and CDR3 sequences of an anti-IL2Rγ V_{H}H antibody selected from the group consisting of DR229, DR230, DR231, DR232, DR233, and DR234. In some embodiments, the anti-IL10Rα V_{H}H antibody comprises a sequence having at least 90% (e.g., 90%, 91%, 92%, 93%, 94%, 95%, 96%, 97%, 98%, 99%, or 100%) identity to a sequence of any one of DR229 (SEQ ID NO:28), DR230 (SEQ ID NO:32), DR231 (SEQ ID NO:36), DR232 (SEQ ID NO:40), DR233 (SEQ ID NO:44), and DR234 (SEQ ID NO:48).

In some embodiments of this aspect, the anti-IL10Rα V_{H}H antibody is at the N-terminus and the anti-IL2Rγ V_{H}H antibody is at the C-terminus. In certain embodiments, the binding protein comprises a sequence having at least 90% (e.g., 90%, 91%, 92%, 93%, 94%, 95%, 96%, 97%, 98%, 99%, or 100%) identity to a sequence of any one of SEQ ID NOS:49-59.

In other embodiments, the anti-IL2Rγ V_{H}H antibody is at the N-terminus and the anti-IL10Rα V_{H}H antibody is at the C-terminus. In some embodiments, the binding protein comprises a sequence with at least 90% (e.g., 90%, 91%, 92%, 93%, 94%, 95%, 96%, 97%, 98%, 99%, or 100%) identity to a sequence of any one of SEQ ID NOS:60 and 61.

In some embodiments, the anti-IL10Rα V_{H}H antibody and the anti-IL2Rγ V_{H}H antibody are joined by a peptide linker. In certain embodiments, the peptide linker comprises between 1 and 50 amino acids.

In some embodiments, the binding protein comprises a sequence with at least 90% (e.g., 90%, 91%, 92%, 93%, 94%, 95%, 96%, 97%, 98%, 99%, or 100%) identity to a sequence of any one of SEQ ID NOS:49-61 or 96-179, optionally without a HHHHHH sequence.

In some embodiments of this aspect, the binding protein is conjugated to an Fc polypeptide or an Fc domain. In some embodiments, the Fc polypeptide or an Fc domain is from an IgG1, IgG2, IgG3 or IgG4. In some embodiments, the IL10Rα/IL2Rγ binding protein comprises SEQ ID NO: 556 or SEQ ID NO:558. In other embodiments, the binding protein is PEGylated.

Also provided is an IL10Rα/IL2Rγ binding protein that specifically binds to IL10Rα and IL2Rγ, comprising an anti-IL10Rα VHH antibody and an anti-IL2Rγ VHH antibody, wherein the IL10Rα/IL2Rγ binding protein is linked to a Fc polypeptide or a Fc domain from an IgG1, IgG2, IgG3 or IgG4.

Also provided is a heterodimeric IL10Rα binding protein/IL2Rγ binding protein pair, the heterodimeric IL10Rα binding protein/IL2Rγ binding protein pair comprising a first polypeptide of the formula #1:

anti-IL10Rα V_{H}H antibody-L1ₐ-UH1-Fc1 [1]

and a second polypeptide of the formula #2:

anti-IL2Rγ V_{H}H antibody-L2_{b}-UH2-Fc2 [2]

wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
FC2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and
   wherein the polypeptide of formula 1 and the polypeptide of formula 2 are linked by at least one interchain disulfide bond, and wherein
   (A) the anti-IL10Rα V_{H}H antibody comprises a complementarity determining region 1 (CDR1) comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (B) the anti-IL10Rα V_{H}H antibody comprises a complementarity determining region 1 (CDR1) comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (C) the anti-IL10Rα V_{H}H antibody comprises a complementarity determining region 1 (CDR1) comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO:11; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (D) the anti-IL10Rα V_{H}H antibody comprises a complementarity determining region 1 (CDR1) comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (E) the anti-IL10Rα V_{H}H antibody comprises a complementarity determining region 1 (CDR1) comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (F) the anti-IL10Rα V_{H}H antibody comprises a complementarity determining region 1 (CDR1) comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23; and
   wherein the anti-IL2Rγ V_{H}H antibody comprises:
   i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
   (G) the anti-IL10Rα V_{H}H antibody comprises:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 in a row of Table 10;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 in a row of Table 10; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 10; and
   the anti-IL2Rγ V_{H}H antibody comprises:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 listed in Table 11 or Table 12;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 listed in Table 11 or Table 12; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 11 or Table 12.

In another aspect, the disclosure provides an isolated nucleic acid encoding the IL10Rα/IL2Rγ binding protein or a heterodimeric IL10Rα binding protein/IL2Rγ binding protein pair described herein.

In another aspect, the disclosure provides an expression vector comprising the nucleic acid described herein.

In another aspect, the disclosure provides an isolated host cell comprising the vector comprising the nucleic acid described herein.

In another aspect, the disclosure provides a pharmaceutical composition comprising the IL10Rα/IL2Rγ binding protein or a heterodimeric IL10Rα binding protein/IL2Rγ binding protein pair described herein and a pharmaceutically acceptable carrier.

In another aspect, the disclosure provides a method of treating a neoplastic disease in a subject in need thereof, comprising administering to the subject a therapeutically effective amount of an IL10Rα/IL2Rγ binding protein or a heterodimeric IL10Rα binding protein/IL2Rγ binding protein pair described herein or a pharmaceutical composition comprising the IL10Rα/IL2Rγ binding protein or a heterodimeric IL10Rα binding protein/IL2Rγ binding protein pair described herein and a pharmaceutically acceptable carrier.

In some embodiments, the method further comprises the administration of a supplementary agent to the subject. In some embodiments, the supplementary agent is selected from the group consisting of a chemotherapeutic agent, an antibody, an immune checkpoint modulators, a TIL, a CAR-T cell, and a physical method.

In some embodiments of this aspect, the neoplastic disease is selected from the group consisting of adenomas, fibromas, hemangiomas, hyperplasia, atypia, metaplasia, dysplasia, carcinomas, leukemias, breast cancers, sarcomas, leukemias, lymphomas, genitourinary cancers, ovarian cancers, urethral cancers, bladder cancers, prostate cancers, gastrointestinal cancers, colon cancers, esophageal cancers, stomach cancers, lung cancers; myelomas; pancreatic cancers; liver cancers; kidney cancers; endocrine cancers; skin cancers; gliomas, neuroblastomas, astrocytomas, myelodysplastic disorders; cervical carcinoma-in-situ; intestinal polyposes; oral leukoplakias; histiocytoses, hyperprofroliferative scars including keloid scars, respiratory system carcinomas, gastrointestinal system carcinomas, genitourinary system carcinomas, testicular carcinomas, breast carcinomas, prostatic carcinomas, endocrine system carcinomas, melanomas, adenocarcinomas, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage, promyeloid leukemia (APML), acute myelogenous leukemia (AML) and chronic myelogenous leukemia (CML), precursor lymphoid neoplasms, mature B-cell neoplasms, mature T-cell neoplasms, Hodgkin's Lymphoma, and immunodeficiency-associated lymphoproliferative disorders, lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). erythroblastic leukemia and acute megakaryoblastic leukemia, malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

In another aspect, the disclosure provides a method of treating an autoimmune or inflammatory disease, disorder, or condition in a subject in need thereof, comprising administering to the subject a therapeutically effective amount of an IL10Rα/IL2Rγ binding protein described herein or a pharmaceutical composition comprising the IL10Rα/IL2Rγ binding protein described herein and a pharmaceutically acceptable carrier.

In some embodiments of this aspect, the method further comprises administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody. In certain embodiments, the therapeutic antibody is an antibody that binds a protein selected from the group consisting of BLyS, CD11a, CD20, CD25, CD3, CD52, IgEIL12/IL23, IL17a, IL1β, IL4Rα, IL5, IL6R, integrin-α4β7, RANKL, TNFα, VEGF-A, and VLA-4.

In some embodiments of this aspect, the disease, disorder or condition is selected from viral infections, Heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

In certain embodiments, the methods described herein do not cause anemia.

In another aspect, the disclosure provides a method to selectively induce activity (e.g., phosphorylation) in one or more of a first cell type over one or more of a second cell type, comprising contacting a population of cells comprising both the first and second cell types with an IL10Rα/IL2Rγ binding protein or a heterodimeric IL10Rα binding protein/IL2Rγ binding protein pair described herein, thereby selectively inducing activity in one or more of the first cell type over one or more of the second cell type.

In some embodiments, the first cell type is CD4⁺ T cells. In some embodiments, the first cell type is CD8⁺ T cells. In some embodiments, the second cell type is NK cells. In some embodiments, the second cell type is B cells. In some embodiments, the second cell type is monocytes. In some embodiments, the first cell type is CD4⁺ T cells, CD8⁺ T cells, B cells, and/or NK cells. In certain embodiments, the second cell type is monocytes.

In other embodiments of this aspect, the activity of the first cell type is at least 1.2 (e.g., at least 1.4, 1.6, 1.8, 2, 2.2, 2.4, 2.6, 2.8, 3, 3.2, 3.4, 3.6, 3.8, 4, 6, 8, 10, 12, 14, 16, 18, or 20) fold more than the activity of the second cell type.

### BRIEF DESCRIPTION OF THE DRAWINGS

FIGS. 1A-1D show different configurations of one or two IL10Rα/IL2Rγ binding proteins conjugated to an Fc domain.

FIGS. 2A-2E show dot plots of screening data for IL10Rα/IL2Rγ binding proteins' induction of pSTAT3 activity in different cell types.

FIGS. 3A-3E show the dose-dependent induction of IL10Rβ/IL2Rγ binding proteins in different cell types.

FIG. 4 of the attached drawings provides a schematic representation of one embodiment of the bivalent binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) interaction of a bivalent binding molecule and a second first receptor subunit comprising an extracellular domain (7), and transmembrane domain (8) and an intracellular domain (9) wherein the intracellular domain of the first receptor (6) and the intracellular domain of the second receptor (9) on of a bivalent binding molecule are within a proximal distance (11).

FIG. 5 of the attached drawings provides a schematic representation of two illustrative configurations of bivalent binding molecules of the present disclosure. Panel A provides a schematic representation of an illustrative single polypeptide chain bivalent binding molecule comprising, from amino to carboxy, a first single domain antibody (1) and a second single domain antibody (3) and a linker (2). Panel B provides a schematic representation of a bivalent binding molecule comprising a first single domain antibody (1) and a second single domain antibody (3) and a linker (2) and a knob-into-hole Fc domain comprising a first subunit which is a Fc knob (13) and a second subunit which is a Fc hole (14) wherein the single domain antibody is stably associated with the Fc domain via a IgG hinge sequence (12).

FIG. 6 of the attached drawings provides a schematic representation of two illustrative configurations of bivalent binding molecules of the present disclosure. Panel A provides a schematic representation of an illustrative bivalent binding molecule comprising a first single domain antibody (1) and a second single domain antibody (3) and a linker (2). Panel B provides a schematic representation of a bivalent binding molecule comprising two polypeptide chains, the first polypeptide chain comprising (from amino to carboxy) a first single domain antibody (1), a linker sequence, a second single domain antibody (3), an IgG hinge sequence (12) and an Fc knob domain (13) and a second polypeptide comprising an Fc hole (14) wherein the first and second polypeptides are in stable association via the interaction of the knob-into-hole Fc domain.

FIG. 7, Panel A provides alternative schematic representations of configurations of the bivalent binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG hinge sequence (12) and a Fc hole subunit (13), wherein the first and second single domain antibodies are in stable associate via the interaction of the knob-into-hole Fc domain.

FIG. 7, Panel B provides a schematic representations of a bivalent binding molecule the binding domains are single domain antibodies associated via transition metal coordinate covalent complex. As illustrated, the bivalent binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

FIGS. 8A-8E provide STAT3 response dose response data in various cell types with the indicated IL10Rα/IL2Rγ binding molecule test articles when tested on CD8 T cells (FIG. 8A), monocytes (FIG. 8B), CD4 T cells (FIG. 8C), B cells (FIG. 8D), and NK cells (FIG. 8E) as more fully described in the Examples.

FIGS. 9A-9E provide STAT3 response dose response data in various cell types with the A2 and H1 IL10Rα/IL2Rγ binding molecules and their Fc conjugated counterparts (H1Fc=DR992, SEQ ID NO:556 and A2 Fc=DR995, SEQ ID NO:558), when tested on CD8 T cells (FIG. 9A), monocytes (FIG. 9B), CD4 T cells (FIG. 9C), B cells (FIG. 9D), and NK cells (FIG. 9E) as more fully described in the Examples.

FIGS. 10A-10D provides the results a monocyte functional assay indicating levels of IL1b expression (FIG. 10A), IL6 (FIG. 10B), IL8 (FIG. 10C) and TNFα (FIG. 10D) in response to varying doses of the A2 and H1 IL10Rα/IL2Rγ binding molecules and their Fc conjugated counterparts (H1Fc=DR992, SEQ ID NO:556 and A2 Fc=DR995, SEQ ID NO:558) as more fully described in the Examples.

FIGS. 11A-11B provides the results CD8 T cell blast functional assay indicating levels of granzyme A expression (FIG. 11A) and Granzyme B expression (FIG. 11B) in response to varying doses of the A2 and H1 IL10Rα/IL2Rγ binding molecules and their Fc conjugated counterparts (H1Fc=DR992, SEQ ID NO:556 and A2 Fc=DR995, SEQ ID NO:558) as more fully described in the Examples.

### DETAILED DESCRIPTION OF THE DISCLOSURE

### I. Introduction

The present disclosure provides compositions useful in the pairing of cellular receptors to generate desirable effects useful in treatment of diseases. In general, binding proteins are provided that comprise a first domain that binds to IL10Rα (also referred to as IL10R1) and a second domain that binds to IL2Rγ, such that upon contacting with a cell expressing IL10Rα and IL2Rγ, the binding protein causes the functional association of IL10Rα and IL2Rγ, thereby resulting in functional dimerization of the receptors and downstream signaling.

Several advantages flow from the binding proteins described herein. Unlike IL10R's natural ligand, IL10, which can trigger both immunosuppressive and immunostimulatory effects on various cell types, the binding proteins described herein can decouple the immunosuppressive and immunostimulatory effects and selectively provide only the desired effect on the desired cell type(s). When IL10 is used as a therapeutic in mammalian, particularly human, subjects, it may also trigger a number of adverse and undesirable effects by a variety of mechanisms including the presence of IL10R on different cell types and the binding to IL10R on the different cell types may result in undesirable effects and/or undesired signaling on cells expressing the IL10 receptor. The present disclosure is directed to methods and compositions that modulate the multiple effects of IL10R binding so that desired therapeutic signaling occurs, particularly in a desired cellular or tissue subtype, while also minimizing undesired activity and/or intracellular signaling.

For example, it is known that IL10 has activities on macrophages (e.g., monocytes) and T cells (e.g., CD4⁺ T cells and CD8⁺ T cells). Macrophages is a cell type that expresses both IL10Rα and IL10Rβ receptors but when activated significantly can result in the phagocytosis of aging red blood cells and resulting in side effects such as anemia in patients receiving IL10 therapy. In some embodiments, the method provided herein uses a binding protein of the present disclosure that binds to IL10Rα and IL2Rγ resulting in the selective activation of T cells relative to activation of macrophages. The selective activation of T cells relative to macrophages is beneficial because IL10-activated macrophages and its associated side effect of anemia can be avoided. Binding proteins as described herein that provide for the selective substantial activation of T cells while providing a minimal activation of macrophages resulting in a molecule which retains the beneficial properties of an native IL10 ligand but results in diminished undesirable side effects relative to the native IL10 ligand.

In some embodiments, the binding molecule that specifically binds to IL10Rα and IL2Rγ has a reduced Eₘₐₓ compared to the Eₘₐₓ of IL10. Eₘₐₓ reflects the maximum response level in a cell type that can be obtained by a ligand (e.g., a binding protein described herein or the native cytokine (e.g., IL10)). In some embodiments, the binding protein that specifically binds to IL10Rα and IL2Rγ described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 10% and 90%, between 10% and 80%, between 1% and 70%, between 1% and 60%, between 1% and 50%, between 1% and 40%, between 1% and 30%, between 1% and 20%, or between 1% and 10%) of the Eₘₐₓ caused by IL10. In some embodiments, by varying the linker length of the binding protein that specifically binds to IL10Rα and IL2Rγ, the Eₘₐₓ of the binding protein can be changed. The binding protein can cause Eₘₐₓ in the most desired cell types, for example, CD8⁺ T cells. In some embodiments, the Eₘₐₓ in CD8⁺ T cells caused by a binding protein that specifically binds to IL10Rα and IL2Rγ is between 1% and 100% (e.g., between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ in other T cells caused by the binding protein. In other embodiments, the Eₘₐₓ of the binding protein that specifically binds to IL10Rα and IL2Rγ is greater (e.g., at least 1%, 5%, 10%, 15%, 20%, 25%, 30%, 35%, 40%, 45%, or 50% greater) than the Eₘₐₓ of the natural ligand.

The level of downstream signaling caused by the binding protein, can also be measured.

In some embodiments, the IL10Rα/IL2Rγ binding molecules described herein are partial agonists. In some embodiments, the binding molecules described herein are designed such that the binding molecules are full agonists. In some embodiments, the binding molecules described herein are designed such that the binding molecules are super agonists.

The present disclosure provides disclosure provides bivalent IL10Rα/IL2Rγ comprising:
a first single domain antibody (sdAb) that specifically binds to the extracellular domain of IL10Rα of the IL10Rα/IL2Rγ (an “anti-IL1Rα sdAb”), and
a second single domain antibody that specifically binds to extracellular domain IL2Rγ of the IL10Rα/IL2Rγ ((an “anti-IL2Rγ sdAb”),
   wherein the anti-IL10Rα sdAb and anti-IL2Rγ sdAb are stably associated, and wherein contacting a cell expressing IL10Rα and IL2Rγ with an effective amount of the bivalent binding molecule results in the dimerization of IL10Rα and IL2Rγ and results in intraceullar signaling. In some embodiments, one or both of the sdAbs is a an scFv. In some embodiments, one or both of the sdAbs is a VHH.

In some embodiments, one sdAb of the IL10Rα/IL2Rγ bivalent binding molecule is an scFv and the other sdAb is a VHH.

In some embodiments, the first and second sdAbs are covalently bound via a chemical linkage.

In some embodiments, the first and second sdAbs are provided as single continuous polypeptide.

In some embodiments, the first and second sdAbs are provided as single continuous polypeptide optionally comprising an intervening polypeptide linker between the amino acid sequences of the first and second sdAbs.

In some embodiments the bivalent binding molecule optionally comprising a linker, is optionally expressed as a fusion protein with an additional amino acid sequence. In some embodiments, the additional amino acid sequence is a purification handle such as a chelating peptide or an additional protein such as a subunit of an Fc molecule.

The disclosure also provides an expression vector comprising a nucleic acid encoding the bispecific binding molecule operably linked to one or more expression control sequences. The disclosure also provides an isolated host cell comprising the expression vector expression vector comprising a nucleic acid encoding the bispecific binding molecule operably linked to one or more expression control sequences functional in the host cell.

In another aspect, the disclosure provides a pharmaceutical composition comprising the IL10Rα/IL2Rγ binding molecule described herein and a pharmaceutically acceptable carrier.

In another aspect, the disclosure provides a method of treating an autoimmune or inflammatory disease, disorder, or condition or a viral infection in a subject in need thereof, comprising administering to the subject a therapeutically effective amount of an IL10Rα/IL2Rγ binding molecule described herein or a pharmaceutical composition described herein.

In some embodiments, the binding proteins described herein are designed such that the binding proteins provide the maximal desired IL10 intracellular signaling from binding to IL10Rα and IL2Rγ on the desired cell types, while providing significantly less IL10 signaling in other undesired cell types. This can be achieved, for example, by selection of binding proteins having differing affinities or causing different Eₘₐₓ for IL10Rα and IL2Rγ as compared to the affinity of IL10 for IL10R. Because different cell types respond to the binding of ligands to its cognate receptor with different sensitivity, by modulating the affinity of the dimeric ligand (or its individual binding moieties) for the IL10 receptor relative to wild-type IL10 binding facilitates the stimulation of desired activities while reducing undesired activities on non-target cells. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be effected by the level of downstream signaling caused by the binding protein, can also be measured.

### II. Definitions

As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, and camelids antibodies (e.g., human antibodies).

The term antibody also includes so called “single-domain antibodies” or “sdAbs,” as well as “heavy chain antibodies” or “V_{H}Hs,” which are further defined herein. V_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in V_{H}H frameworks. Antibodies having a given specificity may also be derived from non-mammalian sources such as V_{H}Hs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings.

The term antibody includes both the parent antibody and its derivatives such as affinity matured, veneered, CDR grafted, humanized, camelized (in the case of V_{H}Hs), or binding molecules comprising binding domains of antibodies (e.g., CDRs) in non-immunoglobulin scaffolds.

The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin. In some embodiments, the antibody is a “full length antibody” comprising variable and constant domains providing binding and effector functions.

The term antibody includes antibody conjugates comprising modifications to prolong duration of action such as fusion proteins or conjugation to polymers (e.g., PEGylated).

As used herein, the term “binding protein” refers to a protein that can bind to one or more cell surface receptors or domains or subunits thereof. In some embodiments, a binding protein specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. In the context of the present disclosure, the numbering of the CDR positions is provided according to Kabat numbering conventions. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variable region of an antibody can follow Kabat numbering unless indicated as determined by a hybrid of Kabat and Chothia numbering schemes.

As used herein, the term “conservative amino acid substitution” refers to an amino acid replacement that changes a given amino acid to a different amino acid with similar biochemical properties (e.g., charge, hydrophobicity, and size). For example, the amino acids in each of the following groups can be considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

As used herein, the term “downstream signaling” refers to the cellular signaling process that is caused by the interaction of two or more cell surface receptors that are brought into proximity of each other.

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains.

As used herein, the term “multimerization” refers to two or more cell surface receptors, or domains or subunits thereof, being brought in close proximity to each other such that the receptors, or domains or subunits thereof, can interact with each other and cause downstream signaling.

As used herein, the terms “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign (2) pre-malignant (or “pre-cancerous”); and (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

As used herein, the terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein, the term “percent (%) sequence identity” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned.

Algorithms that are suitable for determining percent sequence identity and sequence similarity are the BLAST and BLAST 2.0 algorithms, which are described in Altschul et al. (1990) J Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻².

As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g., antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

As used herein, the term “single-domain antibody” or “sdAb” refers to an antibody having a single monomeric variable antibody domain. A sdAb is able to bind selectively to a specific antigen. A V_{H}H antibody, further defined below, is an example of a sdAb.

As used herein, the term “specifically bind” refers to the degree of selectivity or affinity for which one molecule binds to another. In the context of binding pairs (e.g., a binding protein described herein/receptor, a ligand/receptor, antibody/antigen, antibody/ligand, antibody/receptor binding pairs), a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample.

In a particular embodiment, a V_{H}H in a bispecific V_{H}H² binding protein described herein binds to a receptor (e.g., the first receptor or the second receptor of the natural or non-natural receptor pairs) if the equilibrium dissociation constant between the V_{H}H and the receptor is greater than about 10⁶ M, alternatively greater than about 10⁸ M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays.

As used herein, the term “subject”, “recipient”, “individual”, or “patient”, refers to any mammalian subject for whom diagnosis, treatment, or therapy is desired, particularly humans. These terms can also be used interchangeably herein. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. TRI, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

As used herein, the term “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

The terms “treat”, “treating”, treatment” and the like refer to a course of action (such as administering a binding protein described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

As used herein, the term “V_{H}H” is a type of sdAb that has a single monomeric heavy chain variable antibody domain. Such antibodies can be found in or produced from Camelid mammals (e.g., camels, llamas) which are naturally devoid of light chains.

As used herein, the term “V_{H}H²” refers to two V_{H}Hs that are joined together by way of a linker (e.g., a covalent bond or a peptide linker). A “bispecific V_{H}H²” refers to a V_{H}H² that has a first V_{H}H binding to a first receptor, or domain or subunit thereof, and a second V_{H}H binding to a second receptor, or domain or subunit thereof.

### III. Compositions and Methods

The disclosure describes IL10Rα/IL2Rγ binding proteins that bind to IL10Rα and IL2Rγ or domains thereof. The various binding proteins can be screened for binding to IL10Rα and IL2Rγ or domains thereof and for signal transduction in therapeutically relevant cell types.

The binding proteins described herein can specifically bind to IL10Rα and IL2Rγ and can comprise an anti-IL10Rα V_{H}H antibody and an anti-IL2Rγ V_{H}H antibody. The binding proteins described herein are also referred to as anti-IL10Rα/IL2Rγ V_{H}H². The binding protein can cause the multimerization of IL10Rα and IL2Rγ and downstream signaling.

### Anti-IL10Rα V_{H}H Antibody

In some embodiments, the present disclosure provides polypeptides comprising any of the anti-IL10Rα V_{H}H antibodies described herein, e.g., a polypeptide comprising an anti-IL10Rα V_{H}H comprising a CDR1, a CDR2, and a CDR3 selected from Table 1 below. In certain embodiments, the present disclosure provides a polypeptide comprising a set of CDR1, CDR2, and CDR3 (e.g., CDR1, CDR2, and CDR3 described in the same row) selected from a row of Table 1 below. In certain embodiments, the present disclosure provides a polypeptide comprising a sequence having at least 90% (e.g., 90%, 91%, 92%, 93%, 94%, 95%, 96%, 97%, 98%, 99%, or 100%) identity to a sequence of an anti-IL10Rα V_{H}H antibody selected from Table 1 below. In some embodiments, a polypeptide provided by the present disclosure can comprise a dimer or multimer of two or more of anti-IL10Rα V_{H}H antibodies as described in Table 1, in which the anti-IL10Rα V_{H}H antibodies can be the same or different.

In some embodiments, the present disclosure provides an anti-IL10Rα V_{H}H antibody, which may be incorporated into a multivalent binding protein as described herein, comprising one or more of the CDR1s, CD2s, CDR3s or V_{H}H amino acid sequences as listed in Table 1 below. In some embodiments, the anti-IL10Rα V_{H}H antibody can comprise: (1) a CDR1 having a sequence of any one of SEQ ID NOS:1, 5, 9, 13, 17, 21, or 264-269 or a variant thereof that has a sequence having one, two, or three amino acid substitutions relative to a sequence of any one of SEQ ID NOS:1, 5, 9, 13, 17, 21, or 264-269; (2) a CDR2 having a sequence of any one of SEQ ID NOS:2, 6, 10, 14, 18, and 22 or a variant thereof that has a sequence having one, two, or three amino acid substitutions relative to a sequence of any one of SEQ ID NOS:2, 6, 10, 14, 18, and 22; (3) a CDR3 having a sequence of any one of SEQ ID NOS:3, 7, 11, 15, 19, and 23 or a variant thereof that has a sequence having one, two, or three amino acid substitutions relative to a sequence of any one of SEQ ID NOS:3, 7, 11, 15, 19, and 23. In some embodiments, an anti-IL10Rα V_{H}H antibody may be modified for extended half-life (e.g., Fc conjugation, PEGylation) either alone or in the context of a multivalent binding protein as described herein. In some embodiments the moiety providing half-life extension (e.g., PEG, Fc polypeptide, or Fc domain) is conjugated, optionally via a linker, to the N-terminus of the antibody, the C-terminus of the antibody, or an internal amino acid residue (particularly via conjugation to the side chains of lysine or cysteine residues). In some embodiments, the Fc polypeptide or an Fc domain is from an IgG1, IgG2, IgG3 or IgG4.

In some embodiments, the anti-IL10Rα V_{H}H antibody can comprise a set of CDR1, CDR2, and CDR3 (e.g., CDR1, CDR2, and CDR3 described in the same row) selected from a row of Table 1 below. In each set of CDR1, CDR2, and CDR3, (1) the CDR1 can have the indicated sequence in the set or a variant thereof that has a sequence having one, two, or three amino acid substitutions relative to the indicated sequence; (2) the CDR2 can have the indicated sequence in the set or a variant thereof that has a sequence having one, two, or three amino acid substitutions relative to the indicated sequence; (3) the CDR3 can have the indicated sequence in the set or a variant thereof that has a sequence having one, two, or three amino acid substitutions relative to the indicated sequence.

Further, an anti-IL10Rα V_{H}H antibody can comprise CDR1, CDR2, and CDR3 having the sequences of SEQ ID NOS:1-3. Further, an anti-IL10Rα V_{H}H antibody can comprise CDR1, CDR2, and CDR3 having the sequences of SEQ ID NOS:5-7. Further, an anti-IL10Rα V_{H}H antibody can comprise CDR1, CDR2, and CDR3 having the sequences of SEQ ID NOS:9-11. Further, an anti-IL10Rα V_{H}H antibody can comprise CDR1, CDR2, and CDR3 having the sequences of SEQ ID NOS:13-15. Further, an anti-IL10Rα V_{H}H antibody can comprise CDR1, CDR2, and CDR3 having the sequences of SEQ ID NOS:17-19. Further, an anti-IL10Rα V_{H}H antibody can comprise CDR1, CDR2, and CDR3 having the sequences of SEQ ID NOS:21-23.

Further, an anti-IL10Rα V_{H}H antibody can comprise CDR1, CDR2, and CDR3 having the sequences of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO:4. Further, an anti-IL10Rα V_{H}H antibody can comprise CDR1, CDR2, and CDR3 having the sequences of SEQ ID NOS:5-7, respectively, and at least 90% (e.g., 90%, 91%, 92%, 93%, 94%, 95%, 96%, 97%, 98%, 99%, or 100%) identity to the sequence of SEQ ID NO:8. Further, an anti-IL10Rα V_{H}H antibody can comprise CDR1, CDR2, and CDR3 having the sequences of SEQ ID NOS:9-11, respectively, and at least 90% (e.g., 90%, 91%, 92%, 93%, 94%, 95%, 96%, 97%, 98%, 99%, or 100%) identity to the sequence of SEQ ID NO:12. Further, an anti-IL10Rα V_{H}H antibody can comprise CDR1, CDR2, and CDR3 having the sequences of SEQ ID NOS:13-15, respectively, and at least 90% (e.g., 90%, 91%, 92%, 93%, 94%, 95%, 96%, 97%, 98%, 99%, or 100%) identity to the sequence of SEQ ID NO:16. Further, an anti-IL10Rα V_{H}H antibody can comprise CDR1, CDR2, and CDR3 having the sequences of SEQ ID NOS:17-19, respectively, and at least 90% (e.g., 90%, 91%, 92%, 93%, 94%, 95%, 96%, 97%, 98%, 99%, or 100%) identity to the sequence of SEQ ID NO:20. Further, an anti-IL10Rα V_{H}H antibody can comprise CDR1, CDR2, and CDR3 having the sequences of SEQ ID NOS:21-23, respectively, and at least 90% (e.g., 90%, 91%, 92%, 93%, 94%, 95%, 96%, 97%, 98%, 99%, or 100%) identity to the sequence of SEQ ID NO:24.

TABLE 1
Anti-IL10Rα V_{H}H antibody sequences

| | | CDR1 | | | |
|---|---|---|---|---|---|
| V_{H}H | CDR1 | (Chothia/ | | | |
| Ab | (Kabat) | Kabat) | CDR2 | CDR3 | V_{H}H |
| DR235 | IDYMA | YLYSIDY | VIYTAS | VRKTDS | QVQLQESGGGSVQAGGSLRLSCAASRYL |
| | (SEQ ID | MA (SEQ | GATFYP | YLFDAQ | YSIDYMAWFRQSPGKEREPVAVIYTASG |
| | NO: 1) | ID NO: | DSVKG | SFTY | ATFYPDSVKGRFTISQDNAKMTVYLQMN |
| | | 264) | (SEQ | (SEQ | SLKSEDTAMYYCAAVRKTDSYLFDAQSF |
| | | | ID NO: | ID NO: | TYWGQGTQVTVSS (SEQ ID NO: 4) |
| | | | 2) | 3) | |
| DR236 | SYCMG | FTYSSYC | SIDSDG | DLMSTV | QVQLQESGGGSVQAGGSLRLSCAASRFT |
| | (SEQ ID | MG (SEQ | STSYTD | VPGFCG | YSSYCMGWFRQAPGKEREGVASIDSDGS |
| | NO: 5) | ID NO: | SVKG | FLLSAG | TSYTDSVKGRFTISKDNAKNTLYLQMNS |
| | | 265) | (SEQ | MDY | LKPEDTAMYYCALDLMSTVVPGFCGFLL |
| | | | ID NO: | (SEQ | SAGMDYWGKGTQVTVSS (SEQ ID |
| | | | 6) | ID NO: | NO: 8) |
| | | | | 7) | |
| DR237 | MYCMG | YTYSMYC | QINSDG | DSRVYG | QVQLQESGGGSVQAGGSLRLSCAASGYT |
| | (SEQ ID | MG (SEQ | STSYAD | GSWYER | YSMYCMGWFRQAPGKEREGVAQINSDGS |
| | NO: 9) | ID NO: | SVKG | LCGPYT | TSYADSVKGRFTISKDNAKNTLYLQMNS |
| | | 266) | (SEQ | YEYNY | LKPEDTAMYYCAADSRVYGGSWYERLCG |
| | | | ID NO: | (SEQ | PYTYEYNYWGQGTQVTVSS (SEQ |
| | | | 10) | ID NO: | ID NO: 12) |
| | | | | 11) | |
| DR239 | SNCMG | YTYSSNC | TIYTGG | EPLSRV | QVQLQESGGGSVQAGGSLRLSCTVSGYT |
| | (SEQ ID | MG (SEQ | GNTYYA | YGGSCP | YSSNCMGWFRQAPGKEREGVATIYTGGG |
| | NO: 13) | ID NO: | DSVKG | TPTFDY | NTYYADSVKGRFTISQDNAKNTVYLQMN |
| | | 267) | (SEQ | (SEQ | NLKPEDTAMYYCAAEPLSRVYGGSCPTP |
| | | | ID NO: | ID NO: | TFDYWGQGTQVTVSS (SEQ ID NO: |
| | | | 14) | 15) | 16) |
| DR240 | SYCMG | YTYSSYC | VIDSDG | DLGHYR | QVQLQESGGGSVQAGGSLRLSCGASGYT |
| | (SEQ ID | MG (SEQ | STSYAD | PPCGVL | YSSYCMGWFRQVPGKEREGVAVIDSDGS |
| | NO: 17) | ID NO: | SVKG | YLGMDY | TSYADSVKGRFTISKDNGKNTLYLQMNS |
| | | 268) | (SEQ | (SEQ | LKPEDTAMYYCAADLGHYRPPCGVLYLG |
| | | | ID NO: | ID NO: | MDYWGKGTQVTVSS (SEQ ID NO: |
| | | | 18) | 19) | 20) |
| DR241 | SYDMT | YSNCSYD | AIHSDG | DPLHCR | QVQLQESGGGSVQAGGSLRLSCAASGYS |
| | (SEQ ID | MT (SEQ | STRYAD | AHGGSW | NCSYDMTWYRQAPGKEREFVSAIHSDGS |
| | NO: 21) | ID NO: | SVKG | YSVRAN | TRYADSVKGRFFISQDNAKNTVYLQMNS |
| | | 269) | (SEQ | Y (SEQ | LKPEDTAMYYCKTDPLHCRAHGGSWYSV |
| | | | ID NO: | ID NO: | RANYWGQGTQVTVSS (SEQ ID NO: |
| | | | 22) | 23) | 24) |

### Anti-IL2Rγ V_{H}H Antibody

In some embodiments, the present disclosure provides polypeptides comprising any of the anti-IL2Rγ V_{H}H antibodies described herein, e.g., a polypeptide comprising an anti-IL2Rγ V_{H}H comprising a CDR1, a CDR2, and a CDR3 selected from Table 2 below. In certain embodiments, the present disclosure provides a polypeptide comprising a set of CDR1, CDR2, and CDR3 (e.g., CDR1, CDR2, and CDR3 described in the same row) selected from a row of Table 2 below. In certain embodiments, the present disclosure provides a polypeptide comprising a sequence having at least 90% (e.g., 90%, 91%, 92%, 93%, 94%, 95%, 96%, 97%, 98%, 99%, or 100%) identity to a sequence of an anti-IL2Rγ V_{H}H antibody selected from Table 2 below. In some embodiments, a polypeptide provided by the present disclosure can comprise a dimer or multimer of two or more of anti-IL2Rγ V_{H}H antibodies as described in Table 2, in which the anti-IL2Rγ V_{H}H antibodies can be the same or different.

In some embodiments, the present disclosure provides an anti-IL10Rα V_{H}H antibody, which may be incorporated into a multivalent binding protein as described herein, comprising one or more of the CDR1s, CD2s, CDR3s or V_{H}H amino acid sequences as listed in Table 1 below. In some embodiments, the present disclosure provides an anti-IL2Rγ V_{H}H antibody, which may be incorporated into a multivalent binding protein as described herein, comprising one or more of CDR1s, CD2s, CDR3s or V_{H}H amino acid sequences as listed in Table 2 below. In some embodiments, the anti-IL2Rγ V_{H}H antibody can comprise: (1) a CDR1 having a sequence of any one of SEQ ID NOS:25, 29, 33, 37, 41, 45 or 270-275 or a variant thereof that has a sequence having one, two, or three amino acid substitutions relative to a sequence of any one of SEQ ID NOS:25, 29, 33, 37, 41, 45 or 270-275; (2) a CDR2 having a sequence of any one of SEQ ID NOS:26, 30, 34, 38, 42, and 46 or a variant thereof that has a sequence having one, two, or three amino acid substitutions relative to a sequence of any one of SEQ ID NOS:26, 30, 34, 38, 42, and 46; (3) a CDR3 having a sequence of any one of SEQ ID NOS:27, 31, 35, 39, 43, and 47 or a variant thereof that has a sequence having one, two, or three amino acid substitutions relative to a sequence of any one of SEQ ID NOS:27, 31, 35, 39, 43, and 47. In some embodiments, an anti-IL2Rγ V_{H}H may be modified for extended half life (e.g., Fc conjugation, PEGylation) either alone or in the context of a multivalent binding protein as described herein. In some embodiments the moiety providing half-life extension (e.g., PEG, Fc polypeptide, or Fc domain) is conjugated, optionally via a linker, to the N-terminus of the antibody, the C-terminus of the antibody, or an internal amino acid residue (particularly via conjugation to the side chains of lysine or cysteine residues). In some embodiments, the Fc polypeptide or an Fc domain is from an IgG1, IgG2, IgG3 or IgG4.

In some embodiments, the anti-IL2Rγ V_{H}H antibody can comprise a set of CDR1, CDR2, and CDR3 (e.g., CDR1, CDR2, and CDR3 described in the same row) selected from a row of Table 2 below. In each set of CDR1, CDR2, and CDR3, (1) the CDR1 can have the indicated sequence in the set or a variant thereof that has a sequence having one, two, or three amino acid substitutions relative to the indicated sequence; (2) the CDR2 can have the indicated sequence in the set or a variant thereof that has a sequence having one, two, or three amino acid substitutions relative to the indicated sequence; (3) the CDR3 can have the indicated sequence in the set or a variant thereof that has a sequence having one, two, or three amino acid substitutions relative to the indicated sequence.

Further, an anti-IL2Rγ V_{H}H antibody can comprise CDR1, CDR2, and CDR3 having the sequences of SEQ ID NOS:25-27. Further, an anti-IL2Rγ V_{H}H antibody can comprise CDR1, CDR2, and CDR3 having the sequences of SEQ ID NOS:29-31. Further, an anti-IL2Rγ V_{H}H antibody can comprise CDR1, CDR2, and CDR3 having the sequences of SEQ ID NOS:33-35. Further, an anti-IL2Rγ V_{H}H antibody can comprise CDR1, CDR2, and CDR3 having the sequences of SEQ ID NOS:37-39. Further, an anti-IL2Rγ V_{H}H antibody can comprise CDR1, CDR2, and CDR3 having the sequences of SEQ ID NOS:41-43. Further, an anti-IL2Rγ V_{H}H antibody can comprise CDR1, CDR2, and CDR3 having the sequences of SEQ ID NOS:45-47.

Further, an anti-IL2Rγ V_{H}H antibody can comprise CDR1, CDR2, and CDR3 having the sequences of SEQ ID NOS:25-27, respectively, and at least 90% (e.g., 90%, 91%, 92%, 93%, 94%, 95%, 96%, 97%, 98%, 99%, or 100%) identity to the sequence of SEQ ID NO:28. Further, an anti-IL2Rγ V_{H}H antibody can comprise CDR1, CDR2, and CDR3 having the sequences of SEQ ID NOS:29-31, respectively, and at least 90% (e.g., 90%, 91%, 92%, 93%, 94%, 95%, 96%, 97%, 98%, 99%, or 100%) identity to the sequence of SEQ ID NO:32. Further, an anti-IL2Rγ V_{H}H antibody can comprise CDR1, CDR2, and CDR3 having the sequences of SEQ ID NOS:33-35, respectively, and at least 90% (e.g., 90%, 91%, 92%, 93%, 94%, 95%, 96%, 97%, 98%, 99%, or 100%) identity to the sequence of SEQ ID NO:36. Further, an anti-IL2Rγ V_{H}H antibody can comprise CDR1, CDR2, and CDR3 having the sequences of SEQ ID NOS:37-39, respectively, and at least 90% (e.g., 90%, 91%, 92%, 93%, 94%, 95%, 96%, 97%, 98%, 99%, or 100%) identity to the sequence of SEQ ID NO:40. Further, an anti-IL2Rγ V_{H}H antibody can comprise CDR1, CDR2, and CDR3 having the sequences of SEQ ID NOS:41-43, respectively, and at least 90% (e.g., 90%, 91%, 92%, 93%, 94%, 95%, 96%, 97%, 98%, 99%, or 100%) identity to the sequence of SEQ ID NO:44. Further, an anti-IL2Rγ V_{H}H antibody can comprise CDR1, CDR2, and CDR3 having the sequences of SEQ ID NOS:45-47, respectively, and at least 90% (e.g., 90%, 91%, 92%, 93%, 94%, 95%, 96%, 97%, 98%, 99%, or 100%) identity to the sequence of SEQ ID NO:48.

TABLE 2
Anti-IL2Rγ V_{H}H antibody sequences

| | | CDR1 | | | |
|---|---|---|---|---|---|
| V_{H}H | CDR1 | (Chothia/ | | | |
| Ab. | (Kabat) | Kabat) | CDR2 | CDR3 | V_{H}H |
| DR229 | SYPMT | FSFSSYP | TIASDG | GYGDGT | QVQLQESGGGLVQPGGSLRLSCTASGF |
| | (SEQ ID | MT (SEQ | GSTAYA | PA | SFSSYPMTWARQAPGKGLEWVSTIASD |
| | NO: 25) | ID NO: | ASVEG | (SEQ | GGSTAYAASVEGRFTISRDNAKSTLYL |
| | | 270) | (SEQ | ID NO: | QLNSLKTEDTAMYYCTKGYGDGTPAPG |
| | | | ID NO: | 27) | QGTQVTVSS (SEQ ID NO: 28) |
| | | | 26) | | |
| DR230 | SAHMS | FTFSSAH | SIYSGG | NRLHYY | QVQLQESGGGLVQPGGSLRLSCAASGF |
| | (SEQ ID | MS (SEQ | GTFYAD | SDDDSL | TFSSAHMSWVRQAPGKGREWIASIYSG |
| | NO: 29) | ID NO: | SVKG | (SEQ | GGTFYADSVKGRFTISRDNAKNTLYLQ |
| | | 271) | (SEQ | ID NO: | LNSLKAEDTAMYYCATNRLHYYSDDD |
| | | | ID NO: | 31) | SLRGQGTQVTVSS (SEQ ID NO: |
| | | | 30) | | 32) |
| DR231 | DREMN | FTFDDRE | TISSDG | DFMIAI | QVQLQESGGGSVQAGGSLRLSCTASGF |
| | (SEQ ID | MN (SEQ | STYYAD | QAPGAG | TFDDREMNWYRQAPGNECELVSTISSD |
| | NO: 33) | ID NO: | SVKG | C (SEQ | GSTYYADSVKGRFTISQDNAKNTVYLQ |
| | | 272) | (SEQ | ID NO: | MDSVKPEDTAVYYCAADFMIAIQAPGA |
| | | | ID NO: | 35) | GCWGQGTQVTVSS (SEQ ID NO: |
| | | | 34) | | 36) |
| DR232 | CMG | YTSCMG | TIYTRG | GGYSWS | QVQLQESGGGSVQAGGSLRLSCVASGY |
| | (SEQ ID | (SEQ ID | RSIYYA | AGCEFN | TSCMGWFRQAPGKEREAVATIYTRGRS |
| | NO: 37) | NO: 273) | DSVKG | Y (SEQ | IYYADSVKGRFTISQDNAKNTLYLQMN |
| | | | (SEQ | ID NO: | SLKPEDIAMYSCAAGGYSWSAGCEFNY |
| | | | ID NO: | 39) | WGQGTQVTVSS (SEQ ID NO: 40) |
| | | | 38) | | |
| DR233 | DSDMG | FTFDDSD | TISSDG | EPRGYY | QVQLQESGGGSVQAGGSLRLSCTASGF |
| | (SEQ ID | MG (SEQ | STYYAD | SNYGGR | TFDDSDMGWYRQAPGNECELVSTISSD |
| | NO: 41) | ID NO: | SVKG | RECNY | GSTYYADSVKGRFTISQDNAKNTVYLQ |
| | | 274) | (SEQ | (SEQ | MNSLKPEDTAVYYCAAEPRGYYSNYGG |
| | | | ID NO: | ID NO: | RRECNYWGQGTQVTVSS (SEQ ID |
| | | | 42) | 43) | NO: 44) |
| DR234 | SYCMG | YTFSSYC | ALGGGS | AWVACL | QVQLQESGGGSVQAGGSLRLSCVASGY |
| | (SEQ ID | MG (SEQ | TYYADS | EFGGSW | TFSSYCMGWFRQAPGKEREGVAALGGG |
| | NO: 45) | ID NO: | VKG | YDLARY | STYYADSVKGRFTISQDNAKNTLYLQM |
| | | 275) | (SEQ | KH | NSLKPEDTAMYYCAAAWVACLEFGGSW |
| | | | ID NO: | (SEQ | YDLARYKHWGQGTQVTVSS (SEQ |
| | | | 46) | ID NO: | ID NO: 48) |
| | | | | 47) | |

Anti-IL10Rα/IL2Rγ V_{H}H²

An IL10Rα/IL2Rγ binding protein described herein can comprise an anti-IL10Rα V_{H}H antibody selected from Table 1 and an anti-IL2Rγ V_{H}H antibody selected from Table 2. In some embodiments, the N-terminal V_{H}H of the IL2R binding molecule is an anti-IL10Rα V_{H}H antibody and the C-terminal V_{H}H of the IL10Rα/IL2Rγ binding protein is an anti-IL2Rγ V_{H}H antibody, optionally a linker can be used between the two V_{H}H antibodies. In some embodiments, the N-terminal V_{H}H of the IL10Rα/IL2Rγ binding protein is an anti-IL2Rγ V_{H}H antibody and the C-terminal V_{H}H of the IL10Rα/IL2Rγ binding protein is an anti-IL10Rα V_{H}H antibody, optionally a linker can be used between the two V_{H}H antibodies. Examples of linkers (e.g., GGGS (SEQ ID NO:62)) that can be used to fuse the anti-IL10Rα V_{H}H antibody and the anti-IL2Rγ V_{H}H antibody are described in detail further herein. In some embodiments, the IL10Rα/IL2Rγ binding protein may be operably linked to a metal chelating peptide. Chelating peptides include but are not limited to the Ala-Ser-His-His-His-His-His-His (“ASH6”, SEQ ID NO:81) or the His-His-His-His-His-His (“H6”, SEQ ID NO:82) purification handle to facilitate purification of the binding protein by chelating peptide immobilized metal affinity chromatography (“CP-IMAC, as described in U.S. Pat. No. 4,569,794).

Table 3 below further illustrates examples of IL10Rα/IL2Rγ binding proteins described herein that comprise an anti-IL10Rα V_{H}H antibody at the N-terminus and an anti-IL2Rγ V_{H}H antibody at the C-terminus.

In some embodiments, an IL10Rα/IL2Rγ binding protein comprises the V_{H}H sequence of DR235, the V_{H}H sequence of DR233, and has at least 90% (e.g., 90%, 91%, 92%, 93%, 94%, 95%, 96%, 97%, 98%, 99%, or 100%) identity to the sequence of SEQ ID NO:49, optionally without the terminal HHHHHH. In some embodiments, an IL10Rα/IL2Rγ binding protein comprises the V_{H}H sequence of DR235, the V_{H}H sequence of DR234, and has at least 90% (e.g., 90%, 91%, 92%, 93%, 94%, 95%, 96%, 97%, 98%, 99%, or 100%) identity to the sequence of SEQ ID NO:50, optionally without the terminal HHHHHH. In some embodiments, an IL10Rα/IL2Rγ binding protein comprises the V_{H}H sequence of DR236, the V_{H}H sequence of DR231, and has at least 90% (e.g., 90%, 91%, 92%, 93%, 94%, 95%, 96%, 97%, 98%, 99%, or 100%) identity to the sequence of SEQ ID NO:51, optionally without the terminal HHHHHH. In some embodiments, an IL10Rα/IL2Rγ binding protein comprises the V_{H}H sequence of DR236, the V_{H}H sequence of DR232, and has at least 90% (e.g., 90%, 91%, 92%, 93%, 94%, 95%, 96%, 97%, 98%, 99%, or 100%) identity to the sequence of SEQ ID NO:52, optionally without the terminal HHHHHH. In some embodiments, an IL10Rα/IL2Rγ binding protein comprises the V_{H}H sequence of DR236, the V_{H}H sequence of DR234, and has at least 90% (e.g., 90%, 91%, 92%, 93%, 94%, 95%, 96%, 97%, 98%, 99%, or 100%) identity to the sequence of SEQ ID NO:53, optionally without the terminal HHHHHH. In some embodiments, an IL10Rα/IL2Rγ binding protein comprises the V_{H}H sequence of DR237, the V_{H}H sequence of DR233, and has at least 90% (e.g., 90%, 91%, 92%, 93%, 94%, 95%, 96%, 97%, 98%, 99%, or 100%) identity to the sequence of SEQ ID NO:54, optionally without the terminal HHHHHH. In some embodiments, an IL10Rα/IL2Rγ binding protein comprises the V_{H}H sequence of DR240, the V_{H}H sequence of DR231, and has at least 90% (e.g., 90%, 91%, 92%, 93%, 94%, 95%, 96%, 97%, 98%, 99%, or 100%) identity to the sequence of SEQ ID NO:55, optionally without the terminal HHHHHH. In some embodiments, an IL10Rα/IL2Rγ binding protein comprises the V_{H}H sequence of DR240, the V_{H}H sequence of DR232, and has at least 90% (e.g., 90%, 91%, 92%, 93%, 94%, 95%, 96%, 97%, 98%, 99%, or 100%) identity to the sequence of SEQ ID NO:56, optionally without the terminal HHHHHH. In some embodiments, an IL10Rα/IL2Rγ binding protein comprises the V_{H}H sequence of DR240, the V_{H}H sequence of DR234, and has at least 90% (e.g., 90%, 91%, 92%, 93%, 94%, 95%, 96%, 97%, 98%, 99%, or 100%) identity to the sequence of SEQ ID NO:57, optionally without the terminal HHHHHH. In some embodiments, an IL10Rα/IL2Rγ binding protein comprises the V_{H}H sequence of DR241, the V_{H}H sequence of DR231, and has at least 90% (e.g., 90%, 91%, 92%, 93%, 94%, 95%, 96%, 97%, 98%, 99%, or 100%) identity to the sequence of SEQ ID NO:58, optionally without the terminal HHHHHH. In some embodiments, an IL10Rα/IL2Rγ binding protein comprises the V_{H}H sequence of DR241, the V_{H}H sequence of DR234, and has at least 90% (e.g., 90%, 91%, 92%, 93%, 94%, 95%, 96%, 97%, 98%, 99%, or 100%) identity to the sequence of SEQ ID NO:59, optionally without the terminal HHHHHH.

TABLE 3
Anti-IL10Rα/IL2Rγ V_{H}H² (anti-IL10Rα
V_{H}H-linker-anti-IL2Rγ V_{H}H)

| Anti- | | | |
|---|---|---|---|
| IL10Rα/ | N- | C- | |
| IL2Rγ | terminal | terminal | |
| V_{H}H² | V_{H}H | V_{H}H | Anti-IL10Rα/IL2Rγ V_{H}H² |
| DR437 | DR235 | DR233 | QVQLQESGGGSVQAGGSLRLSCAASRYLYSIDYMAW |
| | | | FRQSPGKEREPVAVIYTASGATFYPDSVKGRFTISQ |
| | | | DNAKMTVYLQMNSLKSEDTAMYYCAAVRKTDSYLFD |
| | | | AQSFTYWGQGTQVTVSSGGGSQVQLQESGGGSVQAG |
| | | | GSLRLSCTASGFTFDDSDMGWYRQAPGNECELVSTI |
| | | | SSDGSTYYADSVKGRFTISQDNAKNTVYLQMNSLKP |
| | | | EDTAVYYCAAEPRGYYSNYGGRRECNYWGQGTQVTV |
| | | | SSASHHHHHH (SEQ ID NO: 49) |
| DR438 | DR235 | DR234 | QVQLQESGGGSVQAGGSLRLSCAASRYLYSIDYMAW |
| | | | FRQSPGKEREPVAVIYTASGATFYPDSVKGRFTISQ |
| | | | DNAKMTVYLQMNSLKSEDTAMYYCAAVRKTDSYLFD |
| | | | AQSFTYWGQGTQVTVSSGGGSQVQLQESGGGSVQAG |
| | | | GSLRLSCVASGYTFSSYCMGWFRQAPGKEREGVAAL |
| | | | GGGSTYYADSVKGRFTISQDNAKNTLYLQMNSLKPE |
| | | | DTAMYYCAAAWVACLEFGGSWYDLARYKHWGQGTQV |
| | | | TVSSASHHHHHH (SEQ ID NO: 50) |
| DR441 | DR236 | DR231 | QVQLQESGGGSVQAGGSLRLSCAASRFTYSSYCMGW |
| | | | FRQAPGKEREGVASIDSDGSTSYTDSVKGRFTISKD |
| | | | NAKNTLYLQMNSLKPEDTAMYYCALDLMSTVVPGFC |
| | | | GFLLSAGMDYWGKGTQVTVSSGGGSQVQLQESGGGS |
| | | | VQAGGSLRLSCTASGFTFDDREMNWYRQAPGNECEL |
| | | | VSTISSDGSTYYADSVKGRFTISQDNAKNTVYLQMD |
| | | | SVKPEDTAVYYCAADFMIAIQAPGAGCWGQGTQVTV |
| | | | SSASHHHHHH (SEQ ID NO: 51) |
| DR442 | DR236 | DR232 | QVQLQESGGGSVQAGGSLRLSCAASRFTYSSYCMGW |
| | | | FRQAPGKEREGVASIDSDGSTSYTDSVKGRFTISKD |
| | | | NAKNTLYLQMNSLKPEDTAMYYCALDLMSTVVPGFC |
| | | | GFLLSAGMDYWGKGTQVTVSSGGGSQVQLQESGGGS |
| | | | VQAGGSLRLSCVASGYTSCMGWFRQAPGKEREAVAT |
| | | | IYTRGRSIYYADSVKGRFTISQDNAKNTLYLQMNSL |
| | | | KPEDIAMYSCAAGGYSWSAGCEFNYWGQGTQVTVSS |
| | | | ASHHHHHH (SEQ ID NO: 52) |
| DR444 | DR236 | DR234 | QVQLQESGGGSVQAGGSLRLSCAASRFTYSSYCMGW |
| | | | FRQAPGKEREGVASIDSDGSTSYTDSVKGRFTISKD |
| | | | NAKNTLYLQMNSLKPEDTAMYYCALDLMSTVVPGFC |
| | | | GFLLSAGMDYWGKGTQVTVSSGGGSQVQLQESGGGS |
| | | | VQAGGSLRLSCVASGYTFSSYCMGWFRQAPGKEREG |
| | | | VAALGGGSTYYADSVKGRFTISQDNAKNTLYLQMNS |
| | | | LKPEDTAMYYCAAAWVACLEFGGSWYDLARYKHWGQ |
| | | | GTQVTVSSASHHHHHH (SEQ ID NO: 53) |
| DR449 | DR237 | DR233 | QVQLQESGGGSVQAGGSLRLSCAASGYTYSMYCMGW |
| | | | FRQAPGKEREGVAQINSDGSTSYADSVKGRFTISKD |
| | | | NAKNTLYLQMNSLKPEDTAMYYCAADSRVYGGSWYE |
| | | | RLCGPYTYEYNYWGQGTQVTVSSGGGSQVQLQESGG |
| | | | GSVQAGGSLRLSCTASGFTFDDSDMGWYRQAPGNEC |
| | | | ELVSTISSDGSTYYADSVKGRFTISQDNAKNTVYLQ |
| | | | MNSLKPEDTAVYYCAAEPRGYYSNYGGRRECNYWGQ |
| | | | GTQVTVSSASHHHHHH (SEQ ID NO: 54) |
| DR465 | DR240 | DR231 | QVQLQESGGGSVQAGGSLRLSCGASGYTYSSYCMGW |
| | | | FRQVPGKEREGVAVIDSDGSTSYADSVKGRFTISKD |
| | | | NGKNTLYLQMNSLKPEDTAMYYCAADLGHYRPPCGV |
| | | | LYLGMDYWGKGTQVTVSSGGGSQVQLQESGGGSVQA |
| | | | GGSLRLSCTASGFTFDDREMNWYRQAPGNECELVST |
| | | | ISSDGSTYYADSVKGRFTISQDNAKNTVYLQMDSVK |
| | | | PEDTAVYYCAADFMIAIQAPGAGCWGQGTQVTVSSA |
| | | | SHHHHHH (SEQ ID NO: 55) |
| DR466 | DR240 | DR232 | QVQLQESGGGSVQAGGSLRLSCGASGYTYSSYCMGW |
| | | | FRQVPGKEREGVAVIDSDGSTSYADSVKGRFTISKD |
| | | | NGKNTLYLQMNSLKPEDTAMYYCAADLGHYRPPCGV |
| | | | LYLGMDYWGKGTQVTVSSGGGSQVQLQESGGGSVQA |
| | | | GGSLRLSCVASGYTSCMGWFRQAPGKEREAVATIYT |
| | | | RGRSIYYADSVKGRFTISQDNAKNTLYLQMNSLKPE |
| | | | DIAMYSCAAGGYSWSAGCEFNYWGQGTQVTVSSASH |
| | | | HHHHH (SEQ ID NO: 56) |
| DR468 | DR240 | DR234 | QVQLQESGGGSVQAGGSLRLSCGASGYTYSSYCMGW |
| | | | FRQVPGKEREGVAVIDSDGSTSYADSVKGRFTISKD |
| | | | NGKNTLYLQMNSLKPEDTAMYYCAADLGHYRPPCGV |
| | | | LYLGMDYWGKGTQVTVSSGGGSQVQLQESGGGSVQA |
| | | | GGSLRLSCVASGYTFSSYCMGWFRQAPGKEREGVAA |
| | | | LGGGSTYYADSVKGRFTISQDNAKNTLYLQMNSLKP |
| | | | EDTAMYYCAAAWVACLEFGGSWYDLARYKHWGQGTQ |
| | | | VTVSSASHHHHHH (SEQ ID NO: 57) |
| DR471 | DR241 | DR231 | QVQLQESGGGSVQAGGSLRLSCAASGYSNCSYDMTW |
| | | | YRQAPGKEREFVSAIHSDGSTRYADSVKGRFFISQD |
| | | | NAKNTVYLQMNSLKPEDTAMYYCKTDPLHCRAHGGS |
| | | | WYSVRANYWGQGTQVTVSSGGGSQVQLQESGGGSVQ |
| | | | AGGSLRLSCTASGFTFDDREMNWYRQAPGNECELVS |
| | | | TISSDGSTYYADSVKGRFTISQDNAKNTVYLQMDSV |
| | | | KPEDTAVYYCAADFMIAIQAPGAGCWGQGTQVTVSS |
| | | | ASHHHHHH (SEQ ID NO: 58) |
| DR474 | DR241 | DR234 | QVQLQESGGGSVQAGGSLRLSCAASGYSNCSYDMTW |
| | | | YRQAPGKEREFVSAIHSDGSTRYADSVKGRFFISQD |
| | | | NAKNTVYLQMNSLKPEDTAMYYCKTDPLHCRAHGGS |
| | | | WYSVRANYWGQGTQVTVSSGGGSQVQLQESGGGSVQ |
| | | | AGGSLRLSCVASGYTFSSYCMGWFRQAPGKEREGVA |
| | | | ALGGGSTYYADSVKGRFTISQDNAKNTLYLQMNSLK |
| | | | PEDTAMYYCAAAWVACLEFGGSWYDLARYKHWGQGT |
| | | | QVTVSSASHHHHHH (SEQ ID NO: 59) |

Table 4 below provides illustrative examples of IL10Rα/IL2Rγ binding proteins described herein that comprise an anti-IL2Rγ V_{H}H antibody at the N-terminus and an anti-IL10Rα V_{H}H antibody at the C-terminus.

In some embodiments, an IL10Rα/IL2Rγ binding protein comprises the V_{H}H sequence of DR229, the V_{H}H sequence of DR236, and at least at least 90% (e.g., 90%, 91%, 92%, 93%, 94%, 95%, 96%, 97%, 98%, 99%, or 100%) identity to the sequence of SEQ ID NO:60, optionally without the terminal HHHHHH. In some embodiments, an IL10Rα/IL2Rγ binding protein comprises the V_{H}H sequence of DR229, the V_{H}H sequence of DR239, and at least at least 90% (e.g., 90%, 91%, 92%, 93%, 94%, 95%, 96%, 97%, 98%, 99%, or 100%) identity to the sequence of SEQ ID NO:61, optionally without the terminal HHHHHH.

TABLE 4
Anti-IL10Rα/IL2Rγ V_{H}H² (anti-IL2Rγ
VHH-linker-anti-IL10Rα V_{H}H)

| Anti- | | | |
|---|---|---|---|
| IL 10Rα/ | N- | C- | |
| IL2Rγ | terminal | terminal | |
| V_{H}H² | V_{H}H | V_{H}H | Anti-IL10Rα/IL2Rγ V_{H}H² |
| DR392 | DR229 | DR236 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQGT |
| | | | QVTVSSGGGSQVQLQESGGGSVQAGGSLRLSCAASRF |
| | | | TYSSYCMGWFRQAPGKEREGVASIDSDGSTSYTDSVK |
| | | | GRFTISKDNAKNTLYLQMNSLKPEDTAMYYCALDLMS |
| | | | TVVPGFCGFLLSAGMDYWGKGTQVTVSSASHHHHHH |
| | | | (SEQ ID NO: 60) |
| DR395 | DR229 | DR239 | QVQLQESGGGLVQPGGSLRLSCTASGFSFSSYPMTWA |
| | | | RQAPGKGLEWVSTIASDGGSTAYAASVEGRFTISRDN |
| | | | AKSTLYLQLNSLKTEDTAMYYCTKGYGDGTPAPGQGT |
| | | | QVTVSSGGGSQVQLQESGGGSVQAGGSLRLSCTVSGY |
| | | | TYSSNCMGWFRQAPGKEREGVATIYTGGGNTYYADSV |
| | | | KGRFTISQDNAKNTVYLQMNNLKPEDTAMYYCAAEPL |
| | | | SRVYGGSCPTPTFDYWGQGTQVTVSSASHHHHHH |
| | | | (SEQ ID NO: 61) |

As shown in the illustrative examples of IL10Rα/IL2Rγ binding proteins of Table 3 and Table 4, the IL10Rα/IL2Rγ binding protein sequences contain GGGS (SEQ ID NO:62) as a linker. In some embodiments, the GGGS (SEQ ID NO:62) can be replaced by other linkers as described further herein. Furthermore, the IL10Rα/IL2Rγ binding protein sequences shown in Table 3 and Table 4 may be operably linked to a chelating peptide such as the “ASH6” (SEQ ID NO:81) metal chelating peptide which may be used to facilitate purification via metal affinity chromatography. In some embodiments, this purification handle can be removed or replaced by other purification handles (e.g., H6 (SEQ ID NO:82)).

Further, in each of SEQ ID NOS:96-179 below, each title of the sequence follows the formula “anti-IL10Rα/IL2Rγ V_{H}H² (V_{H}H antibody at the N-terminus-V_{H}H antibody at the C-terminus).” For example, “DR391(DR229-DR235)” refers to the anti-IL10Rα/IL2Rγ V_{H}H² binding protein with DR229 V_{H}H at the N-terminus and DR235 V_{H}H antibody at the C-terminus. In each of SEQ ID NOS:96-179 below, the linker is in bold, and each of CDR1, CDR2, CDR3 of the N-terminal V_{H}H antibody and CDR1, CDR2, CDR3 of the C-terminal V_{H}H antibody is underlined, respectively. An IL10Rα/IL2Rγ binding protein described herein can comprise the V_{H}H sequence of the N-terminal V_{H}H antibody, the V_{H}H sequence of the C-terminal V_{H}H antibody, and at least at least 90% (e.g., 90%, 91%, 92%, 93%, 94%, 95%, 96%, 97%, 98%, 99%, or 100%) identity to a sequence of any one of SEQ ID NOS:96-179. Moreover, the GGGS (SEQ ID NO:62) in each of SEQ ID NOS:96-179 below can be replaced by other linkers as described further herein. The purification handle “ASH6” (SEQ ID NO:81) at the end of each of SEQ ID NOS:96-179 can be removed or replaced by other purification handles (e.g., H6 (SEQ ID NO: 82)).

DR391(DR229-DR235)
>SEQ ID NO: 96
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCAASRYLY
SIDYMAWFRQSPGKEREPVAVIYTASGATFYPDSVKGRFTISQDNAKMTV
YLQMNSLKSEDTAMYYCAAVRKTDSYLFDAQSFTYWGQGTQVTVSSASHH
HHHH;
DR392(DR229-DR236)
>SEQ ID NO: 97
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCAASRFTY
SSYCMGWFRQAPGKEREGVASIDSDGSTSYTDSVKGRFTISKDNAKNTLY
LQMNSLKPEDTAMYYCALDLMSTVVPGFCGFLLSAGMDYWGKGTQVTVSS
ASHHHHHH;
DR393(DR229-DR237)
>SEQ ID NO: 98
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCAASGYTY
SMYCMGWFRQAPGKEREGVAQINSDGSTSYADSVKGRFTISKDNAKNTLY
LQMNSLKPEDTAMYYCAADSRVYGGSWYERLCGPYTYEYNYWGQGTQVTV
SSASHHHHHH;
DR394(DR229-DR238)
>SEQ ID NO: 99
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCAVSGYAY
STYCMGWFRQAPGKEREGVAAIDSGGSTSYADSVKGRFTISKDNAKNTLY
LRMNSLKPEDTAMYYCAAVPPPPDGGSCLFLGPEIKVSKADFRYWGQGTQ
VTVSSASHHHHHH;
DR395(DR229-DR239)
>SEQ ID NO: 100
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCTVSGYTY
SSNCMGWFRQAPGKEREGVATIYTGGGNTYYADSVKGRFTISQDNAKNTV
YLQMNNLKPEDTAMYYCAAEPLSRVYGGSCPTPTFDYWGQGTQVTVSSAS
HHHHHH;
DR396(DR229-DR240)
>SEQ ID NO: 101
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCGASGYTY
SSYCMGWFRQVPGKEREGVAVIDSDGSTSYADSVKGRFTISKDNGKNTLY
LQMNSLKPEDTAMYYCAADLGHYRPPCGVLYLGMDYWGKGTQVTVSSASH
HHHHH;
DR397(DR229-DR241)
>SEQ ID NO: 102
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCAASGYSN
CSYDMTWYRQAPGKEREFVSAIHSDGSTRYADSVKGRFFISQDNAKNTVY
LQMNSLKPEDTAMYYCKTDPLHCRAHGGSWYSVRANYWGQGTQVTVSSAS
HHHHHH;
DR398(DR230-DR235)
>SEQ ID NO: 103
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCAASR
YLYSIDYMAWFRQSPGKEREPVAVIYTASGATFYPDSVKGRFTISQDNAK
MTVYLQMNSLKSEDTAMYYCAAVRKTDSYLFDAQSFTYWGQGTQVTVSSA
SHHHHHH;
DR399(DR230-DR236)
>SEQ ID NO: 104
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCAASR
FTYSSYCMGWFRQAPGKEREGVASIDSDGSTSYTDSVKGRFTISKDNAKN
TLYLQMNSLKPEDTAMYYCALDLMSTVVPGFCGFLLSAGMDYWGKGTQVT
VSSASHHHHHH;
DR400(DR230-DR237)
>SEQ ID NO: 105
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCAASG
YTYSMYCMGWFRQAPGKEREGVAQINSDGSTSYADSVKGRFTISKDNAKN
TLYLQMNSLKPEDTAMYYCAADSRVYGGSWYERLCGPYTYEYNYWGQGTQ
VTVSSASHHHHHH;
DR401(DR230-DR238)
>SEQ ID NO: 106
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCAVSG
YAYSTYCMGWFRQAPGKEREGVAAIDSGGSTSYADSVKGRFTISKDNAKN
TLYLRMNSLKPEDTAMYYCAAVPPPPDGGSCLFLGPEIKVSKADFRYWGQ
GTQVTVSSASHHHHHH;
DR402(DR230-DR239)
>SEQ ID NO: 107
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCTVSG
YTYSSNCMGWFRQAPGKEREGVATIYTGGGNTYYADSVKGRFTISQDNAK
NTVYLQMNNLKPEDTAMYYCAAEPLSRVYGGSCPTPTFDYWGQGTQVTVS
SASHHHHHH;
DR403(DR230-DR240)
>SEQ ID NO: 108
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCGASG
YTYSSYCMGWFRQVPGKEREGVAVIDSDGSTSYADSVKGRFTISKDNGKN
TLYLQMNSLKPEDTAMYYCAADLGHYRPPCGVLYLGMDYWGKGTQVTVSS
ASHHHHHH;
DR404(DR230-DR241)
>SEQ ID NO: 109
QVQLQESGGGLVQPGGSLRLSCAASGFTFSSAHMSWVRQAPGKGREWIAS
IYSGGGTFYADSVKGRFTISRDNAKNTLYLQLNSLKAEDTAMYYCATNRL
HYYSDDDSLRGQGTQVTVSSGGGSQVQLQESGGGSVQAGGSLRLSCAASG
YSNCSYDMTWYRQAPGKEREFVSAIHSDGSTRYADSVKGRFFISQDNAKN
TVYLQMNSLKPEDTAMYYCKTDPLHCRAHGGSWYSVRANYWGQGTQVTVS
SASHHHHHH;
DR405(DR231-DR235)
>SEQ ID NO: 110
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCAAS
RYLYSIDYMAWFRQSPGKEREPVAVIYTASGATFYPDSVKGRFTISQDNA
KMTVYLQMNSLKSEDTAMYYCAAVRKTDSYLFDAQSFTYWGQGTQVTVSS
ASHHHHHH;
DR406(DR231-DR236)
>SEQ ID NO: 111
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCAAS
RFTYSSYCMGWFRQAPGKEREGVASIDSDGSTSYTDSVKGRFTISKDNAK
NTLYLQMNSLKPEDTAMYYCALDLMSTVVPGFCGFLLSAGMDYWGKGTQV
TVSSASHHHHHH;
DR407(DR231-DR237)
>SEQ ID NO: 112
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCAAS
GYTYSMYCMGWFRQAPGKEREGVAQINSDGSTSYADSVKGRFTISKDNAK
NTLYLQMNSLKPEDTAMYYCAADSRVYGGSWYERLCGPYTYEYNYWGQGT
QVTVSSASHHHHHH;
DR408(DR231-DR238)
>SEQ ID NO: 113
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCAVS
GYAYSTYCMGWFRQAPGKEREGVAAIDSGGSTSYADSVKGRFTISKDNAK
NTLYLRMNSLKPEDTAMYYCAAVPPPPDGGSCLFLGPEIKVSKADFRYWG
QGTQVTVSSASHHHHHH;
DR409(DR231-DR239)
>SEQ ID NO: 114
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCTVS
GYTYSSNCMGWFRQAPGKEREGVATIYTGGGNTYYADSVKGRFTISQDNA
KNTVYLQMNNLKPEDTAMYYCAAEPLSRVYGGSCPTPTFDYWGQGTQVTV
SSASHHHHHH;
DR410(DR231-DR240)
>SEQ ID NO: 115
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCGAS
GYTYSSYCMGWFRQVPGKEREGVAVIDSDGSTSYADSVKGRFTISKDNGK
NTLYLQMNSLKPEDTAMYYCAADLGHYRPPCGVLYLGMDYWGKGTQVTVS
SASHHHHHH;
DR411(DR231-DR241)
>SEQ ID NO: 116
QVQLQESGGGSVQAGGSLRLSCTASGFTFDDREMNWYRQAPGNECELVST
ISSDGSTYYADSVKGRFTISQDNAKNTVYLQMDSVKPEDTAVYYCAADFM
IAIQAPGAGCWGQGTQVTVSSGGGSQVQLQESGGGSVQAGGSLRLSCAAS
GYSNCSYDMTWYRQAPGKEREFVSAIHSDGSTRYADSVKGRFFISQDNAK
NTVYLQMNSLKPEDTAMYYCKTDPLHCRAHGGSWYSVRANYWGQGTQVTV
SSASHHHHHH;
DR412(DR232-DR235)
>SEQ ID NO: 117
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCAASRY
LYSIDYMAWFRQSPGKEREPVAVIYTASGATFYPDSVKGRFTISQDNAKM
TVYLQMNSLKSEDTAMYYCAAVRKTDSYLFDAQSFTYWGQGTQVTVSSAS
HHHHHH;
DR413(DR232-DR236)
>SEQ ID NO: 118
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCAASRF
TYSSYCMGWFRQAPGKEREGVASIDSDGSTSYTDSVKGRFTISKDNAKNT
LYLQMNSLKPEDTAMYYCALDLMSTVVPGFCGFLLSAGMDYWGKGTQVTV
SSASHHHHHH;
DR414(DR232-DR237)
>SEQ ID NO: 119
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCAASGY
TYSMYCMGWFRQAPGKEREGVAQINSDGSTSYADSVKGRFTISKDNAKNT
LYLQMNSLKPEDTAMYYCAADSRVYGGSWYERLCGPYTYEYNYWGQGTQV
TVSSASHHHHHH;
DR415(DR232-DR238)
>SEQ ID NO: 120
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCAVSGY
AYSTYCMGWFRQAPGKEREGVAAIDSGGSTSYADSVKGRFTISKDNAKNT
LYLRMNSLKPEDTAMYYCAAVPPPPDGGSCLFLGPEIKVSKADFRYWGQG
TQVTVSSASHHHHHH;
DR416(DR232-DR239)
>SEQ ID NO: 121
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCTVSGY
TYSSNCMGWFRQAPGKEREGVATIYTGGGNTYYADSVKGRFTISQDNAKN
TVYLQMNNLKPEDTAMYYCAAEPLSRVYGGSCPTPTFDYWGQGTQVTVSS
ASHHHHHH;
DR417(DR232-DR240)
>SEQ ID NO: 122
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCGASGY
TYSSYCMGWFRQVPGKEREGVAVIDSDGSTSYADSVKGRFTISKDNGKNT
LYLQMNSLKPEDTAMYYCAADLGHYRPPCGVLYLGMDYWGKGTQVTVSSA
SHHHHHH;
DR418(DR232-DR241)
>SEQ ID NO: 123
QVQLQESGGGSVQAGGSLRLSCVASGYTSCMGWFRQAPGKEREAVATIYT
RGRSIYYADSVKGRFTISQDNAKNTLYLQMNSLKPEDIAMYSCAAGGYSW
SAGCEFNYWGQGTQVTVSSGGGSQVQLQESGGGSVQAGGSLRLSCAASGY
SNCSYDMTWYRQAPGKEREFVSAIHSDGSTRYADSVKGRFFISQDNAKNT
VYLQMNSLKPEDTAMYYCKTDPLHCRAHGGSWYSVRANYWGQGTQVTVSS
ASHHHHHH;
DR419(DR233-DR235)
>SEQ ID NO: 124
QVQLQESGGGSVQAGGSLRLSCTASGFTFDDSDMgWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CAASRYLYSIDYMAWFRQSPGKEREPVAVIYTASGATFYPDSVKGRFTIS
QDNAKMTVYLQMNSLKSEDTAMYYCAAVRKTDSYLFDAQSFTYWGQGTQV
TVSSASHHHHHH;
DR420(DR233-DR236)
>SEQ ID NO: 125
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CAASRFTYSSYCMGWFRQAPGKEREGVASIDSDGSTSYTDSVKGRFTISK
DNAKNTLYLQMNSLKPEDTAMYYCALDLMSTVVPGFCGFLLSAGMDYWGK
GTQVTVSSASHHHHHH;
DR421(DR233-DR237)
>SEQ ID NO: 126
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CAASGYTYSMYCMGWFRQAPGKEREGVAQINSDGSTSYADSVKGRFTISK
DNAKNTLYLQMNSLKPEDTAMYYCAADSRVYGGSWYERLCGPYTYEYNYW
GQGTQVTVSSASHHHHHH;
DR422(DR233-DR238)
>SEQ ID NO: 127
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CAVSGYAYSTYCMGWFRQAPGKEREGVAAIDSGGSTSYADSVKGRFTISK
DNAKNTLYLRMNSLKPEDTAMYYCAAVPPPPDGGSCLFLGPEIKVSKADF
RYWGQGTQVTVSSASHHHHHH;
DR423(DR233-DR239)
>SEQ ID NO: 128
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CTVSGYTYSSNCMGWFRQAPGKEREGVATIYTGGGNTYYADSVKGRFTIS
QDNAKNTVYLQMNNLKPEDTAMYYCAAEPLSRVYGGSCPTPTFDYWGQGT
QVTVSSASHHHHHH;
DR424(DR233-DR240)
>SEQ ID NO: 129
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CGASGYTYSSYCMGWFRQVPGKEREGVAVIDSDGSTSYADSVKGRFTISK
DNGKNTLYLQMNSLKPEDTAMYYCAADLGHYRPPCGVLYLGMDYWGKGTQ
VTVSSASHHHHHH;
DR425(DR233-DR241)
>SEQ ID NO: 130
QVQLQESGGGSVQAGGSLRLSCTASGFTFDDSDMGWYRQAPGNECELVST
ISSDGSTYYADSVKGRFTISQDNAKNTVYLQMNSLKPEDTAVYYCAAEPR
GYYSNYGGRRECNYWGQGTQVTVSSGGGSQVQLQESGGGSVQAGGSLRLS
CAASGYSNCSYDMTWYRQAPGKEREFVSAIHSDGSTRYADSVKGRFFISQ
DNAKNTVYLQMNSLKPEDTAMYYCKTDPLHCRAHGGSWYSVRANYWGQGT
QVTVSSASHHHHHH;
DR426(DR234-DR235)
>SEQ ID NO: 131
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCAASRYLYSIDYMAWFRQSPGKEREPVAVIYTASGATFYPDSVKGRFT
ISQDNAKMTVYLQMNSLKSEDTAMYYCAAVRKTDSYLFDAQSFTYWGQGT
QVTVSSASHHHHHH;
DR427(DR234-DR236)
>SEQ ID NO: 132
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCAASRFTYSSYCMGWFRQAPGKEREGVASIDSDGSTSYTDSVKGRFTI
SKDNAKNTLYLQMNSLKPEDTAMYYCALDLMSTVVPGFCGFLLSAGMDYW
GKGTQVTVSSASHHHHHH;
DR428(DR234-DR237)
>SEQ ID NO: 133
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCAASGYTYSMYCMGWFRQAPGKEREGVAQINSDGSTSYADSVKGRFTI
SKDNAKNTLYLQMNSLKPEDTAMYYCAADSRVYGGSWYERLCGPYTYEYN
YWGQGTQVTVSSASHHHHHH;
DR429(DR234-DR238)
>SEQ ID NO: 134
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCAVSGYAYSTYCMGWFRQAPGKEREGVAAIDSGGSTSYADSVKGRFTI
SKDNAKNTLYLRMNSLKPEDTAMYYCAAVPPPPDGGSCLFLGPEIKVSKA
DFRYWGQGTQVTVSSASHHHHHH;
DR430(DR234-DR239)
>SEQ ID NO: 135
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCTVSGYTYSSNCMGWFRQAPGKEREGVATIYTGGGNTYYADSVKGRFT
ISQDNAKNTVYLQMNNLKPEDTAMYYCAAEPLSRVYGGSCPTPTFDYWGQ
GTQVTVSSASHHHHHH;
DR431(DR234-DR240)
>SEQ ID NO: 136
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCGASGYTYSSYCMGWFRQVPGKEREGVAVIDSDGSTSYADSVKGRFTI
SKDNGKNTLYLQMNSLKPEDTAMYYCAADLGHYRPPCGVLYLGMDYWGKG
TQVTVSSASHHHHHH;
DR432(DR234-DR241)
>SEQ ID NO: 137
QVQLQESGGGSVQAGGSLRLSCVASGYTFSSYCMGWFRQAPGKEREGVAA
LGGGSTYYADSVKGRFTISQDNAKNTLYLQMNSLKPEDTAMYYCAAAWVA
CLEFGGSWYDLARYKHWGQGTQVTVSSGGGSQVQLQESGGGSVQAGGSLR
LSCAASGYSNCSYDMTWYRQAPGKEREFVSAIHSDGSTRYADSVKGRFFI
SQDNAKNTVYLQMNSLKPEDTAMYYCKTDPLHCRAHGGSWYSVRANYWGQ
GTQVTVSSASHHHHHH;
DR433(DR235-DR229)
>SEQ ID NO: 138
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGLVQPGGSLRLS
CTASGFSFSSYPMTWARQAPGKGLEWVSTIASDGGSTAYAASVEGRFTIS
RDNAKSTLYLQLNSLKTEDTAMYYCTKGYGDGTPAPGQGTQVTVSSASHH
HHHH;
DR434(DR235-DR230)
>SEQ ID NO: 139
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGLVQPGGSLRLS
CAASGFTFSsahmsWVRQAPGKGREWIASIYSGGGTFYADSVKGRFTISR
DNAKNTLYLQLNSLKAEDTAMYYCATNRLHYYSDDDSLRGQGTQVTVSSA
SHHHHHH;
DR435(DR235-DR231)
>SEQ ID NO: 140
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGSVQAGGSLRLS
CTASGFTFDDREMNWYRQAPGNECELVSTISSDGSTYYADSVKGRFTISQ
DNAKNTVYLQMDSVKPEDTAVYYCAADFMIAIQAPGAGCWGQGTQVTVSS
ASHHHHHH;
DR436(DR235-DR232)
>SEQ ID NO: 141
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGSVQAGGSLRLS
CVASGYTSCMGWFRQAPGKEREAVATIYTRGRSIYYADSVKGRFTISQDN
AKNTLYLQMNSLKPEDIAMYSCAAGGYSWSAGCEFNYWGQGTQVTVSSAS
HHHHHH;
DR437(DR235-DR233)
>SEQ ID NO: 142
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGSVQAGGSLRLS
CTASGFTFDDSDMGWYRQAPGNECELVSTISSDGSTYYADSVKGRFTISQ
DNAKNTVYLQMNSLKPEDTAVYYCAAEPRGYYSNYGGRRECNYWGQGTQV
TVSSASHHHHHH;
DR438(DR235-DR234)
>SEQ ID NO: 143
QVQLQESGGGSVQAGGSLRLSCAASRYLYSIDYMAWFRQSPGKEREPVAV
IYTASGATFYPDSVKGRFTISQDNAKMTVYLQMNSLKSEDTAMYYCAAVR
KTDSYLFDAQSFTYWGQGTQVTVSSGGGSQVQLQESGGGSVQAGGSLRLS
CVASGYTFSSYCMGWFRQAPGKEREGVAALGGGSTYYADSVKGRFTISQD
NAKNTLYLQMNSLKPEDTAMYYCAAAWVACLEFGGSWYDLARYKHWGQGT
QVTVSSASHHHHHH;
DR439(DR236-DR229)
>SEQ ID NO: 144
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGLVQPGGS
LRLSCTASGFSFSSYPMTWARQAPGKGLEWVSTIASDGGSTAYAASVEGR
FTISRDNAKSTLYLQLNSLKTEDTAMYYCTKGYGDGTPAPGQGTQVTVSS
ASHHHHHH;
DR440(DR236-DR230)
>SEQ ID NO: 145
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGLVQPGGS
LRLSCAASGFTFSSAHMSWVRQAPGKGREWIASIYSGGGTFYADSVKGRF
TISRDNAKNTLYLQLNSLKAEDTAMYYCATNRLHYYSDDDSLRGQGTQVT
VSSASHHHHHH;
DR441(DR236-DR231)
>SEQ ID NO: 146
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGSVQAGGS
LRLSCTASGFTFDDREMNWYRQAPGNECELVSTISSDGSTYYADSVKGRF
TISQDNAKNTVYLQMDSVKPEDTAVYYCAADFMIAIQAPGAGCWGQGTQV
TVSSASHHHHHH;
DR442(DR236-DR232)
>SEQ ID NO: 147
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGSVQAGGS
LRLSCVASGYTSCMGWFRQAPGKEREAVATIYTRGRSIYYADSVKGRFTI
SQDNAKNTLYLQMNSLKPEDIAMYSCAAGGYSWSAGCEFNYWGQGTQVTV
SSASHHHHHH;
DR443(DR236-DR233)
>SEQ ID NO: 148
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGSVQAGGS
LRLSCTASGFTFDDSDMGWYRQAPGNECELVSTISSDGSTYYADSVKGRF
TISQDNAKNTVYLQMNSLKPEDTAVYYCAAEPRGYYSNYGGRRECNYWGQ
GTQVTVSSASHHHHHH;
DR444(DR236-DR234)
>SEQ ID NO: 149
QVQLQESGGGSVQAGGSLRLSCAASRFTYSSYCMGWFRQAPGKEREGVAS
IDSDGSTSYTDSVKGRFTISKDNAKNTLYLQMNSLKPEDTAMYYCALDLM
STVVPGFCGFLLSAGMDYWGKGTQVTVSSGGGSQVQLQESGGGSVQAGGS
LRLSCVASGYTFSSYCMGWFRQAPGKEREGVAALGGGSTYYADSVKGRFT
ISQDNAKNTLYLQMNSLKPEDTAMYYCAAAWVACLEFGGSWYDLARYKHW
GQGTQVTVSSASHHHHHH;
DR445(DR237-DR229)
>SEQ ID NO: 150
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGLVQPG
GSLRLSCTASGFSFSSYPMTWARQAPGKGLEWVSTIASDGGSTAYAASVE
GRFTISRDNAKSTLYLQLNSLKTEDTAMYYCTKGYGDGTPAPGQGTQVTV
SSASHHHHHH;
DR446(DR237-DR230)
>SEQ ID NO: 151
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGLVQPG
GSLRLSCAASGFTFSSAHMSWVRQAPGKGREWIASIYSGGGTFYADSVKG
RFTISRDNAKNTLYLQLNSLKAEDTAMYYCATNRLHYYSDDDSLRGQGTQ
VTVSSASHHHHHH;
DR447(DR237-DR231)
>SEQ ID NO: 152
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGSVQAG
GSLRLSCTASGFTFDDREMNWYRQAPGNECELVSTISSDGSTYYADSVKG
RFTISQDNAKNTVYLQMDSVKPEDTAVYYCAADFMIAIQAPGAGCWGQGT
QVTVSSASHHHHHH;
DR448(DR237-DR232)
>SEQ ID NO: 153
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGSVQAG
GSLRLSCVASGYTSCMGWFRQAPGKEREAVATIYTRGRSIYYADSVKGRF
TISQDNAKNTLYLQMNSLKPEDIAMYSCAAGGYSWSAGCEFNYWGQGTQV
TVSSASHHHHHH;
DR449(DR237-DR233)
>SEQ ID NO: 154
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGSVQAG
GSLRLSCTASGFTFDDSDMGWYRQAPGNECELVSTISSDGSTYYADSVKG
RFTISQDNAKNTVYLQMNSLKPEDTAVYYCAAEPRGYYSNYGGRRECNYW
GQGTQVTVSSASHHHHHH;
DR450(DR237-DR234)
>SEQ ID NO: 155
QVQLQESGGGSVQAGGSLRLSCAASGYTYSMYCMGWFRQAPGKEREGVAQ
INSDGSTSYADSVKGRFTISKDNAKNTLYLQMNSLKPEDTAMYYCAADSR
VYGGSWYERLCGPYTYEYNYWGQGTQVTVSSGGGSQVQLQESGGGSVQAG
GSLRLSCVASGYTFSSYCMGWFRQAPGKEREGVAALGGGSTYYADSVKGR
FTISQDNAKNTLYLQMNSLKPEDTAMYYCAAAWVACLEFGGSWYDLARYK
hWGQGTQVTVSSASHHHHHH;
DR451(DR238-DR229)
>SEQ ID NO: 156
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSGGGSQVQLQESGGGLV
QPGGSLRLSCTASGFSFSSYPMTWARQAPGKGLEWVStIASDGGSTAYAA
SVEGRFTISRDNAKSTLYLQLNSLKTEDTAMYYCTKGYGDGTPAPGQGTQ
VTVSSASHHHHHH;
DR452(DR238-DR230)
>SEQ ID NO: 157
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSgggsQVQLQESGGGLV
QPGGSLRLSCAASGFTFSSAHMSWVRQAPGKGREWIASIYSGGGTFYADS
VKGRFTISRDNAKNTLYLQLNSLKAEDTAMYYCATNRLHYYSDDDSLRGQ
GTQVTVSSASHHHHHH;
DR453(DR238-DR231)
>SEQ ID NO: 158
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSGGGSQVQLQESGGGSV
QAGGSLRLSCTASGFTFDDREMNWYRQAPGNECELVSTISSDGSTYYADS
VKGRFTISQDNAKNTVYLQMDSVKPEDTAVYYCAADFMIAIQAPGAGCWG
QGTQVTVSSASHHHHHH;
DR454(DR238-DR232)
>SEQ ID NO: 159
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSGGGSQVQLQESGGGSV
QAGGSLRLSCVASGYTSCMGWFRQAPGKEREAVATIYTRGRSIYYADSVK
GRFTISQDNAKNTLYLQMNSLKPEDIAMYSCAAGGYSWSAGCEFNYWGQG
TQVTVSSASHHHHHH;
DR455(DR238-DR233)
>SEQ ID NO: 160
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSGGGSQVQLQESGGGSV
QAGGSLRLSCTASGFTFDDSDMGWYRQAPGNECELVSTISSDGSTYYADS
VKGRFTISQDNAKNTVYLQMNSLKPEDTAVYYCAAEPRGYYSNYGGRREC
NYWGQGTQVTVSSASHHHHHH;
DR456(DR238-DR234)
>SEQ ID NO: 161
QVQLQESGGGSVQAGGSLRLSCAVSGYAYSTYCMGWFRQAPGKEREGVAA
IDSGGSTSYADSVKGRFTISKDNAKNTLYLRMNSLKPEDTAMYYCAAVPP
PPDGGSCLFLGPEIKVSKADFRYWGQGTQVTVSSGGGSQVQLQESGGGSV
QAGGSLRLSCVASGYTFSSYCMGWFRQAPGKEREGVAALGGGSTYYADSV
KGRFTISQDNAKNTLYLQMNSLKPEDTAMYYCAAAWVACLEFGGSWYDLA
RYKHWGQGTQVTVSSASHHHHHH;
DR457(DR239-DR229)
>SEQ ID NO: 162
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGLVQPGGSLR
LSCTASGFSFSSYPMTWARQAPGKGLEWVSTIASDGGSTAYAASVEGRFT
ISRDNAKSTLYLQLNSLKTEDTAMYYCTKGYGDGTPAPGQGTQVTVSSAS
HHHHHH;
DR458(DR239-DR230)
>SEQ ID NO: 163
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGLVQPGGSLR
LSCAASGFTFSSAHMSWVRQAPGKGREWIASIYSGGGTFYADSVKGRFTI
SRDNAKNTLYLQLNSLKAEDTAMYYCATNRLHYYSDDDSLRGQGTQVTVS
SASHHHHHH;
DR459(DR239-DR231)
>SEQ ID NO: 164
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGSVQAGGSLR
LSCTASGFTFDDREMNWYRQAPGNECELVSTISSDGSTYYADSVKGRFTI
SQDNAKNTVYLQMDSVKPEDTAVYYCAADFMIAIQAPGAGCWGQGTQVTV
SSASHHHHHH;
DR460(DR239-DR232)
>SEQ ID NO: 165
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGSVQAGGSLR
LSCVASGYTSCMGWFRQAPGKEREAVATIYTRGRSIYYADSVKGRFTISQ
DNAKNTLYLQMNSLKPEDIAMYSCAAGGYSWSAGCEFNYWGQGTQVTVSS
ASHHHHHH;
DR461(DR239-DR233)
>SEQ ID NO: 166
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGSVQAGGSLR
LSCTASGFTFDDSDMGWYRQAPGNECELVSTISSDGSTYYADSVKGRFTI
SQDNAKNTVYLQMNSLKPEDTAVYYCAAEPRGYYSNYGGRRECNYWGQGT
QVTVSSASHHHHHH;
DR462(DR239-DR234)
>SEQ ID NO: 167
QVQLQESGGGSVQAGGSLRLSCTVSGYTYSSNCMGWFRQAPGKEREGVAT
IYTGGGNTYYADSVKGRFTISQDNAKNTVYLQMNNLKPEDTAMYYCAAEP
LSRVYGGSCPTPTFDYWGQGTQVTVSSGGGSQVQLQESGGGSVQAGGSLR
LSCVASGYTFSSYCMGWFRQAPGKEREGVAALGGGSTYYADSVKGRFTIS
QDNAKNTLYLQMNSLKPEDTAMYYCAAAWVACLEFGGSWYDLARYKHWGQ
GTQVTVSSASHHHHHH;
DR463(DR240-DR229)
>SEQ ID NO: 168
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGLVQPGGSLRL
SCTASGFSFSSYPMTWARQAPGKGLEWVSTIASDGGSTAYAASVEGRFTI
SRDNAKSTLYLQLNSLKTEDTAMYYCTKGYGDGTPAPGQGTQVTVSSASH
HHHHH;
DR464(DR240-DR230)
>SEQ ID NO: 169
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGLVQPGGSLRL
SCAASGFTFSSAHMSWVRQAPGKGREWIASIYSGGGTFYADSVKGRFTIS
RDNAKNTLYLQLNSLKAEDTAMYYCATNRLHYYSDDDSLRGQGTQVTVSS
ASHHHHHH;
DR465(DR240-DR231)
>SEQ ID NO: 170
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGSVQAGGSLRL
SCTASGFTFDDREMNWYRQAPGNECELVSTISSDGSTYYADSVKGRFTIS
QDNAKNTVYLQMDSVKPEDTAVYYCAADFMIAIQAPGAGCWGQGTQVTVS
SASHHHHHH;
DR466(DR240-DR232)
>SEQ ID NO: 171
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGSVQAGGSLRL
SCVASGYTSCMGWFRQAPGKEREAVATIYTRGRSIYYADSVKGRFTISQD
NAKNTLYLQMNSLKPEDIAMYSCAAGGYSWSAGCEFNYWGQGTQVTVSSA
SHHHHHH;
DR467(DR240-DR233)
>SEQ ID NO: 172
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGSVQAGGSLRL
SCTASGFTFDDSDMGWYRQAPGNECELVSTISSDGSTYYADSVKGRFTIS
QDNAKNTVYLQMNSLKPEDTAVYYCAAEPRGYYSNYGGRRECNYWGQGTQ
VTVSSASHHHHHH;
DR468(DR240-DR234)
>SEQ ID NO: 173
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGSVQAGGSLRL
SCVASGYTFSSYCMGWFRQAPGKEREGVAALGGGSTYYADSVKGRFTISQ
DNAKNTLYLQMNSLKPEDTAMYYCAAAWVACLEFGGSWYDLARYKHWGQG
TQVTVSSASHHHHHH;
DR469(DR241-DR229)
>SEQ ID NO: 174
QVQLQESGGGSVQAGGSLRLSCAASGYSNCsydmtWYRQAPGKEREFVSa
ihsdgstryadsvkgRFFISQDNAKNTVYLQMNSLKPEDTAMYYCKTdpl
hcrahggswysvranyWGQGTQVTVSSgggsQVQLQESGGGLVQPGGSLR
LSCTASGFSFSsypmtWARQAPGKGLEWVStiasdggstayaasvegRFT
ISRDNAKSTLYLQLNSLKTEDTAMYYCTKgygdgtpaPGQGTQVTVSSAS
HHHHHH;
DR470(DR241-DR230)
>SEQ ID NO: 175
QVQLQESGGGSVQAGGSLRLSCAASGYSNCSYDMTWYRQAPGKEREFVSA
IHSDGSTRYADSVKGRFFISQDNAKNTVYLQMNSLKPEDTAMYYCKTDPL
HCRAHGGSWYSVRANYWGQGTQVTVSSGGGSQVQLQESGGGLVQPGGSLR
LSCAASGFTFSSAHMSWVRQAPGKGREWIASIYSGGGTFYADSVKGRFTI
SRDNAKNTLYLQLNSLKAEDTAMYYCATNRLHYYSDDDSLRGQGTQVTVS
SASHHHHHH;
DR471(DR241-DR231)
>SEQ ID NO: 176
QVQLQESGGGSVQAGGSLRLSCAASGYSNCSYDMTWYRQAPGKEREFVSA
IHSDGSTRYADSVKGRFFISQDNAKNTVYLQMNSLKPEDTAMYYCKTDPL
HCRAHGGSWYSVRANYWGQGTQVTVSSGGGSQVQLQESGGGSVQAGGSLR
LSCTASGFTFDDREMNWYRQAPGNECELVSTISSDGSTYYADSVKGRFTI
SQDNAKNTVYLQMDSVKPEDTAVYYCAADFMIAIQAPGAGCWGQGTQVTV
SSASHHHHHH;
DR472(DR241-DR232)
>SEQ ID NO: 177
QVQLQESGGGSVQAGGSLRLSCAASGYSNCSYDMTWYRQAPGKEREFVSA
IHSDGSTRYADSVKGRFFISQDNAKNTVYLQMNSLKPEDTAMYYCKTDPL
HCRAHGGSWYSVRANYWGQGTQVTVSSGGGSQVQLQESGGGSVQAGGSLR
LSCVASGYTSCMGWFRQAPGKEREAVATIYTRGRSIYYADSVKGRFTISQ
DNAKNTLYLQMNSLKPEDIAMYSCAAGGYSWSAGCEFNYWGQGTQVTVSS
ASHHHHHH;
DR473(DR241-DR233)
>SEQ ID NO: 178
QVQLQESGGGSVQAGGSLRLSCAASGYSNCSYDMTWYRQAPGKEREFVSA
IHSDGSTRYADSVKGRFFISQDNAKNTVYLQMNSLKPEDTAMYYCKTDPL
HCRAHGGSWYSVRANYWGQGTQVTVSSGGGSQVQLQESGGGSVQAGGSLR
LSCTASGFTFDDSDMGWYRQAPGNECELVSTISSDGSTYYADSVKGRFTI
SQDNAKNTVYLQMNSLKPEDTAVYYCAAEPRGYYSNYGGRRECNYWGQGT
QVTVSSASHHHHHH;
DR474(DR241-DR234)
>SEQ ID NO: 179
QVQLQESGGGSVQAGGSLRLSCAASGYSNCSYDMTWYRQAPGKEREFVSA
IHSDGSTRYADSVKGRFFISQDNAKNTVYLQMNSLKPEDTAMYYCKTDPL
HCRAHGGSWYSVRANYWGQGTQVTVSSGGGSQVQLQESGGGSVQAGGSLR
LSCVASGYTFSSYCMGWFRQAPGKEREGVAALGGGSTYYADSVKGRFTIS
QDNAKNTLYLQMNSLKPEDTAMYYCAAAWVACLEFGGSWYDLARYKHWGQ
GTQVTVSSASHHHHHH;

In some embodiments, the binding proteins described herein can include one or more anti-IL10Rα V_{H}H antibodies. When two or more anti-IL10Rα V_{H}H antibodies are present, neighboring antibodies can be conjugated to each other by way of a linker. In some embodiments, the binding proteins described herein can include one or more anti-IL2Rγ V_{H}H antibodies. When two or more anti-IL2Rγ V_{H}H antibodies are present, neighboring antibodies can be conjugated to each other by way of a linker.

In some embodiments, the binding proteins described herein can include one or more anti-IL10Rα V_{H}H antibodies and one or more anti-IL2Rγ V_{H}H antibodies. Neighboring antibodies can be conjugated to each other by way of a linker. In some embodiments, the number of anti-IL10Rα V_{H}H antibodies and the number of anti-IL2Rγ V_{H}H antibodies in a binding protein are the same. In other embodiments, the number of anti-IL10Rα V_{H}H antibodies and the number of anti-IL2Rγ V_{H}H antibodies in a binding protein are different.

In some embodiments, a binding protein described herein can be represented by the following formula:

H₂N—[[V_{H}H #1]ₐ-L_{b}-[[V_{H}H#2]_{c}]]ₓ-COOH

wherein L is a linker, a, b, c are independently selected from 0 or 1, and x is an integer between 1 and 10 (e.g., 1, 2, 3, 4, 5, 6, 7, 8, 9, or 10). In some embodiments, V_{H}H #1 and V_{H}H #2 target the same receptor or subunit thereof. In some embodiments, V_{H}H #1 and V_{H}H #2 target different receptors or subunits thereof. In some embodiments, V_{H}H #1 and V_{H}H #2 can have the same sequence. In other embodiments, V_{H}H #1 and V_{H}H #2 can have different sequences.

In some embodiments, the IL10Rα/IL2Rγ binding protein is linked to an Fc polypeptide or an Fc domain. In some embodiments, the Fc polypeptide (e.g., subunit of an Fc domain) or an Fc domain is from an IgG1, IgG2, IgG3 or IgG4. In some embodiments, the IL10Rα/IL2Rγ binding protein is at least 90 (e.g., 91%, 92%, 93%, 94%, 95%, 96%, 97%, 98%, 99%, or 100%) identical to any one of SEQ ID NOS: 49-61 or 96-179, optionally without the HHHHHH sequence(s) therein.

### A. “Forward Orientation”

In some embodiments, the bivalent IL10Rα/IL2Rγ binding molecule comprises a polypeptide of the structure:

H₂N-[anti-IL10Rα sdAb]-[L]ₓ-[anti-IL2Rγ sdAb]-[TAG]_{y}-COOH

wherein and L is a polypeptide linker of 1-50 amino acids and x=0 or 1, and TAG is a chelating peptide or a subunit of an Fc domain and y=0 or 1.

In some embodiments, a bivalent IL10Rα/IL2Rγ binding molecule of the foregoing structure comprises a polypeptide from amino to carboxy terminus:
(a) an anti-IL10Rα sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 in a row of Table 10;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 in a row of Table 10; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 10; or
   (A) a CDR1 comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; or
   (B) a CDR1 comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; or
   (C) a CDR1 comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO:11; or
   (D) a CDR1 comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; or
   (E) a CDR1 comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; or
(F) a CDR1 comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23; and
(b) optionally, a polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL2Rγ sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 listed in Table 11 or Table 12;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 listed in Table 11 or Table 12; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 11 or Table 12; or
   (A) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   (B) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   (C) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   (D) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   (E) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   (F) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47.

In some embodiments, the bivalent IL10Rα/IL2Rγ binding molecule comprises an anti-IL10Rα sdAb comprising a CDR1, a CDR2, and a CDR3 as listed in a row of Table 10 and an anti-IL2Rγ sdAb comprising a CDR1, a CDR2, and a CDR3 as listed in a row of Table 11 or Table 12.

In some embodiments, the anti-IL10Rα sdAb of the bivalent IL10Rα/IL2Rγ binding molecule comprises a sequence having at least 90% (e.g., 91%, 92%, 93%, 94%, 95%, 96%, 97%, 98%, 99%, or 100%) sequence identity to a sequence of any one the of anti-IL10Rα sdAbs provided in Table 13. In some embodiments, the anti-IL2Rγ sdAb of the bivalent IL10Rα/IL2Rγ binding molecule comprises a sequence having at least 90% (e.g., 91%, 92%, 93%, 94%, 95%, 96%, 97%, 98%, 99%, or 100%) sequence identity to a sequence of any one the of anti-IL2Rγ sdAbs provided in Table 14 or Table 15.

In some embodiments, the subunit of the Fc domain is from an IgG1, IgG2, IgG3 or IgG4. In some embodiments, the subunit of the Fc domain comprises one or more amino acid substitutions to reduce effector function, for example, the subunit of the Fc domain comprises a set of amino acid substitutions selected from the group consisting of: (a) L234A/L235A/P329A (“LALAPA”); L234A/L235A/P329G (“LALAPG”); L234A/L235E/G237A/A330S/P331S (“AEASS”); E233P/L234V/L235A/AG237 (PVAdelG); and L234F/L235E/P331S (“FES”). In some embodiments, the subunit of the Fc domain is modified for multimerization. In some embodiments the subunit of the Fc domain comprises an amino acid substitution at position C220 (EU numbering) of the upper hinge domain to eliminate the sulfhydryl side chain. In some embodiments, the substitution at position C220 is C220S (EU numbering) substitution. In some embodiments the subunit of the Fc domain comprises amino acid substitutions in the Fc domain at positions M428 and/or N434 (EU numbering). In some embodiments the amino acid substitutions at positions M428 and/or N434 are M428L and/or N434S. In some embodiments the subunit of the Fc domain comprises amino acid deletions in the Fc domain at positions G446 and/or K447 (EU numbering).

### B. “Reverse Orientation”

In some embodiments, the bivalent IL10Rα/IL2Rγ binding molecule comprises a polypeptide of the structure:

H₂N-[anti-IL2Rγ sdAb]-[L]ₓ-[anti-IL10Rα sdAb]-[TAG]_{y}-COOH

wherein and L is a polypeptide linker of 1-50 amino acids and x=0 or 1, and TAG is a chelating peptide or a subunit of an Fc domain and y=0 or 1.

In some embodiments, a bivalent IL10Rα/IL2Rγ binding molecule of the foregoing structure comprises a polypeptide from amino to carboxy terminus:
(a) an anti-IL2Rγ sdAb comprising:
   a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 listed in Table 11 or Table 12;
   a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 listed in Table 11 or Table 12; and
   a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 11 or Table 12; or
   (A) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
   (B) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
   (C) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
   (D) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
   (E) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
   (F) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; and
(b) optionally, a polypeptide linker from 1-50 amino acids, alternatively 1-40 amino acids, alternatively 1-30 amino acids, alternatively 1-20 amino acids, alternatively 1-15 amino acids, alternatively 1-10 amino acids, alternatively 1-8 amino acids, alternatively 1-6 amino acids, alternatively 1-4 amino acids; and
(c) an anti-IL10Rα sdAb comprising:
a. a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 in a row of Table 10.
b. a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 in a row of Table 10; and
c. a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 10; or
d. (A) a CDR1 comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; or
e. (B) a CDR1 comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; or
f. (C) a CDR1 comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO:11; or
g. (D) a CDR1 comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; or
h. (E) a CDR1 comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; or
i. (F) a CDR1 comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23.

In some embodiments, the anti-IL2Rγ sdAb comprises a sequence having at least 90% (e.g., 91%, 92%, 93%, 94%, 95%, 96%, 97%, 98%, 99%, or 100%) sequence identity to a sequence listed in a row of Table 14 or Table 15. In certain embodiments, the anti-IL10Rα sdAb comprises a sequence having at least 90% sequence identity to a sequence of any one of listed in a row of Table 13.

In some embodiments, the subunit of the Fc domain is from an IgG1, IgG2, IgG3 or IgG4. In some embodiments, the subunit of the Fc domain comprises one or more amino acid substitutions to reduce effector function, for example, the subunit of the Fc domain comprises a set of amino acid substitutions selected from the group consisting of: (a) L234A/L235A/P329A (“LALAPA”); L234A/L235A/P329G (“LALAPG”); L234A/L235E/G237A/A330S/P331S (“AEASS”); E233P/L234V/L235A/AG237 (PVAdelG); and L234F/L235E/P331S (“FES”). In some embodiments, the subunit of the Fc domain is modified for multimerization. In some embodiments the subunit of the Fc domain comprises an amino acid substitution at position C220 (EU numbering) of the upper hinge domain to eliminate the sulfhydryl side chain. In some embodiments, the substitution at position C220 is C220S (EU numbering) substitution. In some embodiments, the subunit of the Fc domain comprises amino acid substitutions in the Fc domain at positions M428 and/or N434 (EU numbering). In some embodiments, the amino acid substitutions at positions M428 and/or N434 are M428L and/or N434S. In some embodiments, the subunit of the Fc domain comprises amino acid deletions in the Fc domain at positions G446 and/or K447 (EU numbering).

### IV. Single-Domain Antibody and V_{H}H

A single-domain antibody (sdAb) is an antibody containing a single monomeric variable antibody domain. Like a full-length antibody, it is able to bind selectively to a specific antigen. The complementary determining regions (CDRs) of sdAbs are within a single-domain polypeptide. Single-domain antibodies can be engineered from heavy-chain antibodies found in camelids, which are referred to as V_{H}Hs. Cartilaginous fishes also have heavy-chain antibodies (IgNAR, “immunoglobulin new antigen receptor”), from which single-domain antibodies referred to as VNARS can be obtained. The dimeric variable domains from common immunoglobulin G (IgG) from humans or mice can also be split into monomers to make sdAbs. Although most research into sdAbs is currently based on heavy chain variable domains, sdAbs derived from light chains have also been shown to bind specifically to target, see, e.g., Moller et al., J Biol Chem. 285(49):38348-38361, 2010. In some embodiments, a sdAb is composed of a single monomeric light chain variable antibody domain.

A sdAb can be a heavy chain antibody (V_{H}H). A V_{H}H is a type of sdAb that has a single monomeric heavy chain variable antibody domain. Similar to a traditional antibody, a V_{H}H is able to bind selectively to a specific antigen. A binding protein described herein can include two V_{H}Hs (e.g., V_{H}H²) joined together by a linker (e.g., a peptide linker). The binding protein can be a bispecific V_{H}H² that includes a first V_{H}H binding to a first receptor or domain or subunit thereof and a second V_{H}H binding to a second receptor or domain or subunit thereof, in which the two V_{H}Hs are joined by a linker.

An exemplary V_{H}H has a molecular weight of approximately 12-15 kDa which is much smaller than traditional mammalian antibodies (150-160 kDa) composed of two heavy chains and two light chains. V_{H}Hs can be found in or produced from Camelidae mammals (e.g., camels, llamas, dromedary, alpaca, and guanaco) which are naturally devoid of light chains. Descriptions of sdAbs and V_{H}HS can be found in, e.g., De Greve et al., Curr Opin Biotechnol. 61:96-101, 2019; Ciccarese, et al., Front Genet. 10:997, 2019; Chanier and Chames, Antibodies (Basel) 8(1), 2019; and De Vlieger et al., Antibodies (Basel) 8(1), 2018.

To prepare a binding protein that is a bispecific V_{H}H², in some embodiments, the two V_{H}Hs can be synthesized separately, then joined together by a linker. Alternatively, the bispecific V_{H}H² can be synthesized as a fusion protein. V_{H}Hs having different binding activities and receptor targets can be paired to make a bispecific V_{H}H². The binding proteins can be screened for signal transduction on cells carrying one or both relevant receptors.

### V. Linkers

As previously described, the binding domains of the dimeric binding proteins of the present disclosure may be joined contiguously (e.g, the C-terminal amino acid of the first V_{H}H in the binding protein to the N-terminal amino acid of the second V_{H}H in the binding protein) or the binding domains of the binding protein may optionally be joined via a linker. A linker is a linkage between two elements, e.g., protein domains. In a bispecific V_{H}H² binding protein described herein, a linker is a linkage between the two V_{H}Hs in the binding protein. A linker can be a covalent bond or a peptide linker. In some embodiments, the two V_{H}Hs in a binding protein are joined directly (i.e., via a covalent bond). The length of the linker between two V_{H}Hs in a binding protein can be used to modulate the proximity of the two V_{H}Hs of the binding protein. By varying the length of the linker, the overall size and length of the binding protein can be tailored to bind to specific cell receptors or domains or subunits thereof. For example, if the binding protein is designed to bind to two receptors or domains or subunits thereof that are located close to each other on the same cell, then a short linker can be used. In another example, if the binding protein is designed to bind to two receptors or domains or subunits there of that are located on two different cells, then a long linker can be used.

In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). A linker can also be a chemical linker, such as a synthetic polymer, e.g., a polyethylene glycol (PEG) polymer.

In some embodiments, a linker joins the C-terminus of the first V_{H}H in the binding protein to the N-terminus of the second V_{H}H in the binding protein. In other embodiments, a linker joins the C-terminus of the second V_{H}H in the binding protein to the N-terminus of the first V_{H}H in the binding protein.

Suitable peptide linkers are known in the art, and include, for example, peptide linkers containing flexible amino acid residues such as glycine and serine. In certain embodiments, a peptide linker can contain motifs, e.g., multiple or repeating motifs, of GS, GGS, GGGS (SEQ ID NO:62), GGGGS (SEQ ID NO:63), GGGGGS (SEQ ID NO:64), GGSG (SEQ ID NO:65), or SGGG (SEQ ID NO:66). In certain embodiments, a peptide linker can contain 2 to 12 amino acids including motifs of GS, e.g., GS, GSGS (SEQ ID NO:67), GSGSGS (SEQ ID NO:68), GSGSGSGS (SEQ ID NO:69), GSGSGSGSGS (SEQ ID NO:70), or GSGSGSGSGSGS (SEQ ID NO:71). In certain other embodiments, a peptide linker can contain 3 to 12 amino acids including motifs of GGS, e.g., GGS, GGSGGS (SEQ ID NO:72), GGSGGSGGS (SEQ ID NO:73), and GGSGGSGGSGGS (SEQ ID NO:74). In yet other embodiments, a peptide linker can contain 4 to 20 amino acids including motifs of GGSG (SEQ ID NO:65), e.g., GGSGGGSG (SEQ ID NO:75), GGSGGGSGGGSG (SEQ ID NO:76), GGSGGGSGGGSGGGSG (SEQ ID NO:77), or GGSGGGSGGGSGGGSGGGSG (SEQ ID NO:78). In other embodiments, a peptide linker can contain motifs of GGGGS (SEQ ID NO:63), e.g., GGGGSGGGGS (SEQ ID NO:79) or GGGGSGGGGSGGGGS (SEQ ID NO:80).

### Modulation of Activity of Receptor Binding Molecules

In some embodiments, such as to achieve partial agonism or selective activation of particular cell types, the design of the IL10Rα/IL2Rγ binding molecules of the present disclosure may be modulated by structural variations in the design of the receptor binding molecule. This variation in activity may be employed to modulate the binding and activity of the IL10Rα/IL2Rγ binding molecule, for to optimize the activity of the IL10Rα/IL2Rγ binding molecule to achieve partial agonism, selective cell type activation or to provide molecules having increased or decreased binding relative to the cognate ligand for each of the IL10Rα sdAb and IL2Rγ sdAb for their respective receptor subunits. The ability to modulate activity of the IL10Rα/IL2Rγ binding molecules of the present disclosure provides substantial benefits in multiple therapeutic applications. The IL10Rα/IL2Rγ binding molecules of the present disclosure can trigger different levels of downstream signaling in different cell types. For example, by varying the length of the linker between the IL10Rα sdAb antibody and the IL2Rg sdAb antibody in the IL10Rα/IL2Rγ binding molecule, the IL10Rα/IL2Rγ binding molecules provides a higher level of downstream signaling in desired cell types compared to undesired cell types. In other embodiments, different IL10Rα sdAb antibodies with different binding affinities and different IL IL2Rγ sdAb antibodies with different binding affinities can be used to tune the activity of IL10R binding molecule. Further, when the IL10Rα/I IL2Rγ binding molecule is provided as a single a polypeptide, the orientation of the two antibodies in the polypeptide can also be changed to make change the properties of the molecule.

In some embodiments, the IL10Rα/IL2Rγ binding molecules of the present disclosure result in level of downstream signaling in T cells (e.g., CD8⁺ T cells) having an Eₘₐₓ on T cells that is at least 5-fold greater, alternatively 10-fold greater, alternatively 100-fold greater, alternatively at least 1000-fold greater that the Eₘₐₓ of signaling in monocytes.

In one embodiment, the present disclosure provides an IL10Rα/IL2Rγ binding molecule that preferentially activates T cells, in particular CD8⁺ T cells, relative to monocytes. In some embodiments, the IL10Rα/IL2Rγ binding molecules of the present disclosure result in level of downstream signaling in T cells (e.g., CD8⁺ T cells) having an Eₘₐₓ on T cells that is at least 5-fold greater, alternatively 10-fold greater, alternatively 100-fold greater, alternatively at least 1000-fold greater that the Eₘₐₓ of signaling in monocytes.

In some embodiments, it is desired to provide an the IL10Rα/IL2Rγ binding protein has a reduced Eₘₐₓ compared to the Eₘₐₓ caused by IL10, the cognate ligand for the IL10 receptor (i.e. an IL10R binding molecule that is a IL10 partial agonist) with respect to a given cell type. In some embodiments, the IL10Ra/IL2Rg binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 10% and 90%, between 10% and 80%, between 10% and 70%, between 10% and 60%, between 10% and 50%, between 1% and 40%, between 1% and 30%, between 1% and 20%, or between 1% and 10%) of the Eₘₐₓ associated with wildtype hIL10.

### Modulation of Activity by Modulation of Linker Length

In some embodiments, for example by varying the linker length between binding domains of the binding molecule can be employed to modulate the activity of the dimeric binding proteins, both with respect to a particular activity in a given cell types and between cell types. In some embodiments, for example by varying the linker length, an IL10Rα/IL2Rγ binding molecule can cause a higher level of downstream signaling in T cells (e.g., CD8⁺ T cells) compared to the level of downstream signaling in monocytes. The ability to modulate the activity of the IL10Rα/IL2Rγ binding molecule provides a molecule with a higher level of downstream signaling in T cells (e.g., CD8⁺ T cells) compared to the level of downstream signaling in monocytes.

The ability to modulate the activity of the dimeric binding molecule provides a molecule with a higher level of downstream signaling in one particular cell type (e.g., CD8⁺ T cells) compared to the level of downstream in another cell type (e.g. monocytes). A series of representative IL10Ra/IL2Rg dimeric binding molecules were constructed to evaluate and demonstrate the effect of linker length with respect to various biological activities modulated in T cells and monocytes. The results of these studies are presented in Tables 20-29 below which provide details regarding the particular binding protein test article components, linker amino acid sequence and length, the concentrations of the test article evaluated, the cell type used and the resulting biological response measured. Each of these molecules was produced recombinantly and purified in substantial accordance with the examples provided herein. Parameters which were evaluated include pSTAT3 induction in CD8⁺ T cells (Table 20), on pSTAT3 Induction of on CD4 T cells (Table 21) pSTAT3 Induction of in monocytes (Table 22), IFNγ secretion in CD8⁺ T cells (Table 23), Granzyme A secretion in CD8⁺ T cells (Table 24), Granzyme B secretion in CD8⁺ T cells (Table 25), IL9 secretion in CD8⁺ T cells (Table 26), IL-1β secretion in LPS treated monocytes (Table 27), IL6 secretion in LPS treated monocytes (Table 28), and TNF-α secretion in LPS treated monocytes (Table 29). These data demonstrate the ability to modulate the function of the IL10Ra/IL2Rg dimeric binding molecules within a given cell type or to bias function with respect to one cell type or the other by variation of the linker between the binding domains.

Modulation Activity by Modulation of sdAb Binding Affinity(ies):

In some embodiments, the activity and/or specificity of the bivalent IL10Rα/IL2Rγ binding molecule of the present disclosure may be modulated by the respective binding affinities of the sdAbs for their respective receptor subunits. It will be appreciated by one of skill in the art that the binding of the first sdAb of the bivalent IL10Rα/IL2Rγ binding molecule to the first receptor subunit ECD on the cell surface will enhance the probability of a binding interaction between the second sdAb of the bivalent IL10Rα/IL2Rγ binding molecule with the ECD of the second receptor subunit. This cooperative binding effect may result in a bivalent IL10Rα/IL2Rγ binding molecule which has a very high affinity for the receptor and a very slow “off rate” from the receptor [. Typical V_{H}H single domain antibodies have an affinity for their targets of from about 10⁻⁵M to about 10⁻¹⁰M. In those instances such slow off-rate kinetics are desirable in the bivalent IL10Rα/IL2Rγ binding molecule, the selection of sdAbs having high affinities (about 10⁻⁷M to about 10⁻¹⁰M) for incorporation into the bivalent IL10Ra/IL2Rγ binding molecule are favored.

Naturally occurring cytokine ligands typically do not exhibit a similar affinity for each subunit of a heterodimeric receptor. Consequently, in designing a bivalent IL10Rα/IL2Rγ binding molecule, selection of sdAbs for the first and second IL10Rα/IL2Rγ receptor subunit have an affinity similar to (e.g., having an affinity about 10 fold, alternatively about 20 fold, or alternatively about 50 fold higher or lower than) the cognate ligand for the respective receptor subunit may be used.

In some embodiments, the bivalent IL10Rα/IL2Rγ binding molecules of the present disclosure are partial agonists of the IL10Rα/IL2Rγ receptor. As such, the activity of the bivalent binding molecule may be modulated by selecting sdAb which have greater or lesser affinity for either one or both of the IL10Rα/IL2Rγ receptor subunits. As some heterodimeric cytokine receptors are comprised of a “proprietary subunit” (i.e., a subunit which is not naturally a subunit of another multimeric receptor) and a second “common” subunit (such as CD132) which is a shared component of multiple cytokine receptors), selectivity for the formation of such receptor may be enhanced by employing first sdAb which has a higher affinity for the proprietary receptor subunit and second sdAB which exhibits a lower affinity for the common receptor subunit. Additionally, the common receptor subunit may be expressed on a wider variety of cell types than the proprietary receptor subunit. In some embodiments wherein the receptor is a heterodimeric receptor comprising a proprietary subunit and a common subunit, the first sdAb of the bivalent IL10Rα/IL2Rγ binding molecule exhibits a significantly greater (more than 10 times greater, alternatively more than 100 times greater, alternatively more than 1000 times greater) affinity for the proprietary receptor than the second sdAb of the bivalent IL10Rα/IL2Rγ binding molecule for the common receptor subunit. In one embodiment, the present disclosure provides a bivalent IL10Rα/IL2Rγ binding molecule wherein the affinity of the anti-IL10Rα sdAb of has an affinity of more than 10 times greater, alternatively more than 100 times greater, alternatively more than 1000 times greater) affinity anti-IL2Rγ sdAb common receptor subunit.

In one embodiment, the present disclosure provides an IL10Rα/IL2Rγ binding molecule wherein the affinity of the IL10Rα sdAb has a higher affinity for the extracellular domain of IL10Rα than the affinity of the IL2Rg sdAb for the extracellular domain of IL2Rγ. In some embodiments, the present disclosure provides a IL10Rα molecule, wherein the affinity of the IL10Rα sdAb has an affinity for the extracellular domain of IL10Rα of from about 10⁻⁸ to about 10⁻¹⁰ M, alternatively from about 10⁻⁹ to about 10⁻¹⁰M, or alternatively about 10⁻¹⁰ M and the IL2Rγ sdAb an affinity for the extracellular domain of IL2Rγ of from about 10⁻⁶ to about 10⁻⁹ M, alternatively from about 10⁻⁷ to about 10⁻⁹M, alternatively from about 10⁻⁷ to about 10⁻⁸M, alternatively about 10⁻⁹M, alternatively about 10⁻⁸M. In some embodiments, the present disclosure provides a IL10Rα/IL2Rγ binding molecule, wherein the affinity of the IL10Rα sdAb has an affinity for the extracellular domain of IL10Rα of from about 10⁻⁸ to about 10⁻¹⁰ M, alternatively from about 10⁻⁹ to about 10⁻¹⁰M, or alternatively about 10⁻¹⁰ M and the IL2Rg sdAb an affinity for the extracellular domain of IL2Rγ of from about 10⁻⁶ to about 10⁻⁹ M, alternatively from about 10⁻⁷ to about 10⁻⁹M, alternatively from about 10⁻⁷ to about 10⁻⁸M, alternatively about 10⁻⁹M, alternatively about 10⁻⁸M, and the affinity of the IL10Rα sdAb for ECD of IL10Rα is more than 2 fold higher, alternatively more than 5 fold higher, alternatively more than 10 fold higher, alternatively more than 20 fold higher, alternatively more than 40 fold higher, alternatively more than 50 fold higher, alternatively more than 60 fold higher, alternatively more than 70 fold higher, alternatively more than 80 fold higher, alternatively more than 90 fold higher, alternatively more than 100 fold higher, alternatively more than 150 fold higher, alternatively more than 200 fold higher or alternatively more than 500 fold higher than the affinity of the IL2Rγ sdAb for ECD of IL2Rγ.

### VI. Modifications to Extend Duration of Action In Vivo

The binding proteins described herein can be modified to provide for an extended lifetime in vivo and/or extended duration of action in a subject. In some embodiments, the binding protein can be conjugated to carrier molecules to provide desired pharmacological properties such as an extended half-life. In some embodiments, the binding protein can be covalently linked to the Fc domain of IgG, albumin, or other molecules to extend its half-life, e.g., by pegylation, glycosylation, and the like as known in the art.

In some embodiments, the binding protein is conjugated to an Fc polypeptide or an Fc domain (a dimer of two Fc polypeptides), optionally comprising an intervening linker. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The Fc polypeptide or Fc domain useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding protein described herein can be conjugated to the entire Fc polypeptide or Fc domain, or a smaller portion that retains the ability to extend the circulating half-life of a chimeric polypeptide of which it is a part. In addition, full-length or fragmented Fc polypeptide can be variants of the wild-type molecule. In a typical presentation, each Fc polypeptide in an Fc domain can carry a heterologous polypeptide; the two heterologous polypeptides in the Fc domain being the same or different (e.g., one fused to an anti-IL10Rα V_{H}H antibody and the other fused to an anti-IL2Rγ V_{H}H antibody or one or both heterologous polypeptides linked to a anti-IL10Rα V_{H}H antibody/anti-IL2Rγ V_{H}H antibody dimer polypeptide). As indicated, the linkage of the IL10Rα/IL2Rγ bivalent binding molecule to the Fc subunit may incorporate a linker molecule as described below between the bivalent sdAb and Fc subunit. In some embodiments, the IL10Rα/IL2Rγ bivalent binding molecule is expressed as a fusion protein with the Fc domain incorporating an amino acid sequence of a hinge region of an IgG antibody. The Fc domains engineered in accordance with the foregoing may be derived from IgG1, IgG2, IgG3 and IgG4 mammalian IgG species. In some embodiments, the Fc domains may be derived from human IgG1, IgG2, IgG3 and IgG4 IgG species. In some embodiments, the hinge region is the hinge region of an IgG1. In one particular embodiment, the IL10Rα/IL2Rγ bivalent binding is linked to an Fc domain using an human IgG1 hinge domain.

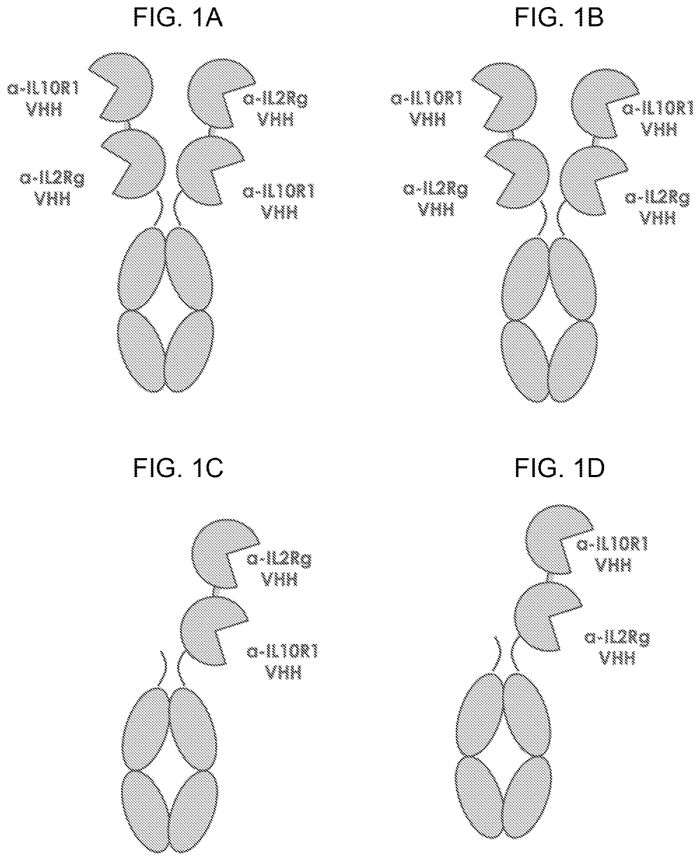

In some embodiments, a bivalent binding molecule of the present disclosure may be conjugated to one (as illustrated in FIGS. 1C and 1D) or both domains of the Fc (as illustrated in FIGS. 1C and 1D). In one embodiment, the Fc domain is an Fc domain that is derived from the human IgG4 IgG4 heavy constant region (UniProt Reference P01861). The use of hIgG4 as the source of the Fc provides advantages such very low FcγR binding thereby reducing the necessity of mutations immunogenicity or effector function. In some embodiments, when hIgG4 is employed as the source of the Fc domain, the hIgG4 Fc may comprise the amino acid substitution S228P (EU numbering) which is useful to stabilize the Fc dimer. Additionally, or alternatively, the hIgG4 Fc may comprise the amino acid substitution N297G (EU numbering) which reduces FcγR binding. Additionally, or alternatively, the hIgG4 Fc may comprise the a deletion of the C-terminal lysine residue (K447del) (EU numbering) which reduces FcγR binding.

In one embodiment, the present disclosure provides a homodimeric binding protein comprised of two of the same IL10Rα/IL2Rg dimeric binding molecules (H1, DR240-G3S-DR231) each attached via an AS linker to a domain of an hIgG4 Fc (comprising the hIgG4 hinge, CH2 and CH3 domains) containing the amino acid substitutions S228P and N297G and the deletion of K447 and having the amino acid sequence:

(SEQ ID NO: 556)
QVQLQESGGGSVQAGGSLRLSCGASGYTYSSYCMGWFRQVPGKEREGVAV
IDSDGSTSYADSVKGRFTISKDNGKNTLYLQMNSLKPEDTAMYYCAADLG
HYRPPCGVLYLGMDYWGKGTQVTVSSGGGSQVQLQESGGGSVQAGGSLRL
SCTASGFTFDDREMNWYRQAPGNECELVSTISSDGSTYYADSVKGRFTIS
QDNAKNTVYLQMDSVKPEDTAVYYCAADFMIAIQAPGAGCWGQGTQVTVS
SASRVESKYGPPCPPCPAPEFLGGPSVFLFPPKPKDTLMISRTPEVTCVV
VDVSQEDPEVQFNWYVDGVEVHNAKTKPREEQFGSTYRVVSVLTVLHQDW
LNGKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLTVD
KSRWQEGNVFSCSVMHEALHNHYTQKSLSLSLG

which is referred to herein as DR992. A nucleic acid sequence encoding DR992 has the DNA sequence

(SEQ ID NO: 557)
CAAGTGCAGCTGCAAGAGAGCGGAGGAGGAAGCGTCCAAGCCGGAGGCTC
TCTGAGGCTGAGCTGTGGAGCCAGCGGCTACACTTACAGCAGCTACTGTA
TGGGCTGGTTTAGGCAAGTGCCCGGCAAGGAGAGAGAGGGCGTGGCCGTG
ATCGATTCCGATGGCAGCACAAGCTACGCTGACAGCGTGAAGGGAAGGTT
CACAATCAGCAAGGACAACGGCAAGAACACACTCTATCTGCAGATGAACA
GCCTCAAGCCAGAGGACACAGCCATGTACTACTGCGCCGCTGATCTGGGC
CACTATAGGCCTCCTTGTGGCGTGCTGTATCTGGGCATGGATTACTGGGG
CAAGGGCACACAAGTGACAGTCTCGAGCGGCGGAGGATCCCAAGTGCAGC
TGCAAGAGAGCGGAGGAGGAAGCGTGCAAGCCGGCGGATCTCTGAGACTC
AGCTGTACTGCCTCCGGCTTCACATTCGACGATAGGGAGATGAACTGGTA
TAGGCAAGCCCCCGGCAATGAGTGCGAGCTGGTGAGCACAATCTCCAGCG
ATGGCAGCACTTACTACGCCGATAGCGTGAAGGGAAGGTTCACTATCTCC
CAAGATAACGCCAAGAACACAGTCTATCTGCAGATGGACTCCGTCAAGCC
AGAGGATACTGCCGTGTACTACTGCGCCGCCGACTTCATGATCGCCATCC
AAGCCCCCGGCGCTGGCTGTTGGGGACAAGGCACTCAAGTGACAGTCTCG
TCTGCTAGCAGAGTGGAATCTAAGTACGGGCCCCCTTGTCCTCCATGTCC
TGCTCCAGAGTTTCTCGGCGGACCCTCCGTGTTCCTGTTTCCTCCAAAGC
CTAAGGACACCCTGATGATCTCTCGGACCCCTGAAGTGACCTGCGTGGTG
GTGGATGTGTCCCAAGAAGATCCCGAGGTGCAGTTCAATTGGTACGTGGA
CGGCGTGGAAGTGCACAACGCCAAGACCAAGCCTAGAGAGGAACAGTTCG
GCTCCACCTACAGAGTGGTGTCCGTGCTGACAGTGCTGCACCAGGATTGG
CTGAACGGCAAAGAGTACAAGTGCAAGGTGTCCAACAAGGGCCTGCCTTC
CAGCATCGAAAAGACCATCTCCAAGGCCAAGGGCCAGCCTAGGGAACCCC
AGGTTTACACCCTGCCTCCAAGCCAAGAGGAAATGACCAAGAACCAGGTG
TCCCTGACCTGCCTGGTCAAGGGCTTCTACCCTTCCGATATCGCCGTGGA
ATGGGAGAGCAATGGCCAGCCTGAGAACAACTACAAGACCACACCTCCTG
TGCTGGACTCCGACGGCTCCTTCTTTCTGTACTCTCGGCTGACCGTGGAC
AAGAGCAGATGGCAAGAGGGCAACGTGTTCTCCTGCTCCGTGATGCACGA
GGCCCTGCACAATCACTACACCCAGAAGTCCCTGTCTCTGTCCCTGGGA

In another embodiment, the present disclosure provides a homodimeric binding protein comprised of two of the same IL10Ra/IL2Rg dimeric binding molecules (A2, DR229-G3S-DR239) each attached to a domain of an IgG4 Fc containing the S228P amino acid substitution having the amino acid sequence:

(SEQ ID No 558)
QVQLQESGGGLVQPGGSLRLSCTASGFSFSSYPMTWARQAPGKGLEWVST
IASDGGSTAYAASVEGRFTISRDNAKSTLYLQLNSLKTEDTAMYYCTKGY
GDGTPAPGQGTQVTVSSGGGSQVQLQESGGGSVQAGGSLRLSCTVSGYTY
SSNCMGWFRQAPGKEREGVATIYTGGGNTYYADSVKGRFTISQDNAKNTV
YLQMNNLKPEDTAMYYCAAEPLSRVYGGSCPTPTFDYWGQGTQVTVSSAS
RVESKYGPPCPPCPAPEFLGGPSVFLFPPKPKDTLMISRTPEVTCVVVDV
SQEDPEVQFNWYVDGVEVHNAKTKPREEQFGSTYRVVSVLTVLHQDWLNG
KEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSR
WQEGNVFSCSVMHEALHNHYTQKSLSLSLG

which is referred to herein as DR995. A nucleic acid sequence encoding DR995 has the DNA sequence

(SEQ ID NO: 559)
CAAGTGCAGCTGCAAGAGAGCGGAGGAGGACTGGTCCAGCCCGGCGGCTC
TCTGAGGCTGAGCTGCACTGCTTCCGGCTTCAGCTTCAGCAGCTACCCTA
TGACATGGGCTAGGCAAGCCCCCGGCAAAGGACTGGAATGGGTGAGCACT
ATTGCCAGCGATGGAGGCAGCACAGCCTACGCTGCCAGCGTGGAGGGAAG
GTTCACAATCTCTAGGGACAATGCCAAGAGCACACTGTATCTGCAGCTGA
ACTCTCTGAAGACAGAGGACACTGCCATGTACTACTGCACTAAGGGCTAC
GGCGATGGCACACCAGCTCCCGGCCAAGGCACACAAGTGACTGTCTCGAG
CGGCGGAGGATCCCAAGTGCAGCTGCAAGAGAGCGGAGGAGGAAGCGTGC
AAGCCGGAGGCTCTCTGAGGCTGAGCTGTACAGTGTCCGGCTACACTTAC
AGCTCCAATTGCATGGGCTGGTTTAGGCAAGCCCCCGGCAAGGAAAGAGA
GGGCGTGGCCACTATCTACACTGGCGGCGGCAACACATACTACGCCGATA
GCGTGAAGGGAAGGTTCACTATCAGCCAAGATAACGCCAAGAACACAGTG
TATCTGCAGATGAACAATCTGAAGCCAGAGGACACTGCCATGTACTACTG
TGCTGCTGAGCCACTGTCTAGGGTGTACGGCGGCAGCTGCCCAACTCCTA
CATTCGACTACTGGGGCCAAGGCACACAAGTGACTGTCTCGTCTGCTAGC
AGAGTGGAATCTAAGTACGGGCCCCCTTGTCCTCCATGTCCTGCTCCAGA
GTTTCTCGGCGGACCCTCCGTGTTCCTGTTTCCTCCAAAGCCTAAGGACA
CCCTGATGATCTCTCGGACCCCTGAAGTGACCTGCGTGGTGGTGGATGTG
TCCCAAGAAGATCCCGAGGTGCAGTTCAATTGGTACGTGGACGGCGTGGA
AGTGCACAACGCCAAGACCAAGCCTAGAGAGGAACAGTTCGGCTCCACCT
ACAGAGTGGTGTCCGTGCTGACAGTGCTGCACCAGGATTGGCTGAACGGC
AAAGAGTACAAGTGCAAGGTGTCCAACAAGGGCCTGCCTTCCAGCATCGA
AAAGACCATCTCCAAGGCCAAGGGCCAGCCTAGGGAACCCCAGGTTTACA
CCCTGCCTCCAAGCCAAGAGGAAATGACCAAGAACCAGGTGTCCCTGACC
TGCCTGGTCAAGGGCTTCTACCCTTCCGATATCGCCGTGGAATGGGAGAG
CAATGGCCAGCCTGAGAACAACTACAAGACCACACCTCCTGTGCTGGACT
CCGACGGCTCCTTCTTTCTGTACTCTCGGCTGACCGTGGACAAGAGCAGA
TGGCAAGAGGGCAACGTGTTCTCCTGCTCCGTGATGCACGAGGCCCTGCA
CAATCACTACACCCAGAAGTCCCTGTCTCTGTCCCTGGGA

Alternatively, the wild-type human IgG4 Fc (hIgG4 hinge-CH2-CH3) may be employed which has the amino acid sequence:

(SEQ ID NO: 560)
RVESKYGPPCPSCPAPEFLGGPSVFLFPPKPKDTLMISRTPEVTCVVVDV
SQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSR
WQEGNVFSCSVMHEALHNHYTQKSLSLSLGK.

In some embodiments the present disclosure provides a heterodimeric Fc comprising at least one anti-IL10Rα V_{H}H antibody and at least one anti-IL2Rγ V_{H}H antibody, wherein anti-IL10Rα V_{H}H antibody/Fc fusion and an anti-IL2Rγ V_{H}H antibody/Fc fusion polypeptides of the heterodimeric Fc are covalently linked via one disulfide bond, optionally two disulfide bonds, optionally three disulfide bonds, or optionally four disulfide bonds. In some embodiments, the anti-IL10Rα V_{H}H antibody/FC fusion and an anti-IL2Rγ V_{H}H antibody/Fc fusion polypeptides are covalently linked via a disulfide bond between the sulfhydryl group of amino acid C226 of the lower hinge domain of the anti-IL10Rα V_{H}H antibody/Fc fusion and the sulfhydryl group of amino acid C226 of the lower hinge domain of the anti-IL2Rγ V_{H}H antibody/Fc fusion. In some embodiments, the two fusions are covalently linked via a disulfide bond between the sulfhydryl group of amino acid C229 of the lower hinge domain of the anti-IL10Rα V_{H}H antibody/Fc fusion and the sulfhydryl group of amino acid C229 of the lower hinge domain of the anti-IL2Rγ V_{H}H antibody/Fc fusion. In some embodiments, a first Fc domain comprises the amino acid substitution S354C, and the second Fc domain comprises the amino acid substitution Y349C. In some embodiments, the heterodimeric Fc comprises a first Fc domain comprising the amino acid substitution S354C and the second Fc domain comprising the amino acid substitution Y349C and wherein the fusions are linked via a disulfide bond between the S354C of the first Fc domain and Y349C of the second Fc domain. In some embodiments, the two polypeptides of the heterodimeric Fc are covalently linked via one or more, optionally two or more optionally three or more disulfide bonds, optionally four or more disulfide bonds between the side chains of the following groups of cystine pairs: (a) C96 of the first Fc fusion and C199 of the second Fc fusion; (b) between C226 of the first Fc fusion and the C226 of the second Fc fusion, (c) between C229 of the first Fc fusion and the C229 of the second Fc fusion; and (d) between S354C of the first Fc fusion comprising a S354C amino acid substitution and Y349C of the second Fc fusion comprising a Y349C amino acid substitution.

In some embodiments the present disclosure provides a heterodimeric Fc wherein either or both of the fusion subunits of the heterodimeric Fc comprise one or more amino acid substitutions to reduce effector function. In some embodiments, the fusion polypeptides comprise a set of amino acid substitutions selected from the group consisting of: (a) L234A/L235A/P329A (“LALAPA”); L234A/L235A/P329G (“LALAPG”); L234A/L235E/G237A/A330S/P331S (“AEASS”); E233P/L234V/L235A/AG237 (PVAdelG); and L234F/L235E/P331S (“FES”).

In some embodiments the present disclosure provides a heterodimeric Fc wherein either or both of the fusion subunits of the heterodimeric Fc comprises an amino acid substitution at position C220 (EU numbering) of the upper hinge domain to eliminate the sulfhydryl side chain. In some embodiments, the substitution at position C220 is C220S (EU numbering) substitution.

In some embodiments the present disclosure provides a heterodimeric Fc wherein either or both of the fusion subunits of the heterodimeric Fc comprises amino acid substitutions in the Fc domain at positions M428 and/or N434 (EU numbering). In some embodiments the amino acid substitutions at positions M428 and/or N434 are M428L and/or N434S.

In some embodiments the present disclosure provides a heterodimeric Fc wherein either or both of the fusion subunits of the heterodimeric Fc comprises amino acid deletions in the Fc domain at positions G446 and/or K447 (EU numbering).

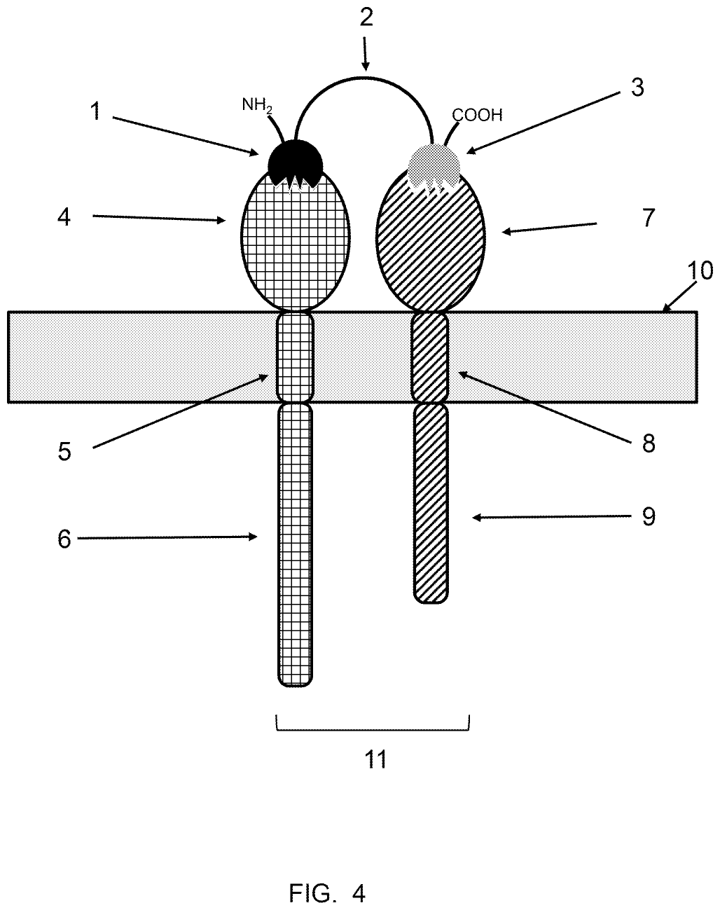

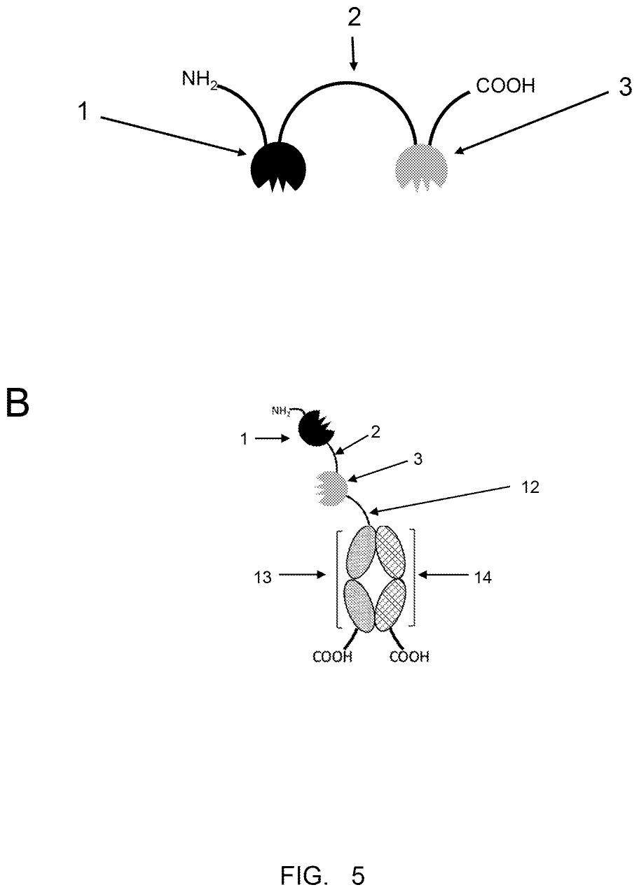

Illustrative examples of Fc formats useful for binding molecules of the present disclosure are provided schematically in FIGS. 4-7 of the attached drawings.

In some embodiments the present disclosure provides a heterodimeric Fc wherein either or both of the fusion subunits of the heterodimeric Fc are PEGylated. In some embodiments, either or both of the fusion subunits are PEGylated via the sulfhydryl side chain of amino acid C220 of the upper hinge.

In some embodiments, the present disclosure provides an expression cassette encoding a heterodimeric Fc comprising a nucleic acid sequence encoding anti-IL10Rα V_{H}H antibody/Fc fusion and an anti-IL2Rγ V_{H}H antibody/Fc fusion polypeptides operably linked to one or more heterologous nucleic acid sequences, wherein the nucleic acid sequences encoding the anti-IL10Rα V_{H}H antibody/Fc fusion and an anti-IL2Rγ V_{H}H antibody/Fc fusion polypeptides are: (a) under the control a single promoter and (b) are linked via an intervening sequence that facilitates co-expression. In some embodiments wherein the nucleic acid sequences encoding the anti-IL10Rα V_{H}H antibody/Fc fusion and an anti-IL2Rγ V_{H}H antibody/Fc fusion polypeptides are linked via an intervening sequence that facilitates co-expression, the nucleic acid sequence encoding the anti-IL10Rα V_{H}H antibody/Fc fusion polypeptide is 5′ relative to the nucleic acid sequence encoding the anti-IL2Rγ V_{H}H antibody/Fc fusion polypeptide. In some embodiments wherein the nucleic acid sequences encoding the anti-IL10Rα V_{H}H antibody/Fc fusion and an anti-IL2Rγ V_{H}H antibody/Fc fusion polypeptides are linked via an intervening sequence that facilitates co-expression, the nucleic acid sequence encoding the anti-IL2Rγ V_{H}H antibody/Fc fusion polypeptide is 5′ relative to the nucleic acid sequence encoding the anti-IL10Rα V_{H}H antibody/Fc fusion polypeptide. In some embodiments, the intervening sequence to facilitate co-expression is an IRES element or a T2A sequence.

In some embodiments, the present disclosure provides an expression cassette encoding a heterodimeric Fc comprising a nucleic acid sequence encoding anti-IL10Rα V_{H}H antibody/Fc fusion and an anti-IL2Rγ V_{H}H antibody/Fc fusion polypeptides operably linked to one or more heterologous nucleic acid sequences, wherein the nucleic acid sequences encoding the anti-IL10Rα V_{H}H antibody/Fc fusion and an anti-IL2Rγ V_{H}H antibody/Fc fusion polypeptides are: (a) under the control a single promoter and (b) are linked via an intervening sequence that facilitates co-expression in a mammalian cell.

The present disclosure further provides a recombinant vector encoding a heterodimeric Fc, the vector comprising a first expression cassette encoding an anti-IL10Rα V_{H}H antibody/Fc fusion polypeptide and a second expression cassette comprising a nucleic acid sequence encoding a anti-IL2Rγ V_{H}H antibody/Fc fusion polypeptide. In some embodiments, the vector is viral vector. In some embodiments, the vector is non-viral vector.

Further provided is a recombinantly modified cell comprising a nucleic acid molecule or vector of the disclosure. In some embodiments, the cell is a prokaryotic cell, such as a bacterial cell. In some embodiments, the cell is a eukaryotic cell, such as a mammalian cell. Also provided is a cell culture comprising at least one recombinantly modified cell of the disclosure, and a culture medium.

In some embodiments, the recombinantly modified cell is transformed with a recombinant vector encoding a heterodimeric Fc, the vector comprising a first expression cassette encoding an anti-IL2Rγ V_{H}H antibody/Fc fusion polypeptide and a second expression cassette comprising a nucleic acid sequence encoding an anti-IL10Rα V_{H}H antibody/Fc fusion polypeptide. In some embodiments, the recombinantly modified cell is transformed with a recombinant vector encoding a heterodimeric Fc, the vector comprising a first expression cassette encoding an anti-IL10Rα V_{H}H antibody/Fc fusion polypeptide and a second expression cassette comprising a nucleic acid sequence encoding a anti-IL2Rγ V_{H}H antibody/Fc fusion polypeptide.

In some embodiments, the recombinantly modified cell is transformed with a first vector comprising a nucleic acid sequence encoding a anti-IL2Rγ V_{H}H antibody/Fc fusion polypeptide operably linked to one or more expression control sequences and a second vector comprising an expression cassette comprising a nucleic acid sequence encoding a anti-IL10Rα V_{H}H antibody/Fc fusion polypeptide operably linked to one or more expression control sequences. In some embodiments, the recombinantly modified cell is transformed with a first vector comprising a nucleic acid sequence encoding a anti-IL10Rα V_{H}H antibody/Fc fusion polypeptide operably linked to one or more expression control sequences and a second vector comprising an expression cassette comprising a nucleic acid sequence encoding a anti-IL2Rγ V_{H}H antibody/Fc fusion polypeptide operably linked to one or more expression control sequences. In some embodiments, the cell is a prokaryotic cell, such as a bacterial cell. In some embodiments, the cell is a eukaryotic cell, such as a mammalian cell. Also provided is a cell culture comprising at least one recombinantly modified cell of the disclosure, and a culture medium.

The present disclosure further provides methods for the recombinant production, isolation, purification and characterization of a heterodimeric Fc. Thus, provided herein is a method for producing a heterodimeric Fc of the disclosure. In some embodiments, the method comprises a) providing one or more recombinantly modified cells comprising a nucleic acid molecule or vector comprising a nucleic acid sequence encoding a heterodimeric Fc as disclosed herein; and b) culturing the one or more cells in a culture medium such that the cells produce the heterodimeric Fc encoded by the nucleic acid sequence.

Also provided is a pharmaceutical composition comprising a heterodimeric Fc of the present disclosure. In some embodiments, the pharmaceutical composition comprises a heterodimeric Fc of the present disclosure and a pharmaceutically acceptable carrier. In some embodiments, the pharmaceutical composition comprises a nucleic acid molecule or vector of the disclosure. In some embodiments, the pharmaceutical composition comprises a recombinantly modified cell of the disclosure. In some embodiments, the recombinantly modified cell is a mammalian cell.

The present disclosure provides a heterodimeric Fc, the heterodimeric Fc comprising a first polypeptide of the formula #1:

anti-IL10Rα V_{H}H antibody-L1ₐ-UH1-Fc1 [1]

and a second polypeptide of the formula #2:

anti-IL2Rγ V_{H}H antibody-L2_{b}-UH2-Fc2 [2]

wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
FC2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and
   wherein the polypeptide of formula 1 and the polypeptide of formula 2 are linked by at least one interchain disulfide bond.

### Upper Hinge:

The heterodimeric Fcs of the present disclosure are heterodimers comprising polypeptides of the formulae [1] and [2], which each incorporate an upper hinge region of a human immunoglobulin molecule. The term “upper hinge” or “UH” refers to an amino acid sequence corresponding to amino acid residues 216-220 (EU numbering) of a human immunoglobulin molecule. In some embodiments, the upper hinge region is a naturally occurring upper hinge region of a human immunoglobulin selected from the LH regions of human IgG1, human IgG2, human IgG3 and human IgG4 upper hinge domains. In some embodiments, the upper hinge region is the upper hinge region of a human IgG1 immunoglobulin. In some embodiments, the upper hinge region is the upper hinge region of a human IgG1 immunoglobulin comprising the pentameric amino acid sequence: EPKSC (SEQ ID NO: 11).

In some embodiments, the upper hinge region contains an unpaired cysteine residue at position 220 (EU numbering) that typically, in a complete immunoglobulin molecule, binds to a cysteine on a light chain. When only the Fc domain is used comprising the hinge domain, the unpaired cysteine in the hinge domain creates the potential of the formation of improper disulfide bonds. Consequently, in some embodiments the cysteine at position 220 (C220, numbered in accordance with EU numbering) is substituted with an amino acid that does not promote disulfide bonding. In some embodiments, the Fc domain comprises a C220S mutation having the amino acid sequence EPKSS.

### Fc1 and Fc2:

The heterodimeric Fcs of the present disclosure are heterodimers comprising polypeptides of the formulae [1] and [2], which each incorporate an Fc region (Fc1 and Fc2) of a human immunoglobulin molecule modified to promote heterodimerization.

As used herein the term “FC” and “Fc monomer” are used interchangeably herein to designate the monomeric polypeptide subunit of an Fc dimer. An Fc comprises an amino acid sequence (from amino to carboxy terminal) comprising a lower hinge domain and the CH2 and CH3 domains of a human immunoglobulin molecule. In some embodiments, the Fc monomer is a polypeptide comprising the lower hinge domain and the CH2 and CH3 domains of a human immunoglobulin molecule domains of human IgG1, human IgG2, human IgG3 and human IgG4 hinge domains. The CH2 domain of hIgG1 corresponds to amino acid residues 231-340 (EU numbering) and is provided as SEQ ID NO: 14. The CH3 domain of hIgG1 corresponds to amino acid residues 341-447(EU numbering).

The polypeptides of the formulae [1] and [2] each incorporate a lower hinge region of a human immunoglobulin. As used herein, the term “lower hinge” or “LH” refers to an amino acid sequence corresponding to amino acid residues 221-229 (EU numbering) of a human immunoglobulin molecule. In some embodiments, the lower hinge region is a naturally occurring lower hinge region of a human immunoglobulin selected from the LH regions of IgG1, IgG2, IgG3 and IgG4 lower hinge domains. In some embodiments, the lower hinge region is the lower hinge region of a human IgG1 immunoglobulin. In some embodiments, the lower hinge region is the lower hinge region of a human IgG1 immunoglobulin comprising the decameric amino acid sequence: DKTHTCPPCP.

In some embodiments, Fc1 and Fc2 are derived from a polypeptide corresponding to amino acids 221-447 (EU numbering) of the human IgG1 immunoglobulin having the amino acid sequence (EU numbering indicated:

```
              230 240 250 260
DKTHTCPPCP APELLGGPSV FLFPPKPKDT LMISRTPEVT
              270 280. 290. 300
CVVVDVSHED PEVKFNWYVD GVEVHNAKTK PREEQYNSTY
              310 320 330 340
RVVSVLTVLH QDWLNGKEYK CKVSNKALPA PIEKTISKAK
              350 360 370 380
GQPREPQVYT LPPSRDELTK NQVSLTCLVK GFYPSDIAVE
              390 400 410 420
WESNGQPENN YKTTPPVLDS DGSFFLYSKL TVDKSRWQQG
              430 440 447
NVFSCSVMHE ALHNHYTQKS LSLSPGK
```

As indicated in above sequence, the C-terminal residue of the wild-type form of the IgG1 Fc domain is a lysine, referred to as K447 in accordance with EU numbering. The K447 is inconsistently removed by the producer cell during recombinant product. As a result, the population of recombinant Fc monomers may be heterogenous in that some fraction of the recombinantly produced Fc monomers will contain K447 and others will not. Such inconsistent proteolytic processing by producer cells may therefore result in a heterogenous population of Fcs. Typically, particularly in the context of human pharmaceutical agents, such heterogeneity of the active pharmaceutical ingredient is to be avoided. Consequently, in addition to modifications to the Fc monomer sequence promote heterodimerization, the present disclosure provides Fc monomers that further comprising a deletion of the C-terminal K447 or a deletion of G446 and K447 and nucleic acid sequences encoding Fc monomers comprising a: (a) a deletion of the lysine residue at position 447 (K447, EU numbering, abbreviated as AK447 or des-K447), or (b) deletion of both the glycine at position 456 (G446 EU numbering, abbreviated as des-G446) and K447 (this double deletion of G446 and K447 being referred to herein as des-G446/des-K447 or AG446/AK447).

### Modifications of Fc Subunits to Promote Heterodimerization

As provided in formulae [1] and [2] above, the Fc1 and Fc2 monomers of the dimeric Fc contain amino acid substitutions that promote heterodimerization between Fc1 and Fc2. A variety of techniques are established for the promotion of heterodimerization of Fc domains. See, e.g. Gillies, et al. United States patent No. Kim, et al., U.S. Pat. No. 11,087,249, issued Aug. 3, 2021. In some embodiments, the modifications to promoter heterodimerization of the Fc1 and Fc2 monomers are the HF-TA mutations and the HA-TF mutations as described in Moore, et al (2011) mAbs 3(6):546-557. The HF-TA method employs the S364H/T394F substitutions on one Fc monomer and the Y349T/F405A substitutions on the complementary Fc monomer. The (HA-TF) method employs the S364H/F405A substitutions on one Fc monomer and the Y349T/T394F substitutions on the complementary Fc monomer. Alternatively, the Fc1 and Fc2 monomers are modified to promote heterodimerization by the ZW1 heterodimerization method which employs the T350V/L351Y/F405A/Y407V substitutions on one Fc monomer and the T350V/T366L/K392L/T394W substitutions on the complementary Fc monomer. Von Kreudenstein, et al (2013) mAbs, 5(5):646-654. Alternatively, the Fc1 and Fc2 monomers are modified to promote heterodimerization by the EW-RVT heterodimerization method which employs the K360E/K409W substitutions on one Fc monomer and the Q347R/D399V/F405T substitutions on the complementary Fc monomer. Choi, et al (2015) Molecular Immunology 65(2):377-83.

In one embodiment, Fc1 and Fc2 are modified to promote heterodimerization by the employment of the “knob-into-hole” (abbreviated KiH) modification as exemplified herein. The KiH modification comprises one or more amino acid substitutions in a first Fc monomer (e.g. Fc1) that create a bulky “knob” domain on a first Fc and one or more amino acid substitutions on a second Fc monomer (e.g. Fc2) that create a complementary pocket or “hole” to receive the “knob” of the first Fc monomer.

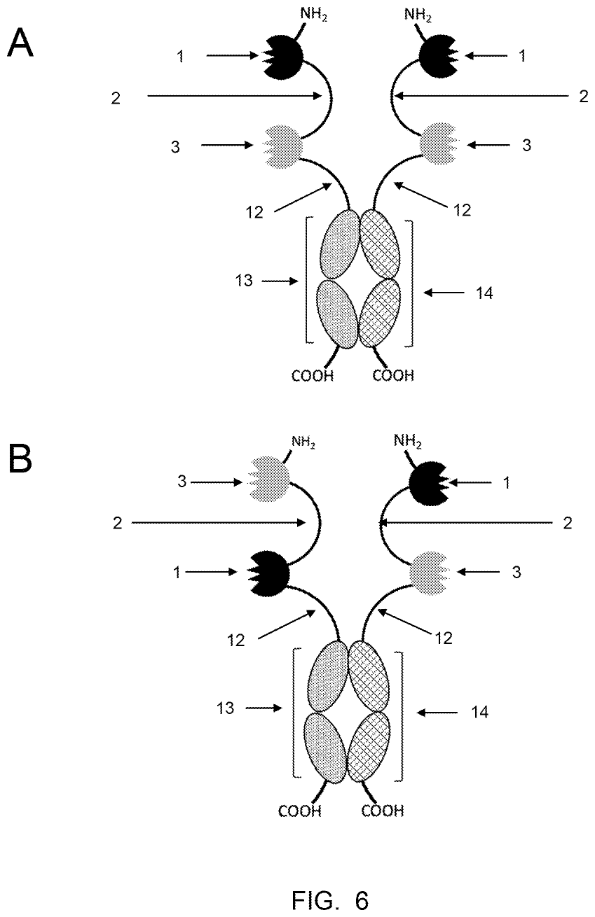

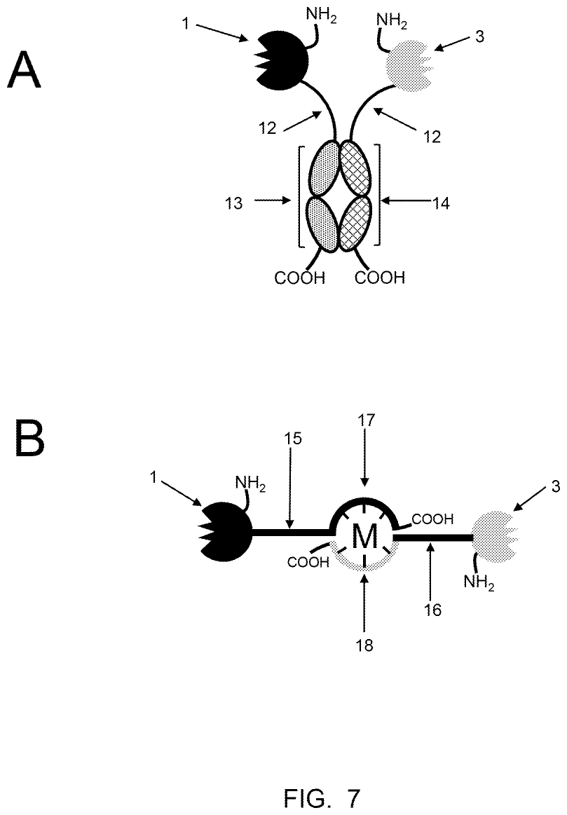

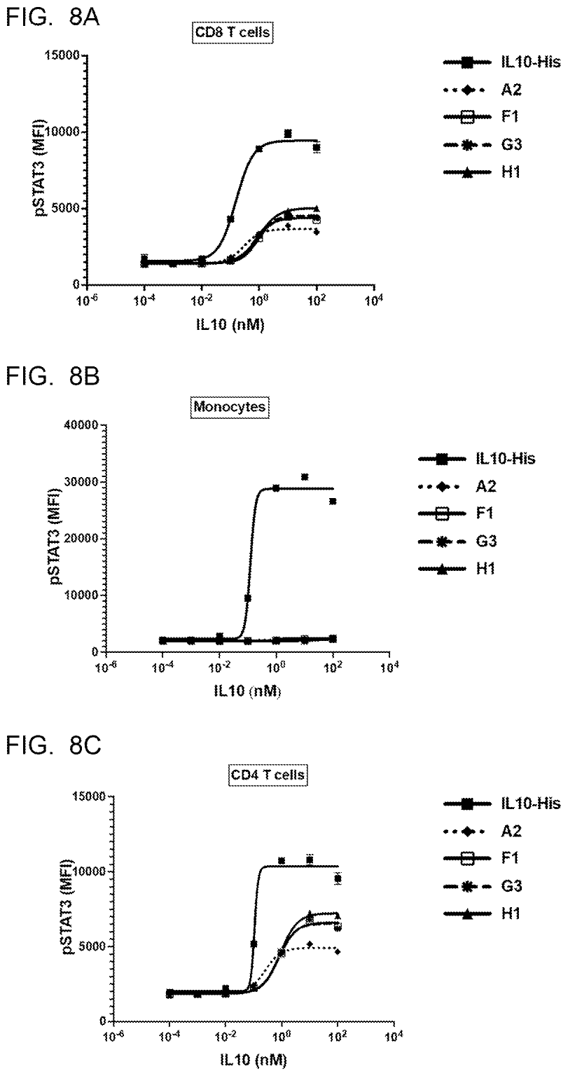

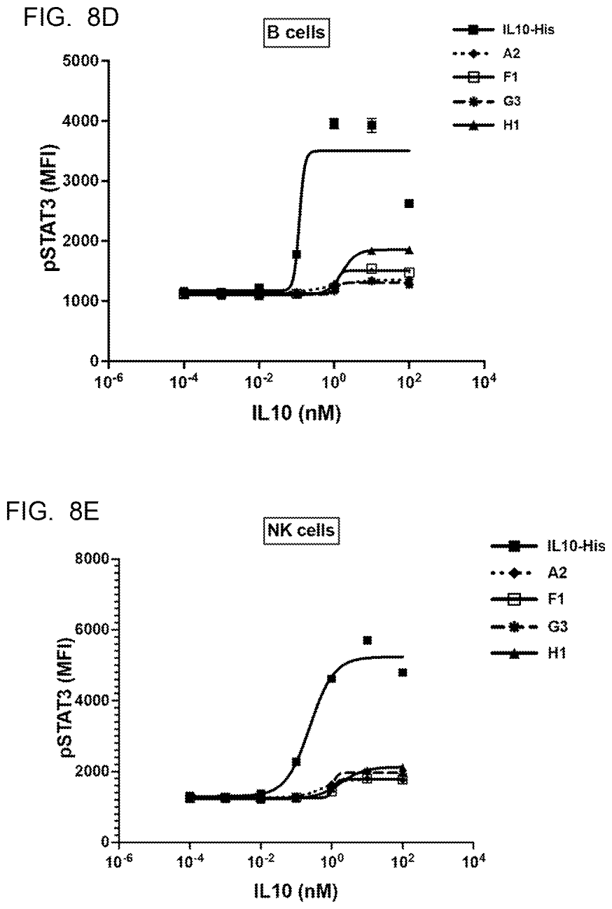

The knob-into-hole format is used to facilitate the expression of a first polypeptide on a first Fc monomer with a “knob” modification and a second polypeptide on the second Fc monomer possessing a “hole” modification to facilitate the expression of heterodimeric polypeptide conjugates. In some embodiments, the IL10Rα/IL2Rγ bivalent binding molecule covalently linked to a single subunit of the Fc as illustrated in FIG. 6, a IL10Rα/IL2Rγ bivalent binding molecule is provided on each of the subunits of the Fc as illustrated in FIG. 7A.

A variety of amino acid substitutions have been established for the creation of complementary knob and hole Fc monomers. See, e.g. Ridgway, et al (1996) Protein Engineering 9(7):617-921; Atwell, et al (1997) J. Mol. Biol. 270:26-35; Carter, et al. U.S. Pat. No. 5,807,706 issued Sep. 15, 1998; Carter, et al U.S. Pat. No. 7,695,936 issued Apr. 13, 2010; Zhao et al. “A new approach to produce IgG4-like bispecific antibodies,” Scientific Reports 11: 18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8: 38. doi:10.3389/fimmu.2017.00038 (2017).

In some embodiments, the Fc domain comprises two Fc monomers wherein the CH3 domain of a first Fc monomer wherein the threonine at (EU numbering) position 366 is modified with a bulky residue (e.g. a T366W) create a “knob” and the substitution, and a second Fc monomer comprising one or more substitutions in complementary residues of the CH3 domain of the second Fc monomer to create a pocket or “hole” to receive the bulky residue, for example by amino acid substitutions such as T366S, L368A, and/or Y407V.

In one embodiment, the Fc1 monomer of formula 1 is a “knob” modified Fc monomer comprising the amino acid substitution T366W and the Fc2 monomer of formula 2 is a “hole” modified Fc comprising the set of amino acid substitutions T366S/L368A/Y407V.

Alternatively, the Fc1 monomer of formula 1 is a “hole” modified Fc monomer comprising the set of amino acid substitutions T366S/L368A/Y407V and the Fc2 monomer of formula 2 is a “knob” modified Fc monomer comprising the amino acid substitution T366W.

An example of an engineered Fc heterodimeric pair comprising complementary KiH modifications is provided in the Table below:

TABLE
Amino Acid Substitution Sets of Complementary
IgG1 KiH Heterodimeric Pairs

| Fc | | Amino Acid |
|---|---|---|
| Dimer | Fc | Substitution Set |
| No. | Monomer | (EU Numbering) |
| 1 | Knob | T366W |
| | Hole | T366S/L368A/Y407V |

As noted, the heterodimeric Fes of the present disclosure are provided as a complementary heterodimeric pair of polypeptides of the formulae [1] and [2] wherein the first and second polypeptide are linked by at least one disulfide bond. In some embodiments, the incorporation of a disulfide bond between the polypeptides of formulae [1] and [2] may be achieved by cysteine substitutions at particular points within the Fc1 and Fc2 domains. In one embodiment, the Fc1 domain of the polypeptide of formula [1] is derived from the Fc domain of hIgG1 comprising an amino acid substitution S354C (EU numbering) and the Fc2 domain of the polypeptide of formula [2] is derived from the Fc domain of hIgG1 comprising an amino acid substitution Y349C (EU numbering) to provide a disulfide bond between the S354C of Fc1 and Y349C of Fc2. Alternatively, the Fc1 domain of the polypeptide of formula [1] is derived from the Fc domain of hIgG1 comprising an amino acid substitution Y349C (EU numbering) and the Fc2 domain of the polypeptide of formula [2] is derived from the Fc domain of hIgG1 comprising an amino acid substitution S354C (EU numbering) to provide a disulfide bond between the S354C of Fc1 and Y349C of Fc2.

Further examples of complementary KiH engineered heterodimeric Fc pairs that may be used in the practice of the present disclosure are provided in the Table below.

TABLE
Knob-into-Hole Fc Dimer Pairs

| Fc | | | | | | |
|---|---|---|---|---|---|---|
| Dimer | Monomer | Monomer | UH | UH SEQ | Fc Amino Acid Substitutions | |
| Pair. | Type | SEQ ID | Sequence | ID | (EU Numbering) | Fc Seq ID |
| 2 | Knob | 19 | wt | 11 | T366W | 17 |
| | Hole | 20 | wt | 11 | T366S/L368A/Y407V | 18 |
| 3 | Knob | 21 | C220S | 12 | T366W | 17 |
| | Hole | 22 | C220S | 12 | T366S/L368A/Y407V | 18 |
| 4 | Knob | 23 | wt | 11 | T366W | 17 |
| | Hole | 24 | wt | 11 | T366S/L368A/Y407V | 18 |
| 5 | Knob | 25 | C220S | 12 | S354C/T366W | 17 |
| | Hole | 26 | C220S | 12 | Y349C/T366S/L368A/Y407V | 18 |

### Additional Fc Modifications

In addition to the modifications to promote heterodimerization of the Fc1 and Fc2 domains, Fc1 and Fc2 may optionally provide additional amino acid modifications that mitigate effector function, or eliminate the glycosylation site at N297 such as N297Q.

### Modifications to Reduce Effector Functions

In some embodiments the amino acid sequence of the Fc1 and/or Fc2 monomers modified to promote heterodimerization may be further modified to reduce effector function. In some embodiments, the Fc domain may be modified to substantially reduce binding to Fc receptors (FcyR and FcR) which reduces or abolishes antibody directed cytotoxicity (ADCC) effector function. Modification of Fc domains to reduce effector function are well known in the art. See, e.g., Wang, et al. (2018) IgG Fc engineering to modulate antibody effector functions, Protein Cell 9(1):63-73. For example, mutation of the lysine residue at position 235 (EU numbering) from leucine (L) to glutamic acid (E) is known to reduce effector function by reducing FcgR and C1q binding. Alegre, et al. (1992) J. Immunology 148:3461-3468.

Additionally, substitution of the two leucine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh et al., (2001) J. Virol 75(24):12161-68. Furthermore, mutation of the proline at position 329 (EU numbering) to alanine (P329A) or glycine, (P329G) mitigates effector function and may be combined with the L234A and L235A substitutions. In some embodiments, the Fc domains (Fc1 and Fc2) of the compositions of the present invention may comprises the amino acid substitutions L234A/L235A/P329A (EU numbering) referred to as the “LALAPA” substitutions or L234A/L235A/P329G (EU numbering) referred to as the “LALAPG” substitutions. In some embodiments, the Fc domains (Fc1 and Fc2) of the compositions of the present disclosure may comprises the amino acid substitutions E233P/L234V/L235A/AG237 (referred to in the scientific literature as the PVAdelG mutation).

In some embodiments, the Fc domains (Fc1 and Fc2) of the compositions of the present disclosure are from hIgG4. In such instances where the Fc domains of the heterodimeric Fc are derived from hIgG4, attenuation of effector function may be achieve by introduction of the S228P and/or the L235E mutations (EU numbering).

Examples of paired KiH Fc dimeric constructs that may be incorporated into the Fcs of the present disclosure are provided in the Table below:

TABLE
Amino Acid Substitution Sets of Complementary IgG1 KiH UH/Fc Heterodimeric
Pairs Comprising Mutations to Reduce Effector Function

| Fc | | |
|---|---|---|
| Dimer | UH Fc | Amino Acid Substitution Set |
| No. | Monomer | (EU Numbering) |
| 6 | Knob | L234A/L235A/P329A/T366W/ΔK447 |
| | Hole | L234A/L235A/P329A/T366S/L368A/Y407V/ΔK447 |
| 7 | Knob | C220S/L234A/L235A/P329A/T366W/ΔK447 |
| | Hole | C220S/L234A/L235A/P329A/T366S/L368A/Y407V/ΔK447 |
| 8 | Knob | L234A/L235A/P329A/S354C/T366W/ΔK447 |
| | Hole | L234A/L235A/P329A/Y349C/T366S/L368A/Y407V/ΔK447 |
| 9 | Knob | C220S/L234A/L235A/P329A/S354C/T366W/ΔK447 |
| | Hole | C220S/L234A/L235A/P329A/Y349C/T366S/L368A/Y407V/ΔK447 |
| 10 | Knob | L234A/L235A/P329G/T366W/ΔK447 |
| | Hole | L234A/L235A/P329G/T366S/L368A/Y407V/ΔK447 |
| 11 | Knob | C220S/L234A/L235G/P329A/T366W/ΔK447 |
| | Hole | C220S/L234A/L235G/P329A/T366S/L368A/Y407V/ΔK447 |
| 12 | Knob | L234A/L235A/P329G/S354C/T366W/ΔK447 |
| | Hole | L234A/L235A/P329G/Y349C/T366S/L368A/Y407V/ΔK447 |
| 13 | Knob | C220S/L234A/L235A/P329G/S354C/T366W/ΔK447 |
| | Hole | C220S/L234A/L235A/P329G/Y349C/T366S/L368A/Y407V/ΔK447 |
| 14 | Knob | L234A/L235E/G237A/A330S/P331S/T366W/ΔK447 |
| | Hole | L234A/L235E/G237A/A330S/P331S/T366S/L368A/Y407V/ΔK447 |
| 15 | Knob | C220S L234A/L235E/G237A/A330S/P331S/T366W/ΔK447 |
| | Hole | C220S/L234A/L235E/G237A/A330S/P331S/T366S/L368A/Y407V/ΔK447 |
| 16 | Knob | L234A/L235E/G237A/A330S/P331S/S354C/T366W/ΔK447 |
| | Hole | L234A/L235E/G237A/A330S/P331S/Y349C/T366S/L368A/Y407V/ΔK447 |
| 17 | Knob | C220S/L234A/L235E/G237A/A330S/P331S/S354C/T366W/ΔK447 |
| | Hole | C220S/L234A/L235E/G237A/A330S/P331S/Y349C/T366S/L368A/Y407V/ΔK447 |
| 18 | Knob | L234F/L235E/P331S/T366W/ΔK447 |
| | Hole | L234F/L235E/P331S/T366S/L368A/Y407V/ΔK447 |
| 19 | Knob | C220S/L234F/L235E/P331S/T366W/ΔK447 |
| | Hole | C220S/L234F/L235E/P331S//L368A/Y407V/ΔK447 |
| 20 | Knob | L234F/L235E/P331S/S354C/T366W/ΔK447 |
| | Hole | L234F/L235E/P331S/Y349C/T366S/L368A/Y407V/ΔK447 |
| 21 | Knob | C220S/S/L234F/L235E/P331S/354C/T366W/ΔK447 |
| | Hole | C220S/L234F/L235E/P331S/Y349C/T366S/L368A/Y407V/ΔK447 |

### Sequence Modifications to Extend Duration of Action:

In some embodiments the amino acid sequence of the Fc1 and/or Fc2 monomers modified to promote heterodimerization may be further modified to incorporate amino acid substitutions which extend the duration of action of the molecule and prevent clearance. In some embodiments, such modifications to the Fc monomer include the amino acid substitutions M428L and N434S (EU numbering) referred to as the “LS” modification. The LS modification may optionally be combined with amino acid substitutions to reduce effector function and provide for disulfide bonds between Fc1 and Fc2. The table below provides exemplary Fc1 and Fc1 heterodimeric pairs possessing complementary sequence modifications to promote heterodimerization that may be employed in the design of the Fc1 and Fc2 polypeptides of the formulae [1] and [2].

The following Table provides exemplary Fc heterodimeric pairs which may be used in the preparation of Fc1 and Fc2 polypeptides of the heterodimeric Fcs of the present disclosure:

TABLE
Amino Acid Substitution Sets of Complementary IgG1 KiH UH/Fc Heterodimeric Pairs
Comprising Mutations to Reduce Effector Function and LS Halflife Extensions

| Fc | | |
|---|---|---|
| Dimer | UH/Fc | Amino Acid Substitution Set |
| No. | Monomer | (EU Numbering) |
| 22 | Knob | L234A/L235A/P329A/T366W/M428L/N434S/ΔK447 |
| | Hole | L234A/L235A/P329A/T366S/L368A/Y407V/M428L/N434S/ΔK447 |
| 23 | Knob | C220S/L234A/L235A/P329A/T366W/M428L/N434S/ΔK447 |
| | Hole | C220S/L234A/L235A/P329A/T366S/L368A/Y407V/M428L/N434S/ΔK447 |
| 24 | Knob | L234A/L235A/P329A/S354C/T366W/M428L/N434S/ΔK447 |
| | Hole | L234A/L235A/P329A/Y349C/T366S/L368A/Y407V/M428L/N434S/ΔK447 |
| 25 | Knob | C220S/L234A/L235A/P329A/S354C/T366W/M428L/N434S/ΔK447 |
| | Hole | C220S/L234A/L235A/P329A/Y349C/T366S/L368A/Y407V/M428L/N434S/ΔK447 |
| 26 | Knob | L234A/L235A/P329G/T366W/M428L/N434S/ΔK447 |
| | Hole | L234A/L235A/P329G/T366S/L368A/Y407V/M428L/N434S/ΔK447 |
| 27 | Knob | C220S/L234A/L235G/P329A/T366W/M428L/N434S/ΔK447 |
| | Hole | C220S/L234A/L235G/P329A/T366S/L368A/Y407V/M428L/N434S/ΔK447 |
| 28 | Knob | L234A/L235A/P329G/S354C/T366W/M428L/N434S/ΔK447 |
| | Hole | L234A/L235A/P329G/Y349C/T366S/L368A/Y407V/M428L/N434S/ΔK447 |
| 29 | Knob | C220S/L234A/L235A/P329G/S354C/T366W/M428L/N434S/ΔK447 |
| | Hole | C220S/L234A/L235A/P329G/Y349C/T366S/L368A/Y407V/M428L/N434S/ΔK447 |
| 30 | Knob | L234A/L235E/G237A/A330S/P331S/T366W/M428L/N434S/ΔK447 |
| | Hole | L234A/L235E/G237A/A330S/P331S/T366S/L368A/Y407V/M428L/N434S/ΔK447 |
| 31 | Knob | C220S L234A/L235E/G237A/A330S/P331S/T366W/M428L/N434S/ΔK447 |
| | Hole | C220S/L234A/L235E/G237A/A330S/P331S/T366S/L368A/Y407V/M428L/N434S/ΔK447 |
| 32 | Knob | L234A/L235E/G237A/A330S/P331S/S354C/T366W/M428L/N434S/ΔK447 |
| | Hole | L234A/L235E/G237A/A330S/P331S/Y349C/T366S/L368A/Y407V/M428L/N434S/ΔK447 |
| 33 | Knob | C220S/L234A/L235E/G237A/A330S/P331S/S354C/T366W/M428L/N434S/ΔK447 |
| | Hole | C220S/L234A/L235E/G237A/A330S/P331S/Y349C/T366S/L368A/Y407V/M428L/N434S/ΔK447 |
| 34 | Knob | L234F/L235E/P331S/T366W/M428L/N434S/ΔK447 |
| | Hole | L234F/L235E/P331S/T366S/L368A/Y407V/M428L/N434S/ΔK447 |
| 35 | Knob | C220S/L234F/L235E/P331S/T366W/M428L/N434S/ΔK447 |
| | Hole | C220S/L234F/L235E/P331S//L368A/Y407V/M428L/N434S/ΔK447 |
| 36 | Knob | L234F/L235E/P331S/S354C/T366W/M428L/N434S/ΔK447 |
| | Hole | Y L234F/L235E/P331S/349C/T366S/L368A/Y407V/M428L/N434S/ΔK447 |
| 37 | Knob | C220S/S/L234F/L235E/P331S/354C/T366W/M428L/N434S/ΔK447 |
| | Hole | C220S/L234F/L235E/P331S/Y349C/T366S/L368A/Y407V/M428L/N434S/ΔK447 |

In some embodiments, the Fc domains (Fc1 and Fc2) of the compositions of the present disclosure are from hIgG4. In such instances where the Fc domains of the heterodimeric Fc are derived from hIgG4, heterodimerization of the Fc1 and Fc2 domains by the introduction of the mutations K370E, K409W and E357N, D399V, F405T (EU numbering) in the complementary Fc sequences that comprise the heterodimeric Fc domain.

### Modifications to Eliminate Glycosylation Sites

In some embodiments the amino acid sequence of the Fc1 and/or Fc2 monomers modified to promote heterodimerization may be further modified to eliminate N-linked or 0-linked glycosylation sites. Aglycosylated variants of Fc domains, particularly of the IgG1 subclass are known to be poor mediators of effector function. Jefferies et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc domains of the compositions of the present disclosure comprise one or modifications to eliminate N- or O linked glycosylation sites. Examples of modifications at N297 to eliminate glycosylation sites in the Fc domain include the amino acid substitutions N297Q and N297G.

In some embodiments, when the binding protein described herein is to be administered in the format of an Fc domain fusion, particularly in those situations when the polypeptides conjugated to each Fc polypeptide of the Fc domain dimer are different, the Fc domain may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fc region (Carter, et al. (2001) Immunol Methods 248, 7-15). The knob-into-hole format is used to facilitate the expression of a first polypeptide (e.g., a first V_{H}H in a binding protein described herein) on a first Fc polypeptide with a “knob” modification and a second polypeptide (e.g., a second V_{H}H in a binding protein described herein) on the second Fc polypeptide with a “hole” modification to facilitate the expression of heterodimeric polypeptide conjugates.

In some embodiments, the binding proteins described herein can have the formats as illustrated in FIGS. 1A-1D. In one example, a first binding protein comprising an anti-IL10Rα V_{H}H antibody at the N-terminus and an anti-IL2Rγ V_{H}H antibody at the C-terminus (e.g., N-terminus-anti-IL10Rα V_{H}H antibody-linker-anti-IL2Rγ V_{H}H antibody-C-terminus) can be fused to a first Fc polypeptide, and a second binding protein comprising an anti-IL2Rγ V_{H}H antibody at the N-terminus and an anti-IL10Rα V_{H}H antibody at the C-terminus (e.g., N-terminus-anti-IL2Rγ V_{H}H antibody-linker-anti-IL10Rα V_{H}H antibody-C-terminus) can be fused to a second Fc polypeptide (FIG. TA), in which the first Fc polypeptide and the second Fc polypeptide form an Fc domain. In this example, one Fc polypeptide can comprise T366W as a knob mutation and the other Fc polypeptide can comprise T366S, L368A, Y407V as hole mutations to promote Fc heterodimer formation.

In another example, two identical binding proteins can each be conjugated to an Fc polypeptide. Two identical binding protein-Fc polypeptide conjugates can then dimerize to form a homodimer (FIG. 1B). In some embodiments, both binding proteins can have an anti-IL10Rα V_{H}H antibody at the N-terminus and an anti-IL2Rγ V_{H}H antibody at the C-terminus (e.g., N-terminus-anti-IL10Rα V_{H}H antibody-linker-anti-IL2Rγ V_{H}H antibody-C-terminus). In other embodiments, both binding proteins can have an anti-IL2Rγ V_{H}H antibody at the N-terminus and an anti-IL10Rα V_{H}H antibody at the C-terminus (e.g., N-terminus-anti-IL2Rγ V_{H}H antibody-linker-anti-IL10Rα V_{H}H antibody-C-terminus).

In yet another example, a binding protein can be conjugated to one of the two Fc polypeptides in an Fc domain (FIGS. 1C and 1D). In this case, one Fc polypeptide can comprise T366W as a knob mutation and the other Fc polypeptide can comprise T366S, L368A, Y407V as hole mutations to promoter heterodimer formation. The binding protein can have an anti-IL10Rα V_{H}H antibody at the N-terminus and an anti-IL2Rγ V_{H}H antibody at the C-terminus (e.g., N-terminus-anti-IL10Rα V_{H}H antibody-linker-anti-IL2Rγ V_{H}H antibody-C-terminus). In other embodiments, the binding protein can have an anti-IL2Rγ V_{H}H antibody at the N-terminus and an anti-IL10Rα V_{H}H antibody at the C-terminus (e.g., N-terminus-anti-IL2Rγ V_{H}H antibody-linker-anti-IL10Rα V_{H}H antibody-C-terminus).

In some embodiments, the binding protein can be conjugated to one or more water-soluble polymers, optionally comprising an intervening linker. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol), polysaccharides), poly-alpha-hydroxy acid), polyvinyl alcohol (PVA), polyphosphazene, polyoxazolines (POZ), poly(N-acryloylmorpholine), or a combination thereof.

In some embodiments, binding protein can be conjugated to one or more polyethylene glycol molecules or “PEGylated.” Although the method or site of PEG attachment to the binding protein may vary, in certain embodiments the PEGylation does not alter, or only minimally alters, the activity of the binding protein. A variety of technologies are available for site specific incorporation of PEG moieties as reviewed in Dozier, J. K. and Distefano, M. D. (2015) “Site Specific Pegylation of Therapeutic Proteins” International Journal of Molecular Science 16(10):25832-25864.

In some embodiments, selective PEGylation of the binding protein, for example, by the incorporation of non-natural amino acids having side chains to facilitate selective PEG conjugation, may be employed. Specific PEGylation sites can be chosen such that PEGylation of the binding protein does not affect its binding to the target receptors.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding protein can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding protein of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding proteins is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the binding protein and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G)n, glycine-alanine polymers, alanine-serine polymers, glycine-serine polymers (for example, (GmSo)n, (GSGGS)n, (GmSoGm)n, (GmSoGmSoGm)n, (GSGGSm)n, (GSGSmG)n and (GGGSm)n, and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers are provided in Section V.

Additional examples of flexible linkers include glycine polymers (G)n or glycine-serine polymers (e.g., (GS)n, (GSGGS)n, (GGGS)n and (GGGGS)n, where n=1 to 50, for example, 1, 2, 3, 4, 5, 6, 7, 8, 9, 10, 10-20, 20-30, 30-50). A multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding protein is acetylated at the N-terminus by enzymatic reaction with N-terminal acetyltransferase and, for example, acetyl CoA. Alternatively, or in addition to N-terminal acetylation, the binding protein can be acetylated at one or more lysine residues, e.g., by enzymatic reaction with a lysine acetyltransferase. See, for example Choudhary et al. (2009) Science 325 (5942):834-840.

In other embodiments, the binding protein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding protein further comprises a C-terminal c-myc epitope tag.

In some embodiments, the binding protein is expressed as a fusion protein with an albumin molecule (e.g., human serum albumin) which is known in the art to facilitate extended exposure in vivo.

In some embodiment, the binding proteins (including fusion proteins of the binding proteins) of the present disclosure are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986. Examples of transition metal chelating polypeptides useful in the practice of the present disclosure are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present disclosure are peptides comprising 3-6 contiguous histidine residues such as a six-histidine peptide (His)₆ and are frequently referred to in the art as “His-tags.”

The foregoing fusion proteins may be readily produced by recombinant DNA methodology by techniques known in the art by constructing a recombinant vector comprising a nucleic acid sequence comprising a nucleic acid sequence encoding the binding protein in frame with a nucleic acid sequence encoding the fusion partner either at the N-terminus or C-terminus of the binding protein, the sequence optionally further comprising a nucleic acid sequence in frame encoding a linker or spacer polypeptide.

### VII. Pharmaceutical Composition

The binding proteins of the present disclosure may be administered to a subject in a pharmaceutically acceptable dosage form. The preferred formulation depends on the intended mode of administration and therapeutic application. Pharmaceutical dosage forms of the binding proteins described herein comprise physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, polymeric amino acids, amino acid copolymers, and lipid aggregates (such as oil droplets or liposomes).

The pharmaceutical compositions may also comprise pharmaceutically-acceptable, non-toxic carriers, excipients, stabilizers, or diluents, which are defined as vehicles commonly used to formulate pharmaceutical compositions for animal or human administration. The diluent is selected so as not to affect the biological activity of the combination. Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations to be used for in vivo administration are typically sterile. Sterilization of the compositions of the present disclosure may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (Langer, Science 249: 1527, 1990 and Hanes, Advanced Drug Delivery Reviews 28: 97-119, 1997). The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Administration of a binding protein described herein may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, intranodal injection, transdermal, transmucosal, iontophoretic delivery, intralymphatic injection (Senti and Kundig (2009) Current Opinions in Allergy and Clinical Immunology 9(6):537-543), intragastric infusion, intraprostatic injection, intravesical infusion (e.g., bladder), respiratory inhalers including nebulizers, intraocular injection, intraabdominal injection, intralesional injection, intraovarian injection, intracerebral infusion or injection, intracerebroventricular injection (ICVI), and the like. In some embodiments, administration includes the administration of the binding protein itself (e.g., parenteral), as well as the administration of a recombinant vector (e.g., viral or non-viral vector) to cause the in situ expression of the binding protein in the subject. Alternatively, a cell, such as a cell isolated from the subject, could also be recombinantly modified to express the binding protein of the present disclosure.

The dosage of the pharmaceutical compositions depends on factors including the route of administration, the disease to be treated, and physical characteristics, e.g., age, weight, general health, of the subject. Typically, the amount of a binding protein contained within a single dose may be an amount that effectively prevents, delays, or treats the disease without inducing significant toxicity. A pharmaceutical composition of the disclosure may include a dosage of a binding protein described herein ranging from 0.01 to 500 mg/kg (e.g., from 0.01 to 450 mg, from 0.01 to 400 mg, from 0.01 to 350 mg, from 0.01 to 300 mg, from 0.01 to 250 mg, from 0.01 to 200 mg, from 0.01 to 150 mg, from 0.01 to 100 mg, from 0.01 to 50 mg, from 0.01 to 10 mg, from 0.01 to 1 mg, from 0.1 to 500 mg/kg, from 1 to 500 mg/kg, from 5 to 500 mg/kg, from 10 to 500 mg/kg, from 50 to 500 mg/kg, from 100 to 500 mg/kg, from 150 to 500 mg/kg, from 200 to 500 mg/kg, from 250 to 500 mg/kg, from 300 to 500 mg/kg, from 350 to 500 mg/kg, from 400 to 500 mg/kg, or from 450 to 500 mg/kg) and, in a more specific embodiment, about 1 to about 100 mg/kg (e.g., about 1 to about 90 mg/kg, about 1 to about 80 mg/kg, about 1 to about 70 mg/kg, about 1 to about 60 mg/kg, about 1 to about 50 mg/kg, about 1 to about 40 mg/kg, about 1 to about 30 mg/kg, about 1 to about 20 mg/kg, about 1 to about 10 mg/kg, about 10 to about 100 mg/kg, about 20 to about 100 mg/kg, about 30 to about 100 mg/kg, about 40 to about 100 mg/kg, about 50 to about 100 mg/kg, about 60 to about 100 mg/kg, about 70 to about 100 mg/kg, about 80 to about 100 mg/kg, or about 90 to about 100 mg/kg). In some embodiments, a pharmaceutical composition of the disclosure may include a dosage of a binding protein described herein ranging from 0.01 to 20 mg/kg (e.g., from 0.01 to 15 mg/kg, from 0.01 to 10 mg/kg, from 0.01 to 8 mg/kg, from 0.01 to 6 mg/kg, from 0.01 to 4 mg/kg, from 0.01 to 2 mg/kg, from 0.01 to 1 mg/kg, from 0.01 to 0.1 mg/kg, from 0.01 to 0.05 mg/kg, from 0.05 to 20 mg/kg, from 0.1 to 20 mg/kg, from 1 to 20 mg/kg, from 2 to 20 mg/kg, from 4 to 20 mg/kg, from 6 to 20 mg/kg, from 8 to 20 mg/kg, from 10 to 20 mg/kg, from 15 to 20 mg/kg). The dosage may be adapted by the physician in accordance with conventional factors such as the extent of the disease and different parameters of the subject.

A pharmaceutical composition containing a binding protein described herein can be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines. A course of therapy may be a single dose or in multiple doses over a period of time. In some embodiments, a single dose is used. In some embodiments, two or more split doses administered over a period of 1, 2, 3, 4, 5, 6, 7, 8, 9, 10, 11, 12, 13, 14, 21, 28, 30, 60, 90, 120 or 180 days are used. Each dose administered in such split dosing protocols may be the same in each administration or may be different. Multi-day dosing protocols over time periods may be provided by the skilled artisan (e.g., physician) monitoring the administration, taking into account the response of the subject to the treatment including adverse effects of the treatment and their modulation as discussed above. In some embodiments, the serum trough concentration of the binding molecule is maintained above a threshold level corresponding to about 0.1 pg/ml, alternatively 0.1 ng/ml, alternatively about 0.5 ng/ml alternatively 1 ng/ml, alternatively 2 ng/ml, for at least 80%, alternatively at least 85%, alternatively at least 90%, alternatively at least 95% of a period of time of at least 24 hours, alternatively 48 hours, alternatively 72 hours, alternatively one week, alternatively 1 month. See, e.g. Mumm, et al. United States Patent Publication US2016/0193300A1 published Jul. 7, 2016.

### VIII. Methods of Use

### Neoplastic Diseases

The present disclosure provides methods of use of binding proteins that bind to IL10Rα and IL2Rγ in the treatment of subjects suffering from a neoplastic disease, disorder, or condition by the administration of a therapeutically effective amount of a binding protein (or nucleic acid encoding a binding protein including recombinant vectors encoding the binding protein) as described herein. IL10 agonists have been identified as useful in the treatment of neoplastic disease as described in Oft, M. (2014) Cancer Immunology Research 2(3):194-199; Naing, et al. (2108) Cancer Cell 34(5):775-791; and Mumm, J. and Oft, M (2013) Bioessays 35(7):623-631.

The compositions and methods of the present disclosure are useful in the treatment of subject suffering from a neoplastic disease characterized by the presence neoplasms, including benign and malignant neoplasms, and neoplastic disease. In certain embodiments, the method does not cause anemia.

Examples benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including, but not limited to, breast cancers, sarcomas (including but not limited to osteosarcomas and angiosarcomas and fibrosarcomas), leukemias, lymphomas, genitourinary cancers (including but not limited to ovarian, urethral, bladder, and prostate cancers), gastrointestinal cancers (including but not limited to colon esophageal and stomach cancers), lung cancers, myelomas, pancreatic cancers, liver cancers, kidney cancers, endocrine cancers, skin cancers, and brain or central and peripheral nervous (CNS) system tumors, malignant or benign, including gliomas and neuroblastomas, astrocytomas, myelodysplastic disorders, cervical carcinoma-in-situ, intestinal polyposes, oral leukoplakias, histiocytoses, hyperprofroliferative scars including keloid scars, hemangiomas, hyperproliferative arterial stenosis, psoriasis, inflammatory arthritis, hyperkeratoses, and papulosquamous eruptions including arthritis.

The term “neoplastic disease” includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML), and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL), and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease, and Reed-Steinberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

### Assessing Anti-Neoplastic Efficacy

The determination of efficacy of the methods of the present disclosure in the treatment of cancer is generally associated with the achievement of one or more art recognized parameters such as reduction in lesions particularly reduction of metastatic lesion, reduction in metastatsis, reduction in tumor volume, improvement in ECOG score, and the like. Determining response to treatment can be assessed through the measurement of biomarker that can provide reproducible information useful in any aspect of binding protein therapy, including the existence and extent of a subject's response to such therapy and the existence and extent of untoward effects caused by such therapy. By way of example, but not limitation, biomarkers include enhancement of IFNγ, and upregulation of granzyme A, granzyme B, and perforin; increase in CD8⁺ T-cell number and function; enhancement of IFNγ, an increase in ICOS expression on CD8⁺ T-cells, enhancement of IL10 expressing T_{Reg} cells. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g., human) subjects.

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of a binding protein described herein and a supplementary agent which is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

Combination of Binding Proteins with Supplementary Therapeutic Agents

The present disclosure provides the for the use of the binding proteins of the present disclosure in combination with one or more additional active agents (“supplementary agents”). Such further combinations are referred to interchangeably as “supplementary combinations” or “supplementary combination therapy” and those therapeutic agents that are used in combination with binding proteins of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the binding proteins.

As used herein, the term “in combination with” when used in reference to the administration of multiple agents to a subject refers to the administration of a first agent at least one additional (i.e. second, third, fourth, fifth, etc.) agent to a subject. For purposes of the present invention, one agent (e.g., a binding protein described herein) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by IV infusion every two weeks or every three weeks while the binding proteins of the present disclosure are typically administered more frequently, e.g. daily, BID, or weekly. However, the administration of the first agent (e.g. pembrolizumab) provides a therapeutic effect over an extended time and the administration of the second agent (e.g., a binding protein described herein) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the binding protein and the supplementary agent(s) are administered or applied sequentially, e.g., where one agent is administered prior to one or more other agents. In other embodiments, the binding protein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

### Chemotherapeutic Agents

In some embodiments, the supplementary agent is a chemotherapeutic agent. In some embodiments the supplementary agent is a “cocktail” of multiple chemotherapeutic agents. The use of IL-10 agents in combination with chemotherapeutic agents is described in Oft, et al., U.S. Pat. No. 9,833,514B2 issued Dec. 5, 2017, the teaching of which is herein incorporated by reference. In some embodiments the chemotherapeutic agent or cocktail is administered in combination with one or more physical methods (e.g., radiation therapy). The term “chemotherapeutic agents” includes but is not limited to alkylating agents such as thiotepa and cyclosphosphamide, alkyl sulfonates such as busulfan, improsulfan and piposulfan, aziridines such as benzodopa, carboquone, meturedopa, and uredopa, ethylenimines and methylamelamines including altretamine, triethylenemelamine, trietylenephosphoramide, triethylenethiophosphaoramide and trimethylolomelamime, nitrogen mustards such as chiorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas such as carmustine, chlorozotocin, fotemustine, lomustine, nimustine, ranimustine, antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, anti-metabolites such as methotrexate and 5-fluorouracil (5-FU), folic acid analogues such as denopterin, methotrexate, pteropterin, trimetrexate, dideazatetrahydrofolic acid, and folinic acid, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, 5-FU, androgens such as calusterone, dromostanolone propionate, epitiostanol, mepitiostane, testolactone, anti-adrenals such as aminoglutethimide, mitotane, trilostane, folic acid replenisher such as frolinic acid, aceglatone, aldophosphamide glycoside, aminolevulinic acid, amsacrine, bestrabucil, bisantrene, edatraxate, defofamine, demecolcine, diaziquone, elformithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, arabinoside (Ara-C), cyclophosphamide, thiotepa, taxoids, e.g., paclitaxel, nab-paclitaxel and doxetaxel, chlorambucil, gemcitabine, 6-thioguanine, mercaptopurine, methotrexate, platinum and platinum coordination complexes such as cisplatin, oxaplatin and carboplatin, vinblastine, etoposide (VP-16), ifosfamide, mitomycin C, mitoxantrone, vincristine, vinorelbine, navelbine, novantrone, teniposide, daunomycin, aminopterin, xeloda, ibandronate, CPT11, topoisomerase inhibitors, difluoromethylornithine (DMFO), retinoic acid, esperamicins, capecitabine, taxanes such as paclitaxel, docetaxel, cabazitaxel, carminomycin, adriamycins such as 4′-epiadriamycin, 4-adriamycin-14-benzoate, adriamycin-14-octanoate, adriamycin-14-naphthaleneacetate, cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is one or more chemical or biological agents identified in the art as useful in the treatment of neoplastic disease, including, but not limited to, a cytokines or cytokine antagonists such as IL12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the binding protein is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

Combination with Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs). The use of IL10 agents in combination with therapeutic antibodies in the treatment of neoplastic diseases is described in Mumm, et al., U.S. Pat. No. 10,618,970B2 issued Apr. 14, 2020.

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Le^{y}, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplementary agents for use in the treatment of neoplastic disease indication include those provided in Table 5 below.

TABLE 5
FDA Antineoplastic Disease Antibodies and Indications

| Name | Tradename(s) | Target; format | Indication |
|---|---|---|---|
| [fam]- | Enhertu | HER2; Humanized IgG1 | HER2⁺ breast cancer |
| trastuzumab | | ADC | |
| deruxtecan | | | |
| Enfortumab | Padcev | Nectin-4; Human IgG1 | Urothelial cancer |
| vedotin | | ADC | |
| Polatuzumab | Polivy | CD79b; Humanized IgG1 | Diffuse large B-cell |
| vedotin | | ADC | lymphoma |
| Cemiplimab | Libtayo | PD-1; Human mAb | Cutaneous squamous cell |
| | | | carcinoma |
| Moxetumomab | Lumoxiti | CD22; Murine IgG1 dsFv | Hairy cell leukemia |
| pasudotox | | immunotoxin | |
| Mogamuizumab | Poteligeo | CCR4; Humanized IgG1 | Cutaneous T cell lymphoma |
| Tildrakizumab | Ilumya | IL23p19; Humanized IgG1 | Plaque psoriasis |
| Ibalizumab | Trogarzo | CD4; Humanized IgG4 | HIV infection |
| Durvalumab | IMFINZI | PD-L1; Human IgG1 | Bladder cancer |
| Inotuzumab | BESPONSA | CD22; Humanized IgG4, | Hematological malignancy |
| ozogamicin | | ADC | |
| Avelumab | Bavencio | PD-L1; Human IgG1 | Merkel cell carcinoma |
| Atezolizumab | Tecentriq | PD-L1; Humanized IgG1 | Bladder cancer |
| Olaratumab | Lartruvo | PDGRFα; Human IgG1 | Soft tissue sarcoma |
| Ixekizumab | Taltz | IL17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Nivolumab | Opdivo | PD1; Human IgG4 | Melanoma, non-small cell |
| | | | lung cancer |
| Blinatumomab | Blincyto | CD19, CD3; Murine | Acute lymphoblastic leukemia |
| | | bispecific tandem scFv | |
| Pembrolizumab | Keytruda | PD1; Humanized IgG4 | Melanoma |
| Ramucirumab | Cyramza | VEGFR2; Human IgG1 | Gastric cancer |
| Siltuximab | Sylvant | IL6; Chimeric IgG1 | Castleman disease |
| Obinutuzumab | Gazyva | CD20; Humanized IgG1; | Chronic lymphocytic |
| | | Glycoengineered | leukemia |
| Ado-trastuzumab | Kadcyla | HER2; Humanized IgG1, | Breast cancer |
| emtansine | | ADC | |
| Pertuzumab | Perjeta | HER2; Humanized IgG1 | Breast Cancer |
| Brentuximab | Adcetris | CD30; Chimeric IgG1, ADC | Hodgkin lymphoma, systemic |
| vedotin | | | anaplastic large cell |
| | | | lymphoma |
| Ipilimumab | Yervoy | CTLA-4; Human IgG1 | Metastatic melanoma |
| Ofatumumab | Arzerra | CD20; Human IgG1 | Chronic lymphocytic |
| | | | leukemia |
| Certolizumab | Cimzia | TNF; Humanized Fab, | Crohn disease |
| pegol | | pegylated | |
| Catumaxomab | Removab | EPCAM/CD3; Rat/mouse | Malignant ascites |
| | | bispecific mAb | |
| Panitumumab | Vectibix | EGFR; Human IgG2 | Colorectal cancer |
| Bevacizumab | Avastin | VEGF; Humanized IgG1 | Colorectal cancer |
| Cetuximab | Erbitux | EGFR; Chimeric IgG1 | Colorectal cancer |
| Tositumomab- | Bexxar | CD20; Murine IgG2a | Non-Hodgkin lymphoma |
| I131 | | | |
| Ibritumomab | Zevalin | CD20; Murine IgG1 | Non-Hodgkin lymphoma |
| tiuxetan | | | |
| Gemtuzumab | Mylotarg | CD33; Humanized IgG4, | Acute myeloid leukemia |
| ozogamicin | | ADC | |
| Trastuzumab | Herceptin | HER2; Humanized IgG1 | Breast cancer |
| Infliximab | Remicade | TNF; Chimeric IgG1 | Crohn disease |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

In some embodiments, where the antibody is a bispecific antibody targeting a first and second tumor antigen such as HER2 and HER3 (abbreviated HER2 x HER3), FAP x DR-5 bispecific antibodies, CEA x CD3 bispecific antibodies, CD20 x CD3 bispecific antibodies, EGFR-EDV-miR16 trispecific antibodies, gp100 x CD3 bispecific antibodies, Ny-eso x CD3 bispecific antibodies, EGFR x cMet bispecific antibodies, BCMA x CD3 bispecific antibodies, EGFR-EDV bispecific antibodies, CLEC12A x CD3 bispecific antibodies, HER2 x HER3 bispecific antibodies, Lgr5 x EGFR bispecific antibodies, PD1 x CTLA-4 bispecific antibodies, CD123 x CD3 bispecific antibodies, gpA33 x CD3 bispecific antibodies, B7-H3 x CD3 bispecific antibodies, LAG-3 x PD1 bispecific antibodies, DLL4 x VEGF bispecific antibodies, Cadherin-P x CD3 bispecific antibodies, BCMA x CD3 bispecific antibodies, DLL4 x VEGF bispecific antibodies, CD20 x CD3 bispecific antibodies, Ang-2 x VEGF-A bispecific antibodies,

CD20 x CD3 bispecific antibodies, CD123 x CD3 bispecific antibodies, SSTR2 X CD3 bispecific antibodies, PD1 x CTLA-4 bispecific antibodies, HER2 x HER2 bispecific antibodies, GPC3 x CD3 bispecific antibodies, PSMA x CD3 bispecific antibodies, LAG-3 x PD-L1 bispecific antibodies, CD38 x CD3 bispecific antibodies, HER2 x CD3 bispecific antibodies, GD2 x CD3 bispecific antibodies, and CD33 x CD3 bispecific antibodies.

Such therapeutic antibodies may be further conjugated to one or more chemotherapeutic agents (e.g., antibody drug conjugates or ADCs) directly or through a linker, especially acid, base or enzymatically labile linkers.

Combination with Physical Methods

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising a binding protein and one or more supplementary agents. In some embodiments, the present disclosure further contemplates the use of a binding protein in combination with surgery (e.g. tumor resection). In some embodiments, the present disclosure further contemplates the use of a binding protein in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The use of IL10 agents in combination with immune checkpoint modulators in the treatment of neoplastic disease is described in Oft, United States Patent Publication US2020/0353050 published Nov. 12, 2020. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” The term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the binding protein is a positive immune checkpoint pathway agonist. In another embodiment, immune checkpoint pathway modulator employed in combination with the binding protein is an immune checkpoint pathway antagonist.

The term “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130.).

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1 VISTA or PD-L1/Tim3 for cancer therapy. [abstract]. In: Proceedings of the 107th Annual Meeting of the American Association for Cancer Research; 2016 Apr. 16-20; New Orleans, LA. Philadelphia (PA): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2,4-oxadiazole and thiadiazole PCT/IB2016/051343 filed Mar. 9, 2016 and published as WO2016142886A2), BMS-1166 and Chupak LS and Zheng X. Compounds useful as immunomodulators. Bristol-Myers Squibb Co. (2015) WO 2015/034820 A1, EP3041822 B1 granted Aug. 9, 2017; WO2015034820 A1; and Chupak, et al. Compounds useful as immunomodulators. Bristol-Myers Squibb Co. (2015) WO 2015/160641 A2. WO 2015/160641 A2, Chupak, et al. Compounds useful as immunomodulators. Bristol-Myers Squibb Co. Sharpe, et al. Modulators of immunoinhibitory receptor PD-1, and methods of use thereof, WO 2011082400 A2 published Jul. 7, 2011; U.S. Pat. No. 7,488,802 (Wyeth) issued Feb. 10, 2009;

In some embodiments, combination of binding proteins described herein and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of binding proteins and an PD1 immune checkpoint modulator is useful in the treatment of tumors characterized by high levels of expression of PDL1, where the tumor has a tumor mutational burden, where there are high levels of CD8⁺ T-cell in the tumor, an immune activation signature associated with IFNγ and the lack of metastatic disease particularly liver metastasis.

In some embodiments, the binding protein is administered in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, the binding protein is administered in combination with an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J Biol. Sci. 8:1420-30).

In some embodiments, the binding protein is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 A1 published Sep. 8, 2016 (F. Hoffman-LaRoche), Karunsky, U.S. Pat. No. 9,631,026 issued Apr. 27, 2017; Karunsky, Sabatos-Peyton, et al. U.S. Pat. No. 8,841,418 isued Sep. 23, 2014; U.S. Pat. No. 9,605,070; Takayanagi, et al., U.S. Pat. No. 8,552,156 issued Oct. 8, 2013.

In some embodiments, the binding protein is administered in combination with an inhibitor of both LAG3 and PD1 as the blockade of LAG3 and PD1 has been suggested to synergistically reverse anergy among tumor-specific CD8⁺ T-cells and virus-specific CD8⁺ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392.

In some embodiments, the binding protein is administered in combination with an A2aR inhibitor. A2aR inhibits T-cell responses by stimulating CD4⁺ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the binding proteins for use in the treatment disorders such as cancer and Parkinson's disease.

In some embodiments, the binding protein is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g., cancer) in a mammalian subject by the administration of a binding protein in combination with an agent(s) that modulate at least one immune checkpoint pathway including immune checkpoint pathway modulators that modulate two, three or more immune checkpoint pathways.

In some embodiments the binding protein is administered in combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of binding proteins in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the binding protein may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a binding protein of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### Cell Therapy Agents and Methods as Supplementary Agents

In some embodiments, the methods of the disclosure may include the combination of the administration of a binding protein with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of a CD25 biased binding protein as described herein.

### CAR-T Cells

In some embodiments of the methods of the present disclosure, the supplementary agent is a “chimeric antigen receptor T-cell” and “CAR-T cell” are used interchangeably to refer to a T-cell that has been recombinantly modified to express a chimeric antigen receptor. The use of IL10 agents in combination with CAR-T cells for the treatment of neoplastic disease is described in Mumm, et al., U.S. Pat. No. 10,195,274 issued Feb. 5, 2019. As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more cell surface molecules (e.g. tumor antigens) expressed on the surface of a target cell. In some embodiments, the ABD is a polypeptide that specifically binds to a cell surface molecule associated with a tumor cell is selected from the group consisting of GD2, BCMA, CD19, CD33, CD38, CD70, GD2, IL3Rα2, CD19, mesothelin, Her2, EpCam, Mucd, ROR1, CD133, CEA, EGRFRVIII, PSCA, GPC3, Pan-ErbB and FAP. In some embodiments, the ABD is an antibody (as defined hereinabove to include molecules such as one or more VHHs, scFvs, etc.) that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Rα2, CD19, mesothelin, Her2, EpCam, Mucd, ROR1, CD133, CEA, EGRFRVIII, PSCA, GPC3, Pan-ErbB and FAP. Examples of CAR-T cells useful as supplementary agents in the practice of the methods of the present disclosure include but are not limited to CAR-T cells expressing CARs comprising an ABD further comprising at least one of: anti-GD2 antibodies, anti-BCMA antibodies, anti-CD19 antibodies, anti-CD33 antibodies, anti-CD38 antibodies, anti-CD70 antibodies, anti-GD2 antibodies and IL3Rα2 antibodies, anti-CD19 antibodies, anti-mesothelin antibodies, anti-Her2 antibodies, anti-EpCam antibodies, anti-Muc antibodies, anti-ROR1 antibodies, anti-CD133 antibodies, anti-CEA antibodies, anti-PSMA antibodies, anti-EGRFRVIII antibodies, anti-PSCA antibodies, anti-GPC3 antibodies, anti-Pan-ErbB antibodies, anti-FAP antibodies,

CARs of CAR-T cells useful in the practice of the methods of the present disclosure further comprise a transmembrane domain joining the ABD (or linker, if employed, see discussion of linkers below) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

In some embodiments, the CAR may also provide a co-stimulatory domain. The term “co-stimulatory domain” (“CSD”) refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved are described in Chen & Flies (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs useful in the practice of the methods of the present disclosure may optionally include one or more polypeptide spacers linking the domains of the CAR, in particular the linkage between the ABD to the transmembrane spanning domain of the CAR. Although not an essential element of the CAR structure, the inclusion of a spacer domain is generally considered desirable to facilitate antigen recognition by the ARD. As used in conjunction with the CAR-T cell technology described herein, the terms “linker”, “linker domain” and “linker region” refer to a polypeptide from about 1 to 100 amino acids in length. Linkers are typically be composed of amino acid residues which permit flexibility of the polypeptide (e.g. glycine and serine) so that the adjacent domains of the CAR are provided greater freedom of movement relative to one another. Although there is no particularly defined length or sequence of amino acids that is necessary for the spacer to achieve its function, but the typical properties of the spacer are flexibility to enable freedom of movement of the ABD to facilitate targeting antigen recognition. Similarly, it has been found that there is there is substantial leniency in spacer length while retaining CAR function. Jensen and Riddell (2014) Immunol. Review 257(1) 127-144. Sequences useful as spacers in the construction of CARs useful in the practice of the present invention include but are not limited to the hinge region of IgG1, the immunoglobulin 1 CH2-CH3 region, IgG4 hinge-CH2-CH3, IgG4 hinge-CH3, and the IgG4 hinge. The hinge and transmembrane domains may be derived from the same molecule such as the hinge and transmembrane domains of CD8-alpha. Imai, et al. (2004) Leukemia 18(4):676-684.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1γ or the CD3 chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3 (signal. Coincidental delivery of the co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3, OX40 or 4-1BB signaling region. In fourth generation, or “armored car” CAR T-cells are further modified to express or block molecules and/or receptors to enhance immune activity such as the expression of IL12, IL18, IL7, and/or IL10; 4-1BB ligand, CD-40 ligand. Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (iPS) CAR-T cells. The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. The term “bispecific or tandem CARs” refers to CARs which include a secondary CAR binding domain that can either amplify or inhibit the activity of a primary CAR. The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility. The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

In some embodiments, the engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

Because the cell product may consist of a subject's own T-cells, the population of the cells to be administered is to the subject is necessarily variable. Consequently, identifying the optimal concentration of the CAR-T cell will be optimized by the caregiver in accordance with the needs of the subject to be treated and monitored by conventional laboratory testing. Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. The engineered cells may be infused to the subject in any physiologically acceptable medium by any convenient route of administration, normally intravascularly, although they may also be introduced by other routes, where the cells may find an appropriate site for growth

If the T cells used in the practice of the present invention are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

### Chemokine and Cytokine Agents as Supplementary Agents:

In some embodiments the binding protein is administered in combination with additional cytokines including but not limited to IL2, IL7, IL12, IL15 (See U.S. Pat. No. 10,398,761 issued Sep. 13, 2019) and IL18 including analogs and variants of each thereof.

### Activation-Induced Cell Death Inhibitors

In some embodiments the binding protein is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the binding proteins described herein include but are not limited to cyclosporin A (Shih, et al., (1989) Nature 339:625-626, IL16 and analogs (including rhIL16, Idziorek, et al., (1998) Clinical and Experimental Immunology 112:84-91), TGFb1 (Genesteir, et al., (1999) J Exp Med 189(2): 231-239), and vitamin E (Li-Weber, et al., (2002) J Clin Investigation 110(5):681-690).

### Physical Methods

In some embodiments, the supplementary agent is an anti-neoplastic physical methods including but not limited to radiotherapy, cryotherapy, hyperthermic therapy, surgery, laser ablation, and proton therapy.

### Immune Diseases

The present disclosure further provides methods of treating a subject suffering from a disease, disorder, or condition by the administration of a therapeutically effective amount of an IL10Rα/IL2Rγ binding protein (or nucleic acid encoding an IL10Rα/IL2Rγ binding protein including recombinant viruses encoding the IL10Rα/IL2Rγ binding protein) of the present disclosure. Disorders amenable to treatment with IL10Rα/IL2Rγ binding proteins (including pharmaceutically acceptable formulations comprising IL10Rα/IL2Rγ binding proteins and/or the nucleic acid molecules that encode them including recombinant viruses encoding such IL10Rα/IL2Rγ binding proteins) of the present disclosure include inflammatory or autoimmune diseases including but not limited to, viral infections (e.g., AIDS, influenza, chronic HCV, chronic viral hepatitis B, C or D), Heliobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome (Seronegativity, Enthesopathy, Arthropathy Syndrome). In certain embodiments, the method does not cause anemia.

Other examples of proliferative and/or differentiative disorders amenable to treatment with IL10Rα/IL2Rγ binding proteins (including pharmaceutically acceptable formulations comprising IL10Rα/IL2Rγ binding proteins and/or the nucleic acid molecules that encode them including recombinant viruses encoding such IL10Rα/IL2Rγ binding proteins) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising IL10Rα/IL2Rγ binding proteins and/or the nucleic acid molecules that encode them including recombinant viruses encoding such IL10Rα/IL2Rγ binding proteins) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

Combination of IL10Rα/IL2Rγ Binding Proteins with Additional Therapeutic Agents for Autoimmune Disease:

The present disclosure provides the for the use of the IL10Rα/IL2Rγ binding proteins of the present disclosure in combination with one or more additional active agents (“supplementary agents”) in the treatment of autoimmune disease. As used herein, the term “supplementary agents” includes agents that can be administered or introduced separately, for example, formulated separately for separate administration (e.g., as may be provided in a kit) and/or therapies that can be administered or introduced in combination with the IL10Rα/IL2Rγ binding proteins.

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g., IL10Rα/IL2Rγ binding protein) is considered to be administered in combination with a second agent (e.g. a therapeutic autoimmune antibody such as Humira®) if the biological effect resulting from the administration of the first agent persists in the subject at the time of administration of the second agent such that the therapeutic effects of the first agent and second agent overlap. For example, the therapeutic antibodies are sometimes administered by IV infusion every two weeks (e.g. adalimumab in the treatment of Crohn's disease) while the IL10Rα/IL2Rγ binding proteins of the present disclosure may be administered more frequently, e.g. daily, BID, or weekly. However, the administration of the first agent (e.g. entaercept) provides a therapeutic effect over an extended time and the administration of the second agent (e.g. an IL10Rα/IL2Rγ binding protein) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IL10Rα/IL2Rγ binding protein and the supplementary agent(s) are administered or applied sequentially, e.g., where one agent is administered prior to one or more other agents. In other embodiments, the IL10Rα/IL2Rγ binding protein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

In some embodiments, the supplementary agent is one or more agents selected from the group consisting of corticosteroids (including but not limited to prednisone, budesonide, prednilisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies. Examples of therapeutic antibodies that may be administered as supplementary agents in combination with the IL10Rα/IL2Rγ binding proteins of the present disclosure in the treatment of autoimmune disease include but are not limited to anti-CD25 antibodies (e.g. daclizumab and basiliximab), anti-VLA-4 antibodies (e.g. natalizumab), anti-CD52 antibodies (e.g. alemtuzumab), anti-CD20 antibodies (e.g. rituximab, ocrelizumab), anti-TNF antibodies (e.g. infliximab, and adalimumab), anti-IL6R antibodies (e.g. tocilizumab), anti-TNFα antibodies (e.g. adalimumab (Humira®), golimumab, and infliximab), anti-integrin-α4β7 antibodies (e.g. vedolizumab), anti-IL17a antibodies (e.g. brodalumab or secukinumab), anti-IL4Rα antibodies (e.g. dupilumab), anti-RANKL antibodies, IL6R antibodies, anti-IL1β antibodies (e.g. canakinumab), anti-CD11a antibodies (e.g. efalizumab), anti-CD3 antibodies (e.g. muramonab), anti-IL5 antibodies (e.g. mepolizumab, reslizumab), anti-BLyS antibodies (e.g. belimumab); and anti-IL12/IL23 antibodies (e.g ustekinumab).

Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplementary agents in combination with the IL10Ra/IL2Rγ binding proteins of the present disclosure (and optionally additional supplementary agents) for the treatment of the indicated autoimmune disease are provided in Table 6.

TABLE 6

| Name | Target | Indication |
|---|---|---|
| belimumab | BLyS | Systemic lupus erythematosus |
| efalizumab | CD11a | Psoriasis |
| ocrelizumab | CD20 | Multiple sclerosis |
| rituximab | CD20 | Multiple sclerosis |
| basiliximab | CD25 | Transplantation rejection |
| daclizumab | CD25 | Transplantation rejection |
| muromonab | CD3 | Transplantation rejection |
| alemtuzumab | CD52 | Multiple sclerosis |
| omalizumab | IgE | Asthma |
| ustekinumab | IL12/IL23 | Plaque psoriasis |
| brodalumab | IL17a | Psoriasis, psoriatic arthritis, ankylosing spondylitis |
| secukinumab | IL17a | Psoriasis, psoriatic arthritis, ankylosing spondylitis |
| ixekizumab | IL17a | Psoriasis, psoriatic arthritis, ankylosing spondylitis |
| canakinumab | IL1ß | Cryopyrin-associated periodic syndrome, tumor necrosis factor |
| | | receptor associated periodic syndrome, hyperimmunoglobulin D |
| | | syndrome, mevalonate kinase deficiency, familial Mediterranean |
| | | fever, rheumatoid arthritis |
| dupilumab | IL4Rα | Asthma, dermatitis |
| mepolizumab | IL5 | Asthma |
| reslizumab | IL5 | Asthma |
| tocilizumab | IL6R | Rheumatoid arthritis |
| vedolizumab | Integrin-α4β7 | Ulcerative colitis, Crohn's disease |
| denosumab | RANKL | Osteoporosis |
| certolizumab | TNFa | Chron's disease, rheumatoid arthritis |
| golimumab | TNFa | Rheumatoid arthritis, psoriatic arthritis, ankylosing spondylitis |
| adalimumab | TNFα | Rheumatoid arthritis, juvenile idiopathic arthritis, psoriatic |
| | | arthritis, ankylosing spondylitis, Crohn's disease, plaque |
| | | psoriasis |
| infliximab | TNFα | Crohn's disease, ulcerative colitis, rheumatoid arthritis, |
| | | ankylosing spondylitis, psoriatic arthritis, plaque psoriasis |
| ranibizumab | VEGF-A | Neovascular age-related macular degeneration, macular edema |
| natalizumab | VLA-4 | Multiple sclerosis, relapsing rultiple sclerosis, Crohn's disease |

The foregoing antibodies useful as supplementary agents in the practice of the methods of the present disclosure may be administered alone or in the form of any antibody drug conjugate (ADC) comprising the antibody, linker, and one or more drugs (e.g. 1, 2, 3, 4, 5, 6, 7, or 8 drugs) or in modified form (e.g. PEGylated).

In some embodiments the supplementary agent is a vaccine. The IL10Rα/IL2Rγ binding proteins of the present invention may be administered to a subject in combination with vaccines as an adjuvant to enhance the immune response to the vaccine in accordance with the teaching of Doyle, et al U.S. Pat. No. 5,800,819 issued Sep. 1, 1998. Examples of vaccines that may be combined with the IL10Rα/IL2Rγ binding proteins of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Gardasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

### Selective Activation

It is known that IL10 has activities on macrophages (e.g., monocytes) and T cells (e.g., CD4⁺ T cells and CD8⁺ T cells). In some embodiments, the method provided herein uses a binding protein of the present disclosure that binds to IL10Rα and IL2Rγ resulting in the selective activation of T cells relative to activation of macrophages. Macrophages is a cell type that expresses both IL10Rα and IL10Rβ receptors but when activated too potently can cause side effects such as anemia. The selective activation of T cells relative to macrophages is beneficial because IL10-activated macrophages can phagocytose aging red blood cells, which manifests itself as anemia in a patient receiving IL10. Binding proteins as described herein that provide for the selective substantial activation of T cells while providing a minimal activation of macrophages can result in a molecule that produces lower side effects, such as anemia, relative to the native IL10 ligand. Other problems and toxicities related to IL10 activation are described in, e.g., Fioranelli and Grazia, J Integr Cardiol 1(1):2-6, 2014. Such problems can be avoided by using a binding protein of the present disclosure that specifically binds to IL10Rα and IL2Rγ.

In some embodiments, provided herein are methods to selectively induce activity in one or more of a first cell type over one or more of a second cell type by contacting a population of cells comprising both the first and second cell types with an IL10Rα/IL2Rγ binding protein described herein. In particular embodiments, the first cell type is CD4⁺ T cells, CD8⁺ T cells, B cells, and/or NK cells and the second cell type is monocytes. In other embodiments, the first cell type is CD4⁺ T cells and/or CD8⁺ T cells and the second cell type is NK cells, B cells, and/or monocytes. In certain embodiments, the activity of the first cell type induced by an IL10Rα/IL2Rγ is at least 1.2 fold more than the activity of the second cell type.

### Dosage

Dosage, toxicity and therapeutic efficacy of such binding proteins or nucleic acids compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with minimal acceptable toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 as determined in cell culture. Such information can be used to more accurately determine useful doses in humans. Levels in plasma may be measured, for example, by high performance liquid chromatography.

As defined herein, a therapeutically effective amount of a subject binding protein (i.e., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered.

In some embodiments, the pharmaceutically acceptable forms of the binding proteins of the present disclosure are administered to a subject in accordance with a “low-dose” treatment protocol as described in Klatzman, et al. U.S. Pat. Nos. 9,669,071 and 10,293,028B2 the entire teachings of which are herein incorporated by reference. Additional low dose protocols are described in Smith, K. A. (1993) Blood 81(6):1414-1423, He, et al., (2016) Nature Medicine 22(9): 991-993

In some embodiments of the present disclosure provides methods and compositions for the treatment and/or prevention of neoplastic diseases, disorders or conditions in a subject by the administration to the subject a therapeutically effective amount of a binding protein of the present disclosure wherein the serum concentration of is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the effective concentration of the binding protein sufficient to promote proliferation of CD3-activated primary human T-cells (e.g., at or above EC₃₀^{PRO}, alternatively at or above EC₂₀^{PRO}, alternatively at or above EC₃₀^{PRO}, alternatively at or above EC₄₀^{PRO}, at or above EC₅₀^{PRO}, alternatively at or above EC₆₀^{PRO}) with respect to such binding protein but at a serum concentration at or below of the effective concentration at a serum concentration of such binding protein sufficient to induce activation of T-cells (e.g., at or below EC₁₀₀^{PRO}, alternatively at or below EC₉₀^{PRO}, alternatively at or below EC₈₀^{PRO}, alternatively at or below EC₇₀^{PRO}, at or below EC₆₀^{PRO}, alternatively at or below EC₅₀^{PRO}) with respect to such binding protein.

In some embodiments of the present disclosure provides methods and compositions for the treatment and/or prevention of neoplastic diseases, disorders or conditions in a subject by the administration to the subject a therapeutically effective amount of a binding protein described herein sufficient to maintain a serum concentration of the binding protein for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer.

In some embodiments of the present disclosure provides methods and compositions for the treatment and/or prevention of neoplastic diseases, disorders or conditions in a subject by the administration to the subject a therapeutically effective amount of a binding protein sufficient to maintain a serum concentration of the binding protein at or above the effective concentration for more than about 50%, alternatively greater than about 60%, alternatively greater than about 70%, alternatively greater than about 80%, alternatively greater than about 90%) of a period of time of at least 24 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer.

In accordance with another aspect, there is provided a method for stimulating the immune system of an animal by administering the binding proteins of the present disclosure. The method is useful to treat disease states where the host immune response is deficient. In treating a subject, a therapeutically effective dose of compound (i.e., active ingredient) is administered. A therapeutically effective dose refers to that amount of the active ingredient that produces amelioration of symptoms or a prolongation of survival of a subject. An effective dose will vary with the characteristics of the binding protein to be administered, the physical characteristics of the subject to be treated, the nature of the disease or condition, and the like. A single administration can range from about 50,000 IU/kg to about 1,000,000 IU/kg or more, more typically about 600,000 IU/kg. This may be repeated several times a day (e.g., 2-3 times per day) for several days (e.g., about 3-5 consecutive days) and then may be repeated one or more times following a period of rest (e.g., about 7-14 days). Thus, an effective dose may comprise only a single administration or many administrations over a period of time (e.g., about 20-30 individual administrations of about 600,000 IU/kg each given over about a 10-20 day period).

The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the binding proteins can include a single treatment or, can include a series of treatments. In one embodiment, the compositions are administered every 8 hours for five days, followed by a rest period of 2 to 14 days, e.g., 9 days, followed by an additional five days of administration every 8 hours. In another embodiment, the compositions are administered every other day for a period of at least 6 days, optionally at least 10 days, optionally at least 14 days, optionally at least 30 days, optionally at least 60 days.

The pharmaceutical compositions can be included in a container, pack, or dispenser together with instructions for administration.

While compounds that exhibit toxic side effects may be used, care should be taken to design a delivery system that targets such compounds to the site of affected tissue in order to minimize potential damage to uninfected cells and, thereby, reduce side effects. Toxicity and therapeutic efficacy of a binding protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LC₅₀/EC₅₀. Binding proteins that exhibit large therapeutic indices are preferred. The data obtained from these cell culture assays and animal studies can be used in formulating a range of dosages suitable for use in humans. The dosage of such mutants lies preferably within a range of circulating concentrations that include the ED₅₀ with little or no toxicity. The dosage may vary within this range depending upon a variety of factors, e.g., the dosage form employed, the route of administration utilized, the condition of the subject, and the like.

A therapeutically effective dose can be estimated initially from cell culture assays by determining an EC₅₀. A dose can then be formulated in animal models to achieve a circulating plasma concentration range that includes the EC₅₀ as determined in cell culture. Such information can be used to more accurately determine useful doses in humans. Levels in plasma may be measured, for example, by HPLC. The exact formulation, route of administration and dosage can be chosen by the individual physician in view of the patient's condition.

The attending physician for patients treated with binding proteins of the present disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### EXAMPLES

### Example 1—pSTAT3 Flow Cytometric Assay

PBMCs were purified from healthy non-smoking donor blood collected in Leukoreduction system chambers using the human Miltenyi MACSprep PBMC isolation kit. The purified PBMCs (500,000 cells per well) were either left unstimulated or were stimulated with 100 nM concentration of WT IL10 or one of the 84 anti-IL10R1/IL2Rγ V_{H}H² for 20 min at 37° C. The cells were fixed with Fix Buffer I (commercially available from BD Biosciences, San Jose CA as Cat #557870) for 15 mins at 37° C. The cells were then washed and permeabilized with chilled Perm Buffer III (commercially available from BD Biosciences, Catalog #558050) overnight at −20° C. The cells were washed to remove the permeabilization buffer and blocked with Human TruStain FcX (commercially available from BioLegend, San Diego CA as catalog number 422301) and mouse serum for 5 minutes at room temperature. The cells were then treated with the antibody cocktail (Table 7 below) for 1 hour at room temperature. Following antibody staining, the cells were washed, fixed and ran on the Cytek® Aurora Spectral flow cytometer (commercially available from Cytek Biosciences, Fremont CA). The data was analyzed using the FlowJo software (commercially available from Becton Dickinson Corp, Franklin Lakes, NJ). The various cell lineages were gated using their lineage markers and the geometric mean fluorescence intensity of pSTAT3 expression was calculated on FlowJo.

TABLE 7
Antibody cocktail

| Fluorochrome | Antibody | Phosphorylation Site | Clone | Vendor | Catalog# |
|---|---|---|---|---|---|
| BV480 | CD4 | | L200 | BD | 566148 |
| | | | | Biosciences | |
| BV605 | CD8a | | RPA-T8 | BioLegend | 301040 |
| BV786 | CD56 | | 5.1H11 | BioLegend | 362550 |
| AF488 | pSTAT3 | Tyr705 | D3A7 | CST | 4323S |
| PE-Cy5 | CD3 | | UCHT1 | BD | 555334 |
| | | | | Biosciences | |
| PerCP-Cy 5.5 | CD14 | | M5E2 | BioLegend | 301824 |
| APC | CD20 | | 1412 | BioLegend | 340516 |

### Example 2. Screening

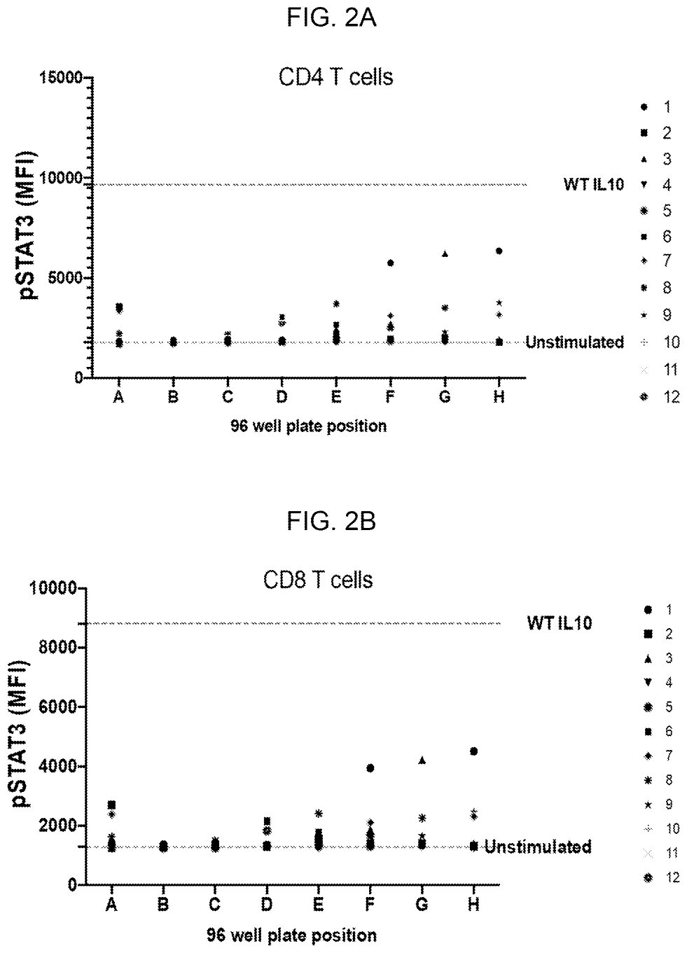

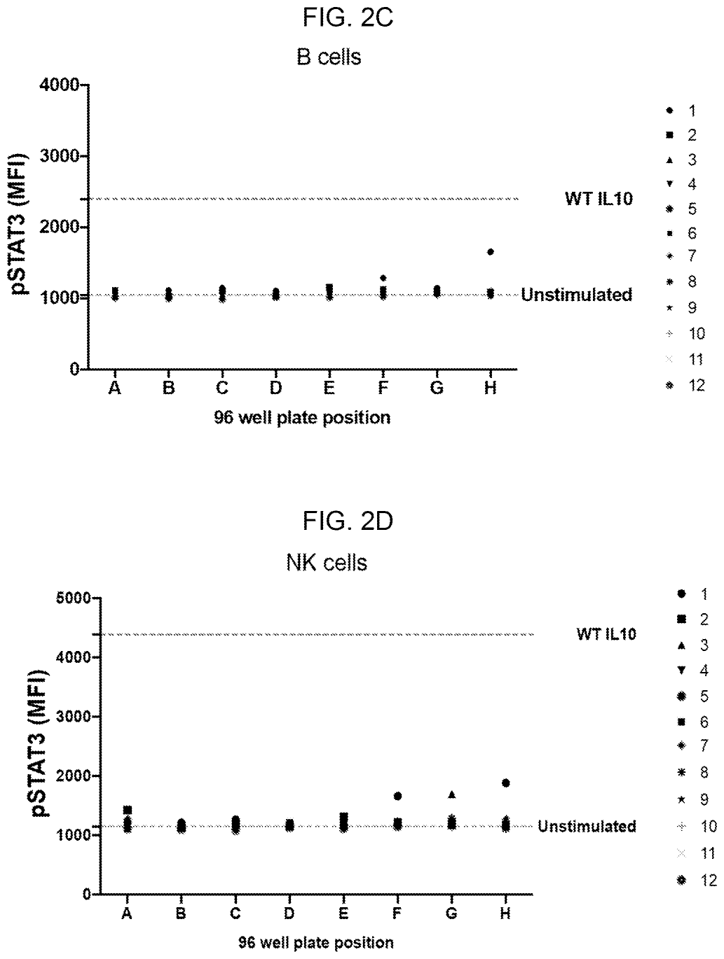

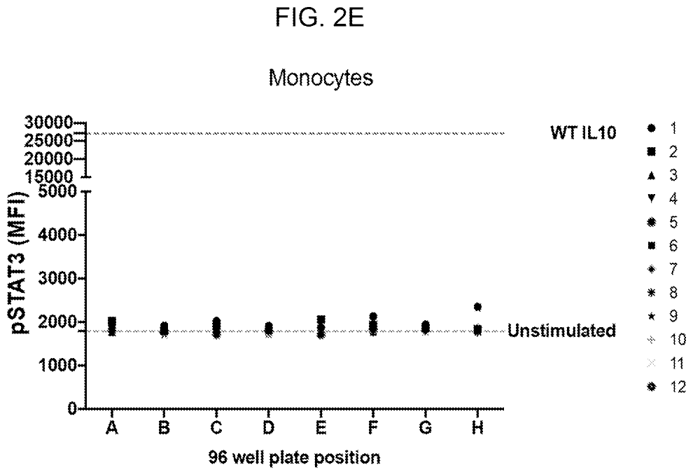

84 anti-IL10Rα/IL2Rγ V_{H}H²s were screened for pSTAT3 activity in the various cell populations that constitute PBMCs (FIGS. 2A-2E). Anti-IL10Rα/IL2Rγ V_{H}H²s that showed at least a 50% increase in pSTAT3 induction over unstimulated control in CD4 T cells were considered as “hits”. The screen yielded 12 hits (See Table 8 below). These 12 anti-IL10Rα/IL2Rγ V_{H}H²s demonstrated nominal pSTAT3 activation in B cells and NK cells, while retaining a robust pSTAT3 activation in CD4⁺ and CD8⁺ T cells and showed minimal pSTAT3 induction in monocytes as compared to the unstimulated control.

TABLE 8
pSTAT3 MFI

| Well | | B | NK | CD4⁺ T | CD8⁺ T | Mono- |
|---|---|---|---|---|---|---|
| ID | Test Article | cells | cells | cells | cells | cytes |
| n/a | Unstimulated | 1045 | 1149 | 1778 | 1288 | 1787 |
| n/a | WT IL10 | 2395 | 4385 | 9665 | 8811 | 27111 |
| F1 | DR441(DR236- | 1284 | 1662 | 5755 | 3945 | 2129 |
| | DR231) | | | | | |
| H1 | DR465(DR240- | 1655 | 1884 | 6349 | 4512 | 2352 |
| | DR231) | | | | | |
| A2 | DR395(DR229- | 1026 | 1427 | 3567 | 2703 | 2020 |
| | DR239) | | | | | |
| F3 | DR449(DR237- | 1094 | 1248 | 2705 | 1876 | 1883 |
| | DR233) | | | | | |
| G3 | DR471(DR241- | 1142 | 1698 | 6235 | 4224 | 1941 |
| | DR231) | | | | | |
| A7 | DR392(DR229- | 1060 | 1282 | 3341 | 2388 | 1886 |
| | DR236) | | | | | |
| F7 | DR442(DR236- | 1047 | 1225 | 3108 | 2101 | 1792 |
| | DR232) | | | | | |
| H7 | DR466(DR240- | 1104 | 1281 | 3166 | 2313 | 1870 |
| | DR232) | | | | | |
| E8 | DR444(DR236- | 1069 | 1273 | 3704 | 2409 | 1830 |
| | DR234) | | | | | |
| G8 | DR468(DR240- | 1080 | 1287 | 3517 | 2261 | 1862 |
| | DR234) | | | | | |
| H9 | DR474(DR241- | 1033 | 1234 | 3769 | 2465 | 2325 |
| | DR234) | | | | | |
| D12 | DR438(DR235- | 1047 | 1174 | 2775 | 1833 | 1806 |
| | DR234) | | | | | |

### Example 3: Dose Response Experiment

Four anti-IL10Rα/IL2Rγ V_{H}H² proteins demonstrating the highest levels of activity as identified from the initial screen based on pSTAT3 induction (Example 2) were further tested in a dose response experiment on B cells, CD4⁺ T cells, NK cells, CD8⁺ T cells, and monocytes. As detailed in Example 1, PBMCs were either left unstimulated or were stimulated with wild-type human IL10 (wt hIL10) or one of the four anti-IL10Rα/IL2Rγ V_{H}H² proteins: (DR395(DR229-DR239), DR441(DR236-DR231), DR471(DR241-DR231), and DR465(DR240-DR231)) over a range of concentrations from 0.0001 nM to 100 nM in ten-fold dilution on B cells, CD4⁺ T cells, NK cells, CD8⁺ T cells, and monocytes. The tables tabulating the pSTAT3 MFI in a dose response experiment in various cell lineages are shown below.

TABLE 9A
B cells pSTAT3 MFI

| IL10 (nM) | Unstimulated | WT- IL10 | A2- DR395(229-239) | F1- DR441(236-231) | G3- DR471(241-231) | H1- DR465(240-231) |
|---|---|---|---|---|---|---|
| 0 | 1193.5 | | | | | |
| 0.0001 | | 1164 | 1126 | 1118 | 1110 | 1108.5 |
| 0.001 | | 1137 | 1131 | 1130.5 | 1125 | 1100 |
| 0.01 | | 1216 | 1137 | 1129 | 1111 | 1087.5 |
| 0.1 | | 1778 | 1153 | 1122 | 1109.5 | 1113.5 |
| 1 | | 3954.5 | 1264.5 | 1220.5 | 1172 | 1289.5 |
| 10 | | 3928 | 1335 | 1539 | 1335 | 1844.5 |
| 100 | | 2624.5 | 1359.5 | 1473.5 | 1277 | 1854.5 |

TABLE 9B
CD4 T cells pSTAT3 MFI

| IL10 (nM) | Unstimulated | WT- IL10 | A2- DR395(229-239) | F1- DR441(236-231) | G3- DR471(241-231) | H1- DR465(240-231) |
|---|---|---|---|---|---|---|
| 0 | 1887 | | | | | |
| 0.0001 | | 1967 | 1803 | 1806 | 1798.5 | 1803 |
| 0.001 | | 1881.5 | 1872 | 1829.5 | 1860 | 1800 |
| 0.01 | | 2223.5 | 1890 | 1879 | 1861.5 | 1846 |
| 0.1 | | 5171 | 2449 | 2235.5 | 2248.5 | 2213.5 |
| 1 | | 10717 | 4573 | 4556.5 | 4599.5 | 4729 |
| 10 | | 10785.5 | 5176 | 6755 | 6804 | 7236.5 |
| 100 | | 9556 | 4668.5 | 6354.5 | 6200 | 7067.5 |

TABLE 9C
NK cells pSTAT3 MFI

| IL10 (nM) | Unstimulated | WT- IL10 | A2- DR395(229-239) | F1- DR441(236-231) | G3- DR471(241-231) | H1- DR465(240-231) |
|---|---|---|---|---|---|---|
| 0 | 1315 | | | | | |
| 0.0001 | | 1314.5 | 1256 | 1248 | 1234 | 1224 |
| 0.001 | | 1274 | 1269.5 | 1261 | 1258 | 1226 |
| 0.01 | | 1376 | 1259.5 | 1268.5 | 1250.5 | 1216 |
| 0.1 | | 2281 | 1307 | 1269.5 | 1260.5 | 1245.5 |
| 1 | | 4617.5 | 1637.5 | 1437.5 | 1533.5 | 1519.5 |
| 10 | | 5700 | 1804.5 | 1797.5 | 1998 | 2039.5 |
| 100 | | 4793.5 | 1757.5 | 1774 | 1935 | 2126 |

TABLE 9D
CD8 T cells pSTAT3 MFI

| IL10 (nM) | Unstimulated | WT- IL10 | A2- DR395(229-239) | F1- DR441(236-231) | G3- DR471(241-231) | H1- DR465(240-231) |
|---|---|---|---|---|---|---|
| 0 | 1446 | | | | | |
| 0.0001 | | 1699.5 | 1376.5 | 1381 | 1395.5 | 1385.5 |
| 0.001 | | 1434.5 | 1421.5 | 1409 | 1423 | 1374.5 |
| 0.01 | | 1722.5 | 1405 | 1432 | 1420.5 | 1376.5 |
| 0.1 | | 4300.5 | 1795.5 | 1605.5 | 1638.5 | 1587.5 |
| 1 | | 8891 | 3319 | 3061.5 | 3281.5 | 3261 |
| 10 | | 9891 | 3878 | 4472.5 | 4614 | 4847 |
| 100 | | 9006 | 3447 | 4257 | 4399 | 5018 |

TABLE 9E
Monocytes pSTAT3 MFI

| IL10 (nM) | Unstimulated | WT- IL10 | A2- DR395(229-239) | F1- DR441(236-231) | G3- DR471(241-231) | H1- DR465(240-231) |
|---|---|---|---|---|---|---|
| 0 | 2116 | | | | | |
| 0.0001 | | 2051 | 2055.5 | 2026.5 | 2008 | 1988 |
| 0.001 | | 2140.5 | 2062.5 | 2041 | 2030.5 | 1974 |
| 0.01 | | 2842 | 2016.5 | 2033 | 1995 | 1946.5 |
| 0.1 | | 9547 | 2019 | 2013.5 | 1988 | 1964 |
| 1 | | 28900.5 | 2209.5 | 2063 | 2032.5 | 2110.5 |
| 10 | | 30917 | 2338 | 2344 | 2228.5 | 2470.5 |
| 100 | | 26589.5 | 2436 | 2293.5 | 2212 | 2469.5 |

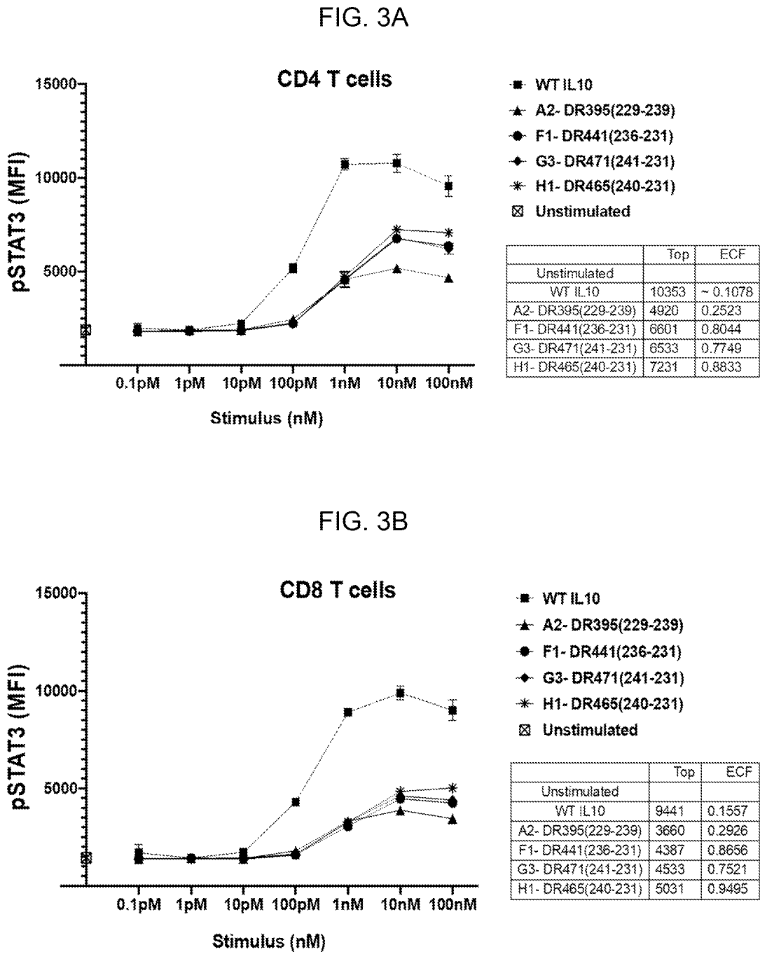

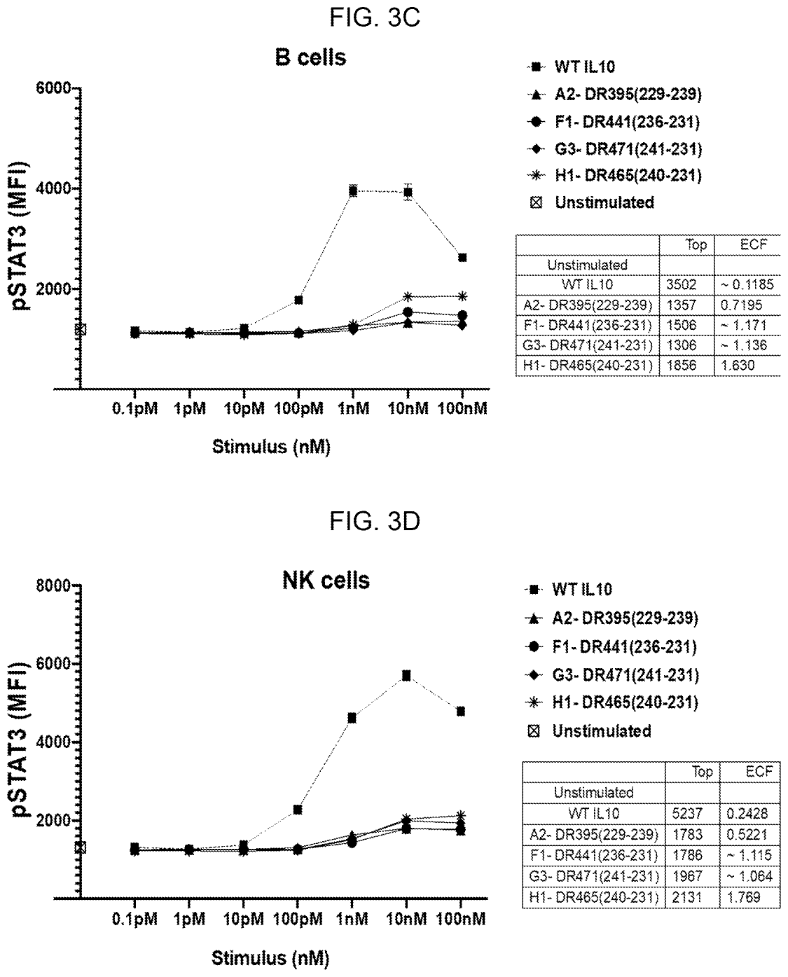

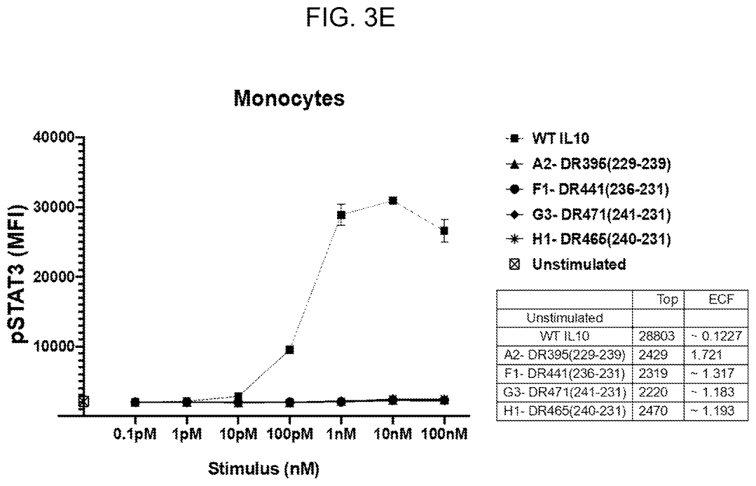

As can be seen from the foregoing data and FIGS. 3A-3E, the anti-IL10Rα/IL2Rγ V_{H}H² proteins of the present disclosure demonstrated a dose dependent induction of pSTAT3 with most induction seen in CD4⁺ T cells and CD8⁺ T cells, followed by NK cells and B cells. Monocytes showed very minimal induction of pSTAT3, especially compared to the levels of pSTAT3 induction seen with WT IL10. The foregoing demonstrates the ability of the IL10Rα/IL2Rγ binding proteins of the present disclosure to provide selective cell type activation, retaining the desirable property of stimulating T cells while having a minimal impact on monocytes.

### Example 4—V_{H}H Generation

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7^{th} immunization.

After immunization, the library was constructed. Briefly, RNA was extracted from blood and transcribed to cDNA. The V_{H}H regions were obtained via two-step PCR, which fragment about 400 bp. The PCR outcomes and the vector of pMECS phagemid were digested with Pst I and Not I, subsequently, ligated to pMECS/Nb recombinant. After ligation, the products were transformed into Escherichia coli (E. coli) TG1 cells by electroporation. Then, the transformants were enriched in growth medium and planted on plates. Finally, the library size was estimated by counting the number of colonies.

Library biopanning was conducted to screen candidates against the antigens after library construction. Phage display technology was applied in this procedure. Positive colonies were identified by PE-ELISA.

### Example 5—Recombinant Production and Purification

Codon optimized DNA inserts were cloned into modified pcDNA3.4 (Genscript) for small scale expression in HEK293 cells in 24 well plates. The binding molecules were purified in substantial accordance with the following procedure. Using a Hamilton Star automated system, 96×4 mL of supernatants in 4×24-well blocks were re-arrayed into 4×96-well, 1 mL blocks. PhyNexus micropipette tips (Biotage, San Jose CA) holding 80 μL of Ni-Excel IMAC resin (Cytiva) are equilibrated wash buffer: PBS pH 7.4, 30 mM imidazole. PhyNexus tips were dipped and cycled through 14 cycles of 1 mL pipetting across all 4×96-well blocks. PhyNexus tips were washed in 2×1 mL blocks holding wash buffer. PhyNexus tips were eluted in 3×0.36 mL blocks holding elution buffer: PBS pH 7.4, 400 mM imidazole. PhyNexus tips were regenerated in 3×1 mL blocks of 0.5 M sodium hydroxide.

The purified protein eluates were quantified using a Biacore® T200 as in substantial accordance with the following procedure. 10 μL of the first 96×0.36 mL eluates were transferred to a Biacore® 96-well microplate and diluted to 60 uL in HBS-EP+ buffer (10 mM Hepes pH 7.4, 150 mM NaCl, 1 mM EDTA, 0.05% Tween 20). Each of the 96 samples was injected on a CM5 series S chip previously functionalized with anti-histidine capture antibody (Cytiva): injection is performed for 18 seconds at 5 μL/min. Capture levels were recorded 60 seconds after buffer wash. A standard curve of known V_{H}H concentrations (270, 90, 30, 10, 3.3, 1.1 pg/mL) was acquired in each of the 4 Biacore chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning).

### Example 6. Evaluation of Binding Affinity of IL10Ra/IL2Rg Dimers Via SPR

All experiments were conducted in 10 mM Hepes, 150 mM NaCl, 0.05% (v/v) Polysorbate 20 (PS20) and 3 mM EDTA (HBS-EP+ buffer) on a Biacore T200 instrument equipped with Protein A or CAP biotin chips (Cytiva). For experiments on Protein A chips, Fc-fused ligands were flowed at 5 μl/min for variable time ranging from 18 to 300 seconds, reaching the capture loads listed in the tables below. Following ligand capture, injections of a 2-fold dilution series of analyte typically comprising at least five concentrations between 1 μM and 1 nM were performed in either high performance or single cycle kinetics mode. Surface regeneration was achieved by flowing 10 mM glycine-HCl, pH 1.5 (60 seconds, 50 L/min). Buffer-subtracted sensograms were processed with Biacore T200 Evaluation Software and globally fit with a 1:1 Langmuir binding model (bulk shift set to zero) to extract kinetics and affinity constants (kₐ, k_{d}, K_{D}). R_{MAX}<100 RU indicates surface density compatible with kinetics analysis. Experiments on CAP chips were performed as described above with an additional capture step of Biotin CAPture reagent (10 seconds, 40 uL/min) performed prior to capture of biotinylated ligands. Calculated Rmax were generated using the equation Rmax=Load (RU) x valency of ligand x (Molecular weight of analyte/Molecular weight of ligand. Surface activity was defined as the ratio experimental/calculated Rmax. The results of these experiments are provided in below for sample information and experimental results.

### Example 7: Dose Response Experiment with Fc Versions

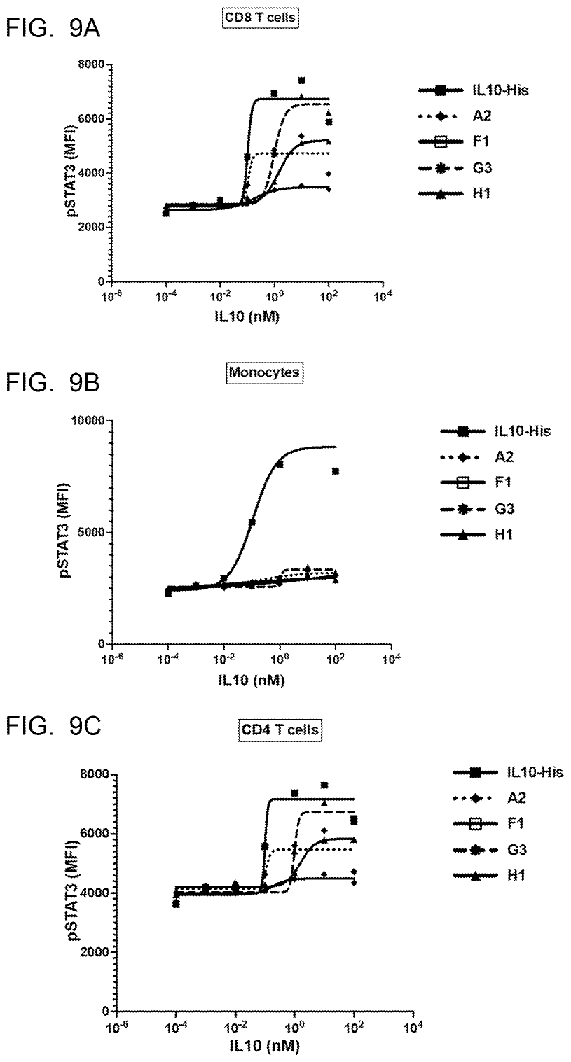

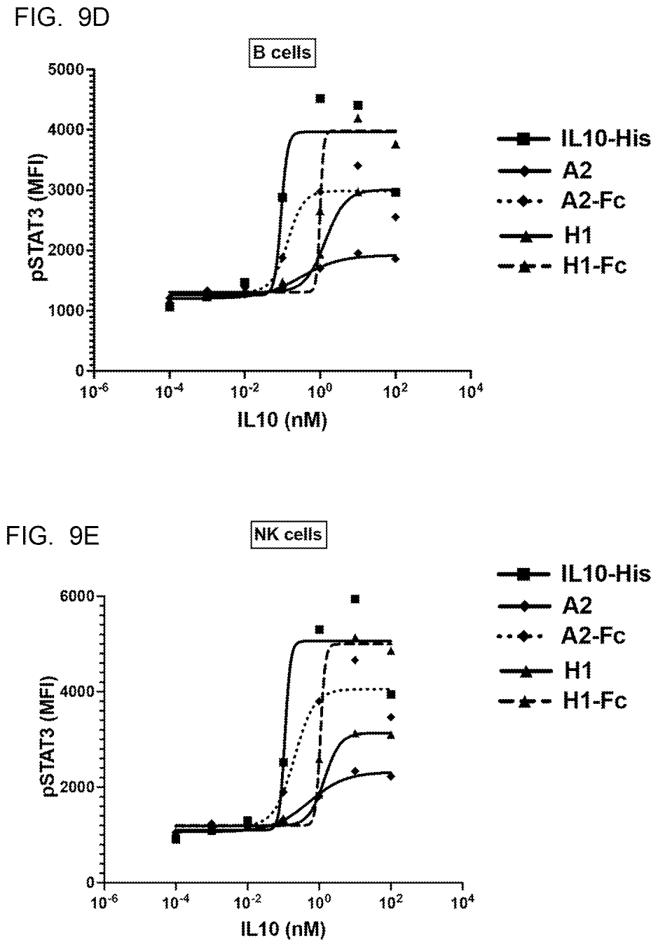

Two of the VHHs and their Fc versions were tested in a dose response curve as described above. As detailed in example 1, PBMCs were either left unstimulated or were stimulated with WT IL10 or one of the 2 IL10R1/2y VHHs [DR395(DR229-DR239), DR465(DR240-DR231) or the Fc versions of the VHHs [DR992 (H1, DR240-DR231), DR995 (A2, DR229-DR239)] at concentrations ranging from 0.1 pM-100 nM for 20 min at 37° C. The staining and analysis was done as described in Example 1. The results are provided in FIGS. 9A-E of the accompanying drawings. The Fc molecules, H1-Fc [DR992, DR240-DR231)] and A2-Fc [DR995 (DR229-DR239)] were more potent as compared to their non-Fc counterparts, H1 [DR465(DR240-DR231)] and A2[DR395(DR229-DR239)] at inducing pSTAT3 signal in CD4 T cells, CD8 T cells, B cells and NK cells. The pSTAT3 signal in monocytes was still very minimal compared to the levels of pSTAT3 induction seen with WT IL10.

### Example 8: Monocyte Functional Assay

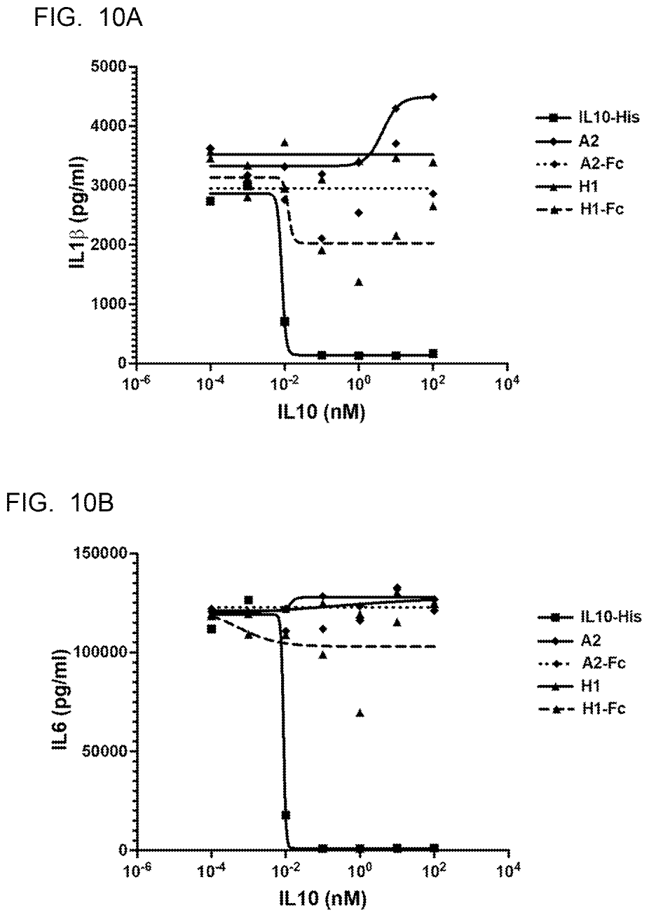

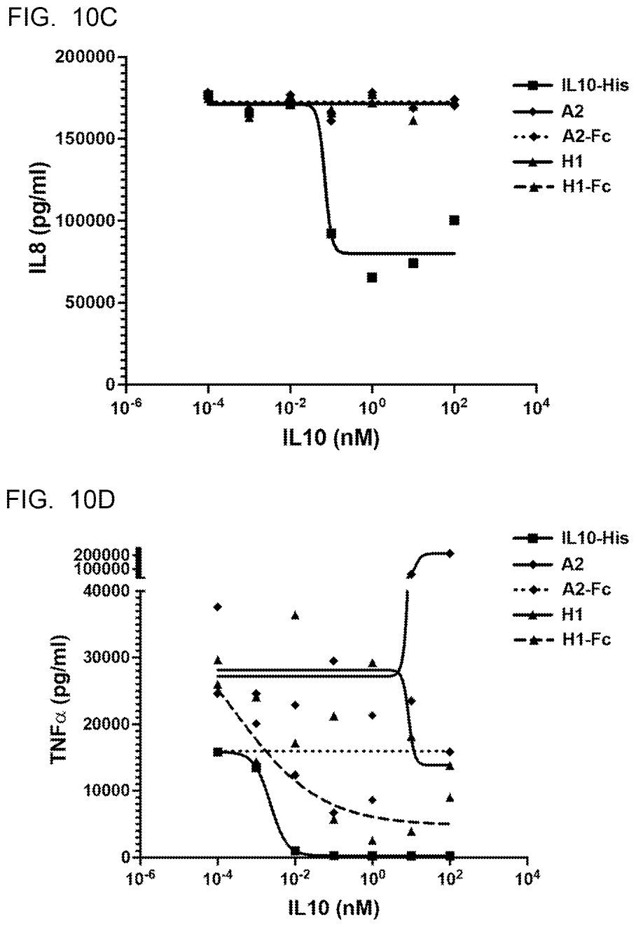

Human monocytes were purified from human PBMCs using CD14 microbeads (Miltenyi Biotech 130-050-201). The purified monocytes were seeded at 100,000 cells per well in a 96-well flat bottom plate and treated with IL10 at concentrations ranging from 0.1 pM-100 nM in complete RPMI medium [RPMI containing 10% FBS and 1× Penicillin/Streptomycin (Gibco, Cat. #15-140-122)] for 48 hours min at 37° C. After the 48-hour treatment, plates were spun down at 400 g for 5 min and supernatants were collected. The supernatants were tested on a Meso Scale discovery assay (Meso Scale Discovery Catalog no. K151A9H) to measure the levels of cytokines IL1b, IL6, IL8 and TNFa. The results of these studies are provided in FIGS. 10A-10D of the accompanying drawings. The IL10R1/2y VHHs, H1 [DR465(DR240-DR231)] and A2[DR395(DR229-DR239)] did not inhibit LPS induced secretion of IL1b, IL6, TNFa and IL8, correlating with the lack of induction of pSTAT3 signaling in the Monocytes. The Fc version of the molecules were slightly more potent at inhibiting secretion of LPS induced IL1b and TNFa production as compared to their non-Fc counterparts, but did not cause complete inhibition even at higher concentrations of stimulus.

### Example 9: CD8 Blast Functional Assay

Human CD8 T cells were purified from human PBMCs by negative selection using the CD8⁺ T cell isolation kit (Milteyi Biotec 130-096-495). Isolated CD8 T cells were then activated using the human CD8 T cell activation/expansion kit (Miltenyi Biotec 130-091-441) for 3 days. The day 3 CD8 T cell blasts were then treated with IL10 at concentrations ranging from 0.1 pM-100 nM in Yssel's medium [(IMDM, Gibco, Cat. #122440-053) containing 0.25% w/v Human Albumin (Sigma, Cat. #A9080), 1×ITS-X (human) (Gibco, Cat. #51500056), 30 mg/L Transferrin (Roche, Cat. #10652202001), 2 mg/L PA BioXtra (Sigma, Cat. #P5585), 1×LA-OA-Albumin (Sigma, Cat. #L9655), 1× Penicillin/Streptomycin (Gibco, Cat. #15-140-122), 1% Human Serum (Gemini, Cat. #507533011)], for 72 hours at 37° C. In the last 5 hours of incubation, cells were treated with 1:1000 Monensin (eBiosciences, Cat. #00-4505-51). After incubation, cells were washed with PBS and stained with Zombie NIR fixable viability dye (Biolegend, Cat. #423105) for 15 minutes at 4′C in the dark. Cells were washed twice in pre-made FACS Buffer (PBS+2% FBS) and then fixed in IC fixation buffer (Invitrogen 00-8222) for 20 min at room temperature. Cells were then spun down and permeabilized with 1× permeabilization buffer (Invitrogen 00-833) for 5 min.

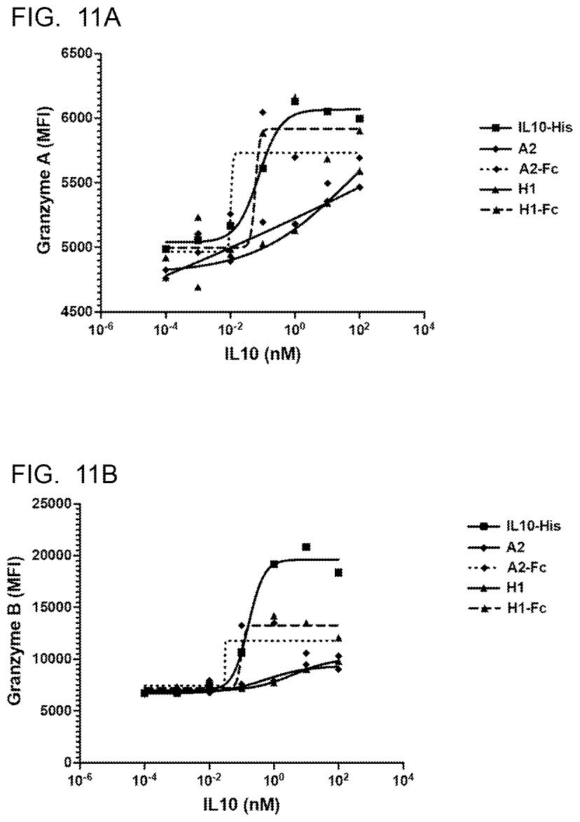

Cells were resuspended in permeabilization buffer, briefly blocked with 1:10 Human TruStain FcX Fc Block (Biolegend, Cat. #422302) and then stained with anti-Granzyme A antibody (Biolegend Cat. #507206), and anti-Granzyme B antibody (BD 562462) for 1 hour at room temperature, in the dark. Cells were then washed with FACS Buffer twice and resuspended in FACS Buffer containing 1% PFA (Electron Microscopy Sciences, Cat. #15710) for at least 10 minutes at room temperature in the dark prior to acquisition on the Cytek Aurora Spectral flow cytometer. The data was analyzed using the FlowJo software. The results of these studies are provided in FIGS. 11A and 11B of the accompanying drawings. [0322] The IL10R1/2y VHHs, H1 [DR465(DR240-DR231)] and A2[DR395(DR229-DR239)] induce Granzyme A production at higher concentration of stimulus and are weak inducers of Granzyme B production. The addition of Fc to the molecule enhanced its activity with H1-Fc and A2-Fc being as potent as the WT in inducing Granzyme A production while also being more potent at inducing Granzyme B production.

TABLE 10
anti-human IL10Ra sdAb CDRs

| | CDR 1 | | | | | |
|---|---|---|---|---|---|---|
| | Kabat/ | SEQ | | SEQ | | SEQ |
| | chothia | ID | | ID | | ID |
| Name | hybrid) | NO: | CDR 2 | NO: | CDR 3 | NO: |
| hIL10Ra_ | YLYSIDYMA | 276 | VIYTASGATFYPDSVKG | 277 | VRKTDSYLFDAQS | 278 |
| VHH1 | | | | | FTY | |
| hIL10Ra_ | YLYSTNYMA | 279 | VIYTASGATLYTDSVKG | 280 | VRKTDSYLFDAQS | 281 |
| VHH2 | | | | | FTY | |
| hIL10Ra_ | YLYSTNYMA | 282 | VIYTASGATLYTDSVKG | 283 | VRKTDSYLFDAQS | 284 |
| VHH3 | | | | | FTY | |
| hIL10Ra_ | YLYSIDYMA | 285 | VIYTASGATFYPDSVKG | 286 | VRKTDSYLFDAQS | 287 |
| VHH4 | | | | | FTY | |
| hIL10Ra_ | YLYSTNYMA | 288 | AIYTASGATLYSDSNKG | 289 | VRKTGSYLFDAQS | 290 |
| VHH5 | | | | | FTY | |
| hIL10Ra_ | FTYSSYCMG | 291 | SIDSDGSTSYTDSVKG | 292 | DLMSTVVPGFCGF | 293 |
| VHH6 | | | | | LLSAGMDY | |
| hIL10Ra_ | YTFNSNCMG | 294 | TIYTGVGSTYYADSVKG | 295 | EPLSRVYGGSCPT | 296 |
| VHH7 | | | | | PTFGY | |
| hIL10Ra_ | YTYSMYCMG | 297 | QINSDGSTSYADSVKG | 298 | DSRVYGGSWYERL | 299 |
| VHH8 | | | | | CGPYTYEYNY | |
| hIL10Ra_ | YAYSTYCMG | 300 | AIDSGGSTSYADSVKG | 301 | VPPPPDGGSCLFL | 302 |
| VHH9 | | | | | GPEIKVSKADFRY | |
| hIL10Ra_ | YLYSIDYMA | 303 | VIYTASGATFYPDSVKG | 304 | VRKTDSYLFDAQS | 305 |
| VHH10 | | | | | FTY | |
| hIL10Ra_ | YTYSSYCMG | 306 | VIDSDGSTSYADSVKG | 307 | DLGHYRPPCGVLY | 308 |
| VHH11 | | | | | LGMDY | |
| hIL10Ra_ | YTYSSNCMG | 309 | TIYTGGGNTYYADSVKG | 310 | EPLSRVYGGSCPT | 311 |
| VHH12 | | | | | PTFDY | |
| hIL10Ra_ | YSYSSNCMG | 312 | TIHTGGGSTYYADSVKG | 313 | EPLSRLYGGSCPT | 314 |
| VHH13 | | | | | PTFGY | |
| hIL10Ra_ | YTYSSYCMG | 315 | VIDSDGSTSYADSVKG | 316 | DLGHYRPPCGVLY | 317 |
| VHH14 | | | | | LGMDY | |
| hIL10Ra_ | YTYSGYCMG | 318 | VIDSDGSTSYADSVKG | 319 | DLGHYRPPCGVLY | 320 |
| VHH15 | | | | | LGMDY | |
| hIL10Ra_ | YTYSNYCMG | 321 | TIDSDGNTSYADSVKG | 322 | DLGHYRPPCGAYY | 323 |
| VHH16 | | | | | YGMDY | |
| hIL10Ra_ | YSNCSYDMT | 324 | AIHSDGSTRYADSVKG | 325 | DPLHCRAHGGSWY | 326 |
| VHH17 | | | | | SVRANY | |
| hIL10Ra_ | YTYNSNCMG | 327 | TIYTGVGSTYYADSVKG | 328 | EPLSRVYGGSCPT | 329 |
| VHH18 | | | | | PTFGY | |

TABLE 11
human anti-IL2Rg sdAb CDRs

| | CDR 1 | SEQ | | SEQ | | SEQ |
|---|---|---|---|---|---|---|
| | (Kabat/ | ID | | ID | | ID |
| Name | chothia) | NO: | CDR 2 | NO: | CDR 3 | NO: |
| hIL2Rg_VHH-1 | FTFDDSDMG | 330 | TISSDGSTYYADSVKG | 331 | DFMIAIQAPGAGC | 332 |
| hIL2Rg_VHH-2 | FSFSSYPMT | 333 | TIASDGGSTAYAASVE | 334 | GYGDGTPA | 335 |
| | | | G | | | |
| hIL2Rg_VHH-3 | FTFDDREMN | 336 | TISSDGSTYYADSVKG | 337 | DFMIAIQAPGAGC | 338 |
| hIL2Rg_VHH-4 | FTFDDSDMG | 339 | TISSDGNTYYTDSVKG | 340 | EPRGYYSNYGGRR | 341 |
| | | | | | ECNY | |
| hIL2Rg_VHH-5 | FSFSSYPMT | 342 | TIASDGGSTAYAASVE | 343 | GYGDGTPA | 344 |
| | | | G | | | |
| hIL2Rg_VHH-6 | FTFSNAHMS | 345 | SIYSGGSTWYADSVKG | 346 | NRLHYYSDDDSL | 347 |
| hIL2Rg_VHH-7 | FTFDDREMN | 348 | TISSDGSTYYADSVKG | 349 | DFMIAIQAPGAGC | 350 |
| hIL2Rg_VHH-8 | YTFSSYCMG | 351 | ALGGGSTYYADSVKG | 352 | AWVACLEFGGSWY | 353 |
| | | | | | DLARYKH | |
| hIL2Rg_VHH-9 | FTFDDSDMG | 354 | TISSDGSTYYADSVKG | 355 | EPRGYYSNYGGRR | 356 |
| | | | | | ECNY | |
| hIL2Rg_VHH-10 | SIYSSAYIG | 357 | GIYTRDGSTAYADSVK | 358 | GRRTKSYVYIFRP | 359 |
| | | | G | | EEYNY | |
| hIL2Rg_VHH-11 | FTFSSAHMS | 360 | SIYSGGGTFYADSVKG | 361 | NRLHYYSDDDSL | 362 |
| hIL2Rg_VHH-12 | FTFSNAHMS | 363 | SIYSGGSTWYADSVKG | 364 | NRLHYYSDDDSL | 365 |
| hIL2Rg_VHH-13 | FIFDDSDMG | 366 | TISSDGSTYYADSVKG | 367 | EPRGYYSNYGGRR | 368 |
| | | | | | ECNY | |
| hIL2Rg_VHH-14 | FTADDSDMG | 369 | TISSDGSTYYADSVKG | 370 | EPRGYYSNYGGRR | 371 |
| | | | | | ECNY | |
| hIL2Rg_VHH-15 | FTFSSAHMS | 372 | SIYSGGGTFYADSVKG | 373 | NRLHYYSDDDSL | 374 |
| hIL2Rg_VHH-16 | FTFSNAHMS | 375 | SIYSGGSTWYADSVKG | 376 | NRLHYYSDDDSL | 377 |
| hIL2Rg_VHH-17 | FTFSNAHMS | 378 | SIYSGGSTWYADSVKG | 379 | NRLHYYSDDDSL | 380 |
| hIL2Rg_VHH-18 | FTFSSYPMT | 381 | TIASDGGSTAYAASVE | 382 | GYGDGTPA | 383 |
| | | | G | | | |
| hIL2Rg_VHH-19 | FTFDDREMN | 384 | TISSDGSTYYADSVKG | 385 | DFMIAIQAPGAGC | 386 |
| hIL2Rg_VHH-20 | FTFDDSDMG | 387 | TISSDGSTYYADSVKG | 388 | EPRGYYSNYGGRR | 389 |
| | | | | | ECNY | |
| hIL2Rg_VHH-21 | YTSCMG | 390 | TIYTRGRSIYYADSVK | 391 | GGYSWSAGCEFNY | 392 |
| | | | G | | | |
| hIL2Rg_VHH-22 | FSFSSYPMT | 393 | TIASDGGSTAYAASVE | 394 | GYGDGTPA | 395 |
| | | | G | | | |
| hIL2Rg_VHH-23 | FSFSSYPMT | 396 | TIASDGGSTAYAASVE | 397 | GYGDGTPA | 398 |
| | | | G | | | |

TABLE 12
mouse anti-IL2Rg sdAb CDRs

| | CDR 1 | SEQ | | SEQ | | SEQ |
|---|---|---|---|---|---|---|
| | (Kabat/ | ID | | ID | | ID |
| Name | chothia) | NO: | CDR 2 | NO: | CDR 3 | NO: |
| mIL2Rg_ | YGYNYIG | 399 | VIYTGGGDT | 400 | SVYACLRGGHDEY | 401 |
| VHH1 | | | YYADSVKG | | AH | |
| mIL2Rg_ | STYANYLMG | 402 | AIYSGGGST | 403 | ASAVKGDKGDIVV | 404 |
| VHH2 | | | YYADSVKG | | VVTGTQRMEYDY | |
| mIL2Rg_ | FTFDESVMS | 405 | IISSDDNTY | 406 | RRRRPVYDSDYEL | 407 |
| VHH3 | | | YDDSVKG | | RPRPLCGDFGV | |
| mIL2Rg_ | LPFDEDDMG | 408 | SISSDGTAY | 409 | GVHRQFGGSSSCG | 410 |
| VHH4 | | | YADSVKG | | DAFYGMDY | |
| mIL2Rg_ | DVYGRNSMA | 411 | VGYSVVTTT | 412 | DGNLWRGLRPSEY | 413 |
| VHH5 | | | YYADSVKG | | TY | |
| mIL2Rg_ | FPYSRYCMG | 414 | AIEPDGSTS | 415 | DERCFYLKDYDLR | 416 |
| VHH6 | | | YADSVKG | | RPAQYRY | |
| mIL2Rg_ | FTFDESDMG | 417 | VITSDDNPY | 418 | RSRQPVYSRDYEL | 419 |
| VHH7 | | | YDDSVKG | | RPRPLCGDFGV | |
| mIL2Rg_ | FTFDDFDMG | 420 | TISDDGSTY | 421 | EGALGSKTNCGWV | 422 |
| VHH8 | | | YADSVKG | | GNFGY | |
| mIL2Rg_ | FTFDDFDMG | 423 | TISDDGSTY | 424 | EGALGSKTNCGWV | 425 |
| VHH9 | | | YADSVKG | | GNFGY | |
| mIL2Rg_ | FTFDDFDMG | 426 | TISDDGSTY | 427 | EGALGSKTNCGWV | 428 |
| VHH10 | | | YADSVKG | | GNFGY | |
| mIL2Rg_ | FTFSDRDMG | 429 | TISDDGSTY | 430 | EGALGSKTNCGWV | 431 |
| VHH11 | | | YADSVKG | | GNFGY | |
| mIL2Rg_ | YGYNYIG | 432 | VIYIGGGDT | 433 | RYCVGSVYACLRG | 434 |
| VHH12 | | | YYADSVKG | | GHDEYAH | |
| mIL2Rg_ | YGYNYIG | 435 | VIYTGGGDT | 436 | RYCVGSVYACLRG | 437 |
| VHH13 | | | YYADSVKG | | GHDEYAH | |
| mIL2Rg_ | FTFDDFDMG | 438 | TISDDGSTY | 439 | EGALGSKTNCGWV | 440 |
| VHH14 | | | YANSVKG | | GNFGY | |
| mIL2Rg_ | FTFDDFDMG | 441 | TISDDGSTY | 442 | EGALGSKMNCGWV | 443 |
| VHH15 | | | YADSVKG | | GNFGY | |

TABLE 13
human anti-IL10Ra VHH Amino Acid Sequences

| | | SEQ |
|---|---|---|
| | | ID |
| Name | VHH Sequence | NO: |
| hIL10Ra_VHH1 | QVQLQESGGGSIQAGGSLRLSCAAS | 444 |
| | RYLYSIDYMAWFRQSPGKEREPVAV | |
| | IYTASGATFYPDSVKGRFTISQDNA | |
| | KMTVYLQMNSLKSEDTAMYYCAAVR | |
| | KTDSYLFDAQSFTYWGQGTQVTVSS | |
| hIL10Ra_VHH2 | QVQLQESGGGSVQAGGSLRLSCVAS | 445 |
| | RYLYSTNYMAWFRQSPGKEREAVAV | |
| | IYTASGATLYTDSVKGRFTISQDNA | |
| | KMTVYLQMNRLKSEDTAMYYCAAVR | |
| | KTDSYLFDAQSFTYWGQGTQVTVSS | |
| hIL10Ra_VHH3 | QVQLQESGGGSIQAGGSLRLSCVAS | 446 |
| | RYLYSTNYMAWFRQSPGKEREAVAV | |
| | IYTASGATLYTDSVKGRFTISQDNA | |
| | KMTVYLQMNRLKSEDTAMYYCAAVR | |
| | KTDSYLFDAQSFTYWGQGTQVTVSS | |
| hIL10Ra_VHH4 | QVQLQESGGGSIQAGGSLRLSCAAS | 447 |
| | RYLYSIDYMAWFRQSPGKEREPAAV | |
| | IYTASGATFYPDSVKGRFTISQDNA | |
| | KMTVYLQMNSLKSEDTAMYYCAAVR | |
| | KTDSYLFDAQSFTYWGQGTQVTVSS | |
| hIL10Ra_VHH5 | QVQLQESGGGSIQAGGSLRLSCVAS | 448 |
| | KYLYSTNYMAWFRQSPGKEREAVAA | |
| | IYTASGATLYSDSNKGRFTISQDNA | |
| | KMTVYLQMNSLKSEDTAMYYCAAVR | |
| | KTGSYLFDAQSFTYWGQGTQVTVSS | |
| hIL10Ra_VHH6 | QVQLQESGGGSVQAGGSLRLSCAAS | 449 |
| | RFTYSSYCMGWFRQAPGKEREGVAS | |
| | IDSDGSTSYTDSVKGRFTISKDNAK | |
| | NTLYLQMNSLKPEDTAMYYCALDLM | |
| | STVVPGFCGFLLSAGMDYWGKGTQV | |
| | TVSS | |
| hIL10Ra_VHH7 | QVQLQESGGGSVQAGGSLRLSCAVS | 450 |
| | GYTFNSNCMGWFRQAPGKEREGVAT | |
| | IYTGVGSTYYADSVKGRFTISQDNA | |
| | KNTVYLQMNSLKPEDTAMYYCAAEP | |
| | LSRVYGGSCPTPTFGYWGQGTQVTV | |
| | SS | |
| hIL10Ra_VHH8 | QVQLQESGGGSVQAGGSLRLSCAAS | 451 |
| | GYTYSMYCMGWFRQAPGKEREGVAQ | |
| | INSDGSTSYADSVKGRFTISKDNAK | |
| | NTLYLQMNSLKPEDTAMYYCAADSR | |
| | VYGGSWYERLCGPYTYEYNYWGQGT | |
| | QVTVSS | |
| hIL10Ra_VHH9 | QVQLQESGGGSVQAGGSLRLSCAVS | 452 |
| | GYAYSTYCMGWFRQAPGKEREGVAA | |
| | IDSGGSTSYADSVKGRFTISKDNAK | |
| | NTLYLRMNSLKPEDTAMYYCAAVPP | |
| | PPDGGSCLFLGPEIKVSKADFRYWG | |
| | QGTQVTVSS | |
| hIL10Ra_VHH10 | QVQLQESGGGSVQAGGSLRLSCAAS | 453 |
| | RYLYSIDYMAWFRQSPGKEREPVAV | |
| | IYTASGATFYPDSVKGRFTISQDNA | |
| | KMTVYLQMNSLKSEDTAMYYCAAVR | |
| | KTDSYLFDAQSFTYWGQGTQVTVSS | |
| hIL10Ra_VHH11 | QVQLQESGGGSVQAGGSLRLSCGAS | 454 |
| | RYTYSSYCMGWFRQAPGKEREGVAV | |
| | IDSDGSTSYADSVKGRFTISKDNGK | |
| | NTLYLQMNSLKPEDTAMYYCAADLG | |
| | HYRPPCGVLYLGMDYWGKGTQVTVS | |
| | S | |
| hIL10Ra_VHH12 | QVQLQESGGGSVQAGGSLRLSCTVS | 455 |
| | GYTYSSNCMGWFRQAPGKEREGVAT | |
| | IYTGGGNTYYADSVKGRFTISQDNA | |
| | KNTVYLQMNNLKPEDTAMYYCAAEP | |
| | LSRVYGGSCPTPTFDYWGQGTQVTV | |
| | SS | |
| hIL10Ra_VHH13 | QVQLQESGGGSVQAGGSLRLSCAVS | 456 |
| | GYSYSSNCMGWFRQAPGKEREGVAT | |
| | IHTGGGSTYYADSVKGRFTISQDNA | |
| | KNTVYLQMNSLKPEDTAMYYCAAEP | |
| | LSRLYGGSCPTPTFGYWGQGTQVTV | |
| | SS | |
| hIL10Ra_VHH14 | QVQLQESGGGSVQAGGSLRLSCGAS | 457 |
| | GYTYSSYCMGWFRQVPGKEREGVAV | |
| | IDSDGSTSYADSVKGRFTISKDNGK | |
| | NTLYLQMNSLKPEDTAMYYCAADLG | |
| | HYRPPCGVLYLGMDYWGKGTQVTVS | |
| | S | |
| hIL10Ra_VHH15 | QVQLQESGGGSVQAGGSLRLSCGAS | 458 |
| | GYTYSGYCMGWFRQAPGKEREGVAV | |
| | IDSDGSTSYADSVKGRFTISKDNGK | |
| | NTLYLQMNSLKPEDTAMYYCAADLG | |
| | HYRPPCGVLYLGMDYWGKGTQVTVS | |
| | S | |
| hIL10Ra_VHH16 | QVQLQESGGGSVQAGGSLRLACAAS | 459 |
| | RYTYSNYCMGWFRQAPGKEREGVAT | |
| | IDSDGNTSYADSVKGRFTISRDNAK | |
| | NTLYLQMNSLKPGDTAMYYCAADLG | |
| | HYRPPCGAYYYGMDYWGKGTQVTVS | |
| | S | |
| hIL10Ra_VHH17 | QVQLQESGGGSVQAGGSLRLSCAAS | 460 |
| | GYSNCSYDMTWYRQAPGKEREFVSA | |
| | IHSDGSTRYADSVKGRFFISQDNAK | |
| | NTVYLQMNSLKPEDTAMYYCKTDPL | |
| | HCRAHGGSWYSVRANYWGQGTQVTV | |
| | SS | |
| hIL10Ra_VHH18 | QVQLQESGGGSVQAGGSLRLSCAVS | 461 |
| | GYTYNSNCMGWFRQAPGKEREGVAT | |
| | IYTGVGSTYYADSVKGRFTISQDNA | |
| | KNTVYLQMNSLKPEDTAMYYCAAEP | |
| | LSRVYGGSCPTPTFGYWGQGTQVTV | |
| | SS | |

TABLE 14
human anti-IL2Rg VHH Amino Acid Sequences

| | | SEQ |
|---|---|---|
| | VHH Sequence | ID |
| Name | (CDRs are underlined) | NO: |
| hIL2Rg_ | QVQLQESGGGSVQAGGSLRLSCAASGFTFDDS | 276 |
| VHH-1 | DMGWYRQAPGNECDLVSTISSDGSTYYADSVK | |
| | GRFTISQDNAKNTVYLQMDSVKPEDTAVYYCA | |
| | ADFMIAIQAPGAGCWGQGTQVTVSS | |
| hIL2Rg_ | QVQLQESGGGSVPAGGSLKLSCAASGFSFSSY | 277 |
| VHH-2 | PMTWARQAPGKGLEWVSTIASDGGSTAYAASV | |
| | EGRFTISRDNAKSTLYLQLNSLKTEDTAMYYC | |
| | TKGYGDGTPAPGQGTQVTVSS | |
| hIL2Rg_ | QVQLQESGGGSVQTGGSLRLSCTASGFTFDDR | 278 |
| VHH-3 | EMNWYRQAPGNECELVSTISSDGSTYYADSVK | |
| | GRFTISQDNAKNTVYLQMDSVKPEDTAVYYCA | |
| | ADFMIAIQAPGAGCWGQGTQVTVSS | |
| hIL2Rg_ | QVQLQESGGGSVQAGGSLRLSCTASGFTFDDS | 279 |
| VHH-4 | DMGWYRQAPGNECELVSTISSDGNTYYTDSVK | |
| | GRFTISQDNAKNTVYLQMNSLGPEDTAVYYCA | |
| | AEPRGYYSNYGGRRECNYWGQGTQVTVSS | |
| hIL2Rg_ | QVQLQESGGGSVQAGGSLRLSCAASGFSFSSY | 280 |
| VHH-5 | PMTWARQAPGKGLEWVSTIASDGGSTAYAASV | |
| | EGRFTISRDNAKSTLYLQLNSLKTEDTAMYYC | |
| | TKGYGDGTPAPGQGTQVTVSS | |
| hIL2Rg_ | QVQLQESGGGAVQAGGSLRLSCAASGFTFSNA | 281 |
| VHH-6 | HMSWVRQAPGKGREWISSIYSGGSTWYADSVK | |
| | GRFTISRDNSKNTLYLQLNSLKTEDTAMYYCA | |
| | ENRLHYYSDDDSLRGQGTQVTVSS | |
| hIL2Rg_ | QVQLQESGGGLVQPGGSLRLSCAASGFTFDDR | 282 |
| VHH-7 | EMNWYRQAPGNECELVSTISSDGSTYYADSVK | |
| | GRFTISQDNAKNTVYLQMDSVKPEDTAVYYCA | |
| | ADFMIAIQAPGAGCWGQGTQVTVSS | |
| hIL2Rg_ | QVQLQESGGGSVQAGGSLRLSCVASGYTFSSY | 283 |
| VHH-8 | CMGWFRQAPGKEREGVAALGGGSTYYADSVKG | |
| | RFTISQDNAKNTLYLQMNSLKPEDTAMYYCAA | |
| | AWVACLEFGGSWYDLARYKHWGQGTQVTVSS | |
| hIL2Rg_ | QVQLQESGGGSVQAGGSLRLSCTASGFTFDDS | 284 |
| VHH-9 | DMGWYRQAPGGECELVTISSDGSTYYADSVKG | |
| | RFTISQDNAKNTVYLQMNSLKPEDTAVYYCAA | |
| | EPRGYYSNYGGRRECNYWGQGTQVTVSS | |
| hIL2Rg_ | QVQLQESGGGSVQAGGSLRLSCAASGSIYSSA | 285 |
| VHH-10 | YIGWFRQAPGKKREGVAGIYTRDGSTAYADSV | |
| | KGRFTISQDSAKKTVYLQMNSLKPEDTAMYYC | |
| | AAGRRTKSYVYIFRPEEYNYWGQGTQVTVSS | |
| hIL2Rg_ | QVQLQESGGGSVQAGGSLRLSCAASGFTFSSA | 286 |
| VHH-11 | HMSWVRQAPGKGREWIASIYSGGGTFYADSVK | |
| | GRFTISRDNAKNTLYLQLNSLKTEDTAMYYCA | |
| | TNRLHYYSDDDSLRGQGTQVTVSS | |
| hIL2Rg_ | QVQLQESGGGSVQAGGSLRLSCAASGFTFSNA | 287 |
| VHH-12 | HMSWVRQAPGKGREWISSIYSGGSTWYADSVK | |
| | GRFTISRDNSKNTLYLQLNSLKTEDTAMYYCA | |
| | ENRLHYYSDDDSLRGQGTQVTVSS | |
| hIL2Rg_ | QVQLQESGGGSVQAGGSLRLSCTASRFIFDDS | 288 |
| VHH-13 | DMGWYRQAPGNECELVSTISSDGSTYYADSVK | |
| | GRFTISRDNAKNTVYLQMNSLKPEDTAVYYCA | |
| | AEPRGYYSNYGGRRECNYWGQGTQVTVSS | |
| hIL2Rg_ | QVQLQESGGGSVQAGGSLKLSCTVSGFTADDS | 289 |
| VHH-14 | DMGWYRQGPGNECELVTISSDGSTYYADSVKG | |
| | RFTISQDNAKNTVYLQMNSLKPEDTAVYYCAA | |
| | EPRGYYSNYGGRRECNYWGQGTQVTVSS | |
| hIL2Rg_ | QVQLQESGGGLVQPGGSLRLSCAASGFTFSSA | 290 |
| VHH-15 | HMSWVRQAPGKGREWIASIYSGGGTFYADSVK | |
| | GRFTISRDNAKNTLYLQLNSLKAEDTAMYYCA | |
| | TNRLHYYSDDDSLRGQGTQVTVSS | |
| hIL2Rg_ | QVQLQESGGGLVQPGGSLRLSCVASGFTFSNA | 291 |
| VHH-16 | HMSWVRQAPGKGREWISSIYSGGSTWYADSVK | |
| | GRFTISRDNSKNTLYLQLNSLKTEDTAMYYCA | |
| | ENRLHYYSDDDSLRGQGTQVTVSS | |
| hIL2Rg_ | QVQLQESGGGLVQPGGSLRLSCAASGFTFSNA | 292 |
| VHH-17 | HMSWVRQAPGKGREWISSIYSGGSTWYADSVK | |
| | GRFTISRDNSKNTLYLQLNSLKTEDTAMYYCA | |
| | ENRLHYYSDDDSLRGQGTQVTVSS | |
| hIL2Rg_ | QVQLQESGGGLVQPGGSLRLSCAASGFTFSSY | 293 |
| VHH-18 | PMTWARQAPGKGLEWVSTIASDGGSTAYAASV | |
| | EGRFTISRDNAKSTLYLQLNSLKTEDTAMYYC | |
| | TKGYGDGTPAPGQGTQVTVSS | |
| hIL2Rg_ | QVQLQESGGGSVQAGGSLRLSCTASGFTFDDR | 294 |
| VHH-19 | EMNWYRQAPGNECELVSTISSDGSTYYADSVK | |
| | GRFTISQDNAKNTVYLQMDSVKPEDTAVYYCA | |
| | ADFMIAIQAPGAGCWGQGTQVTVSS | |
| hIL2Rg_ | QVQLQESGGGSVQAGGSLRLSCTASGFTFDDS | 295 |
| VHH-20 | DMGWYRQAPGNECELVSTISSDGSTYYADSVK | |
| | GRFTISQDNAKNTVYLQMNSLKPEDTAVYYCA | |
| | AEPRGYYSNYGGRRECNYWGQGTQVTVSS | |
| hIL2Rg_ | QVQLQESGGGSVQAGGSLRLSCVASGYTSCMG | 296 |
| VHH-21 | WFRQAPGKEREAVATIYTRGRSIYYADSVKGR | |
| | FTISQDNAKNTLYLQMNSLKPEDIAMYSCAAG | |
| | GYSWSAGCEFNYWGQGTQVTVSS | |
| hIL2Rg_ | QVQLQESGGGLVQPGGSLRLSCTASGFSFSSY | 297 |
| VHH-22 | PMTWARQAPGKGLEWVSTIASDGGSTAYAASV | |
| | EGRFTISRDNAKSTLYLQLNSLKTEDTAMYYC | |
| | TKGYGDGTPAPGQGTQVTVSS | |
| hIL2Rg_ | QVQLQESGGGLVQPGGSLRLSCAASGFSFSSY | 298 |
| VHH-23 | PMTWARQAPGKGLEWVSTIASDGGSTAYAASV | |
| | EGRFTISRDNAKSTLYLQLNSLKTEDTAMYYC | |
| | TKGYGDGTPAPGQGTQVTVSS | |

TABLE 15
murine anti-IL2Rg VHH Amino Acid Sequences

| | VHH AA Sequence | SEQ ID |
|---|---|---|
| Name | (CDRs Underlined) | NO: |
| mIL2Rg_ | QVQLQESGGGSVLAGGSLRLSCVASGYGYNYIGWFRQTPGKERE | 299 |
| VHH1 | GVAVIYTGGGDTYYADSVKGRFTASRDNAKSTLYLQMNSLEPED | |
| | TAMYYGVARYCVGSVYACLRGGHDEYAHWGQGTQVTVSS | |
| mIL2Rg_ | QVQLQESGGGSVQPGGSLRLSCAASGSTYANYLMGWFRQAPGKE | 300 |
| VHH2 | REGVAAIYSGGGSTYYADSVKGRFTISQDNAKNTLYLQMNSLKP | |
| | EDTAMYYCAAASAVKGDKGDIVVVVTGTQRMEYDYWGHGTQVTV | |
| | SS | |
| mIL2Rg_ | QVQLQESGGGSVQAGASLRLSCSVSGFTFDESVMSWLRQGPGNE | 301 |
| VHH3 | CDAVAIISSDDNTYYDDSVKGRFTISEDNAKNMVYLQMNSLKPE | |
| | DTAVYYCAARRRRPVYDSDYELRPRPLCGDFGVWGQGTQVTVSS | |
| mIL2Rg_ | QVQLQESGGGSVQAGGSLRLSCIGSGLPFDEDDMGWYRQAPGNE | 302 |
| VHH4 | CELVSSISSDGTAYYADSVKGRFTISRDNAKNTVLLQMNSLKPE | |
| | DTAVYYCAAGVHRQFGGSSSCGDAFYGMDYWGKGTQVTVSS | |
| mIL2Rg_ | QVQLQESGGGSVQAGGSLRLSCVASGDVYGRNSMAWFRQAPGKE | 303 |
| VHH5 | REGVAVGYSVVTTTYYADSVKGRFTISEDNDKNTVYLEMNSLKP | |
| | EDTAMYYCAADGNLWRGLRPSEYTYWGQGTQVTVSS | |
| mIL2Rg_ | QVQLQESGGGSVQAGGSLRLSCATSGFPYSRYCMGWFRQAPGKE | 304 |
| VHH6 | REGVAAIEPDGSTSYADSVKGRFTISQDNAVNTLYLQMNNLKPE | |
| | DTAMYYCAADERCFYLKDYDLRRPAQYRYWGQGTQVTVSS | |
| mIL2Rg_ | QVQLQESGGGLVQPGGSLRLSCTVSGFTFDESDMGWLRQNPGNE | 305 |
| VHH7 | CGVVSVITSDDNPYYDDSVKGRFTISEDNAKNMVYLQMNSLKPE | |
| | DTGVYYCATRSRQPVYSRDYELRPRPLCGDFGVWGQGTQVTVSS | |
| mIL2Rg_ | QVQLQESGGGSVQAGGSLRLSCTASGFTFDDFDMGWYRQAPGNE | 306 |
| VHH8 | CELVSTISDDGSTYYADSVKGRSSISRDNAKNTVYLQMNRLKPE | |
| | DTGVYYCAAEGALGSKTNCGWVGNFGYWGQGTQVTVSS | |
| mIL2Rg_ | QVQLQESGGGSVQAGGSLRLSCAASGFTFDDFDMGWYRQAPGNE | 307 |
| VHH9 | CELVSTISDDGSTYYADSVKGRSSISRDNAKNTVYLQMNSLKPE | |
| | DTAVYYCAAEGALGSKTNCGWVGNFGYWGQGTQVTVSS | |
| mIL2Rg_ | QVQLQESGGGLVQPGGSLRLSCAASGFTFDDFDMGWYRQAPGNE | 308 |
| VHH10 | CELVSTISDDGSTYYADSVKGRSSISRDNAKSTVYLQMNRLKPE | |
| | DTGVYYCAAEGALGSKTNCGWVGNFGYWGQGTQVTVSS | |
| mIL2Rg_ | QVQLQESGGGLVQPGGSLKLSCAASGFTFSDRDMGWYRQAPGNE | 309 |
| VHH11 | CERVSTISDDGSTYYADSVKGRSSISRDNAKNTVYLQMNSLKPE | |
| | DTAVYYCAAEGALGSKTNCGWVGNFGYWGQGTQVTVSS | |
| mIL2Rg_ | QVQLQESGGGSVLAGGSLRLSCVASGYGYNYIGWFRQTPGKERE | 310 |
| VHH12 | GVAVIYIGGGDTYYADSVKGRFTASRDNAKSTLYLQMNSLEPED | |
| | TAMYYCVARYCVGSVYACLRGGHDEYAHWGQGTQVTVSS | |
| mIL2Rg_ | QVQLQESGGGSVLAGGSLRLSCVASGYGYNYIGWFRQTPGKERE | 311 |
| VHH13 | GVAVIYTGGGDTYYADSVKGRFTASRDNAKSTLYLQMNSLEPED | |
| | TAMYYCVARYCVGSVYACLRGGHDEYAHWGQGTQVTVSS | |
| mIL2Rg_ | QVQLQESGGGSVQAGGSLRLSCAASGFTFDDFDMGWYRQAPGNE | 312 |
| VHH14 | CELVSTISDDGSTYYANSVKGRSSISRDNAKNMVYLQMNSLKPE | |
| | DTAVYYCAAEGALGSKTNCGWVGNFGYWGQGTQVTVSS | |
| mIL2Rg_ | QVQLQESGGGSVQAGGSLRLSCTASGFTFDDFDMGWYRQAPGNE | 313 |
| VHH15 | CELVSTISDDGSTYYADSVKGRSSISRDNAKNTVYLQMNRLKPE | |
| | DTGVYYCAAEGALGSKMNCGWVGNFGYWGQGTQVTVSS | |

TABLE 16
anti-IL10Ra sdAb VHH DNA SEQUENCE

| hIL10Ra VHH DNA Sequences | | SEQ ID |
|---|---|---|
| Name | Sequence | NO: |
| hIL10Ra_VHH1 | CAGGTTCAGCTTCAGGAGTCCGGTGGAGGCTCCA | 276 |
| | TCCAGGCCGGGGGCTCTCTCCGCCTGTCTTGCGCC | |
| | GCTTCCAGATACCTCTACAGTATCGACTACATGG | |
| | CTTGGTTTCGTCAGAGCCCAGGAAAAGAGCGGGA | |
| | ACCCGTGGCAGTAATCTACACTGCCTCAGGTGCC | |
| | ACATTTTACCCCGACTCTGTCAAGGGCAGGTTCA | |
| | CCATCTCTCAGGATAATGCCAAGATGACAGTGTA | |
| | CTTGCAGATGAACTCCCTGAAATCTGAGGATACC | |
| | GCTATGTATTACTGTGCCGCAGTGCGCAAGACCG | |
| | ATTCTTACCTGTTCGACGCTCAGAGTTTTACCTAC | |
| | TGGGGCCAGGGCACTCAGGTCACCGTCAGCAGC | |
| hIL10Ra_VHH2 | CAGGTGCAGTTGCAGGAGTCCGGCGGGGGTTCCG | 277 |
| | TGCAAGCAGGCGGATCTCTGCGCCTGTCCTGCGT | |
| | GGCCTCTCGTTATTTGTATAGCACCAACTACATGG | |
| | CTTGGTTCCGTCAGTCCCCAGGCAAAGAGCGCGA | |
| | AGCCGTAGCCGTAATCTATACGGCCTCTGGGGCA | |
| | ACACTCTATACCGACTCAGTGAAGGGACGCTTCA | |
| | CGATTTCCCAAGACAATGCAAAGATGACCGTGTA | |
| | CTTGCAGATGAACCGCCTGAAGAGCGAGGACACG | |
| | GCTATGTATTACTGCGCAGCCGTGCGCAAGACCG | |
| | ACTCCTACTTGTTTGACGCTCAGTCCTTCACTTAT | |
| | TGGGGCCAGGGTACACAGGTCACCGTGAGCAGT | |
| hIL10Ra_VHH3 | CAAGTACAGCTCCAGGAGAGCGGCGGTGGATCTA | 278 |
| | TCCAAGCAGGGGGTAGCCTTAGGTTGTCCTGTGT | |
| | GGCGTCCAGATACCTGTATAGCACGAACTACATG | |
| | GCATGGTTCAGACAGTCCCCAGGCAAGGAACGCG | |
| | AGGCAGTCGCCGTTATTTACACTGCATCTGGGGC | |
| | CACCCTCTATACGGACAGCGTGAAGGGAAGGTTT | |
| | ACAATCTCCCAGGACAACGCGAAGATGACCGTGT | |
| | ACCTTCAGATGAACCGCCTGAAGTCCGAGGACAC | |
| | CGCCATGTATTACTGTGCAGCGGTGCGCAAGACC | |
| | GACAGCTATCTGTTCGACGCGCAGTCATTCACTTA | |
| | TTGGGGCCAAGGAACCCAAGTGACCGTCAGCTCA | |
| hIL10Ra_VHH4 | CAGGTGCAGCTCCAAGAGTCCGGGGGAGGCTCTA | 279 |
| | TCCAGGCGGGAGGCAGTCTGCGCTTGTCCTGCGC | |
| | CGCAAGTCGTTATCTGTACTCCATTGATTACATGG | |
| | CATGGTTCCGCCAGTCCCCAGGTAAGGAACGTGA | |
| | ACCTGCCGCTGTGATCTACACCGCTTCTGGAGCA | |
| | ACCTTTTATCCTGATAGCGTTAAGGGTCGCTTCAC | |
| | CATCTCTCAGGATAACGCCAAAATGACAGTGTAC | |
| | CTCCAGATGAACAGCCTGAAGTCTGAGGACACTG | |
| | CCATGTACTATTGTGCGGCTGTGCGCAAGACCGA | |
| | CTCCTATCTGTTTGATGCACAGAGCTTTACCTATT | |
| | GGGGTCAGGGCACCCAGGTGACTGTGTCTAGC | |
| hIL10Ra_VHH5 | CAGGTCCAGTTGCAGGAGTCCGGTGGAGGTTCCA | 280 |
| | TCCAGGCGGGTGGGTCCCTTCGTCTCTCCTGCGTG | |
| | GCCTCTAAGTACCTGTATTCAACCAACTACATGG | |
| | CATGGTTCAGACAGTCTCCCGGCAAAGAGCGTGA | |
| | GGCAGTGGCCGCGATCTATACAGCTTCTGGGGCC | |
| | ACCCTGTACTCTGATTCCAATAAGGGAAGGTTCA | |
| | CTATCTCACAGGATAACGCCAAAATGACCGTCTA | |
| | CCTTCAGATGAACAGCCTCAAGTCTGAAGACACG | |
| | GCAATGTATTACTGTGCAGCCGTGCGCAAAACTG | |
| | GGAGCTACCTGTTTGACGCTCAGTCTTTCACTTAT | |
| | TGGGGCCAGGGTACGCAGGTGACAGTCTCTTCT | |
| hIL10Ra_VHH6 | CAGGTGCAACTCCAGGAGAGCGGAGGCGGTTCTG | 281 |
| | TTCAGGCAGGAGGTTCCCTGAGACTGTCCTGTGC | |
| | CGCGTCTCGCTTTACGTATTCATCCTACTGCATGG | |
| | GATGGTTCAGACAAGCGCCGGGGAAAGAAAGGG | |
| | AAGGCGTGGCCTCCATTGACTCCGACGGCTCAAC | |
| | TTCATACACTGATAGCGTGAAAGGCCGGTTCACC | |
| | ATCTCTAAGGACAACGCGAAGAACACCCTGTATC | |
| | TCCAGATGAACAGCCTCAAGCCTGAGGATACTGC | |
| | CATGTACTATTGCGCACTCGACCTGATGTCTACTG | |
| | TGGTCCCAGGCTTCTGCGGGTTCCTGCTCTCTGCT | |
| | GGCATGGACTACTGGGGGAAGGGCACTCAGGTA | |
| | ACGGTTAGCTCC | |
| hIL10Ra_VHH7 | CAGGTGCAGCTTCAGGAATCTGGCGGGGGCTCCG | 282 |
| | TGCAGGCCGGGGGCTCCCTCAGACTTTCCTGTGC | |
| | CGTCTCCGGTTACACATTTAACAGTAACTGTATGG | |
| | GCTGGTTCCGCCAGGCACCAGGCAAGGAGAGGG | |
| | AAGGTGTGGCCACAATCTATACTGGTGTTGGGAG | |
| | TACGTACTATGCTGATTCCGTGAAAGGTCGCTTCA | |
| | CAATTTCCCAGGACAACGCGAAGAACACTGTGTA | |
| | CTTGCAGATGAATAGCCTGAAGCCTGAAGATACC | |
| | GCAATGTATTACTGCGCTGCCGAGCCACTCTCCC | |
| | GCGTATATGGTGGAAGTTGCCCCACCCCCACTTTC | |
| | GGTTACTGGGGCCAGGGCACTCAAGTGACCGTGT | |
| | CCTCT | |
| hIL10Ra_VHH8 | CAGGTTCAGCTTCAGGAGTCTGGGGGCGGTTCAG | 283 |
| | TGCAGGCTGGCGGTTCTCTCCGCCTGTCCTGCGCT | |
| | GCCAGCGGCTATACTTACAGCATGTACTGCATGG | |
| | GCTGGTTCCGGCAAGCCCCCGGCAAAGAGCGTGA | |
| | GGGCGTCGCTCAAATCAACAGCGACGGGTCAACC | |
| | AGCTACGCCGATTCTGTCAAGGGCAGATTTACTA | |
| | TCAGCAAGGACAACGCCAAAAACACACTGTACCT | |
| | CCAGATGAACTCTTTGAAGCCTGAGGACACCGCG | |
| | ATGTATTACTGCGCCGCTGACAGCCGCGTGTACG | |
| | GTGGCAGCTGGTATGAGAGGCTGTGCGGCCCGTA | |
| | CACCTACGAGTACAACTATTGGGGACAGGGCACG | |
| | CAGGTGACAGTTAGCTCC | |
| hIL10Ra_VHH9 | CAGGTGCAACTGCAAGAGAGTGGCGGAGGCTCC | 284 |
| | GTCCAGGCTGGAGGTTCCCTGCGGCTGTCTTGCG | |
| | CCGTCAGCGGCTACGCATATTCCACTTACTGTATG | |
| | GGTTGGTTCCGCCAGGCCCCTGGAAAGGAACGCG | |
| | AGGGTGTTGCCGCTATTGATAGCGGAGGCTCCAC | |
| | ATCCTATGCGGACTCCGTGAAAGGTCGTTTCACC | |
| | ATCTCCAAGGATAACGCCAAGAACACTCTGTACC | |
| | TGCGCATGAACTCTCTGAAGCCTGAGGACACTGC | |
| | CATGTATTACTGCGCCGCTGTGCCCCCTCCACCCG | |
| | ACGGGGGCTCTTGTCTGTTTCTTGGCCCGGAGATC | |
| | AAGGTGTCCAAGGCTGATTTCCGTTATTGGGGCC | |
| | AGGGAACTCAAGTCACCGTGTCTTCC | |
| hIL10Ra_VHH10 | CAGGTCCAGCTCCAGGAGTCCGGTGGAGGCTCCG | 285 |
| | TTCAGGCCGGTGGCAGCTTGCGTCTGAGCTGCGC | |
| | GGCTTCAAGATACCTGTACTCCATTGATTACATGG | |
| | CATGGTTCCGTCAGTCTCCTGGCAAGGAGCGCGA | |
| | GCCCGTCGCTGTGATCTATACCGCCAGCGGAGCC | |
| | ACGTTCTACCCTGATTCCGTCAAGGGCCGCTTCAC | |
| | CATTAGCCAAGACAACGCTAAGATGACGGTGTAC | |
| | CTCCAAATGAATAGCCTGAAAAGCGAGGACACA | |
| | GCGATGTATTACTGCGCCGCTGTTAGGAAAACTG | |
| | ATAGTTACCTGTTCGATGCACAGTCTTTCACTTAC | |
| | TGGGGGCAGGGCACCCAAGTTACCGTCTCCTCT | |
| hIL10Ra_VHH11 | CAGGTGCAGCTCCAGGAATCTGGAGGGGGCAGTG | 286 |
| | TGCAGGCCGGGGGCTCCCTGCGCTTGAGCTGTGG | |
| | AGCCAGCCGCTACACGTATTCCAGTTACTGTATG | |
| | GGCTGGTTCAGACAAGCTCCGGGTAAGGAGAGA | |
| | GAGGGAGTTGCCGTAATTGATTCTGACGGGTCCA | |
| | CTAGCTATGCGGATTCAGTCAAGGGCCGGTTCAC | |
| | CATCAGCAAGGACAATGGTAAGAACACACTGTAC | |
| | CTGCAAATGAACAGCCTGAAGCCCGAGGACACCG | |
| | CCATGTACTATTGTGCCGCTGATCTCGGACATTAC | |
| | CGCCCTCCCTGCGGTGTGCTCTATCTCGGGATGGA | |
| | CTATTGGGGTAAGGGCACCCAGGTGACCGTGTCC | |
| | TCT | |
| hIL10Ra_VHH12 | CAGGTGCAGCTCCAGGAAAGCGGCGGGGGTAGC | 287 |
| | GTTCAAGCAGGTGGGTCCCTGCGCTTGAGCTGTA | |
| | CTGTGTCCGGCTACACCTACTCAAGCAACTGCAT | |
| | GGGATGGTTCCGTCAGGCCCCTGGCAAGGAACGC | |
| | GAAGGCGTGGCTACTATCTACACCGGCGGTGGCA | |
| | ACACTTATTACGCCGACTCCGTTAAGGGGCGTTTC | |
| | ACTATCAGCCAAGACAACGCCAAGAACACCGTGT | |
| | ATCTGCAAATGAATAACCTGAAGCCTGAAGACAC | |
| | CGCCATGTATTACTGTGCTGCCGAGCCCCTTTCCC | |
| | GCGTTTACGGCGGTTCTTGTCCTACCCCTACCTTT | |
| | GACTACTGGGGTCAGGGAACACAGGTGACAGTGT | |
| | CCAGT | |
| hIL10Ra_VHH13 | CAAGTCCAACTCCAGGAATCTGGGGGAGGCTCCG | 288 |
| | TACAGGCTGGCGGTTCCCTTCGTCTGTCCTGTGCT | |
| | GTGTCAGGGTACTCCTACTCCAGTAACTGTATGG | |
| | GCTGGTTCCGGCAAGCCCCCGGAAAGGAGCGCGA | |
| | GGGCGTGGCTACCATCCACACAGGGGGCGGTTCC | |
| | ACATATTACGCCGATAGTGTCAAGGGCCGCTTCA | |
| | CCATTAGTCAGGACAACGCCAAGAATACCGTTTA | |
| | CCTTCAAATGAACTCTTTGAAACCTGAGGACACT | |
| | GCGATGTATTACTGTGCGGCAGAGCCTTTGTCCC | |
| | GCCTGTACGGGGGATCTTGTCCGACCCCGACTTTC | |
| | GGGTACTGGGGACAGGGCACCCAGGTGACAGTGT | |
| | CCTCC | |
| hIL10Ra_VHH14 | CAGGTGCAGTTGCAGGAAAGCGGGGGTGGCAGC | 289 |
| | GTCCAAGCCGGTGGCAGCCTGCGTCTGTCCTGCG | |
| | GTGCCTCCGGCTATACTTACTCCAGCTATTGCATG | |
| | GGTTGGTTCCGCCAAGTGCCAGGAAAGGAGCGTG | |
| | AGGGGGTGGCTGTAATTGATTCAGATGGGTCAAC | |
| | AAGCTACGCTGACAGCGTTAAAGGTCGCTTCACC | |
| | ATCAGTAAGGACAACGGCAAGAACACCCTCTACC | |
| | TGCAAATGAACTCCCTGAAGCCGGAGGATACCGC | |
| | AATGTATTACTGTGCCGCTGACTTGGGACACTAC | |
| | CGCCCTCCGTGCGGTGTGCTTTATCTGGGCATGGA | |
| | TTACTGGGGTAAGGGAACCCAAGTGACGGTGTCT | |
| | TCT | |
| hIL10Ra_VHH15 | CAGGTACAACTCCAGGAGTCTGGCGGTGGGTCCG | 290 |
| | TGCAGGCAGGTGGCAGCCTTCGCCTCTCCTGCGG | |
| | GGCCTCCGGGTACACCTATAGTGGCTACTGCATG | |
| | GGGTGGTTCAGGCAAGCCCCCGGTAAGGAACGTG | |
| | AGGGAGTTGCTGTGATTGATTCAGATGGGTCCAC | |
| | GAGTTACGCTGACTCCGTGAAAGGTAGGTTCACA | |
| | ATCTCCAAAGATAATGGCAAGAACACCCTCTACC | |
| | TTCAGATGAATAGCCTGAAGCCAGAAGACACCGC | |
| | CATGTATTACTGTGCTGCCGACCTGGGACACTATC | |
| | GCCCTCCGTGCGGGGTCCTGTACTTGGGCATGGA | |
| | CTATTGGGGCAAGGGGACCCAGGTGACTGTGTCC | |
| | TCT | |
| hIL10Ra_VHH16 | CAGGTGCAGTTGCAGGAATCCGGTGGAGGCTCTG | 291 |
| | TTCAGGCCGGGGGCTCTCTCCGCCTGGCCTGCGC | |
| | AGCCTCCAGGTATACTTACAGCAACTACTGCATG | |
| | GGGTGGTTTCGCCAGGCTCCGGGCAAAGAGCGTG | |
| | AGGGAGTGGCTACTATTGATTCCGATGGAAACAC | |
| | CAGCTACGCCGATAGCGTGAAGGGCAGATTTACT | |
| | ATCAGCAGAGATAACGCTAAAAACACGTTGTACC | |
| | TCCAGATGAACTCACTCAAGCCGGGGGACACAGC | |
| | TATGTATTACTGCGCAGCCGATCTGGGTCACTACC | |
| | GCCCGCCCTGCGGCGCATATTACTATGGCATGGA | |
| | CTACTGGGGCAAGGGCACCCAGGTGACCGTGTCC | |
| | AGT | |
| hIL10Ra_VHH17 | CAGGTGCAGCTCCAAGAGTCTGGCGGGGGTTCCG | 292 |
| | TGCAAGCCGGTGGCTCACTCAGGTTGAGTTGCGC | |
| | AGCCAGCGGCTATAGCAACTGTTCCTATGACATG | |
| | ACTTGGTATCGCCAGGCCCCTGGCAAAGAGCGTG | |
| | AGTTCGTGTCAGCTATTCACTCCGACGGCTCCACT | |
| | CGTTATGCGGACTCTGTGAAGGGCCGGTTTTTCAT | |
| | CTCCCAGGACAACGCTAAAAACACTGTCTATTTG | |
| | CAGATGAACTCTCTGAAACCCGAAGATACCGCCA | |
| | TGTACTATTGCAAAACCGATCCTCTGCATTGTCGC | |
| | GCCCACGGCGGGAGTTGGTACTCTGTGCGGGCCA | |
| | ACTATTGGGGCCAGGGCACCCAGGTCACCGTGTC | |
| | CTCA | |
| hIL10Ra_VHH18 | CAGGTACAACTCCAGGAGTCTGGCGGTGGCAGCG | 293 |
| | TGCAGGCAGGCGGAAGCCTGAGGCTGTCCTGCGC | |
| | TGTATCTGGCTACACTTATAATTCCAACTGCATGG | |
| | GTTGGTTTCGGCAGGCTCCAGGTAAGGAGCGCGA | |
| | GGGCGTCGCCACCATTTATACAGGTGTTGGCAGC | |
| | ACATATTACGCCGACAGCGTGAAGGGAAGGTTCA | |
| | CCATCTCCCAAGACAATGCGAAAAACACAGTGTA | |
| | TCTCCAGATGAATAGCCTGAAGCCCGAGGACACG | |
| | GCTATGTATTACTGCGCTGCCGAGCCACTGAGCA | |
| | GAGTGTATGGGGGCAGCTGTCCTACACCCACTTT | |
| | CGGCTATTGGGGTCAAGGCACCCAGGTTACAGTC | |
| | AGCTCC | |

TABLE 17
anti-IL2Rg VHH DNA sequences

| Name | Sequence | SEQ ID NO: |
|---|---|---|
| hIL2Rg_VHH-1 | CAGGTCCAGCTCCAGGAGAGCGGGGG | 294 |
| | CGGTTCTGTGCAAGCCGGAGGCTCATT | |
| | GAGACTCTCATGCGCTGCAAGTGGTTT | |
| | TACCTTCGATGACAGCGATATGGGATG | |
| | GTATCGTCAGGCTCCGGGCAATGAGTG | |
| | TGATCTGGTCTCCACTATCTCCTCTGAT | |
| | GGTTCCACATACTATGCTGACTCTGTCA | |
| | AGGGGCGCTTTACCATCTCCCAAGATA | |
| | ATGCCAAGAACACCGTGTACCTTCAGA | |
| | TGGATTCAGTTAAGCCCGAGGACACAG | |
| | CCGTCTATTACTGCGCTGCGGATTTTAT | |
| | GATTGCCATCCAAGCTCCCGGAGCGGG | |
| | ATGCTGGGGCCAGGGAACCCAGGTCAC | |
| | TGTGAGCAGT | |
| hIL2Rg_VHH-2 | CAGGTGCAGTTGCAGGAGTCCGGCGGG | 295 |
| | GGTTCTGTGCCAGCGGGTGGGAGCCTC | |
| | AAGCTCTCCTGTGCCGCTTCCGGCTTCT | |
| | CATTCTCCTCTTACCCTATGACCTGGGC | |
| | ACGCCAAGCGCCCGGCAAGGGACTGG | |
| | AATGGGTGTCCACCATTGCTTCCGATG | |
| | GCGGTAGTACAGCCTACGCCGCGTCAG | |
| | TGGAGGGTCGGTTCACGATCAGCCGGG | |
| | ACAACGCGAAGAGCACACTCTACCTCC | |
| | AGCTGAACTCTCTGAAGACCGAGGACA | |
| | CCGCCATGTACTATTGCACAAAGGGCT | |
| | ACGGCGACGGCACCCCGGCACCCGGCC | |
| | AGGGCACCCAGGTGACAGTCTCTTCC | |
| hIL2Rg_VHH-3 | CAGGTGCAGTTGCAGGAAAGTGGTGGA | 296 |
| | GGGAGTGTGCAGACTGGGGGCTCTCTC | |
| | CGCCTCAGCTGCACAGCCTCTGGATTT | |
| | ACCTTCGATGATCGCGAGATGAACTGG | |
| | TATCGCCAGGCTCCGGGAAACGAGTGC | |
| | GAACTGGTGTCTACAATCAGTTCTGAC | |
| | GGGTCCACCTATTACGCTGATAGTGTC | |
| | AAGGGCCGCTTCACTATCTCTCAGGAC | |
| | AACGCGAAGAACACCGTTTACTTGCAG | |
| | ATGGATAGCGTGAAGCCTGAAGATACA | |
| | GCGGTGTATTACTGCGCTGCCGACTTT | |
| | ATGATTGCCATCCAGGCACCGGGGGCG | |
| | GGGTGTTGGGGACAGGGAACTCAGGTG | |
| | ACTGTGTCCTCC | |
| hIL2Rg_VHH-4 | CAGGTTCAACTCCAAGAGAGTGGTGGC | 297 |
| | GGAAGCGTGCAGGCGGGCGGTTCTCTG | |
| | CGTCTGAGTTGCACTGCCAGCGGATTT | |
| | ACCTTCGACGATTCCGACATGGGATGG | |
| | TACAGACAGGCCCCTGGTAACGAGTGC | |
| | GAACTCGTGAGTACTATCAGCTCCGAC | |
| | GGCAACACCTATTACACCGATTCTGTG | |
| | AAGGGCAGGTTCACCATCTCCCAGGAC | |
| | AACGCTAAGAACACTGTGTACCTGCAA | |
| | ATGAATAGCCTGGGACCCGAGGACACA | |
| | GCGGTCTATTACTGCGCGGCAGAGCCG | |
| | CGCGGCTATTACAGCAACTACGGCGGT | |
| | AGACGCGAGTGCAACTACTGGGGGCA | |
| | GGGGACGCAAGTGACTGTCTCCTCC | |
| hIL2Rg_VHH-5 | CAAGTGCAGCTTCAGGAGTCCGGGGGT | 298 |
| | GGCAGCGTCCAGGCTGGGGGCAGCTTG | |
| | CGCCTGTCTTGCGCTGCGTCTGGGTTCA | |
| | GCTTTAGCTCCTACCCTATGACCTGGGC | |
| | TAGACAGGCCCCCGGCAAGGGGCTGG | |
| | AGTGGGTGAGTACAATCGCCTCCGACG | |
| | GAGGTAGTACGGCCTACGCAGCGTCCG | |
| | TCGAGGGTCGCTTCACCATCAGCCGGG | |
| | ATAACGCTAAGTCCACCCTGTACCTTC | |
| | AGCTCAATTCTCTCAAAACGGAGGATA | |
| | CCGCCATGTACTATTGCACCAAGGGAT | |
| | ATGGCGACGGCACCCCAGCTCCTGGAC | |
| | AGGGCACACAGGTCACCGTTAGCTCC | |
| hIL2Rg_VHH-6 | CAGGTCCAGCTTCAGGAGTCTGGCGGG | 299 |
| | GGCGCAGTACAGGCAGGGGGTTCTCTG | |
| | CGTCTGTCCTGCGCCGCGTCCGGCTTTA | |
| | CTTTCAGCAACGCACACATGAGTTGGG | |
| | TGCGCCAAGCGCCCGGCAAGGGCCGG | |
| | GAATGGATCAGTAGCATCTACAGTGGA | |
| | GGCAGCACATGGTACGCCGACTCTGTT | |
| | AAGGGTCGTTTTACGATCTCTCGTGAC | |
| | AACTCCAAGAACACTTTGTACCTCCAG | |
| | CTCAATTCTCTCAAGACCGAGGACACC | |
| | GCGATGTACTATTGTGCCGAGAACAGG | |
| | CTGCACTACTATTCCGACGATGACTCTC | |
| | TCAGGGGCCAGGGAACTCAAGTTACCG | |
| | TGTCCAGC | |
| hIL2Rg_VHH-7 | CAAGTGCAGCTCCAAGAGAGTGGTGGC | 300 |
| | GGGCTGGTTCAGCCAGGGGGCAGCTTG | |
| | AGACTCTCCTGCGCAGCTTCAGGCTTT | |
| | ACCTTCGATGACCGTGAGATGAACTGG | |
| | TATCGTCAGGCCCCAGGCAACGAGTGT | |
| | GAGCTGGTTAGCACGATTTCTTCCGAC | |
| | GGTTCCACCTATTACGCCGACTCTGTG | |
| | AAGGGACGTTTCACTATCTCCCAGGAC | |
| | AATGCCAAGAACACCGTGTACCTCCAG | |
| | ATGGACAGCGTGAAGCCGGAGGATACT | |
| | GCTGTGTATTACTGCGCTGCCGACTTTA | |
| | TGATCGCCATCCAGGCCCCTGGCGCGG | |
| | GTTGCTGGGGCCAGGGCACTCAGGTGA | |
| | CCGTGTCTTCC | |
| hIL2Rg_VHH-8 | CAAGTGCAACTGCAAGAGTCCGGCGGT | 301 |
| | GGATCTGTGCAGGCCGGAGGCAGCCTG | |
| | CGGCTGAGCTGTGTAGCTTCCGGGTAT | |
| | ACCTTTAGCTCATACTGTATGGGCTGGT | |
| | TTCGTCAGGCCCCCGGTAAGGAGCGCG | |
| | AGGGCGTGGCCGCTCTTGGTGGAGGCT | |
| | CCACCTATTACGCCGATTCCGTGAAGG | |
| | GCAGGTTTACTATCTCCCAGGACAACG | |
| | CGAAGAATACGCTCTATCTCCAGATGA | |
| | ATAGCCTGAAGCCCGAGGATACAGCTA | |
| | TGTATTACTGTGCTGCCGCTTGGGTAGC | |
| | CTGCCTGGAGTTCGGTGGCTCCTGGTA | |
| | CGATCTGGCACGGTACAAACATTGGGG | |
| | GCAGGGCACCCAGGTCACCGTGTCTAG | |
| | C | |
| hIL2Rg_VHH-9 | CAGGTCCAGTTGCAGGAATCTGGGGGC | 302 |
| | GGTTCCGTACAAGCAGGTGGCTCCCTT | |
| | CGGTTGAGCTGTACCGCATCCGGCTTT | |
| | ACTTTCGACGATAGCGATATGGGCTGG | |
| | TATCGTCAGGCCCCAGGGGGCGAGTGC | |
| | GAGCTGGTTACAATCTCCTCTGACGGC | |
| | AGTACCTATTACGCAGACTCCGTCAAG | |
| | GGCAGGTTCACTATCAGTCAGGACAAT | |
| | GCAAAGAACACTGTGTATCTCCAGATG | |
| | AACTCTCTGAAGCCAGAAGATACTGCC | |
| | GTGTATTACTGCGCTGCGGAACCGAGA | |
| | GGCTATTACTCTAATTATGGCGGGCGT | |
| | CGGGAGTGTAATTATTGGGGACAGGGA | |
| | ACCCAGGTGACCGTGTCCTCC | |
| hIL2Rg_VHH-10 | CAGGTGCAGCTCCAGGAGAGTGGCGG | 303 |
| | AGGCTCCGTGCAGGCTGGGGGCTCTCT | |
| | GCGTCTGAGCTGTGCCGCAAGCGGTAG | |
| | CATTTACAGCTCTGCCTACATCGGGTG | |
| | GTTTCGTCAAGCGCCGGGCAAAAAGCG | |
| | CGAAGGCGTGGCCGGAATCTACACGCG | |
| | CGATGGCTCCACCGCTTATGCTGACAG | |
| | CGTTAAGGGACGTTTTACGATCAGCCA | |
| | GGACTCTGCCAAAAAGACTGTGTATCT | |
| | CCAGATGAACTCCCTGAAACCTGAGGA | |
| | CACAGCCATGTATTACTGCGCCGCTGG | |
| | CCGCCGTACAAAGAGCTATGTTTACAT | |
| | CTTTCGCCCCGAAGAGTACAACTACTG | |
| | GGGCCAGGGAACCCAAGTGACTGTGTC | |
| | CAGT | |
| hIL2Rg_VHH-11 | CAGGTTCAGTTGCAGGAGTCCGGCGGA | 304 |
| | GGCAGCGTGCAGGCCGGAGGCTCCTTG | |
| | CGCTTGTCCTGTGCGGCTTCTGGCTTCA | |
| | CCTTCTCATCTGCTCACATGAGTTGGGT | |
| | GCGTCAGGCCCCAGGGAAAGGTCGCG | |
| | AGTGGATTGCCTCCATCTACAGCGGTG | |
| | GGGGCACTTTTTATGCGGACAGCGTGA | |
| | AGGGCCGCTTTACCATCAGCCGTGACA | |
| | ACGCTAAGAACACCCTGTATCTCCAAC | |
| | TCAATTCCCTCAAGACCGAGGATACAG | |
| | CGATGTACTATTGTGCAACCAACCGCC | |
| | TTCACTATTACTCCGACGATGACAGCC | |
| | TGCGCGGACAGGGGACCCAGGTGACG | |
| | GTGTCCAGC | |
| hIL2Rg_VHH-12 | CAGGTGCAACTCCAGGAAAGTGGCGG | 305 |
| | AGGCTCAGTGCAGGCAGGTGGCTCTCT | |
| | CCGCCTTTCCTGCGCTGCCAGCGGATTC | |
| | ACCTTCTCTAACGCTCACATGAGCTGG | |
| | GTTCGTCAGGCTCCCGGCAAAGGCCGT | |
| | GAATGGATTAGCTCCATCTATAGTGGC | |
| | GGAAGTACTTGGTACGCAGATAGCGTC | |
| | AAGGGCCGCTTCACTATTAGTCGGGAT | |
| | AACTCCAAGAACACTCTGTACCTCCAG | |
| | CTGAACTCATTGAAAACCGAGGACACG | |
| | GCTATGTACTATTGTGCTGAGAACAGG | |
| | CTGCACTATTACTCCGACGATGACTCTC | |
| | TGAGGGGTCAGGGCACCCAGGTGACCG | |
| | TCAGCTCC | |
| hIL2Rg_VHH-13 | CAGGTCCAACTCCAGGAGTCCGGCGGA | 306 |
| | GGCAGCGTGCAGGCTGGAGGCTCTCTC | |
| | CGCCTGAGCTGCACAGCTTCCAGATTC | |
| | ATCTTCGATGACTCCGACATGGGCTGG | |
| | TATCGCCAGGCTCCAGGGAACGAGTGC | |
| | GAACTGGTGAGCACCATCTCTTCAGAC | |
| | GGTAGCACCTATTACGCCGACAGTGTG | |
| | AAGGGGCGCTTCACCATCTCCCGCGAC | |
| | AATGCTAAAAATACGGTGTATCTCCAG | |
| | ATGAACTCCCTCAAACCGGAGGACACA | |
| | GCTGTATATTACTGTGCTGCGGAACCA | |
| | CGGGGCTACTATAGCAACTATGGTGGA | |
| | AGGCGCGAGTGCAACTACTGGGGTCAG | |
| | GGCACACAGGTGACGGTTTCCTCC | |
| hIL2Rg_VHH-14 | CAGGTGCAGCTCCAGGAGAGCGGCGGT | 307 |
| | GGCTCCGTGCAGGCTGGTGGCAGCCTG | |
| | AAGCTGTCCTGCACCGTGAGTGGCTTC | |
| | ACAGCCGACGATTCTGATATGGGCTGG | |
| | TATCGCCAAGGCCCCGGCAATGAGTGC | |
| | GAGCTGGTAACCATTAGCTCAGACGGC | |
| | TCTACATACTATGCCGATTCTGTTAAGG | |
| | GCCGCTTTACTATCTCACAGGATAATG | |
| | CCAAGAACACAGTGTACTTGCAGATGA | |
| | ACTCTCTGAAACCGGAAGACACAGCTG | |
| | TGTATTACTGTGCTGCGGAGCCTAGAG | |
| | GGTATTACAGCAATTACGGGGGCCGGA | |
| | GAGAGTGTAACTATTGGGGGCAGGGCA | |
| | CCCAAGTGACCGTTTCCTCC | |
| hIL2Rg_VHH-15 | CAGGTCCAGCTTCAGGAATCTGGGGGC | 308 |
| | GGTCTCGTGCAGCCCGGCGGGTCCCTG | |
| | CGTCTGTCTTGTGCTGCGAGCGGCTTCA | |
| | CGTTCTCAAGTGCCCACATGAGCTGGG | |
| | TAAGGCAGGCACCGGGCAAGGGGCGC | |
| | GAGTGGATTGCAAGCATCTATTCAGGC | |
| | GGGGGCACATTCTACGCCGACAGCGTG | |
| | AAGGGACGTTTTACAATCTCCAGAGAT | |
| | AACGCAAAGAACACTCTCTACCTCCAA | |
| | CTCAACTCCTTGAAGGCGGAAGATACT | |
| | GCAATGTATTACTGTGCTACTAACCGT | |
| | CTTCATTATTACTCTGACGATGACTCCC | |
| | TGCGGGGGCAGGGTACACAGGTGACA | |
| | GTGAGTTCC | |
| hIL2Rg_VHH-16 | CAGGTGCAGCTGCAAGAATCTGGTGGA | 309 |
| | GGGCTGGTCCAGCCTGGGGGCTCCCTG | |
| | CGCCTCTCATGTGTCGCATCTGGCTTCA | |
| | CCTTCAGCAACGCCCACATGAGCTGGG | |
| | TTCGCCAAGCCCCTGGGAAGGGCCGGG | |
| | AGTGGATCTCCAGTATCTATTCCGGCG | |
| | GAAGCACTTGGTATGCAGACAGCGTCA | |
| | AAGGACGGTTCACTATTTCTCGTGATA | |
| | ATTCTAAGAACACCCTGTACCTTCAGC | |
| | TGAACAGCCTGAAGACCGAGGACACTG | |
| | CTATGTACTATTGTGCTGAGAATCGCCT | |
| | GCATTACTATAGCGACGATGACAGTCT | |
| | GCGCGGACAGGGGACCCAGGTCACCGT | |
| | GTCCTCT | |
| hIL2Rg_VHH-17 | CAGGTTCAGTTGCAGGAATCAGGAGGC | 310 |
| | GGTCTGGTGCAGCCTGGGGGCTCTCTG | |
| | CGTCTCTCCTGCGCCGCTTCCGGCTTCA | |
| | CATTCTCCAACGCCCACATGAGCTGGG | |
| | TCCGCCAGGCCCCTGGGAAGGGCCGCG | |
| | AGTGGATCTCCAGTATCTACAGCGGGG | |
| | GCTCCACTTGGTACGCAGACAGCGTCA | |
| | AAGGGAGGTTTACCATTAGCCGTGACA | |
| | ATTCTAAGAACACATTGTATTTGCAGC | |
| | TGAACTCTCTTAAAACCGAGGACACCG | |
| | CCATGTACTATTGTGCTGAGAACAGGC | |
| | TCCACTATTACTCAGACGATGACTCAC | |
| | TTCGCGGGCAGGGAACCCAGGTCACCG | |
| | TCTCCTCT | |
| hIL2Rg_VHH-18 | CAAGTCCAGCTCCAGGAAAGCGGCGGT | 311 |
| | GGCCTGGTGCAACCTGGCGGGTCTCTG | |
| | CGCTTGTCATGCGCTGCCTCCGGCTTCA | |
| | CCTTCTCATCTTACCCTATGACCTGGGC | |
| | GCGTCAGGCTCCCGGCAAGGGATTGGA | |
| | GTGGGTGTCTACTATTGCCTCCGACGG | |
| | TGGCAGCACGGCCTACGCAGCGTCTGT | |
| | AGAAGGACGCTTCACAATTAGCAGAGA | |
| | CAACGCAAAATCTACTTTGTACCTTCA | |
| | GCTCAACAGCCTGAAGACCGAAGACAC | |
| | AGCTATGTATTACTGCACAAAAGGCTA | |
| | CGGGGACGGCACGCCAGCGCCTGGAC | |
| | AGGGGACACAGGTGACCGTATCTTCT | |
| hIL2Rg_VHH-19 | CAGGTGCAGTTGCAGGAATCAGGGGGT | 312 |
| | GGCTCTGTGCAGGCCGGGGGCTCCCTG | |
| | CGTCTGTCCTGTACTGCGAGCGGCTTC | |
| | ACCTTTGATGACCGCGAGATGAACTGG | |
| | TATCGCCAGGCTCCGGGGAACGAGTGC | |
| | GAACTCGTGTCTACAATTAGCTCCGAT | |
| | GGTTCAACATACTATGCTGATTCTGTCA | |
| | AAGGTCGCTTTACCATCTCACAGGACA | |
| | ACGCCAAGAACACCGTCTACCTCCAGA | |
| | TGGACTCTGTGAAGCCTGAAGATACCG | |
| | CCGTATACTATTGCGCCGCTGACTTTAT | |
| | GATTGCCATTCAGGCTCCGGGTGCTGG | |
| | ATGCTGGGGTCAGGGGACTCAGGTGAC | |
| | CGTGTCTTCA | |
| hIL2Rg_VHH-20 | CAAGTGCAGTTGCAGGAAAGCGGCGGT | 313 |
| | GGGTCCGTGCAAGCCGGAGGTTCTCTC | |
| | CGCCTGTCTTGCACTGCCTCAGGTTTTA | |
| | CCTTCGACGATTCCGATATGGGCTGGT | |
| | ACAGGCAGGCTCCCGGCAATGAGTGCG | |
| | AGCTGGTGTCTACGATCTCAAGTGATG | |
| | GCTCCACCTACTATGCCGATAGCGTAA | |
| | AAGGAAGGTTTACTATTAGCCAGGATA | |
| | ACGCGAAGAACACGGTGTACCTCCAGA | |
| | TGAACAGTCTCAAGCCGGAGGATACTG | |
| | CCGTGTATTACTGTGCTGCCGAGCCGC | |
| | GTGGCTATTACTCCAACTACGGTGGCA | |
| | GACGTGAATGCAATTACTGGGGACAGG | |
| | GTACTCAGGTTACCGTGTCCTCT | |
| hIL2Rg_VHH-21 | CAGGTTCAACTTCAGGAATCCGGGGGC | 314 |
| | GGTTCCGTGCAAGCCGGGGGTAGCCTG | |
| | CGTCTGTCTTGCGTGGCCAGCGGCTAT | |
| | ACCTCCTGTATGGGTTGGTTTCGGCAG | |
| | GCTCCTGGGAAGGAGCGCGAAGCCGTG | |
| | GCGACCATCTACACACGGGGCCGCAGC | |
| | ATCTATTACGCTGACAGTGTGAAGGGC | |
| | CGCTTCACCATCTCCCAGGATAACGCC | |
| | AAGAATACCCTGTATCTGCAAATGAAC | |
| | TCCCTGAAGCCTGAGGACATCGCCATG | |
| | TATTCCTGCGCAGCTGGAGGGTACTCA | |
| | TGGTCCGCTGGGTGCGAGTTTAATTATT | |
| | GGGGCCAAGGAACCCAGGTGACCGTCT | |
| | CCTCA | |
| hIL2Rg_VHH-22 | CAAGTGCAGCTCCAGGAGTCTGGCGGG | 315 |
| | GGCCTGGTTCAGCCTGGTGGGTCCCTG | |
| | CGCCTGTCTTGCACGGCTTCCGGCTTTA | |
| | GCTTCTCCTCATATCCAATGACCTGGGC | |
| | ACGCCAGGCTCCTGGTAAGGGCCTGGA | |
| | GTGGGTCTCCACCATCGCCTCTGATGG | |
| | TGGGTCAACTGCCTATGCTGCCTCCGTC | |
| | GAGGGTAGATTCACAATCAGCAGAGAC | |
| | AACGCCAAATCCACGCTGTACCTGCAA | |
| | CTCAACTCCTTGAAGACCGAGGACACA | |
| | GCTATGTATTACTGTACCAAAGGCTAC | |
| | GGCGACGGCACTCCTGCTCCCGGACAG | |
| | GGGACCCAGGTGACTGTGTCTAGC | |
| hIL2Rg_VHH-23 | CAGGTCCAACTTCAGGAAAGCGGGGGT | 316 |
| | GGACTGGTACAGCCAGGGGGCAGTCTG | |
| | CGCCTGTCCTGTGCCGCAAGCGGGTTT | |
| | TCTTTCTCCAGTTACCCCATGACCTGGG | |
| | CTCGCCAAGCACCTGGAAAGGGACTGG | |
| | AGTGGGTGTCTACTATTGCGTCAGATG | |
| | GTGGGAGTACGGCTTACGCCGCGAGCG | |
| | TGGAGGGTCGTTTTACGATCAGTAGGG | |
| | ACAACGCCAAAAGCACTCTGTACCTCC | |
| | AGCTTAACAGCCTGAAGACCGAGGACA | |
| | CCGCCATGTATTACTGTACCAAGGGCT | |
| | ACGGAGACGGCACCCCTGCGCCGGGGC | |
| | AAGGCACCCAGGTGACCGTAAGTTCA | |

TABLE 18
murine anti-IL2Rg VHH DNA sequences

| Name | DNA Sequence | SEQ ID NO: |
|---|---|---|
| mIL2Rg_VHH1 | CAGGTGCAACTCCAGGAGTCCGGCGGGGGCTCCGT | 317 |
| | GCTGGCTGGCGGATCTTTGAGGCTGTCTTGCGTGG | |
| | CTTCTGGCTATGGCTATAATTACATCGGCTGGTTCC | |
| | GTCAGACACCCGGCAAGGAGCGCGAAGGGGTGGC | |
| | GGTCATTTACACAGGGGGTGGGGACACTTATTACG | |
| | CCGACTCCGTCAAGGGTAGGTTTACCGCTAGTCGC | |
| | GATAATGCCAAAAGTACGCTGTACCTGCAAATGAA | |
| | CAGCTTGGAGCCAGAGGACACCGCCATGTATTACG | |
| | GAGTGGCTCGCTACTGTGTGGGCAGTGTGTACGCT | |
| | TGCCTGCGCGGAGGCCACGACGAGTACGCACACTG | |
| | GGGCCAGGGAACCCAGGTGACAGTGTCTAGC | |
| mIL2Rg_VHH2 | CAGGTGCAGCTCCAGGAGTCTGGGGGTGGCAGCGT | 318 |
| | CCAGCCAGGTGGCTCATTGAGACTGTCTTGTGCTG | |
| | CATCTGGCTCCACCTACGCTAATTACCTGATGGGA | |
| | TGGTTCAGGCAGGCCCCTGGTAAGGAGCGTGAGG | |
| | GCGTGGCCGCTATCTATTCTGGCGGTGGGTCCACC | |
| | TACTATGCTGACTCCGTCAAGGGACGCTTCACTAT | |
| | TTCTCAAGACAATGCCAAGAACACTTTGTACTTGC | |
| | AAATGAACTCACTCAAACCTGAGGACACCGCGATG | |
| | TACTATTGCGCAGCGGCATCCGCAGTGAAGGGAGA | |
| | CAAAGGGGATATCGTGGTAGTTGTGACCGGCACCC | |
| | AGCGTATGGAGTACGACTACTGGGGACATGGCACC | |
| | CAGGTGACAGTTAGCTCC | |
| mIL2Rg_VHH3 | CAGGTACAGTTGCAGGAGAGTGGTGGGGGTTCCGT | 319 |
| | CCAGGCCGGTGCCTCTCTTCGCCTCAGTTGTAGCGT | |
| | GAGCGGTTTCACGTTCGACGAGTCAGTGATGTCCT | |
| | GGTTGCGCCAGGGTCCCGGCAATGAGTGCGACGCG | |
| | GTCGCTATTATCAGCTCCGATGACAACACCTATTA | |
| | CGACGATAGCGTGAAAGGCCGCTTTACCATCTCCG | |
| | AGGACAACGCCAAAAACATGGTGTATCTGCAAAT | |
| | GAACTCACTGAAGCCGGAAGACACCGCAGTGTACT | |
| | ATTGCGCCGCGCGTCGGCGCAGACCTGTGTACGAT | |
| | TCCGATTATGAACTCCGGCCACGTCCGCTGTGTGG | |
| | CGATTTCGGCGTGTGGGGCCAGGGGACCCAGGTGA | |
| | CGGTCTCCTCC | |
| mIL2Rg_VHH4 | CAGGTGCAGCTCCAGGAATCTGGCGGGGGCTCTGT | 320 |
| | GCAGGCTGGTGGCTCCCTTCGCCTGTCCTGTATTGG | |
| | CTCCGGTCTTCCTTTCGACGAGGATGACATGGGCT | |
| | GGTATCGCCAGGCCCCTGGGAATGAGTGTGAATTG | |
| | GTCAGCTCAATCTCCAGTGACGGCACCGCCTATTA | |
| | CGCCGATTCCGTCAAGGGACGCTTCACTATCTCCA | |
| | GAGACAACGCCAAGAACACTGTGCTGTTGCAGATG | |
| | AACTCCCTGAAGCCCGAGGATACCGCTGTCTATTA | |
| | CTGCGCAGCCGGGGTCCACAGACAGTTCGGCGGTT | |
| | CCAGTTCCTGCGGCGACGCCTTCTACGGCATGGAT | |
| | TACTGGGGCAAGGGAACTCAGGTCACAGTGTCTTC | |
| | C | |
| mIL2Rg_VHH5 | CAGGTTCAGCTTCAGGAGTCCGGCGGGGGCTCCGT | 321 |
| | ACAGGCAGGGGGCTCACTGCGTCTTTCCTGTGTGG | |
| | CGAGTGGCGACGTGTATGGCCGTAACAGCATGGCT | |
| | TGGTTCCGGCAGGCACCTGGAAAGGAACGCGAGG | |
| | GCGTTGCAGTTGGGTATTCCGTAGTGACAACCACT | |
| | TACTATGCCGACAGTGTGAAGGGCCGGTTTACGAT | |
| | CTCAGAGGACAACGATAAAAACACAGTGTACCTG | |
| | GAGATGAACTCCCTGAAGCCGGAAGACACTGCTAT | |
| | GTATTACTGCGCTGCCGATGGCAACCTGTGGCGCG | |
| | GACTCAGGCCCTCCGAGTACACTTATTGGGGTCAG | |
| | GGCACCCAGGTGACCGTTTCAAGT | |
| mIL2Rg_VHH6 | CAGGTCCAGCTTCAGGAGTCAGGTGGCGGTAGTGT | 322 |
| | CCAGGCAGGCGGTAGCCTGCGCCTTAGCTGTGCTA | |
| | CATCCGGCTTCCCTTACTCACGCTATTGTATGGGCT | |
| | GGTTCAGGCAAGCTCCCGGTAAAGAGCGCGAGGG | |
| | AGTGGCAGCCATCGAGCCTGACGGGAGCACATCTT | |
| | ATGCTGACTCTGTAAAGGGGCGTTTCACCATCTCT | |
| | CAGGACAACGCCGTTAATACACTGTACTTGCAAAT | |
| | GAATAACCTGAAGCCCGAGGACACAGCTATGTATT | |
| | ACTGCGCAGCCGACGAGCGTTGCTTCTATTTGAAG | |
| | GACTATGACCTCAGAAGGCCAGCCCAGTACCGCTA | |
| | CTGGGGGCAGGGCACCCAGGTTACCGTGTCATCT | |
| mIL2Rg_VHH7 | CAGGTGCAGTTGCAGGAGAGTGGCGGTGGCCTCGT | 323 |
| | GCAGCCTGGCGGAAGCCTCCGTCTGAGCTGCACTG | |
| | TGTCCGGCTTCACTTTCGACGAGAGCGACATGGGC | |
| | TGGCTGAGGCAGAACCCTGGTAACGAGTGCGGCGT | |
| | TGTGAGTGTCATCACGTCTGATGACAACCCATACT | |
| | ATGATGACAGCGTCAAGGGCCGCTTTACTATCTCC | |
| | GAGGATAACGCCAAGAACATGGTGTACCTCCAGAT | |
| | GAACTCACTGAAGCCCGAGGATACCGGCGTTTATT | |
| | ACTGTGCAACCAGGAGCCGTCAGCCTGTGTACTCA | |
| | CGCGATTACGAGCTGCGGCCCCGCCCCCTCTGTGG | |
| | AGACTTTGGTGTGTGGGGCCAGGGCACCCAGGTGA | |
| | CTGTTTCCAGC | |
| mIL2Rg_VHH8 | CAGGTGCAGTTGCAGGAGAGTGGAGGGGGCTCAG | 324 |
| | TGCAGGCTGGCGGGTCCTTGCGTCTGTCTTGCACC | |
| | GCCTCTGGCTTCACCTTCGATGACTTCGATATGGGT | |
| | TGGTATCGCCAGGCTCCAGGGAACGAGTGCGAATT | |
| | GGTCAGCACTATCAGCGACGATGGCTCAACATATT | |
| | ACGCCGACTCTGTGAAGGGACGGTCTAGCATTAGC | |
| | CGGGACAACGCAAAGAACACCGTCTATCTCCAGAT | |
| | GAACCGCTTGAAGCCTGAGGATACCGGAGTCTATT | |
| | ACTGCGCCGCTGAGGGCGCGTTGGGCTCCAAGACT | |
| | AATTGTGGCTGGGTGGGCAACTTCGGATATTGGGG | |
| | CCAGGGAACACAGGTTACCGTTTCCAGC | |
| mIL2Rg_VHH9 | CAGGTGCAGTTGCAGGAGTCTGGAGGCGGTTCCGT | 325 |
| | TCAGGCCGGGGGCTCTCTGCGCCTGTCCTGCGCTG | |
| | CCTCCGGGTTTACATTTGACGATTTCGATATGGGCT | |
| | GGTATCGCCAGGCCCCTGGCAACGAGTGCGAACTG | |
| | GTGTCTACTATCTCCGATGACGGCTCAACCTACTAT | |
| | GCAGACTCCGTAAAGGGCAGATCCAGCATCTCCCG | |
| | CGACAATGCCAAAAACACTGTGTACCTCCAGATGA | |
| | ACTCCCTCAAGCCTGAGGATACGGCGGTGTACTAT | |
| | TGTGCTGCCGAGGGTGCGCTCGGTAGCAAGACTAA | |
| | TTGCGGCTGGGTGGGCAACTTCGGGTACTGGGGTC | |
| | AGGGGACCCAGGTAACCGTGTCTTCT | |
| mIL2Rg_VHH10 | CAGGTGCAGTTGCAGGAAAGCGGTGGGGGCCTGG | 326 |
| | TGCAGCCCGGAGGCAGCCTGCGCTTGAGCTGCGCT | |
| | GCCTCTGGCTTCACATTCGATGACTTCGATATGGG | |
| | CTGGTATCGTCAAGCACCCGGAAACGAGTGCGAGC | |
| | TGGTGAGTACAATCAGTGATGACGGATCTACCTAC | |
| | TATGCCGACAGCGTCAAGGGAAGATCCAGCATCA | |
| | GTCGCGACAACGCCAAGAGCACCGTTTACCTCCAG | |
| | ATGAACCGCCTCAAGCCTGAGGACACAGGAGTCTA | |
| | TTACTGTGCTGCGGAGGGGGCCTTGGGCAGCAAGA | |
| | CTAACTGTGGATGGGTGGGAAACTTCGGGTATTGG | |
| | GGTCAGGGTACACAGGTCACAGTGTCTTCA | |
| mIL2Rg_VHH11 | CAAGTTCAGCTTCAGGAAAGTGGGGGCGGGCTGGT | 327 |
| | GCAGCCAGGGGGTTCCCTGAAGCTGAGCTGCGCTG | |
| | CCTCTGGGTTTACATTCTCTGATCGCGACATGGGCT | |
| | GGTATCGCCAAGCGCCGGGCAATGAATGCGAAAG | |
| | AGTGAGTACTATTTCTGACGATGGTTCTACTTACTA | |
| | TGCTGACTCCGTGAAGGGCCGTAGCTCCATTTCCA | |
| | GGGACAACGCGAAGAACACCGTATACCTCCAGAT | |
| | GAACTCTCTGAAGCCCGAGGACACCGCTGTGTATT | |
| | ACTGCGCTGCCGAGGGGGCTCTCGGCTCAAAGACC | |
| | AACTGCGGATGGGTCGGTAACTTCGGCTACTGGGG | |
| | CCAGGGCACCCAAGTGACAGTCTCCTCC | |
| mIL2Rg_VHH12 | CAGGTCCAGTTGCAGGAGAGCGGGGGTGGAAGCG | 328 |
| | TCCTCGCCGGAGGGAGCCTCCGTTTGAGCTGCGTC | |
| | GCCTCAGGCTACGGCTACAATTACATCGGATGGTT | |
| | CAGACAGACGCCTGGTAAAGAGCGGGAAGGCGTC | |
| | GCCGTGATTTATATCGGTGGCGGAGACACCTATTA | |
| | CGCTGACTCAGTGAAGGGGCGTTTCACCGCAAGCC | |
| | GGGACAACGCTAAGAGCACCCTGTACCTCCAGATG | |
| | AACTCTCTCGAACCTGAGGACACTGCAATGTATTA | |
| | CTGCGTGGCTCGTTACTGCGTCGGGAGTGTCTACG | |
| | CCTGCCTGAGGGGGGGGCATGATGAGTATGCCCAC | |
| | TGGGGACAAGGAACACAGGTGACTGTCTCCAGT | |
| mIL2Rg_VHH13 | CAGGTTCAGCTCCAGGAGTCTGGTGGCGGTTCCGT | 329 |
| | GCTGGCCGGGGGCTCTCTGCGCCTGTCTTGTGTCG | |
| | CCTCAGGGTACGGCTATAACTACATTGGCTGGTTC | |
| | AGACAGACCCCTGGGAAAGAGCGGGAGGGTGTGG | |
| | CTGTCATTTACACCGGCGGAGGCGACACCTACTAT | |
| | GCCGATTCAGTTAAGGGCAGGTTTACCGCGAGCCG | |
| | TGACAACGCGAAGTCTACTCTGTACCTGCAAATGA | |
| | ACAGCCTGGAACCTGAGGATACTGCGATGTACTAT | |
| | TGTGTGGCCCGGTACTGCGTAGGCTCAGTGTATGC | |
| | CTGCCTGCGCGGGGGTCACGACGAGTACGCACACT | |
| | GGGGACAGGGAACTCAGGTCACCGTGTCTAGC | |
| mIL2Rg_VHH14 | CAGGTGCAACTCCAGGAGTCCGGCGGGGGCTCCGT | 330 |
| | CCAAGCTGGTGGCTCACTGAGGCTTAGCTGTGCTG | |
| | CCTCCGGCTTTACTTTCGACGATTTCGACATGGGTT | |
| | GGTATCGCCAGGCTCCGGGCAATGAGTGCGAGCTG | |
| | GTCTCTACCATTTCCGATGACGGCTCTACCTACTAT | |
| | GCCAACAGTGTTAAGGGTAGGTCTTCCATCTCCCG | |
| | CGACAACGCTAAGAATATGGTGTACTTGCAGATGA | |
| | ACTCTCTGAAGCCTGAGGACACTGCTGTCTACTAT | |
| | TGCGCTGCCGAAGGTGCCCTGGGCTCAAAGACTAA | |
| | TTGCGGCTGGGTCGGTAACTTTGGCTACTGGGGTC | |
| | AGGGGACTCAGGTGACCGTCAGCTCC | |
| mIL2Rg_VHH15 | CAGGTCCAGTTGCAGGAAAGCGGCGGGGGCTCTGT | 331 |
| | TCAGGCAGGCGGAAGCCTTCGTCTGTCCTGTACTG | |
| | CCAGTGGTTTCACCTTTGATGACTTTGACATGGGCT | |
| | GGTATCGGCAAGCCCCCGGAAACGAGTGCGAGCT | |
| | GGTATCCACCATTTCCGATGACGGGTCCACGTACT | |
| | ATGCTGATAGCGTGAAGGGCAGGTCTTCCATCAGC | |
| | CGGGACAACGCCAAGAACACAGTGTATTTGCAGAT | |
| | GAACCGCCTCAAGCCAGAAGACACCGGGGTATATT | |
| | ACTGTGCAGCGGAAGGTGCCCTGGGTAGCAAGAT | |
| | GAACTGCGGATGGGTGGGTAATTTTGGATACTGGG | |
| | GCCAGGGCACGCAGGTTACAGTGTCCAGC | |

TABLE 19
Anti-hIL10Ra/hIL2Rg dual VHH binding to human and cynomolgus IL2Rg-Fc

| | | | | | | | Calc. | |
|---|---|---|---|---|---|---|---|---|
| | | k_{ON} | k_{OFF} | Affinity | Rmax | Load | Rmax | Surface |
| Analyte | Ligand | (1/Ms) | (1/s) | (nM) | (RU) | (RU) | (RU) | Activity |
| A2, | hIL2Rg-Fc | 2.0E+06 | 7.8E−03 | 3.9 | 3.4 | 99 | 74 | 5% |
| DR359 | (SinoBiological | | | | | | | |
| | cat#10555) | | | | | | | |
| | cIL2Rg-Fc | 8.0E+05 | 2.6E−03 | 3.2 | 1.9 | 153 | 115 | 2% |
| | (lot#P210115SV5) | | | | | | | |
| A7, | hIL2Rg-Fc | 1.7E+06 | 6.2E−03 | 3.6 | 3.2 | 99 | 74 | 4% |
| DR392 | (SinoBiological | | | | | | | |
| | cat#10555) | | | | | | | |
| | cIL2Rg-Fc | 1.6E+06 | 4.0E−03 | 2.4 | 1.5 | 161 | 120 | 1% |
| | (lot#P210115SV5) | | | | | | | |
| D6, | hIL2Rg-Fc | 1.9E+05 | 5.1E−03 | 27.5 | 1.9 | 99 | 74 | 3% |
| DR437 | (SinoBiological | | | | | | | |
| | cat#10555) | | | | | | | |
| | cIL2Rg-Fc | 2.9E+05 | 2.2E−03 | 7.8 | 1.3 | 160 | 119 | 1% |
| | (lot#P210115SV5) | | | | | | | |
| D12, | hIL2Rg-Fc | 3.9E+05 | 1.0E−03 | 2.7 | 3.4 | 99 | 74 | 5% |
| DR438 | (SinoBiological | | | | | | | |
| | cat#10555) | | | | | | | |
| | cIL2Rg-Fc | 5.6E+05 | 1.2E−03 | 2.2 | 1.5 | 163 | 122 | 1% |
| | (lot#P210115SV5) | | | | | | | |
| E8, | hIL2Rg-Fc | 3.8E+05 | 2.6E−03 | 6.9 | 3.4 | 99 | 74 | 5% |
| DR444 | (SinoBiological | | | | | | | |
| | cat#10555) | | | | | | | |
| | cIL2Rg-Fc | 1.5E+05 | 7.7E−04 | 5.3 | 1.8 | 161 | 120 | 2% |
| | (lot#P210115SV5) | | | | | | | |
| F1, | hIL2Rg-Fc | 4.7E+05 | 5.3E−03 | 11.1 | 3.1 | 99 | 74 | 4% |
| DR441 | (SinoBiological | | | | | | | |
| | cat#10555) | | | | | | | |
| | cIL2Rg-Fc | 3.4E+05 | 2.8E−03 | 8.3 | 1.6 | 162 | 121 | 1% |
| | (lot#P210115SV5) | | | | | | | |
| F3, | hIL2Rg-Fc | 2.2E+05 | 6.5E−03 | 30.4 | 1.5 | 98 | 73 | 2% |
| DR449 | (SinoBiological | | | | | | | |
| | cat#10555) | | | | | | | |
| | cIL2Rg-Fc | 2.1E+05 | 4.8E−03 | 22.6 | 0.9 | 163 | 122 | 1% |
| | (lot#P210115SV5) | | | | | | | |
| F7, | hIL2Rg-Fc | 2.0E+06 | 4.2E−02 | 21.5 | 3.5 | 98 | 73 | 5% |
| DR442 | (SinoBiological | | | | | | | |
| | cat#10555) | | | | | | | |
| | cIL2Rg-Fc | 7.9E+04 | 3.3E−03 | 41.3 | 7.9 | 162 | 121 | 7% |
| | (lot#P210115SV5) | | | | | | | |
| G3, | hIL2Rg-Fc | 3.4E+05 | 5.7E−03 | 16.6 | 3.6 | 98 | 73 | 5% |
| DR471 | (SinoBiological | | | | | | | |
| | cat#10555) | | | | | | | |
| | cIL2Rg-Fc | 3.6E+05 | 4.8E−03 | 13.4 | 1.6 | 164 | 122 | 1% |
| | (lot#P210115SV5) | | | | | | | |
| G8, | hIL2Rg-Fc | 1.7E+05 | 1.5E−03 | 8.5 | 4.2 | 98 | 73 | 6% |
| DR468 | (SinoBiological | | | | | | | |
| | cat#10555) | | | | | | | |
| | cIL2Rg-Fc | 2.4E+05 | 1.0E−03 | 4.3 | 1.8 | 164 | 122 | 1% |
| | (lot#P210115SV5) | | | | | | | |
| H1, | hIL2Rg-Fc | 2.5E+05 | 3.7E−03 | 14.5 | 2.6 | 98 | 73 | 4% |
| DR465 | (SinoBiological | | | | | | | |
| | cat#10555) | | | | | | | |
| | cIL2Rg-Fc | 7.0E+05 | 2.2E−03 | 3.2 | 0.9 | 163 | 122 | 1% |
| | (lot#P210115SV5) | | | | | | | |
| H7, | hIL2Rg-Fc | 1.2E+06* | 2.6E−02 | 20.8 | 4.2 | 97 | 73 | 6% |
| DR466 | (SinoBiological | | | | | | | |
| | cat#10555) | | | | | | | |
| | cIL2Rg-Fc | 7.5E+04* | 3.7E−03 | 49.9 | 11.5 | 164 | 122 | 9% |
| | (lot#P210115SV5) | | | | | | | |
| H9, | hIL2Rg-Fc | 3.1E+05 | 9.9E−04 | 3.2 | 4.1 | 97 | 73 | 6% |
| DR474 | (SinoBiological | | | | | | | |
| | cat#10555) | | | | | | | |
| | cIL2Rg-Fc | 3.3E+05 | 9.3E−04 | 2.9 | 2.3 | 163 | 121 | 2% |
| | (lot#P210115SV5) | | | | | | | |

TABLE 20
Effect of Linker Length IL10Ra/IL10Rg VHH dimers on pSTAT3 Induction of on CD8 T cell

| | | | Concentration of | |
|---|---|---|---|---|
| VHH1-VHH2 ID | Linker | Tag | molecule (μM) | pSTAT3 (MFI) |
| hIL10Ra_VHH14- | GGGS | Fc | 0 | 6751 |
| hIL2Rg_VHH19 | | | | |
| hIL10Ra_VHH14- | GGGS | Fc | 0.0001 | 7538 |
| hIL2Rg_VHH19 | | | | |
| hIL10Ra_VHH14- | GGGS | Fc | 0.001 | 7889 |
| hIL2Rg_VHH19 | | | | |
| hIL10Ra_VHH14- | GGGS | Fc | 0.01 | 7802 |
| hIL2Rg_VHH19 | | | | |
| hIL10Ra_VHH14- | GGGS | Fc | 0.1 | 7330 |
| hIL2Rg_VHH19 | | | | |
| hIL10Ra_VHH14- | GGGS | Fc | 1 | 9316 |
| hIL2Rg_VHH19 | | | | |
| hIL10Ra_VHH14- | GGGS | Fc | 10 | 12256 |
| hIL2Rg_VHH19 | | | | |
| hIL10Ra_VHH14- | GGGS | Fc | 100 | 13188 |
| hIL2Rg_VHH19 | | | | |
| hIL10Ra_VHH14- | GGGS | his | 0 | 6751 |
| hIL2Rg_VHH19 | | | | |
| hIL10Ra_VHH14- | GGGS | his | 0.0001 | 7207 |
| hIL2Rg_VHH19 | | | | |
| hIL10Ra_VHH14- | GGGS | his | 0.001 | 7461 |
| hIL2Rg_VHH19 | | | | |
| hIL10Ra_VHH14- | GGGS | his | 0.01 | 7195 |
| hIL2Rg_VHH19 | | | | |
| hIL10Ra_VHH14- | GGGS | his | 0.1 | 6584 |
| hIL2Rg_VHH19 | | | | |
| hIL10Ra_VHH14- | GGGS | his | 1 | 6495 |
| hIL2Rg_VHH19 | | | | |
| hIL10Ra_VHH14- | GGGS | his | 10 | 7934 |
| hIL2Rg_VHH19 | | | | |
| hIL10Ra_VHH14- | GGGS | his | 100 | 9967 |
| hIL2Rg_VHH19 | | | | |
| hIL10Ra_VHH14- | No linker | his | 0 | 6751 |
| hIL2Rg_VHH19 | | | | |
| hIL10Ra_VHH14- | No linker | his | 0.0001 | 7383 |
| hIL2Rg_VHH19 | | | | |
| hIL10Ra_VHH14- | No linker | his | 0.001 | 7742 |
| hIL2Rg_VHH19 | | | | |
| hIL10Ra_VHH14- | No linker | his | 0.01 | 7799 |
| hIL2Rg_VHH19 | | | | |
| hIL10Ra_VHH14- | No linke | his | 0.1 | 6775 |
| hIL2Rg_VHH19 | | | | |
| hIL10Ra_VHH14- | No linker | his | 1 | 7725 |
| hIL2Rg_VHH19 | | | | |
| hIL10Ra_VHH14- | No linker | his | 10 | 7486 |
| hIL2Rg_VHH19 | | | | |
| hIL10Ra_VHH14- | No linker | his | 100 | 6202 |
| hIL2Rg_VHH19 | | | | |
| hIL10Ra_VHH14- | GS | his | 0 | 6751 |
| hIL2Rg_VHH19 | | | | |
| hIL10Ra_VHH14- | GS | his | 0.0001 | 7402 |
| hIL2Rg_VHH19 | | | | |
| hIL10Ra_VHH14- | GS | his | 0.001 | 8109 |
| hIL2Rg_VHH19 | | | | |
| hIL10Ra_VHH14- | GS | his | 0.01 | 8114 |
| hIL2Rg_VHH19 | | | | |
| hIL10Ra_VHH14- | GS | his | 0.1 | 7101 |
| hIL2Rg_VHH19 | | | | |
| hIL10Ra_VHH14- | GS | his | 1 | 8204 |
| hIL2Rg_VHH19 | | | | |
| hIL10Ra_VHH14- | GS | his | 10 | 8411 |
| hIL2Rg_VHH19 | | | | |
| hIL10Ra_VHH14- | GS | his | 100 | 7905 |
| hIL2Rg_VHH19 | | | | |
| hIL10Ra_VHH14- | GGSGGS | his | 0 | 6751 |
| hIL2Rg_VHH19 | | | | |
| hIL10Ra_VHH14- | GGSGGS | his | 0.0001 | 7955 |
| hIL2Rg_VHH19 | | | | |
| hIL10Ra_VHH14- | GGSGGS | his | 0.001 | 8498 |
| hIL2Rg_VHH19 | | | | |
| hIL10Ra_VHH14- | GGSGGS | his | 0.01 | 7658 |
| hIL2Rg_VHH19 | | | | |
| hIL10Ra_VHH14- | GGSGGS | his | 0.1 | 8156 |
| hIL2Rg_VHH19 | | | | |
| hIL10Ra_VHH14- | GGSGGS | his | 1 | 7715 |
| hIL2Rg_VHH19 | | | | |
| hIL10Ra_VHH14- | GGSGGS | his | 10 | 8296 |
| hIL2Rg_VHH19 | | | | |
| hIL10Ra_VHH14- | GGSGGS | his | 100 | 7001 |
| hIL2Rg_VHH19 | | | | |
| hIL10Ra_VHH14- | GGGSGGGS | his | 0 | 6751 |
| hIL2Rg_VHH19 | | | | |
| hIL10Ra_VHH14- | GGGSGGGS | his | 0.0001 | 7059 |
| hIL2Rg_VHH19 | | | | |
| hIL10Ra_VHH14- | GGGSGGGS | his | 0.001 | 7522 |
| hIL2Rg_VHH19 | | | | |
| hIL10Ra_VHH14- | GGGSGGGS | his | 0.01 | 6331 |
| hIL2Rg_VHH19 | | | | |
| hIL10Ra_VHH14- | GGGSGGGS | his | 0.1 | 7073 |
| hIL2Rg_VHH19 | | | | |
| hIL10Ra_VHH14- | GGGSGGGS | his | 1 | 7872 |
| hIL2Rg_VHH19 | | | | |
| hIL10Ra_VHH14- | GGGSGGGS | his | 10 | 8431 |
| hIL2Rg_VHH19 | | | | |
| hIL10Ra_VHH14- | GGGSGGGS | his | 100 | 7974 |
| hIL2Rg_VHH19 | | | | |
| hIL10Ra_VHH14- | GGSGGSGGSG | his | 0 | 6751 |
| hIL2Rg_VHH19 | | | | |
| hIL10Ra_VHH14- | GGSGGSGGSG | his | 0.0001 | 7840 |
| hIL2Rg_VHH19 | | | | |
| hIL10Ra_VHH14- | GGSGGSGGSG | his | 0.001 | 7516 |
| hIL2Rg_VHH19 | | | | |
| hIL10Ra_VHH14- | GGSGGSGGSG | his | 0.01 | 6922 |
| hIL2Rg_VHH19 | | | | |
| hIL10Ra_VHH14- | GGSGGSGGSG | his | 0.1 | 6782 |
| hIL2Rg_VHH19 | | | | |
| hIL10Ra_VHH14- | GGSGGSGGSG | his | 1 | 6479 |
| hIL2Rg_VHH19 | | | | |
| hIL10Ra_VHH14- | GGSGGSGGSG | his | 10 | 7329 |
| hIL2Rg_VHH19 | | | | |
| hIL10Ra_VHH14- | GGSGGSGGSG | his | 100 | 6928 |
| hIL2Rg_VHH19 | | | | |

TABLE 21
Effect of Linker Length IL10Ra/IL10Rg VHH dimers on pSTAT3 Induction of on CD4 T cells

| | | | Concentration of | |
|---|---|---|---|---|
| VHH1-VHH2 ID | Linker | Tag | molecule (μ M) | pSTAT3 (MFI) |
| hIL10Ra_VHH14- | GGGS | Fc | 0 | 10291 |
| hIL2Rg_VHH19 | | | | |
| hIL10Ra_VHH14- | GGGS | Fc | 0.0001 | 12636 |
| hIL2Rg_VHH19 | | | | |
| hIL10Ra_VHH14- | GGGS | Fc | 0.001 | 12627 |
| hIL2Rg_VHH19 | | | | |
| hIL10Ra_VHH14- | GGGS | Fc | 0.01 | 12526 |
| hIL2Rg_VHH19 | | | | |
| hIL10Ra_VHH14- | GGGS | Fc | 0.1 | 11340 |
| hIL2Rg_VHH19 | | | | |
| hIL10Ra_VHH14- | GGGS | Fc | 1 | 13578 |
| hIL2Rg_VHH19 | | | | |
| hIL10Ra_VHH14- | GGGS | Fc | 10 | 14841 |
| hIL2Rg_VHH19 | | | | |
| hIL10Ra_VHH14- | GGGS | Fc | 100 | 16045 |
| hIL2Rg_VHH19 | | | | |
| hIL10Ra_VHH14- | GGGS | his | 0 | 10291 |
| hIL2Rg_VHH19 | | | | |
| hIL10Ra_VHH14- | GGGS | his | 0.0001 | 11924 |
| hIL2Rg_VHH19 | | | | |
| hIL10Ra_VHH14- | GGGS | his | 0.001 | 12086 |
| hIL2Rg_VHH19 | | | | |
| hIL10Ra_VHH14- | GGGS | his | 0.01 | 11654 |
| hIL2Rg_VHH19 | | | | |
| hIL10Ra_VHH14- | GGGS | his | 0.1 | 9670 |
| hIL2Rg_VHH19 | | | | |
| hIL10Ra_VHH14- | GGGS | his | 1 | 9591 |
| hIL2Rg_VHH19 | | | | |
| hIL10Ra_VHH14- | GGGS | his | 10 | 11382 |
| hIL2Rg_VHH19 | | | | |
| hIL10Ra_VHH14- | GGGS | his | 100 | 14725 |
| hIL2Rg_VHH19 | | | | |
| hIL10Ra_VHH14- | No linker | his | 0 | 10291 |
| hIL2Rg_VHH19 | | | | |
| hIL10Ra_VHH14- | No linker | his | 0.0001 | 12174 |
| hIL2Rg_VHH19 | | | | |
| hIL10Ra_VHH14- | No linker | his | 0.001 | 12882 |
| hIL2Rg_VHH19 | | | | |
| hIL10Ra_VHH14- | No linker | his | 0.01 | 12460 |
| hIL2Rg_VHH19 | | | | |
| hIL10Ra_VHH14- | No linker | his | 0.1 | 11004 |
| hIL2Rg_VHH19 | | | | |
| hIL10Ra_VHH14- | No linker | his | 1 | 12144 |
| hIL2Rg_VHH19 | | | | |
| hIL10Ra_VHH14- | No linker | his | 10 | 11846 |
| hIL2Rg_VHH19 | | | | |
| hIL10Ra_VHH14- | No linker | his | 100 | 9036 |
| hIL2Rg_VHH19 | | | | |
| hIL10Ra_VHH14- | GS | his | 0 | 10291 |
| hIL2Rg_VHH19 | | | | |
| hIL10Ra_VHH14- | GS | his | 0.0001 | 11707 |
| hIL2Rg_VHH19 | | | | |
| hIL10Ra_VHH14- | GS | his | 0.001 | 12776 |
| hIL2Rg_VHH19 | | | | |
| hIL10Ra_VHH14- | GS | his | 0.01 | 13621 |
| hIL2Rg_VHH19 | | | | |
| hIL10Ra_VHH14- | GS | his | 0.1 | 10942 |
| hIL2Rg_VHH19 | | | | |
| hIL10Ra_VHH14- | GS | his | 1 | 12949 |
| hIL2Rg_VHH19 | | | | |
| hIL10Ra_VHH14- | GS | his | 10 | 12563 |
| hIL2Rg_VHH19 | | | | |
| hIL10Ra_VHH14- | GS | his | 100 | 11613 |
| hIL2Rg_VHH19 | | | | |
| hIL10Ra_VHH14- | GGSGGS | his | 0 | 10291 |
| hIL2Rg_VHH19 | | | | |
| hIL10Ra_VHH14- | GGSGGS | his | 0.0001 | 13197 |
| hIL2Rg_VHH19 | | | | |
| hIL10Ra_VHH14- | GGSGGS | his | 0.001 | 13876 |
| hIL2Rg_VHH19 | | | | |
| hIL10Ra_VHH14- | GGSGGS | his | 0.01 | 12787 |
| hIL2Rg_VHH19 | | | | |
| hIL10Ra_VHH14- | GGSGGS | his | 0.1 | 13577 |
| hIL2Rg_VHH19 | | | | |
| hIL10Ra_VHH14- | GGSGGS | his | 1 | 12991 |
| hIL2Rg_VHH19 | | | | |
| hIL10Ra_VHH14- | GGSGGS | his | 10 | 11840 |
| hIL2Rg_VHH19 | | | | |
| hIL10Ra_VHH14- | GGSGGS | his | 100 | 10166 |
| hIL2Rg_VHH19 | | | | |
| hIL10Ra_VHH14- | GGGSGGGS | his | 0 | 10291 |
| hIL2Rg_VHH19 | | | | |
| hIL10Ra_VHH14- | GGGSGGGS | his | 0.0001 | 10704 |
| hIL2Rg_VHH19 | | | | |
| hIL10Ra_VHH14- | GGGSGGGS | his | 0.0013 | 12050 |
| hIL2Rg_VHH19 | | | | |
| hIL10Ra_VHH14- | GGGSGGGS | his | 0.01 | 9547 |
| hIL2Rg_VHH19 | | | | |
| hIL10Ra_VHH14- | GGGSGGGS | his | 0.1 | 11269 |
| hIL2Rg_VHH19 | | | | |
| hIL10Ra_VHH14- | GGGSGGGS | his | 1 | 12658 |
| hIL2Rg_VHH19 | | | | |
| hIL10Ra_VHH14- | GGGSGGGS | his | 10 | 13170 |
| hIL2Rg_VHH19 | | | | |
| hIL10Ra_VHH14- | GGGSGGGS | his | 100 | 11553 |
| hIL2Rg_VHH19 | | | | |
| hIL10Ra_VHH14- | GGSGGSGGSG | his | 0 | 10291 |
| hIL2Rg_VHH19 | | | | |
| hIL10Ra_VHH14- | GGSGGSGGSG | his | 0.0001 | 12805 |
| hIL2Rg_VHH19 | | | | |
| hIL10Ra_VHH14- | GGSGGSGGSG | his | 0.001 | 11826 |
| hIL2Rg_VHH19 | | | | |
| hIL10Ra_VHH14- | GGSGGSGGSG | his | 0.01 | 11130 |
| hIL2Rg_VHH19 | | | | |
| hIL10Ra_VHH14- | GGSGGSGGSG | his | 0.1 | 10217 |
| hIL2Rg_VHH19 | | | | |
| hIL10Ra_VHH14- | GGSGGSGGSG | his | 1 | 9972 |
| hIL2Rg_VHH19 | | | | |
| hIL10Ra_VHH14- | GGSGGSGGSG | his | 10 | 11400 |
| hIL2Rg_VHH19 | | | | |
| hIL10Ra_VHH14- | GGSGGSGGSG | his | 100 | 10324 |
| hIL2Rg_VHH19 | | | | |

TABLE 22
Effect of Linker Length IL10Ra/IL10Rg VHH dimers on pSTAT3 Induction of in monocytes

| | | | Concentration of | |
|---|---|---|---|---|
| VHH1-VHH2 ID | Linker | Tag | molecule (μM) | pSTAT3 (MFI) |
| hIL10Ra_VHH14- | GGGS | Fc | 0 | 4610 |
| hIL2Rg_VHH19 | | | | |
| hIL10Ra_VHH14- | GGGS | Fc | 0.0001 | 4373 |
| hIL2Rg_VHH19 | | | | |
| hIL10Ra_VHH14- | GGGS | Fc | 0.001 | 4737 |
| hIL2Rg_VHH19 | | | | |
| hIL10Ra_VHH14- | GGGS | Fc | 0.01 | 4417 |
| hIL2Rg_VHH19 | | | | |
| hIL10Ra_VHH14- | GGGS | Fc | 0.1 | 4808 |
| hIL2Rg_VHH19 | | | | |
| hIL10Ra_VHH14- | GGGS | Fc | 1 | 5687 |
| hIL2Rg_VHH19 | | | | |
| hIL10Ra_VHH14- | GGGS | Fc | 10 | 6688 |
| hIL2Rg_VHH19 | | | | |
| hIL10Ra_VHH14- | GGGS | Fc | 100 | 6026 |
| hIL2Rg_VHH19 | | | | |
| hIL10Ra_VHH14- | GGGS | his | 0 | 4610 |
| hIL2Rg_VHH19 | | | | |
| hIL10Ra_VHH14- | GGGS | his | 0.0001 | 4151 |
| hIL2Rg_VHH19 | | | | |
| hIL10Ra_VHH14- | GGGS | his | 0.001 | 4510 |
| hIL2Rg_VHH19 | | | | |
| hIL10Ra_VHH14- | GGGS | his | 0.01 | 4357 |
| hIL2Rg_VHH19 | | | | |
| hIL10Ra_VHH14- | GGGS | his | 0.1 | 4277 |
| hIL2Rg_VHH19 | | | | |
| hIL10Ra_VHH14- | GGGS | his | 1 | 4640 |
| hIL2Rg_VHH19 | | | | |
| hIL10Ra_VHH14- | GGGS | his | 10 | 4842 |
| hIL2Rg_VHH19 | | | | |
| hIL10Ra_VHH14- | GGGS | his | 100 | 4877 |
| hIL2Rg_VHH19 | | | | |
| hIL10Ra_VHH14- | No linker | his | 0 | 4610 |
| hIL2Rg_VHH19 | | | | |
| hIL10Ra_VHH14- | No linker | his | 0.0001 | 3969 |
| hIL2Rg_VHH19 | | | | |
| hIL10Ra_VHH14- | No linker | his | 0.001 | 4602 |
| hIL2Rg_VHH19 | | | | |
| hIL10Ra_VHH14- | No linker | his | 0.01 | 4425 |
| hIL2Rg_VHH19 | | | | |
| hIL10Ra_VHH14- | No linker | his | 0.1 | 4423 |
| hIL2Rg_VHH19 | | | | |
| hIL10Ra_VHH14- | No linker | his | 1 | 4480 |
| hIL2Rg_VHH19 | | | | |
| hIL10Ra_VHH14- | No linker | his | 10 | 4331 |
| hIL2Rg_VHH19 | | | | |
| hIL10Ra_VHH14- | No linker | his | 100 | 4139 |
| hIL2Rg_VHH19 | | | | |
| hIL10Ra_VHH14- | GS | his | 0 | 4610 |
| hIL2Rg_VHH19 | | | | |
| hIL10Ra_VHH14- | GS | his | 0.0001 | 4035 |
| hIL2Rg_VHH19 | | | | |
| hIL10Ra_VHH14- | GS | his | 0.001 | 4821 |
| hIL2Rg_VHH19 | | | | |
| hIL10Ra_VHH14- | GS | his | 0.01 | 4012 |
| hIL2Rg_VHH19 | | | | |
| hIL10Ra_VHH14- | GS | his | 0.1 | 4432 |
| hIL2Rg_VHH19 | | | | |
| hIL10Ra_VHH14- | GS | his | 1 | 4791 |
| hIL2Rg_VHH19 | | | | |
| hIL10Ra_VHH14- | GS | his | 10 | 4934 |
| hIL2Rg_VHH19 | | | | |
| hIL10Ra_VHH14- | GS | his | 100 | 4930 |
| hIL2Rg_VHH19 | | | | |
| hIL10Ra_VHH14- | GGSGGS | his | 0 | 4610 |
| hIL2Rg_VHH19 | | | | |
| hIL10Ra_VHH14- | GGSGGS | his | 0.0001 | 4217 |
| hIL2Rg_VHH19 | | | | |
| hIL10Ra_VHH14- | GGSGGS | his | 0.001 | 4677 |
| hIL2Rg_VHH19 | | | | |
| hIL10Ra_VHH14- | GGSGGS | his | 0.01 | 4563 |
| hIL2Rg_VHH19 | | | | |
| hIL10Ra_VHH14- | GGSGGS | his | 0.1 | 4468 |
| hIL2Rg_VHH19 | | | | |
| hIL10Ra_VHH14- | GGSGGS | his | 1 | 4752 |
| hIL2Rg_VHH19 | | | | |
| hIL10Ra_VHH14- | GGSGGS | his | 10 | 4894 |
| hIL2Rg_VHH19 | | | | |
| hIL10Ra_VHH14- | GGSGGS | his | 100 | 4726 |
| hIL2Rg_VHH19 | | | | |
| hIL10Ra_VHH14- | GGGSGGGS | his | 0 | 4610 |
| hIL2Rg_VHH19 | | | | |
| hIL10Ra_VHH14- | GGGSGGGS | his | 0.0001 | 4666 |
| hIL2Rg_VHH19 | | | | |
| hIL10Ra_VHH14- | GGGSGGGS | his | 0.001 | 4821 |
| hIL2Rg_VHH19 | | | | |
| hIL10Ra_VHH14- | GGGSGGGS | his | 0.01 | 4356 |
| hIL2Rg_VHH19 | | | | |
| hIL10Ra_VHH14- | GGGSGGGS | his | 0.1 | 4425 |
| hIL2Rg_VHH19 | | | | |
| hIL10Ra_VHH14- | GGGSGGGS | his | 1 | 4760 |
| hIL2Rg_VHH19 | | | | |
| hIL10Ra_VHH14- | GGGSGGGS | his | 10 | 4879 |
| hIL2Rg_VHH19 | | | | |
| hIL10Ra_VHH14- | GGGSGGGS | his | 100 | 4617 |
| hIL2Rg_VHH19 | | | | |
| hIL10Ra_VHH14- | GGSGGSGGSG | his | 0 | 4610 |
| hIL2Rg_VHH19 | | | | |
| hIL10Ra_VHH14- | GGSGGSGGSG | his | 0.0001 | 4232 |
| hIL2Rg_VHH19 | | | | |
| hIL10Ra_VHH14- | GGSGGSGGSG | his | 0.001 | 4709 |
| hIL2Rg_VHH19 | | | | |
| hIL10Ra_VHH14- | GGSGGSGGSG | his | 0.01 | 4701 |
| hIL2Rg_VHH19 | | | | |
| hIL10Ra_VHH14- | GGSGGSGGSG | his | 0.1 | 4694 |
| hIL2Rg_VHH19 | | | | |
| hIL10Ra_VHH14- | GGSGGSGGSG | his | 1 | 4622 |
| hIL2Rg_VHH19 | | | | |
| hIL10Ra_VHH14- | GGSGGSGGSG | his | 10 | 4546 |
| hIL2Rg_VHH19 | | | | |
| hIL10Ra_VHH14- | GGSGGSGGSG | his | 100 | 4862 |
| hIL2Rg_VHH19 | | | | |

TABLE 23
Activity of VHH dimers on
CD8 T cell IFNγ secretion.

| | | | Concen- | |
|---|---|---|---|---|
| | | | tration | |
| | | | of | |
| | | | molecule | IFNγ |
| VHH1 - VHH2 ID | Linker | Tag | (μM) | (pg/mL) |
| hIL10Ra_VHH14 - | GGGS | Fc | 0 | 122093 |
| hIL2Rg_VHH19 | | | | |
| hIL10Ra_VHH14 - | GGGS | Fc | 0.0001 | 103635 |
| hIL2Rg_VHH19 | | | | |
| hIL10Ra_VHH14 - | GGGS | Fc | 0.001 | 112903 |
| hIL2Rg_VHH19 | | | | |
| hIL10Ra_VHH14 - | GGGS | Fc | 0.01 | 131153 |
| hIL2Rg_VHH19 | | | | |
| hIL10Ra_VHH14 - | GGGS | Fc | 0.1 | 144485 |
| hIL2Rg_VHH19 | | | | |
| hIL10Ra_VHH14 - | GGGS | Fc | 1 | 165539 |
| hIL2Rg_VHH19 | | | | |
| hIL10Ra_VHH14 - | GGGS | Fc | 10 | 156982 |
| hIL2Rg_VHH19 | | | | |
| hIL10Ra_VHH14 - | GGGS | Fc | 100 | 147964 |
| hIL2Rg_VHH19 | | | | |
| hIL10Ra_VHH14 - | GGGS | his | 0 | 122093 |
| hIL2Rg_VHH19 | | | | |
| hIL10Ra_VHH14 - | GGGS | his | 0.0001 | 96799 |
| hIL2Rg_VHH19 | | | | |
| hIL10Ra_VHH14 - | GGGS | his | 0.001 | 99579 |
| hIL2Rg_VHH19 | | | | |
| hIL10Ra_VHH14 - | GGGS | his | 0.01 | 106757 |
| hIL2Rg_VHH19 | | | | |
| hIL10Ra_VHH14 - | GGGS | his | 0.1 | 114181 |
| hIL2Rg_VHH19 | | | | |
| hIL10Ra_VHH14 - | GGGS | his | 1 | 130484 |
| hIL2Rg_VHH19 | | | | |
| hIL10Ra_VHH14 - | GGGS | his | 10 | 133684 |
| hIL2Rg_VHH19 | | | | |
| hIL10Ra_VHH14 - | GGGS | his | 100 | 134443 |
| hIL2Rg_VHH19 | | | | |
| hIL10Ra_VHH14 - | No linker | his | 0 | 122093 |
| hIL2Rg_VHH19 | | | | |
| hIL10Ra_VHH14 - | No linker | his | 0.0001 | 101945 |
| hIL2Rg_VHH19 | | | | |
| hIL10Ra_VHH14 - | No linker | his | 0.001 | 103568 |
| hIL2Rg_VHH19 | | | | |
| hIL10Ra_VHH14 - | No linker | his | 0.01 | 111251 |
| hIL2Rg_VHH19 | | | | |
| hIL10Ra_VHH14 - | No linker | his | 0.1 | 111247 |
| hIL2Rg_VHH19 | | | | |
| hIL10Ra_VHH14 - | No linker | his | 1 | 122585 |
| hIL2Rg_VHH19 | | | | |
| hIL10Ra_VHH14 - | No linker | his | 10 | 130574 |
| hIL2Rg_VHH19 | | | | |
| hIL10Ra_VHH14 - | No linker | his | 100 | 133923 |
| hIL2Rg_VHH19 | | | | |
| hIL10Ra_VHH14 - | GS | his | 0 | 122093 |
| hIL2Rg_VHH19 | | | | |
| hIL10Ra_VHH14 - | GS | his | 0.0001 | 91703 |
| hIL2Rg_VHH19 | | | | |
| hIL10Ra_VHH14 - | GS | his | 0.001 | 105021 |
| hIL2Rg_VHH19 | | | | |
| hIL10Ra_VHH14 - | GS | his | 0.01 | 122227 |
| hIL2Rg_VHH19 | | | | |
| hIL10Ra_VHH14 - | GS | his | 0.1 | 128960 |
| hIL2Rg_VHH19 | | | | |
| hIL10Ra_VHH14 - | GS | his | 1 | 133736 |
| hIL2Rg_VHH19 | | | | |
| hIL10Ra_VHH14 - | GS | his | 10 | 142927 |
| hIL2Rg_VHH19 | | | | |
| hIL10Ra_VHH14 - | GS | his | 100 | 143369 |
| hIL2Rg_VHH19 | | | | |
| hIL10Ra_VHH14 - | GGSGGS | his | 0 | 122093 |
| hIL2Rg_VHH19 | | | | |
| hIL10Ra_VHH14 - | GGSGGS | his | 0.0001 | 94677 |
| hIL2Rg_VHH19 | | | | |
| hIL10Ra_VHH14 - | GGSGGS | his | 0.001 | 109945 |
| hIL2Rg_VHH19 | | | | |
| hIL10Ra_VHH14 - | GGSGGS | his | 0.01 | 116978 |
| hIL2Rg_VHH19 | | | | |
| hIL10Ra_VHH14 - | GGSGGS | his | 0.1 | 121735 |
| hIL2Rg_VHH19 | | | | |
| hIL10Ra_VHH14 - | GGSGGS | his | 1 | 143818 |
| hIL2Rg_VHH19 | | | | |
| hIL10Ra_VHH14 - | GGSGGS | his | 10 | 149494 |
| hIL2Rg_VHH19 | | | | |
| hIL10Ra_VHH14 - | GGSGGS | his | 100 | 126891 |
| hIL2Rg_VHH19 | | | | |
| hIL10Ra_VHH14 - | GGGSGGGS | his | 0 | 122093 |
| hIL2Rg_VHH19 | | | | |
| hIL10Ra_VHH14 - | GGGSGGGS | his | 0.0001 | 95530 |
| hIL2Rg_VHH19 | | | | |
| hIL10Ra_VHH14 - | GGGSGGGS | his | 0.001 | 107757 |
| hIL2Rg_VHH19 | | | | |
| hIL10Ra_VHH14 - | GGGSGGGS | his | 0.01 | 101956 |
| hIL2Rg_VHH19 | | | | |
| hIL10Ra_VHH14 - | GGGSGGGS | his | 0.1 | 121526 |
| hIL2Rg_VHH19 | | | | |
| hIL10Ra_VHH14 - | GGGSGGGS | his | 1 | 141710 |
| hIL2Rg_VHH19 | | | | |
| hIL10Ra_VHH14 - | GGGSGGGS | his | 10 | 143032 |
| hIL2Rg_VHH19 | | | | |
| hIL10Ra_VHH14 - | GGGSGGGS | his | 100 | 119598 |
| hIL2Rg_VHH19 | | | | |
| hIL10Ra_VHH14 - | GGSGGSGGSG | his | 0 | 122093 |
| hIL2Rg_VHH19 | | | | |
| hIL10Ra_VHH14 - | GGSGGSGGSG | his | 0.0001 | 90061 |
| hIL2Rg_VHH19 | | | | |
| hIL10Ra_VHH14 - | GGSGGSGGSG | his | 0.001 | 125238 |
| hIL2Rg_VHH19 | | | | |
| hIL10Ra_VHH14 - | GGSGGSGGSG | his | 0.01 | 126735 |
| hIL2Rg_VHH19 | | | | |
| hIL10Ra_VHH14 - | GGSGGSGGSG | his | 0.1 | 129834 |
| hIL2Rg_VHH19 | | | | |
| hIL10Ra_VHH14 - | GGSGGSGGSG | his | 1 | 147457 |
| hIL2Rg_VHH19 | | | | |
| hIL10Ra_VHH14 - | GGSGGSGGSG | his | 10 | 154053 |
| hIL2Rg_VHH19 | | | | |
| hIL10Ra_VHH14 - | GGSGGSGGSG | his | 100 | 140732 |
| hIL2Rg_VHH19 | | | | |

TABLE 24
Activity of VHH dimers on CD8 T
cell Granzyme A secretion.

| | | | Concen- | |
|---|---|---|---|---|
| | | | tration | |
| | | | of | Gran- |
| | | | molecule | zyme A |
| VHH1 - VHH2 ID | Linker | Tag | (μM) | (pg/mL) |
| hIL10Ra_VHH14 - | GGGS | Fc | 0 | 25242 |
| hIL2Rg_VHH19 | | | | |
| hIL10Ra_VHH14 - | GGGS | Fc | 0.0001 | 22096 |
| hIL2Rg_VHH19 | | | | |
| hIL10Ra_VHH14 - | GGGS | Fc | 0.001 | 25929 |
| hIL2Rg_VHH19 | | | | |
| hIL10Ra_VHH14 - | GGGS | Fc | 0.01 | 27649 |
| hIL2Rg_VHH19 | | | | |
| hIL10Ra_VHH14 - | GGGS | Fc | 0.1 | 29961 |
| hIL2Rg_VHH19 | | | | |
| hIL10Ra_VHH14 - | GGGS | Fc | 1 | 29754 |
| hIL2Rg_VHH19 | | | | |
| hIL10Ra_VHH14 - | GGGS | Fc | 10 | 28484 |
| hIL2Rg_VHH19 | | | | |
| hIL10Ra_VHH14 - | GGGS | Fc | 100 | 25731 |
| hIL2Rg_VHH19 | | | | |
| hIL10Ra_VHH14 - | GGGS | his | 0 | 25242 |
| hIL2Rg_VHH19 | | | | |
| hIL10Ra_VHH14 - | GGGS | his | 0.0001 | 19701 |
| hIL2Rg_VHH19 | | | | |
| hIL10Ra_VHH14 - | GGGS | his | 0.001 | 20912 |
| hIL2Rg_VHH19 | | | | |
| hIL10Ra_VHH14 - | GGGS | his | 0.01 | 22320 |
| hIL2Rg_VHH19 | | | | |
| hIL10Ra_VHH14 - | GGGS | his | 0.1 | 23735 |
| hIL2Rg_VHH19 | | | | |
| hIL10Ra_VHH14 - | GGGS | his | 1 | 26393 |
| hIL2Rg_VHH19 | | | | |
| hIL10Ra_VHH14 - | GGGS | his | 10 | 26515 |
| hIL2Rg_VHH19 | | | | |
| hIL10Ra_VHH14 - | GGGS | his | 100 | 27258 |
| hIL2Rg_VHH19 | | | | |
| hIL10Ra_VHH14 - | No linker | his | 0 | 25242 |
| hIL2Rg_VHH19 | | | | |
| hIL10Ra_VHH14 - | No linker | his | 0.0001 | 19660 |
| hIL2Rg_VHH19 | | | | |
| hIL10Ra_VHH14 - | No linker | his | 0.001 | 22316 |
| hIL2Rg_VHH19 | | | | |
| hIL10Ra_VHH14 - | No linker | his | 0.01 | 22237 |
| hIL2Rg_VHH19 | | | | |
| hIL10Ra_VHH14 - | No linker | his | 0.1 | 22894 |
| hIL2Rg_VHH19 | | | | |
| hIL10Ra_VHH14 - | No linker | his | 1 | 24422 |
| hIL2Rg_VHH19 | | | | |
| hIL10Ra_VHH14 - | No linker | his | 10 | 26020 |
| hIL2Rg_VHH19 | | | | |
| hIL10Ra_VHH14 - | No linker | his | 100 | 27507 |
| hIL2Rg_VHH19 | | | | |
| hIL10Ra_VHH14 - | GS | his | 0 | 25242 |
| hIL2Rg_VHH19 | | | | |
| hIL10Ra_VHH14 - | GS | his | 0.0001 | 19871 |
| hIL2Rg_VHH19 | | | | |
| hIL10Ra_VHH14 - | GS | his | 0.001 | 22696 |
| hIL2Rg_VHH19 | | | | |
| hIL10Ra_VHH14 - | GS | his | 0.01 | 25887 |
| hIL2Rg_VHH19 | | | | |
| hIL10Ra_VHH14 - | GS | his | 0.1 | 26102 |
| hIL2Rg_VHH19 | | | | |
| hIL10Ra_VHH14 - | GS | his | 1 | 27877 |
| hIL2Rg_VHH19 | | | | |
| hIL10Ra_VHH14 - | GS | his | 10 | 28634 |
| hIL2Rg_VHH19 | | | | |
| hIL10Ra_VHH14 - | GS | his | 100 | 28383 |
| hIL2Rg_VHH19 | | | | |
| hIL10Ra_VHH14 - | GGSGGS | his | 0 | 25242 |
| hIL2Rg_VHH19 | | | | |
| hIL10Ra_VHH14 - | GGSGGS | his | 0.0001 | 20762 |
| hIL2Rg_VHH19 | | | | |
| hIL10Ra_VHH14 - | GGSGGS | his | 0.001 | 22985 |
| hIL2Rg_VHH19 | | | | |
| hIL10Ra_VHH14 - | GGSGGS | his | 0.01 | 25327 |
| hIL2Rg_VHH19 | | | | |
| hIL10Ra_VHH14 - | GGSGGS | his | 0.1 | 27132 |
| hIL2Rg_VHH19 | | | | |
| hIL10Ra_VHH14 - | GGSGGS | his | 1 | 28702 |
| hIL2Rg_VHH19 | | | | |
| hIL10Ra_VHH14 - | GGSGGS | his | 10 | 29363 |
| hIL2Rg_VHH19 | | | | |
| hIL10Ra_VHH14 - | GGSGGS | his | 100 | 25620 |
| hIL2Rg_VHH19 | | | | |
| hIL10Ra_VHH14 - | GGGSGGGS | his | 0 | 25242 |
| hIL2Rg_VHH19 | | | | |
| hIL10Ra_VHH14 - | GGGSGGGS | his | 0.0001 | 19660 |
| hIL2Rg_VHH19 | | | | |
| hIL10Ra_VHH14 - | GGGSGGGS | his | 0.001 | 22300 |
| hIL2Rg_VHH19 | | | | |
| hIL10Ra_VHH14 - | GGGSGGGS | his | 0.01 | 22652 |
| hIL2Rg_VHH19 | | | | |
| hIL10Ra_VHH14 - | GGGSGGGS | his | 0.1 | 26036 |
| hIL2Rg_VHH19 | | | | |
| hIL10Ra_VHH14 - | GGGSGGGS | his | 1 | 30060 |
| hIL2Rg_VHH19 | | | | |
| hIL10Ra_VHH14 - | GGGSGGGS | his | 10 | 29261 |
| hIL2Rg_VHH19 | | | | |
| hIL10Ra_VHH14 - | GGGSGGGS | his | 100 | 24800 |
| hIL2Rg_VHH19 | | | | |
| hIL10Ra_VHH14 - | GGSGGSGGSG | his | 0 | 25242 |
| hIL2Rg_VHH19 | | | | |
| hIL10Ra_VHH14 - | GGSGGSGGSG | his | 0.0001 | 20606 |
| hIL2Rg_VHH19 | | | | |
| hIL10Ra_VHH14 - | GGSGGSGGSG | his | 0.001 | 25240 |
| hIL2Rg_VHH19 | | | | |
| hIL10Ra_VHH14 - | GGSGGSGGSG | his | 0.01 | 28085 |
| hIL2Rg_VHH19 | | | | |
| hIL10Ra_VHH14 - | GGSGGSGGSG | his | 0.1 | 29307 |
| hIL2Rg_VHH19 | | | | |
| hIL10Ra_VHH14 - | GGSGGSGGSG | his | 1 | 30958 |
| hIL2Rg_VHH19 | | | | |
| hIL10Ra_VHH14 - | GGSGGSGGSG | his | 10 | 31489 |
| hIL2Rg_VHH19 | | | | |
| hIL10Ra_VHH14 - | GGSGGSGGSG | his | 100 | 29905 |
| hIL2Rg_VHH19 | | | | |

TABLE 25
Activity of VHH dimers on CD8 T cell
Granzyme B secretion.

| | | | Concen- | |
|---|---|---|---|---|
| | | | tration | |
| | | | of | Gran- |
| | | | molecule | zyme B |
| VHH1 - VHH2 ID | Linker | Tag | (μM) | (pg/mL) |
| hIL10Ra_VHH14 - | GGGS | Fc | 0 | 77743 |
| hIL2Rg_VHH19 | | | | |
| hIL10Ra_VHH14 - | GGGS | Fc | 0.0001 | 54536 |
| hIL2Rg_VHH19 | | | | |
| hIL10Ra_VHH14 - | GGGS | Fc | 0.001 | 60933 |
| hIL2Rg_VHH19 | | | | |
| hIL10Ra_VHH14 - | GGGS | Fc | 0.01 | 78976 |
| hIL2Rg_VHH19 | | | | |
| hIL10Ra_VHH14 - | GGGS | Fc | 0.1 | 108176 |
| hIL2Rg_VHH19 | | | | |
| hIL10Ra_VHH14 - | GGGS | Fc | 1 | 134135 |
| hIL2Rg_VHH19 | | | | |
| hIL10Ra_VHH14 - | GGGS | Fc | 10 | 151338 |
| hIL2Rg_VHH19 | | | | |
| hIL10Ra_VHH14 - | GGGS | Fc | 100 | 93281 |
| hIL2Rg_VHH19 | | | | |
| hIL10Ra_VHH14 - | GGGS | his | 0 | 77743 |
| hIL2Rg_VHH19 | | | | |
| hIL10Ra_VHH14 - | GGGS | his | 0.0001 | 49291 |
| hIL2Rg_VHH19 | | | | |
| hIL10Ra_VHH14 - | GGGS | his | 0.001 | 45559 |
| hIL2Rg_VHH19 | | | | |
| hIL10Ra_VHH14 - | GGGS | his | 0.01 | 48088 |
| hIL2Rg_VHH19 | | | | |
| hIL10Ra_VHH14 - | GGGS | his | 0.1 | 55281 |
| hIL2Rg_VHH19 | | | | |
| hIL10Ra_VHH14 - | GGGS | his | 1 | 72656 |
| hIL2Rg_VHH19 | | | | |
| hIL10Ra_VHH14 - | GGGS | his | 10 | 84685 |
| hIL2Rg_VHH19 | | | | |
| hIL10Ra_VHH14 - | GGGS | his | 100 | 94573 |
| hIL2Rg_VHH19 | | | | |
| hIL10Ra_VHH14 - | No linker | his | 0 | 77743 |
| hIL2Rg_VHH19 | | | | |
| hIL10Ra_VHH14 - | No linker | his | 0.0001 | 49323 |
| hIL2Rg_VHH19 | | | | |
| hIL10Ra_VHH14 - | No linker | his | 0.001 | 47261 |
| hIL2Rg_VHH19 | | | | |
| hIL10Ra_VHH14 - | No linker | his | 0.01 | 44212 |
| hIL2Rg_VHH19 | | | | |
| hIL10Ra_VHH14 - | No linker | his | 0.1 | 54822 |
| hIL2Rg_VHH19 | | | | |
| hIL10Ra_VHH14 - | No linker | his | 1 | 56290 |
| hIL2Rg_VHH19 | | | | |
| hIL10Ra_VHH14 - | No linker | his | 10 | 67061 |
| hIL2Rg_VHH19 | | | | |
| hIL10Ra_VHH14 - | No linker | his | 100 | 74310 |
| hIL2Rg_VHH19 | | | | |
| hIL10Ra_VHH14 - | GS | his | 0 | 77743 |
| hIL2Rg_VHH19 | | | | |
| hIL10Ra_VHH14 - | GS | his | 0.0001 | 49205 |
| hIL2Rg_VHH19 | | | | |
| hIL10Ra_VHH14 - | GS | his | 0.001 | 49379 |
| hIL2Rg_VHH19 | | | | |
| hIL10Ra_VHH14 - | GS | his | 0.01 | 55456 |
| hIL2Rg_VHH19 | | | | |
| hIL10Ra_VHH14 - | GS | his | 0.1 | 58033 |
| hIL2Rg_VHH19 | | | | |
| hIL10Ra_VHH14 - | GS | his | 1 | 80216 |
| hIL2Rg_VHH19 | | | | |
| hIL10Ra_VHH14 - | GS | his | 10 | 86955 |
| hIL2Rg_VHH19 | | | | |
| hIL10Ra_VHH14 - | GS | his | 100 | 99876 |
| hIL2Rg_VHH19 | | | | |
| hIL10Ra_VHH14 - | GGSGGS | his | 0 | 77743 |
| hIL2Rg_VHH19 | | | | |
| hIL10Ra_VHH14 - | GGSGGS | his | 0.0001 | 54496 |
| hIL2Rg_VHH19 | | | | |
| hIL10Ra_VHH14 - | GGSGGS | his | 0.001 | 50920 |
| hIL2Rg_VHH19 | | | | |
| hIL10Ra_VHH14 - | GGSGGS | his | 0.01 | 54056 |
| hIL2Rg_VHH19 | | | | |
| hIL10Ra_VHH14 - | GGSGGS | his | 0.1 | 62317 |
| hIL2Rg_VHH19 | | | | |
| hIL10Ra_VHH14 - | GGSGGS | his | 1 | 77563 |
| hIL2Rg_VHH19 | | | | |
| hIL10Ra_VHH14 - | GGSGGS | his | 10 | 89980 |
| hIL2Rg_VHH19 | | | | |
| hIL10Ra_VHH14 - | GGSGGS | his | 100 | 77902 |
| hIL2Rg_VHH19 | | | | |
| hIL10Ra_VHH14 - | GGGSGGGS | his | 0 | 77743 |
| hIL2Rg_VHH19 | | | | |
| hIL10Ra_VHH14 - | GGGSGGGS | his | 0.0001 | 52639 |
| hIL2Rg_VHH19 | | | | |
| hIL10Ra_VHH14 - | GGGSGGGS | his | 0.001 | 45454 |
| hIL2Rg_VHH19 | | | | |
| hIL10Ra_VHH14 - | GGGSGGGS | his | 0.01 | 47256 |
| hIL2Rg_VHH19 | | | | |
| hIL10Ra_VHH14 - | GGGSGGGS | his | 0.1 | 62245 |
| hIL2Rg_VHH19 | | | | |
| hIL10Ra_VHH14 - | GGGSGGGS | his | 1 | 80475 |
| hIL2Rg_VHH19 | | | | |
| hIL10Ra_VHH14 - | GGGSGGGS | his | 10 | 83472 |
| hIL2Rg_VHH19 | | | | |
| hIL10Ra_VHH14 - | GGGSGGGS | his | 100 | 78110 |
| hIL2Rg_VHH19 | | | | |
| hIL10Ra_VHH14 - | GGSGGSGGSG | his | 0 | 77743 |
| hIL2Rg_VHH19 | | | | |
| hIL10Ra_VHH14 - | GGSGGSGGSG | his | 0.0001 | 68889 |
| hIL2Rg_VHH19 | | | | |
| hIL10Ra_VHH14 - | GGSGGSGGSG | his | 0.001 | 70609 |
| hIL2Rg_VHH19 | | | | |
| hIL10Ra_VHH14 - | GGSGGSGGSG | his | 0.01 | 70990 |
| hIL2Rg_VHH19 | | | | |
| hIL10Ra_VHH14 - | GGSGGSGGSG | his | 0.1 | 79349 |
| hIL2Rg_VHH19 | | | | |
| hIL10Ra_VHH14 - | GGSGGSGGSG | his | 1 | 111535 |
| hIL2Rg_VHH19 | | | | |
| hIL10Ra_VHH14 - | GGSGGSGGSG | his | 10 | 125459 |
| hIL2Rg_VHH19 | | | | |
| hIL10Ra_VHH14 - | GGSGGSGGSG | his | 100 | 106090 |
| hIL2Rg_VHH19 | | | | |

TABLE 26
Activity of VHH dimers on CD8 T
cell IL-9 secretion.

| | | | Concen- | |
|---|---|---|---|---|
| | | | tration | |
| | | | of | |
| | | | molecule | IL-9 |
| VHH1 - VHH2 ID | Linker | Tag | (μM) | (pg/mL) |
| hIL10Ra_VHH14 - | GGGS | Fc | 0 | 1348 |
| hIL2Rg_VHH19 | | | | |
| hIL10Ra_VHH14 - | GGGS | Fc | 0.0001 | 1055 |
| hIL2Rg_VHH19 | | | | |
| hIL10Ra_VHH14 - | GGGS | Fc | 0.001 | 1116 |
| hIL2Rg_VHH19 | | | | |
| hIL10Ra_VHH14 - | GGGS | Fc | 0.01 | 1162 |
| hIL2Rg_VHH19 | | | | |
| hIL10Ra_VHH14 - | GGGS | Fc | 0.1 | 1424 |
| hIL2Rg_VHH19 | | | | |
| hIL10Ra_VHH14 - | GGGS | Fc | 1 | 1782 |
| hIL2Rg_VHH19 | | | | |
| hIL10Ra_VHH14 - | GGGS | Fc | 10 | 2146 |
| hIL2Rg_VHH19 | | | | |
| hIL10Ra_VHH14 - | GGGS | Fc | 100 | 1926 |
| hIL2Rg_VHH19 | | | | |
| hIL10Ra_VHH14 - | GGGS | his | 0 | 1348 |
| hIL2Rg_VHH19 | | | | |
| hIL10Ra_VHH14 - | GGGS | his | 0.0001 | 941 |
| hIL2Rg_VHH19 | | | | |
| hIL10Ra_VHH14 - | GGGS | his | 0.001 | 1001 |
| hIL2Rg_VHH19 | | | | |
| hIL10Ra_VHH14 - | GGGS | his | 0.01 | 1117 |
| hIL2Rg_VHH19 | | | | |
| hIL10Ra_VHH14 - | GGGS | his | 0.1 | 1338 |
| hIL2Rg_VHH19 | | | | |
| hIL10Ra_VHH14 - | GGGS | his | 1 | 1399 |
| hIL2Rg_VHH19 | | | | |
| hIL10Ra_VHH14 - | GGGS | his | 10 | 1685 |
| hIL2Rg_VHH19 | | | | |
| hIL10Ra_VHH14 - | GGGS | his | 100 | 1707 |
| hIL2Rg_VHH19 | | | | |
| hIL10Ra_VHH14 - | No linker | his | 0 | 1348 |
| hIL2Rg_VHH19 | | | | |
| hIL10Ra_VHH14 - | No linker | his | 0.0001 | 933 |
| hIL2Rg_VHH19 | | | | |
| hIL10Ra_VHH14 - | No linker | his | 0.001 | 1125 |
| hIL2Rg_VHH19 | | | | |
| hIL10Ra_VHH14 - | No linker | his | 0.01 | 1141 |
| hIL2Rg_VHH19 | | | | |
| hIL10Ra_VHH14 - | No linker | his | 0.1 | 1066 |
| hIL2Rg_VHH19 | | | | |
| hIL10Ra_VHH14 - | No linker | his | 1 | 1216 |
| hIL2Rg_VHH19 | | | | |
| hIL10Ra_VHH14 - | No linker | his | 10 | 1438 |
| hIL2Rg_VHH19 | | | | |
| hIL10Ra_VHH14 - | No linker | his | 100 | 1666 |
| hIL2Rg_VHH19 | | | | |
| hIL10Ra_VHH14 - | GS | his | 0 | 1348 |
| hIL2Rg_VHH19 | | | | |
| hIL10Ra_VHH14 - | GS | his | 0.0001 | 918 |
| hIL2Rg_VHH19 | | | | |
| hIL10Ra_VHH14 - | GS | his | 0.001 | 998 |
| hIL2Rg_VHH19 | | | | |
| hIL10Ra_VHH14 - | GS | his | 0.01 | 1072 |
| hIL2Rg_VHH19 | | | | |
| hIL10Ra_VHH14 - | GS | his | 0.1 | 1228 |
| hIL2Rg_VHH19 | | | | |
| hIL10Ra_VHH14 - | GS | his | 1 | 1608 |
| hIL2Rg_VHH19 | | | | |
| hIL10Ra_VHH14 - | GS | his | 10 | 1729 |
| hIL2Rg_VHH19 | | | | |
| hIL10Ra_VHH14 - | GS | his | 100 | 1882 |
| hIL2Rg_VHH19 | | | | |
| hIL10Ra_VHH14 - | GGSGGS | his | 0 | 1348 |
| hIL2Rg_VHH19 | | | | |
| hIL10Ra_VHH14 - | GGSGGS | his | 0.0001 | 951 |
| hIL2Rg_VHH19 | | | | |
| hIL10Ra_VHH14 - | GGSGGS | his | 0.001 | 1006 |
| hIL2Rg_VHH19 | | | | |
| hIL10Ra_VHH14 - | GGSGGS | his | 0.01 | 1144 |
| hIL2Rg_VHH19 | | | | |
| hIL10Ra_VHH14 - | GGSGGS | his | 0.1 | 1090 |
| hIL2Rg_VHH19 | | | | |
| hIL10Ra_VHH14 - | GGSGGS | his | 1 | 1436 |
| hIL2Rg_VHH19 | | | | |
| hIL10Ra_VHH14 - | GGSGGS | his | 10 | 1604 |
| hIL2Rg_VHH19 | | | | |
| hIL10Ra_VHH14 - | GGSGGS | his | 100 | 1698 |
| hIL2Rg_VHH19 | | | | |
| hIL10Ra_VHH14 - | GGGSGGGS | his | 0 | 1348 |
| hIL2Rg_VHH19 | | | | |
| hIL10Ra_VHH14 - | GGGSGGGS | his | 0.0001 | 988 |
| hIL2Rg_VHH19 | | | | |
| hIL10Ra_VHH14 - | GGGSGGGS | his | 0.001 | 1053 |
| hIL2Rg_VHH19 | | | | |
| hIL10Ra_VHH14 - | GGGSGGGS | his | 0.01 | 1007 |
| hIL2Rg_VHH19 | | | | |
| hIL10Ra_VHH14 - | GGGSGGGS | his | 0.1 | 1305 |
| hIL2Rg_VHH19 | | | | |
| hIL10Ra_VHH14 - | GGGSGGGS | his | 1 | 1381 |
| hIL2Rg_VHH19 | | | | |
| hIL10Ra_VHH14 - | GGGSGGGS | his | 10 | 1885 |
| hIL2Rg_VHH19 | | | | |
| hIL10Ra_VHH14 - | GGGSGGGS | his | 100 | 1700 |
| hIL2Rg_VHH19 | | | | |
| hIL10Ra_VHH14 - | GGSGGSGGSG | his | 0 | 1348 |
| hIL2Rg_VHH19 | | | | |
| hIL10Ra_VHH14 - | GGSGGSGGSG | his | 0.0001 | 1007 |
| hIL2Rg_VHH19 | | | | |
| hIL10Ra_VHH14 - | GGSGGSGGSG | his | 0.001 | 1315 |
| hIL2Rg_VHH19 | | | | |
| hIL10Ra_VHH14 - | GGSGGSGGSG | his | 0.01 | 1391 |
| hIL2Rg_VHH19 | | | | |
| hIL10Ra_VHH14 - | GGSGGSGGSG | his | 0.1 | 1449 |
| hIL2Rg_VHH19 | | | | |
| hIL10Ra_VHH14 - | GGSGGSGGSG | his | 1 | 1588 |
| hIL2Rg_VHH19 | | | | |
| hIL10Ra_VHH14 - | GGSGGSGGSG | his | 10 | 2052 |
| hIL2Rg_VHH19 | | | | |
| hIL10Ra_VHH14 - | GGSGGSGGSG | his | 100 | 1965 |
| hIL2Rg_VHH19 | | | | |

TABLE 27
Activity of VHH dimers on LPS treated Monocyte IL-1B secretion.

| | | | Concentration | |
|---|---|---|---|---|
| VHH1-VHH2 | | | of | IL-1β |
| ID | Linker | Tag | molecule (μM) | (pg/mL) |
| hIL10Ra_VHH14- | GGGS | Fc | 0 | 6244 |
| hIL2Rg_VHH19 | | | | |
| hIL10Ra_VHH14- | GGGS | Fc | 0 | 6880 |
| hIL2Rg_VHH19 | | | (LPS only) | |
| hIL10Ra_VHH14- | GGGS | Fc | 0.0001 | 6290 |
| hIL2Rg_VHH19 | | | | |
| hIL10Ra_VHH14- | GGGS | Fc | 0.001 | 5571 |
| hIL2Rg_VHH19 | | | | |
| hIL10Ra_VHH14- | GGGS | Fc | 0.01 | 5259 |
| hIL2Rg_VHH19 | | | | |
| hIL10Ra_VHH14- | GGGS | Fc | 0.1 | 4018 |
| hIL2Rg_VHH19 | | | | |
| hIL10Ra_VHH14- | GGGS | Fc | 1 | 3665 |
| hIL2Rg_VHH19 | | | | |
| hIL10Ra_VHH14- | GGGS | Fc | 10 | 4143 |
| hIL2Rg_VHH19 | | | | |
| hIL10Ra_VHH14- | GGGS | Fc | 100 | 6478 |
| hIL2Rg_VHH19 | | | | |
| hIL10Ra_VHH14- | GGGS | his | 0 | 6244 |
| hIL2Rg_VHH19 | | | | |
| hIL10Ra_VHH14- | GGGS | his | 0 | 6880 |
| hIL2Rg_VHH19 | | | (LPS only) | |
| hIL10Ra_VHH14- | GGGS | his | 0.0001 | 6424 |
| hIL2Rg_VHH19 | | | | |
| hIL10Ra_VHH14- | GGGS | his | 0.001 | 6644 |
| hIL2Rg_VHH19 | | | | |
| hIL10Ra_VHH14- | GGGS | his | 0.01 | 5763 |
| hIL2Rg_VHH19 | | | | |
| hIL10Ra_VHH14- | GGGS | his | 0.1 | 6124 |
| hIL2Rg_VHH19 | | | | |
| hIL10Ra_VHH14- | GGGS | his | 1 | 5354 |
| hIL2Rg_VHH19 | | | | |
| hIL10Ra_VHH14- | GGGS | his | 10 | 5951 |
| hIL2Rg_VHH19 | | | | |
| hIL10Ra_VHH14- | GGGS | his | 100 | 6580 |
| hIL2Rg_VHH19 | | | | |
| hIL10Ra_VHH14- | No linker | his | 0 | 6244 |
| hIL2Rg_VHH19 | | | | |
| hIL10Ra_VHH14- | No linker | his | 0 | 6880 |
| hIL2Rg_VHH19 | | | (LPS only) | |
| hIL10Ra_VHH14- | No linker | his | 0.0001 | 5541 |
| hIL2Rg_VHH19 | | | | |
| hIL10Ra_VHH14- | No linker | his | 0.001 | 5549 |
| hIL2Rg_VHH19 | | | | |
| hIL10Ra_VHH14- | No linker | his | 0.01 | 5373 |
| hIL2Rg_VHH19 | | | | |
| hIL10Ra_VHH14- | No linker | his | 0.1 | 5290 |
| hIL2Rg_VHH19 | | | | |
| hIL10Ra_VHH14- | No linker | his | 1 | 5801 |
| hIL2Rg_VHH19 | | | | |
| hIL10Ra_VHH14- | No linker | his | 10 | 5792 |
| hIL2Rg_VHH19 | | | | |
| hIL10Ra_VHH14- | No linker | his | 100 | 7100 |
| hIL2Rg_VHH19 | | | | |
| hIL10Ra_VHH14- | GS | his | 0 | 6244 |
| hIL2Rg_VHH19 | | | | |
| hIL10Ra_VHH14- | GS | his | 0 | 6880 |
| hIL2Rg_VHH19 | | | (LPS only) | |
| hIL10Ra_VHH14- | GS | his | 0.0001 | 5964 |
| hIL2Rg_VHH19 | | | | |
| hIL10Ra_VHH14- | GS | his | 0.001 | 6266 |
| hIL2Rg_VHH19 | | | | |
| hIL10Ra_VHH14- | GS | his | 0.01 | 5065 |
| hIL2Rg_VHH19 | | | | |
| hIL10Ra_VHH14- | GS | his | 0.1 | 6592 |
| hIL2Rg_VHH19 | | | | |
| hIL10Ra_VHH14- | GS | his | 1 | 5765 |
| hIL2Rg_VHH19 | | | | |
| hIL10Ra_VHH14- | GS | his | 10 | 6707 |
| hIL2Rg_VHH19 | | | | |
| hIL10Ra_VHH14- | GS | his | 100 | 8142 |
| hIL2Rg_VHH19 | | | | |
| hIL10Ra_VHH14- | GGSGGS | his | 0 | 6244 |
| hIL2Rg_VHH19 | | | | |
| hIL10Ra_VHH14- | GGSGGS | his | 0 | 6880 |
| hIL2Rg_VHH19 | | | (LPS only) | |
| hIL10Ra_VHH14- | GGSGGS | his | 0.0001 | 6288 |
| hIL2Rg_VHH19 | | | | |
| hIL10Ra_VHH14- | GGSGGS | his | 0.001 | 5849 |
| hIL2Rg_VHH19 | | | | |
| hIL10Ra_VHH14- | GGSGGS | his | 0.01 | 6103 |
| hIL2Rg_VHH19 | | | | |
| hIL10Ra_VHH14- | GGSGGS | his | 0.1 | 4955 |
| hIL2Rg_VHH19 | | | | |
| hIL10Ra_VHH14- | GGSGGS | his | 1 | 5780 |
| hIL2Rg_VHH19 | | | | |
| hIL10Ra_VHH14- | GGSGGS | his | 10 | 6425 |
| hIL2Rg_VHH19 | | | | |
| hIL10Ra_VHH14- | GGSGGS | his | 100 | 7844 |
| hIL2Rg_VHH19 | | | | |
| hIL10Ra_VHH14- | GGGSGGGS | his | 0 | 6244 |
| hIL2Rg_VHH19 | | | | |
| hIL10Ra_VHH14- | GGGSGGGS | his | 0 | 6880 |
| hIL2Rg_VHH19 | | | (LPS only) | |
| hIL10Ra_VHH14- | GGGSGGGS | his | 0.0001 | 5950 |
| hIL2Rg_VHH19 | | | | |
| hIL10Ra_VHH14- | GGGSGGGS | his | 0.001 | 6200 |
| hIL2Rg_VHH19 | | | | |
| hIL10Ra_VHH14- | GGGSGGGS | his | 0.01 | 4959 |
| hIL2Rg_VHH19 | | | | |
| hIL10Ra_VHH14- | GGGSGGGS | his | 0.1 | 5594 |
| hIL2Rg_VHH19 | | | | |
| hIL10Ra_VHH14- | GGGSGGGS | his | 1 | 5672 |
| hIL2Rg_VHH19 | | | | |
| hIL10Ra_VHH14- | GGGSGGGS | his | 10 | 6017 |
| hIL2Rg_VHH19 | | | | |
| hIL10Ra_VHH14- | GGGSGGGS | his | 100 | 6843 |
| hIL2Rg_VHH19 | | | | |
| hIL10Ra_VHH14- | GGSGGSGGSG | his | 0 | 6244 |
| hIL2Rg_VHH19 | | | | |
| hIL10Ra_VHH14- | GGSGGSGGSG | his | 0 | 6880 |
| hIL2Rg_VHH19 | | | (LPS only) | |
| hIL10Ra_VHH14- | GGSGGSGGSG | his | 0.0001 | 5894 |
| hIL2Rg_VHH19 | | | | |
| hIL10Ra_VHH14- | GGSGGSGGSG | his | 0.001 | 5389 |
| hIL2Rg_VHH19 | | | | |
| hIL10Ra_VHH14- | GGSGGSGGSG | his | 0.01 | 6018 |
| hIL2Rg_VHH19 | | | | |
| hIL10Ra_VHH14- | GGSGGSGGSG | his | 0.1 | 5241 |
| hIL2Rg_VHH19 | | | | |
| hIL10Ra_VHH14- | GGSGGSGGSG | his | 1 | 5680 |
| hIL2Rg_VHH19 | | | | |
| hIL10Ra_VHH14- | GGSGGSGGSG | his | 10 | 7124 |
| hIL2Rg_VHH19 | | | | |
| hIL10Ra_VHH14- | GGSGGSGGSG | his | 100 | 8781 |
| hIL2Rg_VHH19 | | | | |

TABLE 28
Activity of VHH dimers on LPS
treated Monocyte IL-6 secretion.

| | | | Concen- | |
|---|---|---|---|---|
| | | | tration | |
| | | | of | |
| | | | molecule | IL-6 |
| VHH1 - VHH2 ID | Linker | Tag | (μM) | (pg/mL) |
| hIL10Ra_VHH14 - | GGGS | Fc | 0 | 31269 |
| hIL2Rg_VHH19 | | | | |
| hIL10Ra_VHH14 - | GGGS | Fc | 0 (LPS | 45404 |
| hIL2Rg_VHH19 | | | only) | |
| hIL10Ra_VHH14 - | GGGS | Fc | 0.0001 | 36196 |
| hIL2Rg_VHH19 | | | | |
| hIL10Ra_VHH14 - | GGGS | Fc | 0.001 | 30932 |
| hIL2Rg_VHH19 | | | | |
| hIL10Ra_VHH14 - | GGGS | Fc | 0.01 | 27747 |
| hIL2Rg_VHH19 | | | | |
| hIL10Ra_VHH14 - | GGGS | Fc | 0.1 | 24512 |
| hIL2Rg_VHH19 | | | | |
| hIL10Ra_VHH14 - | GGGS | Fc | 1 | 21794 |
| hIL2Rg_VHH19 | | | | |
| hIL10Ra_VHH14 - | GGGS | Fc | 10 | 31465 |
| hIL2Rg_VHH19 | | | | |
| hIL10Ra_VHH14 - | GGGS | Fc | 100 | 44297 |
| hIL2Rg_VHH19 | | | | |
| hIL10Ra_VHH14 - | GGGS | his | 0 | 31269 |
| hIL2Rg_VHH19 | | | | |
| hIL10Ra_VHH14 - | GGGS | his | 0 (LPS | 45404 |
| hIL2Rg_VHH19 | | | only) | |
| hIL10Ra_VHH14 - | GGGS | his | 0.0001 | 39223 |
| hIL2Rg_VHH19 | | | | |
| hIL10Ra_VHH14 - | GGGS | his | 0.001 | 41699 |
| hIL2Rg_VHH19 | | | | |
| hIL10Ra_VHH14 - | GGGS | his | 0.01 | 33346 |
| hIL2Rg_VHH19 | | | | |
| hIL10Ra_VHH14 - | GGGS | his | 0.1 | 32608 |
| hIL2Rg_VHH19 | | | | |
| hIL10Ra_VHH14 - | GGGS | his | 1 | 24493 |
| hIL2Rg_VHH19 | | | | |
| hIL10Ra_VHH14 - | GGGS | his | 10 | 30616 |
| hIL2Rg_VHH19 | | | | |
| hIL10Ra_VHH14 - | GGGS | his | 100 | 32878 |
| hIL2Rg_VHH19 | | | | |
| hIL10Ra_VHH14 - | No linker | his | 0 | 31269 |
| hIL2Rg_VHH19 | | | | |
| hIL10Ra_VHH14 - | No linker | his | 0 (LPS | 45404 |
| hIL2Rg_VHH19 | | | only) | |
| hIL10Ra_VHH14 - | No linker | his | 0.0001 | 30458 |
| hIL2Rg_VHH19 | | | | |
| hIL10Ra_VHH14 - | No linker | his | 0.001 | 31485 |
| hIL2Rg_VHH19 | | | | |
| hIL10Ra_VHH14 - | No linker | his | 0.01 | 30025 |
| hIL2Rg_VHH19 | | | | |
| hIL10Ra_VHH14 - | No linker | his | 0.1 | 37420 |
| hIL2Rg_VHH19 | | | | |
| hIL10Ra_VHH14 - | No linker | his | 1 | 27897 |
| hIL2Rg_VHH19 | | | | |
| hIL10Ra_VHH14 - | No linker | his | 10 | 33304 |
| hIL2Rg_VHH19 | | | | |
| hIL10Ra_VHH14 - | No linker | his | 100 | 36593 |
| hIL2Rg_VHH19 | | | | |
| hIL10Ra_VHH14 - | GS | his | 0 | 31269 |
| hIL2Rg_VHH19 | | | | |
| hIL10Ra_VHH14 - | GS | his | 0 (LPS | 45404 |
| hIL2Rg_VHH19 | | | only) | |
| hIL10Ra_VHH14 - | GS | his | 0.0001 | 32766 |
| hIL2Rg_VHH19 | | | | |
| hIL10Ra_VHH14 - | GS | his | 0.001 | 42398 |
| hIL2Rg_VHH19 | | | | |
| hIL10Ra_VHH14 - | GS | his | 0.01 | 19882 |
| hIL2Rg_VHH19 | | | | |
| hIL10Ra_VHH14 - | GS | his | 0.1 | 41496 |
| hIL2Rg_VHH19 | | | | |
| hIL10Ra_VHH14 - | GS | his | 1 | 29074 |
| hIL2Rg_VHH19 | | | | |
| hIL10Ra_VHH14 - | GS | his | 10 | 47742 |
| hIL2Rg_VHH19 | | | | |
| hIL10Ra_VHH14 - | GS | his | 100 | 50764 |
| hIL2Rg_VHH19 | | | | |
| hIL10Ra_VHH14 - | GGSGGS | his | 0 | 31269 |
| hIL2Rg_VHH19 | | | | |
| hIL10Ra_VHH14 - | GGSGGS | his | 0 (LPS | 45404 |
| hIL2Rg_VHH19 | | | only) | |
| hIL10Ra_VHH14 - | GGSGGS | his | 0.0001 | 34223 |
| hIL2Rg_VHH19 | | | | |
| hIL10Ra_VHH14 - | GGSGGS | his | 0.001 | 33794 |
| hIL2Rg_VHH19 | | | | |
| hIL10Ra_VHH14 - | GGSGGS | his | 0.01 | 30119 |
| hIL2Rg_VHH19 | | | | |
| hIL10Ra_VHH14 - | GGSGGS | his | 0.1 | 27763 |
| hIL2Rg_VHH19 | | | | |
| hIL10Ra_VHH14 - | GGSGGS | his | 1 | 26000 |
| hIL2Rg_VHH19 | | | | |
| hIL10Ra_VHH14 - | GGSGGS | his | 10 | 38429 |
| hIL2Rg_VHH19 | | | | |
| hIL10Ra_VHH14 - | GGSGGS | his | 100 | 48903 |
| hIL2Rg_VHH19 | | | | |
| hIL10Ra_VHH14 - | GGGSGGGS | his | 0 | 31269 |
| hIL2Rg_VHH19 | | | | |
| hIL10Ra_VHH14 - | GGGSGGGS | his | 0 (LPS | 45404 |
| hIL2Rg_VHH19 | | | only) | |
| hIL10Ra_VHH14 - | GGGSGGGS | his | 0.0001 | 36784 |
| hIL2Rg_VHH19 | | | | |
| hIL10Ra_VHH14 - | GGGSGGGS | his | 0.001 | 46305 |
| hIL2Rg_VHH19 | | | | |
| hIL10Ra_VHH14 - | GGGSGGGS | his | 0.01 | 21646 |
| hIL2Rg_VHH19 | | | | |
| hIL10Ra_VHH14 - | GGGSGGGS | his | 0.1 | 35779 |
| hIL2Rg_VHH19 | | | | |
| hIL10Ra_VHH14 - | GGGSGGGS | his | 1 | 27065 |
| hIL2Rg_VHH19 | | | | |
| hIL10Ra_VHH14 - | GGGSGGGS | his | 10 | 29227 |
| hIL2Rg_VHH19 | | | | |
| hIL10Ra_VHH14 - | GGGSGGGS | his | 100 | 36634 |
| hIL2Rg_VHH19 | | | | |
| hIL10Ra_VHH14 - | GGSGGSGGSG | his | 0 | 31269 |
| hIL2Rg_VHH19 | | | | |
| hIL10Ra_VHH14 - | GGSGGSGGSG | his | 0 (LPS | 45404 |
| hIL2Rg_VHH19 | | | only) | |
| hIL10Ra_VHH14 - | GGSGGSGGSG | his | 0.0001 | 32788 |
| hIL2Rg_VHH19 | | | | |
| hIL10Ra_VHH14 - | GGSGGSGGSG | his | 0.001 | 41119 |
| hIL2Rg_VHH19 | | | | |
| hIL10Ra_VHH14 - | GGSGGSGGSG | his | 0.01 | 37396 |
| hIL2Rg_VHH19 | | | | |
| hIL10Ra_VHH14 - | GGSGGSGGSG | his | 0.1 | 35343 |
| hIL2Rg_VHH19 | | | | |
| hIL10Ra_VHH14 - | GGSGGSGGSG | his | 1 | 29405 |
| hIL2Rg_VHH19 | | | | |
| hIL10Ra_VHH14 - | GGSGGSGGSG | his | 10 | 49333 |
| hIL2Rg_VHH19 | | | | |
| hIL10Ra_VHH14 - | GGSGGSGGSG | his | 100 | 55000 |
| hIL2Rg_VHH19 | | | | |

TABLE 29
Activity of VHH dimers on LPS treated
Monocyte TNF-α secretion.

| | | | Concen- | |
|---|---|---|---|---|
| | | | tration | |
| | | | of | |
| | | | molecule | TNF-α |
| VHH1 - VHH2 ID | Linker | Tag | (μM) | (pg/mL) |
| hIL10Ra_VHH14 - | GGGS | Fc | 0 | 7455 |
| hIL2Rg_VHH19 | | | | |
| hIL10Ra_VHH14 - | GGGS | Fc | 0 (LPS | 10138 |
| hIL2Rg_VHH19 | | | only) | |
| hIL10Ra_VHH14 - | GGGS | Fc | 0.0001 | 6234 |
| hIL2Rg_VHH19 | | | | |
| hIL10Ra_VHH14 - | GGGS | Fc | 0.001 | 5120 |
| hIL2Rg_VHH19 | | | | |
| hIL10Ra_VHH14 - | GGGS | Fc | 0.01 | 5443 |
| hIL2Rg_VHH19 | | | | |
| hIL10Ra_VHH14 - | GGGS | Fc | 0.1 | 3960 |
| hIL2Rg_VHH19 | | | | |
| hIL10Ra_VHH14 - | GGGS | Fc | 1 | 2790 |
| hIL2Rg_VHH19 | | | | |
| hIL10Ra_VHH14 - | GGGS | Fc | 10 | 3887 |
| hIL2Rg_VHH19 | | | | |
| hIL10Ra_VHH14 - | GGGS | Fc | 100 | 6253 |
| hIL2Rg_VHH19 | | | | |
| hIL10Ra_VHH14 - | GGGS | his | 0 | 7455 |
| hIL2Rg_VHH19 | | | | |
| hIL10Ra_VHH14 - | GGGS | his | 0 (LPS | 10138 |
| hIL2Rg_VHH19 | | | only) | |
| hIL10Ra_VHH14 - | GGGS | his | 0.0001 | 8435 |
| hIL2Rg_VHH19 | | | | |
| hIL10Ra_VHH14 - | GGGS | his | 0.001 | 6253 |
| hIL2Rg_VHH19 | | | | |
| hIL10Ra_VHH14 - | GGGS | his | 0.01 | 5501 |
| hIL2Rg_VHH19 | | | | |
| hIL10Ra_VHH14 - | GGGS | his | 0.1 | 5631 |
| hIL2Rg_VHH19 | | | | |
| hIL10Ra_VHH14 - | GGGS | his | 1 | 4741 |
| hIL2Rg_VHH19 | | | | |
| hIL10Ra_VHH14 - | GGGS | his | 10 | 6068 |
| hIL2Rg_VHH19 | | | | |
| hIL10Ra_VHH14 - | GGGS | his | 100 | 7228 |
| hIL2Rg_VHH19 | | | | |
| hIL10Ra_VHH14 - | No linker | his | 0 | 7455 |
| hIL2Rg_VHH19 | | | | |
| hIL10Ra_VHH14 - | No linker | his | 0 (LPS | 10138 |
| hIL2Rg_VHH19 | | | only) | |
| hIL10Ra_VHH14 - | No linker | his | 0.0001 | 6656 |
| hIL2Rg_VHH19 | | | | |
| hIL10Ra_VHH14 - | No linker | his | 0.001 | 5247 |
| hIL2Rg_VHH19 | | | | |
| hIL10Ra_VHH14 - | No linker | his | 0.01 | 5400 |
| hIL2Rg_VHH19 | | | | |
| hIL10Ra_VHH14 - | No linker | his | 0.1 | 7258 |
| hIL2Rg_VHH19 | | | | |
| hIL10Ra_VHH14 - | No linker | his | 1 | 5004 |
| hIL2Rg_VHH19 | | | | |
| hIL10Ra_VHH14 - | No linker | his | 10 | 6609 |
| hIL2Rg_VHH19 | | | | |
| hIL10Ra_VHH14 - | No linker | his | 100 | 7923 |
| hIL2Rg_VHH19 | | | | |
| hIL10Ra_VHH14 - | GS | his | 0 | 7455 |
| hIL2Rg_VHH19 | | | | |
| hIL10Ra_VHH14 - | GS | his | 0 (LPS | 10138 |
| hIL2Rg_VHH19 | | | only) | |
| hIL10Ra_VHH14 - | GS | his | 0.0001 | 6800 |
| hIL2Rg_VHH19 | | | | |
| hIL10Ra_VHH14 - | GS | his | 0.001 | 6768 |
| hIL2Rg_VHH19 | | | | |
| hIL10Ra_VHH14 - | GS | his | 0.01 | 4996 |
| hIL2Rg_VHH19 | | | | |
| hIL10Ra_VHH14 - | GS | his | 0.1 | 5787 |
| hIL2Rg_VHH19 | | | | |
| hIL10Ra_VHH14 - | GS | his | 1 | 5153 |
| hIL2Rg_VHH19 | | | | |
| hIL10Ra_VHH14 - | GS | his | 10 | 6176 |
| hIL2Rg_VHH19 | | | | |
| hIL10Ra_VHH14 - | GS | his | 100 | 8029 |
| hIL2Rg_VHH19 | | | | |
| hIL10Ra_VHH14 - | GGSGGS | his | 0 | 7455 |
| hIL2Rg_VHH19 | | | | |
| hIL10Ra_VHH14 - | GGSGGS | his | 0 (LPS | 10138 |
| hIL2Rg_VHH19 | | | only) | |
| hIL10Ra_VHH14 - | GGSGGS | his | 0.0001 | 7239 |
| hIL2Rg_VHH19 | | | | |
| hIL10Ra_VHH14 - | GGSGGS | his | 0.001 | 6237 |
| hIL2Rg_VHH19 | | | | |
| hIL10Ra_VHH14 - | GGSGGS | his | 0.01 | 6102 |
| hIL2Rg_VHH19 | | | | |
| hIL10Ra_VHH14 - | GGSGGS | his | 0.1 | 5731 |
| hIL2Rg_VHH19 | | | | |
| hIL10Ra_VHH14 - | GGSGGS | his | 1 | 5739 |
| hIL2Rg_VHH19 | | | | |
| hIL10Ra_VHH14 - | GGSGGS | his | 10 | 6621 |
| hIL2Rg_VHH19 | | | | |
| hIL10Ra_VHH14 - | GGSGGS | his | 100 | 8850 |
| hIL2Rg_VHH19 | | | | |
| hIL10Ra_VHH14 - | GGGSGGGS | his | 0 | 7455 |
| hIL2Rg_VHH19 | | | | |
| hIL10Ra_VHH14 - | GGGSGGGS | his | 0 (LPS | 10138 |
| hIL2Rg_VHH19 | | | only) | |
| hIL10Ra_VHH14 - | GGGSGGGS | his | 0.0001 | 6786 |
| hIL2Rg_VHH19 | | | | |
| hIL10Ra_VHH14 - | GGGSGGGS | his | 0.001 | 7202 |
| hIL2Rg_VHH19 | | | | |
| hIL10Ra_VHH14 - | GGGSGGGS | his | 0.01 | 4699 |
| hIL2Rg_VHH19 | | | | |
| hIL10Ra_VHH14 - | GGGSGGGS | his | 0.1 | 6822 |
| hIL2Rg_VHH19 | | | | |
| hIL10Ra_VHH14 - | GGGSGGGS | his | 1 | 5620 |
| hIL2Rg_VHH19 | | | | |
| hIL10Ra_VHH14 - | GGGSGGGS | his | 10 | 6187 |
| hIL2Rg_VHH19 | | | | |
| hIL10Ra_VHH14 - | GGGSGGGS | his | 100 | 8372 |
| hIL2Rg_VHH19 | | | | |
| hIL10Ra_VHH14 - | GGSGGSGGSG | his | 0 | 7455 |
| hIL2Rg_VHH19 | | | | |
| hIL10Ra_VHH14 - | GGSGGSGGSG | his | 0 (LPS | 10138 |
| hIL2Rg_VHH19 | | | only) | |
| hIL10Ra_VHH14 - | GGSGGSGGSG | his | 0.0001 | 7366 |
| hIL2Rg_VHH19 | | | | |
| hIL10Ra_VHH14 - | GGSGGSGGSG | his | 0.001 | 7535 |
| hIL2Rg_VHH19 | | | | |
| hIL10Ra_VHH14 - | GGSGGSGGSG | his | 0.01 | 8008 |
| hIL2Rg_VHH19 | | | | |
| hIL10Ra_VHH14 - | GGSGGSGGSG | his | 0.1 | 8891 |
| hIL2Rg_VHH19 | | | | |
| hIL10Ra_VHH14 - | GGSGGSGGSG | his | 1 | 7561 |
| hIL2Rg_VHH19 | | | | |
| hIL10Ra_VHH14 - | GGSGGSGGSG | his | 10 | 8465 |
| hIL2Rg_VHH19 | | | | |
| hIL10Ra_VHH14 - | GGSGGSGGSG | his | 100 | 10573 |
| hIL2Rg_VHH19 | | | | |

TABLE 30
Activity of VHH dimers on LPS treated
Monocyte IL-8 secretion.

| | | | Concen- | |
|---|---|---|---|---|
| | | | tration | |
| | | | of | |
| | | | molecule | IL-8 |
| VHH1 - VHH2 ID | Linker | Tag | (μM) | (pg/mL) |
| hIL10Ra_VHH14 - | GGGS | Fc | 0 | 68763 |
| hIL2Rg_VHH19 | | | | |
| hIL10Ra_VHH14 - | GGGS | Fc | 0 (LPS) | 68829 |
| hIL2Rg_VHH19 | | | only | |
| hIL10Ra_VHH14 - | GGGS | Fc | 0.0001 | 73149 |
| hIL2Rg_VHH19 | | | | |
| hIL10Ra_VHH14 - | GGGS | Fc | 0.001 | 68963 |
| hIL2Rg_VHH19 | | | | |
| hIL10Ra_VHH14 - | GGGS | Fc | 0.01 | 72977 |
| hIL2Rg_VHH19 | | | | |
| hIL10Ra_VHH14 - | GGGS | Fc | 0.1 | 69358 |
| hIL2Rg_VHH19 | | | | |
| hIL10Ra_VHH14 - | GGGS | Fc | 1 | 72648 |
| hIL2Rg_VHH19 | | | | |
| hIL10Ra_VHH14 - | GGGS | Fc | 10 | 68592 |
| hIL2Rg_VHH19 | | | | |
| hIL10Ra_VHH14 - | GGGS | Fc | 100 | 71843 |
| hIL2Rg_VHH19 | | | | |
| hIL10Ra_VHH14 - | GGGS | his | 0 | 68763 |
| hIL2Rg_VHH19 | | | | |
| hIL10Ra_VHH14 - | GGGS | his | 0 (LPS | 68829 |
| hIL2Rg_VHH19 | | | only) | |
| hIL10Ra_VHH14 - | GGGS | his | 0.0001 | 72537 |
| hIL2Rg_VHH19 | | | | |
| hIL10Ra_VHH14 - | GGGS | his | 0.001 | 69567 |
| hIL2Rg_VHH19 | | | | |
| hIL10Ra_VHH14 - | GGGS | his | 0.01 | 71836 |
| hIL2Rg_VHH19 | | | | |
| hIL10Ra_VHH14 - | GGGS | his | 0.1 | 67444 |
| hIL2Rg_VHH19 | | | | |
| hIL10Ra_VHH14 - | GGGS | his | 1 | 71572 |
| hIL2Rg_VHH19 | | | | |
| hIL10Ra_VHH14 - | GGGS | his | 10 | 69052 |
| hIL2Rg_VHH19 | | | | |
| hIL10Ra_VHH14 - | GGGS | his | 100 | 71296 |
| hIL2Rg_VHH19 | | | | |
| hIL10Ra_VHH14 - | No linker | his | 0 | 68763 |
| hIL2Rg_VHH19 | | | | |
| hIL10Ra_VHH14 - | No linker | his | 0 (LPS | 68829 |
| hIL2Rg_VHH19 | | | only) | |
| hIL10Ra_VHH14 - | No linker | his | 0.0001 | 73472 |
| hIL2Rg_VHH19 | | | | |
| hIL10Ra_VHH14 - | No linker | his | 0.001 | 69342 |
| hIL2Rg_VHH19 | | | | |
| hIL10Ra_VHH14 - | No linker | his | 0.01 | 73018 |
| hIL2Rg_VHH19 | | | | |
| hIL10Ra_VHH14 - | No linker | his | 0.1 | 69553 |
| hIL2Rg_VHH19 | | | | |
| hIL10Ra_VHH14 - | No linker | his | 1 | 72874 |
| hIL2Rg_VHH19 | | | | |
| hIL10Ra_VHH14 - | No linker | his | 10 | 69630 |
| hIL2Rg_VHH19 | | | | |
| hIL10Ra_VHH14 - | No linker | his | 100 | 71764 |
| hIL2Rg_VHH19 | | | | |
| hIL10Ra_VHH14 - | GS | his | 0 | 68763 |
| hIL2Rg_VHH19 | | | | |
| hIL10Ra_VHH14 - | GS | his | 0 (LPS | 68829 |
| hIL2Rg_VHH19 | | | only) | |
| hIL10Ra_VHH14 - | GS | his | 0.0001 | 73590 |
| hIL2Rg_VHH19 | | | | |
| hIL10Ra_VHH14 - | GS | his | 0.001 | 69641 |
| hIL2Rg_VHH19 | | | | |
| hIL10Ra_VHH14 - | GS | his | 0.01 | 70913 |
| hIL2Rg_VHH19 | | | | |
| hIL10Ra_VHH14 - | GS | his | 0.1 | 69034 |
| hIL2Rg_VHH19 | | | | |
| hIL10Ra_VHH14 - | GS | his | 1 | 71858 |
| hIL2Rg_VHH19 | | | | |
| hIL10Ra_VHH14 - | GS | his | 10 | 68712 |
| hIL2Rg_VHH19 | | | | |
| hIL10Ra_VHH14 - | GS | his | 100 | 72069 |
| hIL2Rg_VHH19 | | | | |
| hIL10Ra_VHH14 - | GGSGGS | his | 0 | 68763 |
| hIL2Rg_VHH19 | | | | |
| hIL10Ra_VHH14 - | GGSGGS | his | 0 (LPS | 68829 |
| hIL2Rg_VHH19 | | | only) | |
| hIL10Ra_VHH14 - | GGSGGS | his | 0.0001 | 73805 |
| hIL2Rg_VHH19 | | | | |
| hIL10Ra_VHH14 - | GGSGGS | his | 0.001 | 69583 |
| hIL2Rg_VHH19 | | | | |
| hIL10Ra_VHH14 - | GGSGGS | his | 0.01 | 72588 |
| hIL2Rg_VHH19 | | | | |
| hIL10Ra_VHH14 - | GGSGGS | his | 0.1 | 68146 |
| hIL2Rg_VHH19 | | | | |
| hIL10Ra_VHH14 - | GGSGGS | his | 1 | 72922 |
| hIL2Rg_VHH19 | | | | |
| hIL10Ra_VHH14 - | GGSGGS | his | 10 | 69402 |
| hIL2Rg_VHH19 | | | | |
| hIL10Ra_VHH14 - | GGSGGS | his | 100 | 72996 |
| hIL2Rg_VHH19 | | | | |
| hIL10Ra_VHH14 - | GGGSGGGS | his | 0 | 68763 |
| hIL2Rg_VHH19 | | | | |
| hIL10Ra_VHH14 - | GGGSGGGS | his | 0 (LPS | 68829 |
| hIL2Rg_VHH19 | | | only) | |
| hIL10Ra_VHH14 - | GGGSGGGS | his | 0.0001 | 72738 |
| hIL2Rg_VHH19 | | | | |
| hIL10Ra_VHH14 - | GGGSGGGS | his | 0.001 | 69165 |
| hIL2Rg_VHH19 | | | | |
| hIL10Ra_VHH14 - | GGGSGGGS | his | 0.01 | 70771 |
| hIL2Rg_VHH19 | | | | |
| hIL10Ra_VHH14 - | GGGSGGGS | his | 0.1 | 67136 |
| hIL2Rg_VHH19 | | | | |
| hIL10Ra_VHH14 - | GGGSGGGS | his | 1 | 71990 |
| hIL2Rg_VHH19 | | | | |
| hIL10Ra_VHH14 - | GGGSGGGS | his | 10 | 68600 |
| hIL2Rg_VHH19 | | | | |
| hIL10Ra_VHH14 - | GGGSGGGS | his | 100 | 72121 |
| hIL2Rg_VHH19 | | | | |
| hIL10Ra_VHH14 - | GGSGGSGGSG | his | 0 | 68763 |
| hIL2Rg_VHH19 | | | | |
| hIL10Ra_VHH14 - | GGSGGSGGSG | his | 0 (LPS | 68829 |
| hIL2Rg_VHH19 | | | only) | |
| hIL10Ra_VHH14 - | GGSGGSGGSG | his | 0.0001 | 73256 |
| hIL2Rg_VHH19 | | | | |
| hIL10Ra_VHH14 - | GGSGGSGGSG | his | 0.001 | 68734 |
| hIL2Rg_VHH19 | | | | |
| hIL10Ra_VHH14 - | GGSGGSGGSG | his | 0.01 | 73317 |
| hIL2Rg_VHH19 | | | | |
| hIL10Ra_VHH14 - | GGSGGSGGSG | his | 0.1 | 68370 |
| hIL2Rg_VHH19 | | | | |
| hIL10Ra_VHH14 - | GGSGGSGGSG | his | 1 | 72889 |
| hIL2Rg_VHH19 | | | | |
| hIL10Ra_VHH14 - | GGSGGSGGSG | his | 10 | 69439 |
| hIL2Rg_VHH19 | | | | |
| hIL10Ra_VHH14 - | GGSGGSGGSG | his | 100 | 72622 |
| hIL2Rg_VHH19 | | | | |

NUCLEIC ACID SEQUENCES

>SEQ ID NO: 83; DR392(DR229-DR236)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTGCAAGCCGGAGGCTCTCTGAGGCTGAGCTGTGCCG
CCTCTAGGTTCACATACAGCAGCTACTGCATGGGCTGGTTCAGACAAGCCCCCGG
CAAAGAGAGAGAAGGCGTGGCCAGCATCGATAGCGATGGCTCCACTAGCTACAC
TGACAGCGTGAAGGGAAGGTTCACTATCAGCAAGGACAACGCCAAGAACACTCT
GTATCTGCAGATGAACTCTCTGAAGCCAGAGGACACAGCCATGTACTACTGTGCC
CTCGATCTGATGAGCACAGTGGTGCCCGGCTTCTGTGGCTTTCTGCTGAGCGCTG
GCATGGATTACTGGGGCAAGGGCACTCAAGTGACTGTCTCGTCTGCTAGCCACCA
TCACCATCACCAC
>SEQ ID NO: 84; DR395(DR229-DR239)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTGCAAGCCGGAGGCTCTCTGAGGCTGAGCTGTACA
GTGTCCGGCTACACTTACAGCTCCAATTGCATGGGCTGGTTTAGGCAAGCCCCCG
GCAAGGAAAGAGAGGGCGTGGCCACTATCTACACTGGCGGCGGCAACACATACT
ACGCCGATAGCGTGAAGGGAAGGTTCACTATCAGCCAAGATAACGCCAAGAACA
CAGTGTATCTGCAGATGAACAATCTGAAGCCAGAGGACACTGCCATGTACTACT
GTGCTGCTGAGCCACTGTCTAGGGTGTACGGCGGCAGCTGCCCAACTCCTACATT
CGACTACTGGGGCCAAGGCACACAAGTGACTGTCTCGTCTGCTAGCCACCATCAC
CATCACCAC
>SEQ ID NO: 85; DR437(DR235-DR233)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGCGGAAGCGTGCAAGCTGGA
GGATCTCTGAGGCTGAGCTGCACAGCCAGCGGCTTCACTTTCGATGACAGCGAC
ATGGGCTGGTATAGGCAAGCCCCCGGCAATGAGTGTGAGCTGGTGAGCACAATC
AGCAGCGACGGCTCCACTTACTACGCCGACAGCGTCAAGGGAAGGTTCACAATC
AGCCAAGATAACGCCAAGAACACTGTGTATCTGCAGATGAACTCTCTGAAGCCA
GAGGACACAGCCGTGTACTACTGTGCTGCCGAGCCTAGGGGCTACTATAGCAAC
TACGGCGGAAGGAGGGAGTGCAATTACTGGGGCCAAGGCACACAAGTGACAGT
CTCGTCTGCTAGCCACCATCACCATCACCAC
>SEQ ID NO: 86; DR438(DR235-DR234)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGAAGCGTGCAAGCCGGA
GGCTCTCTGAGGCTGAGCTGTGTGGCTAGTGGCTACACTTTCAGCAGCTACTGCA
TGGGCTGGTTCAGACAAGCCCCCGGCAAGGAAAGGGAAGGAGTGGCCGCTCTGG
GCGGAGGAAGCACATACTACGCTGACAGCGTGAAGGGAAGGTTCACAATCAGCC
AAGATAACGCCAAGAACACACTGTATCTGCAGATGAACTCTCTGAAGCCAGAGG
ACACAGCCATGTACTACTGTGCCGCTGCTTGGGTCGCTTGTCTGGAGTTCGGCGG
CAGCTGGTACGATCTGGCTAGGTACAAGCACTGGGGCCAAGGCACACAAGTGAC
AGTCTCGTCTGCTAGCCACCATCACCATCACCAC
>SEQ ID NO: 87; DR441(DR236-DR231)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGAAGCG
TGCAAGCCGGCGGATCTCTGAGACTCAGCTGTACTGCCTCCGGCTTCACATTCGA
CGATAGGGAGATGAACTGGTATAGGCAAGCCCCCGGCAATGAGTGCGAGCTGGT
GAGCACAATCTCCAGCGATGGCAGCACTTACTACGCCGATAGCGTGAAGGGAAG
GTTCACTATCTCCCAAGATAACGCCAAGAACACAGTCTATCTGCAGATGGACTCC
GTCAAGCCAGAGGATACTGCCGTGTACTACTGCGCCGCCGACTTCATGATCGCCA
TCCAAGCCCCCGGCGCTGGCTGTTGGGGACAAGGCACTCAAGTGACAGTCTCGT
CTGCTAGCCACCATCACCATCACCAC
>SEQ ID NO: 88; DR442(DR236-DR232)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGCAGCG
TGCAAGCCGGAGGCTCTCTGAGACTGAGCTGTGTGGCTAGTGGCTACACAAGCT
GCATGGGCTGGTTTAGGCAAGCCCCCGGCAAGGAGAGAGAGGCCGTGGCCACAA
TCTACACTAGGGGAAGGAGCATCTACTACGCCGACAGCGTGAAAGGAAGGTTCA
CAATCAGCCAAGATAACGCCAAGAACACTCTGTATCTGCAGATGAACAGCCTCA
AGCCAGAGGACATCGCCATGTATAGCTGTGCTGCTGGCGGCTATAGCTGGAGCG
CTGGCTGCGAGTTCAATTACTGGGGCCAAGGCACACAAGTGACTGTCTCGTCTGC
TAGCCACCATCACCATCACCAC
>SEQ ID NO: 89; DR444(DR236-DR234)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGAAGCG
TGCAAGCCGGAGGCTCTCTGAGGCTGAGCTGTGTGGCTAGTGGCTACACTTTCAG
CAGCTACTGCATGGGCTGGTTCAGACAAGCCCCCGGCAAGGAAAGGGAAGGAGT
GGCCGCTCTGGGCGGAGGAAGCACATACTACGCTGACAGCGTGAAGGGAAGGTT
CACAATCAGCCAAGATAACGCCAAGAACACACTGTATCTGCAGATGAACTCTCT
GAAGCCAGAGGACACAGCCATGTACTACTGTGCCGCTGCTTGGGTCGCTTGTCTG
GAGTTCGGCGGCAGCTGGTACGATCTGGCTAGGTACAAGCACTGGGGCCAAGGC
ACACAAGTGACAGTCTCGTCTGCTAGCCACCATCACCATCACCAC
>SEQ ID NO: 90; DR449(DR237-DR233)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGCG
GAAGCGTGCAAGCTGGAGGATCTCTGAGGCTGAGCTGCACAGCCAGCGGCTTCA
CTTTCGATGACAGCGACATGGGCTGGTATAGGCAAGCCCCCGGCAATGAGTGTG
AGCTGGTGAGCACAATCAGCAGCGACGGCTCCACTTACTACGCCGACAGCGTCA
AGGGAAGGTTCACAATCAGCCAAGATAACGCCAAGAACACTGTGTATCTGCAGA
TGAACTCTCTGAAGCCAGAGGACACAGCCGTGTACTACTGTGCTGCCGAGCCTA
GGGGCTACTATAGCAACTACGGCGGAAGGAGGGAGTGCAATTACTGGGGCCAAG
GCACACAAGTGACAGTCTCGTCTGCTAGCCACCATCACCATCACCAC
>SEQ ID NO: 91; DR465(DR240-DR231)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGAAGCGTGCAAGC
CGGCGGATCTCTGAGACTCAGCTGTACTGCCTCCGGCTTCACATTCGACGATAGG
GAGATGAACTGGTATAGGCAAGCCCCCGGCAATGAGTGCGAGCTGGTGAGCACA
ATCTCCAGCGATGGCAGCACTTACTACGCCGATAGCGTGAAGGGAAGGTTCACT
ATCTCCCAAGATAACGCCAAGAACACAGTCTATCTGCAGATGGACTCCGTCAAG
CCAGAGGATACTGCCGTGTACTACTGCGCCGCCGACTTCATGATCGCCATCCAAG
CCCCCGGCGCTGGCTGTTGGGGACAAGGCACTCAAGTGACAGTCTCGTCTGCTAG
CCACCATCACCATCACCAC
>SEQ ID NO: 92; DR466(DR240-DR232)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGCAGCGTGCAAGC
CGGAGGCTCTCTGAGACTGAGCTGTGTGGCTAGTGGCTACACAAGCTGCATGGG
CTGGTTTAGGCAAGCCCCCGGCAAGGAGAGAGAGGCCGTGGCCACAATCTACAC
TAGGGGAAGGAGCATCTACTACGCCGACAGCGTGAAAGGAAGGTTCACAATCAG
CCAAGATAACGCCAAGAACACTCTGTATCTGCAGATGAACAGCCTCAAGCCAGA
GGACATCGCCATGTATAGCTGTGCTGCTGGCGGCTATAGCTGGAGCGCTGGCTGC
GAGTTCAATTACTGGGGCCAAGGCACACAAGTGACTGTCTCGTCTGCTAGCCACC
ATCACCATCACCAC
>SEQ ID NO: 93; DR468(DR240-DR234)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGAAGCGTGCAAGC
CGGAGGCTCTCTGAGGCTGAGCTGTGTGGCTAGTGGCTACACTTTCAGCAGCTAC
TGCATGGGCTGGTTCAGACAAGCCCCCGGCAAGGAAAGGGAAGGAGTGGCCGCT
CTGGGCGGAGGAAGCACATACTACGCTGACAGCGTGAAGGGAAGGTTCACAATC
AGCCAAGATAACGCCAAGAACACACTGTATCTGCAGATGAACTCTCTGAAGCCA
GAGGACACAGCCATGTACTACTGTGCCGCTGCTTGGGTCGCTTGTCTGGAGTTCG
GCGGCAGCTGGTACGATCTGGCTAGGTACAAGCACTGGGGCCAAGGCACACAAG
TGACAGTCTCGTCTGCTAGCCACCATCACCATCACCAC
>SEQ ID NO: 94; DR471(DR241-DR231)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGAAGCGTGC
AAGCCGGCGGATCTCTGAGACTCAGCTGTACTGCCTCCGGCTTCACATTCGACGA
TAGGGAGATGAACTGGTATAGGCAAGCCCCCGGCAATGAGTGCGAGCTGGTGAG
CACAATCTCCAGCGATGGCAGCACTTACTACGCCGATAGCGTGAAGGGAAGGTT
CACTATCTCCCAAGATAACGCCAAGAACACAGTCTATCTGCAGATGGACTCCGTC
AAGCCAGAGGATACTGCCGTGTACTACTGCGCCGCCGACTTCATGATCGCCATCC
AAGCCCCCGGCGCTGGCTGTTGGGGACAAGGCACTCAAGTGACAGTCTCGTCTG
CTAGCCACCATCACCATCACCAC
>SEQ ID NO: 95; DR474(DR241-DR234)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGAAGCGTGC
AAGCCGGAGGCTCTCTGAGGCTGAGCTGTGTGGCTAGTGGCTACACTTTCAGCAG
CTACTGCATGGGCTGGTTCAGACAAGCCCCCGGCAAGGAAAGGGAAGGAGTGGC
CGCTCTGGGCGGAGGAAGCACATACTACGCTGACAGCGTGAAGGGAAGGTTCAC
AATCAGCCAAGATAACGCCAAGAACACACTGTATCTGCAGATGAACTCTCTGAA
GCCAGAGGACACAGCCATGTACTACTGTGCCGCTGCTTGGGTCGCTTGTCTGGAG
TTCGGCGGCAGCTGGTACGATCTGGCTAGGTACAAGCACTGGGGCCAAGGCACA
CAAGTGACAGTCTCGTCTGCTAGCCACCATCACCATCACCAC
>SEQ ID NO: 180; DR391(DR229-DR235)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTGCAAGCCGGAGGCTCTCTGAGACTGAGCTGTGCCG
CCTCTAGGTATCTGTACAGCATCGACTACATGGCTTGGTTCAGACAGAGCCCCGG
CAAGGAGAGGGAGCCAGTGGCTGTCATCTACACTGCCTCCGGCGCCACATTCTAT
CCAGATAGCGTGAAGGGAAGGTTCACTATCAGCCAAGATAACGCCAAGATGACA
GTGTATCTGCAGATGAACTCTCTGAAGAGCGAGGACACTGCCATGTACTACTGTG
CCGCCGTGAGGAAGACAGATAGCTACCTCTTCGACGCCCAGAGCTTCACATACT
GGGGCCAAGGCACACAAGTGACAGTCTCGTCTGCTAGCCACCATCACCATCACC
AC
>SEQ ID NO: 181; DR392(DR229-DR236)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTGCAAGCCGGAGGCTCTCTGAGGCTGAGCTGTGCCG
CCTCTAGGTTCACATACAGCAGCTACTGCATGGGCTGGTTCAGACAAGCCCCCGG
CAAAGAGAGAGAAGGCGTGGCCAGCATCGATAGCGATGGCTCCACTAGCTACAC
TGACAGCGTGAAGGGAAGGTTCACTATCAGCAAGGACAACGCCAAGAACACTCT
GTATCTGCAGATGAACTCTCTGAAGCCAGAGGACACAGCCATGTACTACTGTGCC
CTCGATCTGATGAGCACAGTGGTGCCCGGCTTCTGTGGCTTTCTGCTGAGCGCTG
GCATGGATTACTGGGGCAAGGGCACTCAAGTGACTGTCTCGTCTGCTAGCCACCA
TCACCATCACCAC
>SEQ ID NO: 182; DR393(DR229-DR237)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGTCCGGAGGAGGCAGCGTCCAAGCCGGAGGCTCTCTGAGGCTGAGCTGTGCTG
CCAGCGGCTACACTTACAGCATGTACTGCATGGGCTGGTTCAGACAAGCCCCCG
GCAAGGAAAGAGAGGGCGTGGCCCAGATCAATAGCGATGGCAGCACAAGCTAC
GCCGACAGCGTGAAGGGAAGGTTCACTATCTCCAAGGACAACGCCAAGAACACT
CTGTATCTGCAGATGAACTCTCTGAAGCCAGAGGACACTGCCATGTACTACTGCG
CTGCCGATTCTAGGGTGTACGGCGGCAGCTGGTATGAGAGGCTCTGCGGCCCTTA
CACATACGAGTACAACTACTGGGGCCAAGGCACACAAGTGACTGTCTCGTCTGC
TAGCCACCATCACCATCACCAC
>SEQ ID NO: 183; DR394(DR229-DR238)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGCGGAGGAAGCGTGCAAGCCGGAGGATCTCTGAGACTGAGCTGCGCTG
TGAGCGGCTACGCCTACTCCACATACTGCATGGGCTGGTTTAGGCAAGCCCCCGG
CAAAGAGAGAGAGGGCGTGGCTGCTATCGATAGCGGCGGCAGCACAAGCTACG
CCGATAGCGTGAAGGGAAGGTTCACAATCAGCAAGGACAACGCCAAGAACACA
CTGTATCTGAGGATGAACTCTCTGAAGCCAGAGGACACAGCCATGTACTACTGTG
CTGCTGTGCCTCCTCCTCCAGATGGCGGCAGCTGTCTGTTTCTGGGACCAGAGAT
CAAGGTCAGCAAGGCCGATTTTAGGTACTGGGGCCAAGGCACACAAGTGACAGT
CTCGTCTGCTAGCCACCATCACCATCACCAC
>SEQ ID NO: 184; DR395(DR229-DR239)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTGCAAGCCGGAGGCTCTCTGAGGCTGAGCTGTACA
GTGTCCGGCTACACTTACAGCTCCAATTGCATGGGCTGGTTTAGGCAAGCCCCCG
GCAAGGAAAGAGAGGGCGTGGCCACTATCTACACTGGCGGCGGCAACACATACT
ACGCCGATAGCGTGAAGGGAAGGTTCACTATCAGCCAAGATAACGCCAAGAACA
CAGTGTATCTGCAGATGAACAATCTGAAGCCAGAGGACACTGCCATGTACTACT
GTGCTGCTGAGCCACTGTCTAGGGTGTACGGCGGCAGCTGCCCAACTCCTACATT
CGACTACTGGGGCCAAGGCACACAAGTGACTGTCTCGTCTGCTAGCCACCATCAC
CATCACCAC
>SEQ ID NO: 185; DR396(DR229-DR240)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTCCAAGCCGGAGGCTCTCTGAGGCTGAGCTGTGGA
GCCAGCGGCTACACTTACAGCAGCTACTGTATGGGCTGGTTTAGGCAAGTGCCCG
GCAAGGAGAGAGAGGGCGTGGCCGTGATCGATTCCGATGGCAGCACAAGCTACG
CTGACAGCGTGAAGGGAAGGTTCACAATCAGCAAGGACAACGGCAAGAACACA
CTCTATCTGCAGATGAACAGCCTCAAGCCAGAGGACACAGCCATGTACTACTGC
GCCGCTGATCTGGGCCACTATAGGCCTCCTTGTGGCGTGCTGTATCTGGGCATGG
ATTACTGGGGCAAGGGCACACAAGTGACAGTCTCGTCTGCTAGCCACCATCACC
ATCACCAC
>SEQ ID NO: 186; DR397(DR229-DR241)
CAAGTGCAGCTGCAAGAGAGCGGAGGAGGACTGGTCCAGCCCGGCGGCTCTCTG
AGGCTGAGCTGCACTGCTTCCGGCTTCAGCTTCAGCAGCTACCCTATGACATGGG
CTAGGCAAGCCCCCGGCAAAGGACTGGAATGGGTGAGCACTATTGCCAGCGATG
GAGGCAGCACAGCCTACGCTGCCAGCGTGGAGGGAAGGTTCACAATCTCTAGGG
ACAATGCCAAGAGCACACTGTATCTGCAGCTGAACTCTCTGAAGACAGAGGACA
CTGCCATGTACTACTGCACTAAGGGCTACGGCGATGGCACACCAGCTCCCGGCC
AAGGCACACAAGTGACTGTCTCGAGCGGCGGAGGATCCCAAGTGCAGCTGCAAG
AGAGCGGAGGAGGAAGCGTCCAAGCCGGAGGCTCTCTGAGACTGAGCTGTGCCG
CCAGCGGCTACTCCAACTGCAGCTACGACATGACTTGGTATAGGCAAGCCCCCG
GCAAGGAGAGGGAGTTCGTGTCCGCCATCCACAGCGACGGCAGCACTAGATACG
CCGACAGCGTGAAGGGAAGGTTCTTCATCAGCCAAGATAACGCCAAGAACACAG
TGTATCTGCAGATGAACTCCCTCAAGCCAGAGGACACTGCCATGTACTACTGCAA
GACAGACCCACTGCACTGCAGAGCCCATGGCGGCAGCTGGTATAGCGTGAGGGC
CAACTACTGGGGCCAAGGCACACAAGTGACAGTCTCGTCTGCTAGCCACCATCA
CCATCACCAC
>SEQ ID NO: 187; DR398(DR230-DR235)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGAGGAGGAAGCGTGCAAGCCGGAGGCTCTCTGAGACTG
AGCTGTGCCGCCTCTAGGTATCTGTACAGCATCGACTACATGGCTTGGTTCAGAC
AGAGCCCCGGCAAGGAGAGGGAGCCAGTGGCTGTCATCTACACTGCCTCCGGCG
CCACATTCTATCCAGATAGCGTGAAGGGAAGGTTCACTATCAGCCAAGATAACG
CCAAGATGACAGTGTATCTGCAGATGAACTCTCTGAAGAGCGAGGACACTGCCA
TGTACTACTGTGCCGCCGTGAGGAAGACAGATAGCTACCTCTTCGACGCCCAGA
GCTTCACATACTGGGGCCAAGGCACACAAGTGACAGTCTCGTCTGCTAGCCACC
ATCACCATCACCAC
>SEQ ID NO: 188; DR399(DR230-DR236)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGAGGAGGAAGCGTGCAAGCCGGAGGCTCTCTGAGGCTG
AGCTGTGCCGCCTCTAGGTTCACATACAGCAGCTACTGCATGGGCTGGTTCAGAC
AAGCCCCCGGCAAAGAGAGAGAAGGCGTGGCCAGCATCGATAGCGATGGCTCC
ACTAGCTACACTGACAGCGTGAAGGGAAGGTTCACTATCAGCAAGGACAACGCC
AAGAACACTCTGTATCTGCAGATGAACTCTCTGAAGCCAGAGGACACAGCCATG
TACTACTGTGCCCTCGATCTGATGAGCACAGTGGTGCCCGGCTTCTGTGGCTTTCT
GCTGAGCGCTGGCATGGATTACTGGGGCAAGGGCACTCAAGTGACTGTCTCGTCT
GCTAGCCACCATCACCATCACCAC
>SEQ ID NO: 189; DR400(DR230-DR237)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGTCCGGAGGAGGCAGCGTCCAAGCCGGAGGCTCTCTGAGGCTGA
GCTGTGCTGCCAGCGGCTACACTTACAGCATGTACTGCATGGGCTGGTTCAGACA
AGCCCCCGGCAAGGAAAGAGAGGGCGTGGCCCAGATCAATAGCGATGGCAGCA
CAAGCTACGCCGACAGCGTGAAGGGAAGGTTCACTATCTCCAAGGACAACGCCA
AGAACACTCTGTATCTGCAGATGAACTCTCTGAAGCCAGAGGACACTGCCATGT
ACTACTGCGCTGCCGATTCTAGGGTGTACGGCGGCAGCTGGTATGAGAGGCTCTG
CGGCCCTTACACATACGAGTACAACTACTGGGGCCAAGGCACACAAGTGACTGT
CTCGTCTGCTAGCCACCATCACCATCACCAC
>SEQ ID NO: 190; DR401(DR230-DR238)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGCGGAGGAAGCGTGCAAGCCGGAGGATCTCTGAGACTG
AGCTGCGCTGTGAGCGGCTACGCCTACTCCACATACTGCATGGGCTGGTTTAGGC
AAGCCCCCGGCAAAGAGAGAGAGGGCGTGGCTGCTATCGATAGCGGCGGCAGC
ACAAGCTACGCCGATAGCGTGAAGGGAAGGTTCACAATCAGCAAGGACAACGCC
AAGAACACACTGTATCTGAGGATGAACTCTCTGAAGCCAGAGGACACAGCCATG
TACTACTGTGCTGCTGTGCCTCCTCCTCCAGATGGCGGCAGCTGTCTGTTTCTGGG
ACCAGAGATCAAGGTCAGCAAGGCCGATTTTAGGTACTGGGGCCAAGGCACACA
AGTGACAGTCTCGTCTGCTAGCCACCATCACCATCACCAC
>SEQ ID NO: 191; DR402(DR230-DR239)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGAGGAGGAAGCGTGCAAGCCGGAGGCTCTCTGAGGCTG
AGCTGTACAGTGTCCGGCTACACTTACAGCTCCAATTGCATGGGCTGGTTTAGGC
AAGCCCCCGGCAAGGAAAGAGAGGGCGTGGCCACTATCTACACTGGCGGCGGCA
ACACATACTACGCCGATAGCGTGAAGGGAAGGTTCACTATCAGCCAAGATAACG
CCAAGAACACAGTGTATCTGCAGATGAACAATCTGAAGCCAGAGGACACTGCCA
TGTACTACTGTGCTGCTGAGCCACTGTCTAGGGTGTACGGCGGCAGCTGCCCAAC
TCCTACATTCGACTACTGGGGCCAAGGCACACAAGTGACTGTCTCGTCTGCTAGC
CACCATCACCATCACCAC
>SEQ ID NO: 192; DR403(DR230-DR240)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGAGGAGGAAGCGTCCAAGCCGGAGGCTCTCTGAGGCTG
AGCTGTGGAGCCAGCGGCTACACTTACAGCAGCTACTGTATGGGCTGGTTTAGGC
AAGTGCCCGGCAAGGAGAGAGAGGGCGTGGCCGTGATCGATTCCGATGGCAGCA
CAAGCTACGCTGACAGCGTGAAGGGAAGGTTCACAATCAGCAAGGACAACGGC
AAGAACACACTCTATCTGCAGATGAACAGCCTCAAGCCAGAGGACACAGCCATG
TACTACTGCGCCGCTGATCTGGGCCACTATAGGCCTCCTTGTGGCGTGCTGTATC
TGGGCATGGATTACTGGGGCAAGGGCACACAAGTGACAGTCTCGTCTGCTAGCC
ACCATCACCATCACCAC
>SEQ ID NO: 193; DR404(DR230-DR241)
CAAGTGCAGCTGCAAGAGAGCGGAGGAGGACTGGTGCAGCCCGGCGGCTCTCTG
AGGCTGAGCTGTGCTGCCAGCGGCTTCACTTTCAGCAGCGCTCACATGAGCTGGG
TGAGGCAAGCCCCCGGCAAAGGAAGGGAGTGGATCGCCTCCATCTACAGCGGCG
GCGGAACATTCTACGCCGACAGCGTGAAGGGAAGGTTCACAATCTCTAGGGACA
ACGCCAAGAACACACTGTATCTGCAGCTGAACTCTCTGAAGGCCGAGGACACTG
CCATGTACTACTGCGCCACTAATAGGCTGCACTACTACAGCGACGATGATTCTCT
GAGGGGCCAAGGCACACAAGTGACAGTCTCGAGCGGCGGAGGATCCCAAGTGC
AGCTGCAAGAGAGCGGAGGAGGAAGCGTCCAAGCCGGAGGCTCTCTGAGACTG
AGCTGTGCCGCCAGCGGCTACTCCAACTGCAGCTACGACATGACTTGGTATAGGC
AAGCCCCCGGCAAGGAGAGGGAGTTCGTGTCCGCCATCCACAGCGACGGCAGCA
CTAGATACGCCGACAGCGTGAAGGGAAGGTTCTTCATCAGCCAAGATAACGCCA
AGAACACAGTGTATCTGCAGATGAACTCCCTCAAGCCAGAGGACACTGCCATGT
ACTACTGCAAGACAGACCCACTGCACTGCAGAGCCCATGGCGGCAGCTGGTATA
GCGTGAGGGCCAACTACTGGGGCCAAGGCACACAAGTGACAGTCTCGTCTGCTA
GCCACCATCACCATCACCAC
>SEQ ID NO: 194; DR405(DR231-DR235)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGAGGAGGAAGCGTGCAAGCCGGAGGCTCTCTGAG
ACTGAGCTGTGCCGCCTCTAGGTATCTGTACAGCATCGACTACATGGCTTGGTTC
AGACAGAGCCCCGGCAAGGAGAGGGAGCCAGTGGCTGTCATCTACACTGCCTCC
GGCGCCACATTCTATCCAGATAGCGTGAAGGGAAGGTTCACTATCAGCCAAGAT
AACGCCAAGATGACAGTGTATCTGCAGATGAACTCTCTGAAGAGCGAGGACACT
GCCATGTACTACTGTGCCGCCGTGAGGAAGACAGATAGCTACCTCTTCGACGCCC
AGAGCTTCACATACTGGGGCCAAGGCACACAAGTGACAGTCTCGTCTGCTAGCC
ACCATCACCATCACCAC
>SEQ ID NO: 195; DR406(DR231-DR236)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGAGGAGGAAGCGTGCAAGCCGGAGGCTCTCTGAG
GCTGAGCTGTGCCGCCTCTAGGTTCACATACAGCAGCTACTGCATGGGCTGGTTC
AGACAAGCCCCCGGCAAAGAGAGAGAAGGCGTGGCCAGCATCGATAGCGATGG
CTCCACTAGCTACACTGACAGCGTGAAGGGAAGGTTCACTATCAGCAAGGACAA
CGCCAAGAACACTCTGTATCTGCAGATGAACTCTCTGAAGCCAGAGGACACAGC
CATGTACTACTGTGCCCTCGATCTGATGAGCACAGTGGTGCCCGGCTTCTGTGGC
TTTCTGCTGAGCGCTGGCATGGATTACTGGGGCAAGGGCACTCAAGTGACTGTCT
CGTCTGCTAGCCACCATCACCATCACCAC
>SEQ ID NO: 196; DR407(DR231-DR237)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGTCCGGAGGAGGCAGCGTCCAAGCCGGAGGCTCTCTGAGG
CTGAGCTGTGCTGCCAGCGGCTACACTTACAGCATGTACTGCATGGGCTGGTTCA
GACAAGCCCCCGGCAAGGAAAGAGAGGGCGTGGCCCAGATCAATAGCGATGGC
AGCACAAGCTACGCCGACAGCGTGAAGGGAAGGTTCACTATCTCCAAGGACAAC
GCCAAGAACACTCTGTATCTGCAGATGAACTCTCTGAAGCCAGAGGACACTGCC
ATGTACTACTGCGCTGCCGATTCTAGGGTGTACGGCGGCAGCTGGTATGAGAGG
CTCTGCGGCCCTTACACATACGAGTACAACTACTGGGGCCAAGGCACACAAGTG
ACTGTCTCGTCTGCTAGCCACCATCACCATCACCAC
>SEQ ID NO: 197; DR408(DR231-DR238)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGCGGAGGAAGCGTGCAAGCCGGAGGATCTCTGAG
ACTGAGCTGCGCTGTGAGCGGCTACGCCTACTCCACATACTGCATGGGCTGGTTT
AGGCAAGCCCCCGGCAAAGAGAGAGAGGGCGTGGCTGCTATCGATAGCGGCGG
CAGCACAAGCTACGCCGATAGCGTGAAGGGAAGGTTCACAATCAGCAAGGACA
ACGCCAAGAACACACTGTATCTGAGGATGAACTCTCTGAAGCCAGAGGACACAG
CCATGTACTACTGTGCTGCTGTGCCTCCTCCTCCAGATGGCGGCAGCTGTCTGTTT
CTGGGACCAGAGATCAAGGTCAGCAAGGCCGATTTTAGGTACTGGGGCCAAGGC
ACACAAGTGACAGTCTCGTCTGCTAGCCACCATCACCATCACCAC
>SEQ ID NO: 198; DR409(DR231-DR239)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGAGGAGGAAGCGTGCAAGCCGGAGGCTCTCTGAG
GCTGAGCTGTACAGTGTCCGGCTACACTTACAGCTCCAATTGCATGGGCTGGTTT
AGGCAAGCCCCCGGCAAGGAAAGAGAGGGCGTGGCCACTATCTACACTGGCGGC
GGCAACACATACTACGCCGATAGCGTGAAGGGAAGGTTCACTATCAGCCAAGAT
AACGCCAAGAACACAGTGTATCTGCAGATGAACAATCTGAAGCCAGAGGACACT
GCCATGTACTACTGTGCTGCTGAGCCACTGTCTAGGGTGTACGGCGGCAGCTGCC
CAACTCCTACATTCGACTACTGGGGCCAAGGCACACAAGTGACTGTCTCGTCTGC
TAGCCACCATCACCATCACCAC
>SEQ ID NO: 199; DR410(DR231-DR240)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGAGGAGGAAGCGTCCAAGCCGGAGGCTCTCTGAG
GCTGAGCTGTGGAGCCAGCGGCTACACTTACAGCAGCTACTGTATGGGCTGGTTT
AGGCAAGTGCCCGGCAAGGAGAGAGAGGGCGTGGCCGTGATCGATTCCGATGGC
AGCACAAGCTACGCTGACAGCGTGAAGGGAAGGTTCACAATCAGCAAGGACAA
CGGCAAGAACACACTCTATCTGCAGATGAACAGCCTCAAGCCAGAGGACACAGC
CATGTACTACTGCGCCGCTGATCTGGGCCACTATAGGCCTCCTTGTGGCGTGCTG
TATCTGGGCATGGATTACTGGGGCAAGGGCACACAAGTGACAGTCTCGTCTGCT
AGCCACCATCACCATCACCAC
>SEQ ID NO: 200; DR411(DR231-DR241)
CAAGTGCAGCTGCAAGAGAGCGGAGGAGGAAGCGTGCAAGCCGGCGGATCTCT
GAGACTCAGCTGTACTGCCTCCGGCTTCACATTCGACGATAGGGAGATGAACTG
GTATAGGCAAGCCCCCGGCAATGAGTGCGAGCTGGTGAGCACAATCTCCAGCGA
TGGCAGCACTTACTACGCCGATAGCGTGAAGGGAAGGTTCACTATCTCCCAAGA
TAACGCCAAGAACACAGTCTATCTGCAGATGGACTCCGTCAAGCCAGAGGATAC
TGCCGTGTACTACTGCGCCGCCGACTTCATGATCGCCATCCAAGCCCCCGGCGCT
GGCTGTTGGGGACAAGGCACTCAAGTGACAGTCTCGAGCGGCGGAGGATCCCAA
GTGCAGCTGCAAGAGAGCGGAGGAGGAAGCGTCCAAGCCGGAGGCTCTCTGAG
ACTGAGCTGTGCCGCCAGCGGCTACTCCAACTGCAGCTACGACATGACTTGGTAT
AGGCAAGCCCCCGGCAAGGAGAGGGAGTTCGTGTCCGCCATCCACAGCGACGGC
AGCACTAGATACGCCGACAGCGTGAAGGGAAGGTTCTTCATCAGCCAAGATAAC
GCCAAGAACACAGTGTATCTGCAGATGAACTCCCTCAAGCCAGAGGACACTGCC
ATGTACTACTGCAAGACAGACCCACTGCACTGCAGAGCCCATGGCGGCAGCTGG
TATAGCGTGAGGGCCAACTACTGGGGCCAAGGCACACAAGTGACAGTCTCGTCT
GCTAGCCACCATCACCATCACCAC
>SEQ ID NO: 201; DR412(DR232-DR235)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGAGGAGGAAGCGTGCAAGCCGGAGGCTCTCTGAGACTGA
GCTGTGCCGCCTCTAGGTATCTGTACAGCATCGACTACATGGCTTGGTTCAGACA
GAGCCCCGGCAAGGAGAGGGAGCCAGTGGCTGTCATCTACACTGCCTCCGGCGC
CACATTCTATCCAGATAGCGTGAAGGGAAGGTTCACTATCAGCCAAGATAACGC
CAAGATGACAGTGTATCTGCAGATGAACTCTCTGAAGAGCGAGGACACTGCCAT
GTACTACTGTGCCGCCGTGAGGAAGACAGATAGCTACCTCTTCGACGCCCAGAG
CTTCACATACTGGGGCCAAGGCACACAAGTGACAGTCTCGTCTGCTAGCCACCAT
CACCATCACCAC
>SEQ ID NO: 202; DR413(DR232-DR236)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGAGGAGGAAGCGTGCAAGCCGGAGGCTCTCTGAGGCTGA
GCTGTGCCGCCTCTAGGTTCACATACAGCAGCTACTGCATGGGCTGGTTCAGACA
AGCCCCCGGCAAAGAGAGAGAAGGCGTGGCCAGCATCGATAGCGATGGCTCCAC
TAGCTACACTGACAGCGTGAAGGGAAGGTTCACTATCAGCAAGGACAACGCCAA
GAACACTCTGTATCTGCAGATGAACTCTCTGAAGCCAGAGGACACAGCCATGTA
CTACTGTGCCCTCGATCTGATGAGCACAGTGGTGCCCGGCTTCTGTGGCTTTCTG
CTGAGCGCTGGCATGGATTACTGGGGCAAGGGCACTCAAGTGACTGTCTCGTCTG
CTAGCCACCATCACCATCACCAC
>SEQ ID NO: 203; DR414(DR232-DR237)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGTCCGGAGGAGGCAGCGTCCAAGCCGGAGGCTCTCTGAGGCTGAG
CTGTGCTGCCAGCGGCTACACTTACAGCATGTACTGCATGGGCTGGTTCAGACAA
GCCCCCGGCAAGGAAAGAGAGGGCGTGGCCCAGATCAATAGCGATGGCAGCAC
AAGCTACGCCGACAGCGTGAAGGGAAGGTTCACTATCTCCAAGGACAACGCCAA
GAACACTCTGTATCTGCAGATGAACTCTCTGAAGCCAGAGGACACTGCCATGTAC
TACTGCGCTGCCGATTCTAGGGTGTACGGCGGCAGCTGGTATGAGAGGCTCTGCG
GCCCTTACACATACGAGTACAACTACTGGGGCCAAGGCACACAAGTGACTGTCT
CGTCTGCTAGCCACCATCACCATCACCAC
>SEQ ID NO: 204; DR415(DR232-DR238)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGCGGAGGAAGCGTGCAAGCCGGAGGATCTCTGAGACTGA
GCTGCGCTGTGAGCGGCTACGCCTACTCCACATACTGCATGGGCTGGTTTAGGCA
AGCCCCCGGCAAAGAGAGAGAGGGCGTGGCTGCTATCGATAGCGGCGGCAGCA
CAAGCTACGCCGATAGCGTGAAGGGAAGGTTCACAATCAGCAAGGACAACGCCA
AGAACACACTGTATCTGAGGATGAACTCTCTGAAGCCAGAGGACACAGCCATGT
ACTACTGTGCTGCTGTGCCTCCTCCTCCAGATGGCGGCAGCTGTCTGTTTCTGGG
ACCAGAGATCAAGGTCAGCAAGGCCGATTTTAGGTACTGGGGCCAAGGCACACA
AGTGACAGTCTCGTCTGCTAGCCACCATCACCATCACCAC
>SEQ ID NO: 205; DR416(DR232-DR239)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGAGGAGGAAGCGTGCAAGCCGGAGGCTCTCTGAGGCTGA
GCTGTACAGTGTCCGGCTACACTTACAGCTCCAATTGCATGGGCTGGTTTAGGCA
AGCCCCCGGCAAGGAAAGAGAGGGCGTGGCCACTATCTACACTGGCGGCGGCAA
CACATACTACGCCGATAGCGTGAAGGGAAGGTTCACTATCAGCCAAGATAACGC
CAAGAACACAGTGTATCTGCAGATGAACAATCTGAAGCCAGAGGACACTGCCAT
GTACTACTGTGCTGCTGAGCCACTGTCTAGGGTGTACGGCGGCAGCTGCCCAACT
CCTACATTCGACTACTGGGGCCAAGGCACACAAGTGACTGTCTCGTCTGCTAGCC
ACCATCACCATCACCAC
>SEQ ID NO: 206; DR417(DR232-DR240)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGAGGAGGAAGCGTCCAAGCCGGAGGCTCTCTGAGGCTGA
GCTGTGGAGCCAGCGGCTACACTTACAGCAGCTACTGTATGGGCTGGTTTAGGCA
AGTGCCCGGCAAGGAGAGAGAGGGCGTGGCCGTGATCGATTCCGATGGCAGCAC
AAGCTACGCTGACAGCGTGAAGGGAAGGTTCACAATCAGCAAGGACAACGGCA
AGAACACACTCTATCTGCAGATGAACAGCCTCAAGCCAGAGGACACAGCCATGT
ACTACTGCGCCGCTGATCTGGGCCACTATAGGCCTCCTTGTGGCGTGCTGTATCT
GGGCATGGATTACTGGGGCAAGGGCACACAAGTGACAGTCTCGTCTGCTAGCCA
CCATCACCATCACCAC
>SEQ ID NO: 207; DR418(DR232-DR241)
CAAGTGCAGCTGCAAGAGAGCGGAGGAGGCAGCGTGCAAGCCGGAGGCTCTCT
GAGACTGAGCTGTGTGGCTAGTGGCTACACAAGCTGCATGGGCTGGTTTAGGCA
AGCCCCCGGCAAGGAGAGAGAGGCCGTGGCCACAATCTACACTAGGGGAAGGA
GCATCTACTACGCCGACAGCGTGAAAGGAAGGTTCACAATCAGCCAAGATAACG
CCAAGAACACTCTGTATCTGCAGATGAACAGCCTCAAGCCAGAGGACATCGCCA
TGTATAGCTGTGCTGCTGGCGGCTATAGCTGGAGCGCTGGCTGCGAGTTCAATTA
CTGGGGCCAAGGCACACAAGTGACTGTCTCGAGCGGCGGAGGATCCCAAGTGCA
GCTGCAAGAGAGCGGAGGAGGAAGCGTCCAAGCCGGAGGCTCTCTGAGACTGA
GCTGTGCCGCCAGCGGCTACTCCAACTGCAGCTACGACATGACTTGGTATAGGCA
AGCCCCCGGCAAGGAGAGGGAGTTCGTGTCCGCCATCCACAGCGACGGCAGCAC
TAGATACGCCGACAGCGTGAAGGGAAGGTTCTTCATCAGCCAAGATAACGCCAA
GAACACAGTGTATCTGCAGATGAACTCCCTCAAGCCAGAGGACACTGCCATGTA
CTACTGCAAGACAGACCCACTGCACTGCAGAGCCCATGGCGGCAGCTGGTATAG
CGTGAGGGCCAACTACTGGGGCCAAGGCACACAAGTGACAGTCTCGTCTGCTAG
CCACCATCACCATCACCAC
>SEQ ID NO: 208; DR419(DR233-DR235)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGAGGAGGAAGCGTGCAAGCC
GGAGGCTCTCTGAGACTGAGCTGTGCCGCCTCTAGGTATCTGTACAGCATCGACT
ACATGGCTTGGTTCAGACAGAGCCCCGGCAAGGAGAGGGAGCCAGTGGCTGTCA
TCTACACTGCCTCCGGCGCCACATTCTATCCAGATAGCGTGAAGGGAAGGTTCAC
TATCAGCCAAGATAACGCCAAGATGACAGTGTATCTGCAGATGAACTCTCTGAA
GAGCGAGGACACTGCCATGTACTACTGTGCCGCCGTGAGGAAGACAGATAGCTA
CCTCTTCGACGCCCAGAGCTTCACATACTGGGGCCAAGGCACACAAGTGACAGT
CTCGTCTGCTAGCCACCATCACCATCACCAC
>SEQ ID NO: 209; DR420(DR233-DR236)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGAGGAGGAAGCGTGCAAGCC
GGAGGCTCTCTGAGGCTGAGCTGTGCCGCCTCTAGGTTCACATACAGCAGCTACT
GCATGGGCTGGTTCAGACAAGCCCCCGGCAAAGAGAGAGAAGGCGTGGCCAGC
ATCGATAGCGATGGCTCCACTAGCTACACTGACAGCGTGAAGGGAAGGTTCACT
ATCAGCAAGGACAACGCCAAGAACACTCTGTATCTGCAGATGAACTCTCTGAAG
CCAGAGGACACAGCCATGTACTACTGTGCCCTCGATCTGATGAGCACAGTGGTG
CCCGGCTTCTGTGGCTTTCTGCTGAGCGCTGGCATGGATTACTGGGGCAAGGGCA
CTCAAGTGACTGTCTCGTCTGCTAGCCACCATCACCATCACCAC
>SEQ ID NO: 210; DR421(DR233-DR237)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGTCCGGAGGAGGCAGCGTCCAAGCC
GGAGGCTCTCTGAGGCTGAGCTGTGCTGCCAGCGGCTACACTTACAGCATGTACT
GCATGGGCTGGTTCAGACAAGCCCCCGGCAAGGAAAGAGAGGGCGTGGCCCAG
ATCAATAGCGATGGCAGCACAAGCTACGCCGACAGCGTGAAGGGAAGGTTCACT
ATCTCCAAGGACAACGCCAAGAACACTCTGTATCTGCAGATGAACTCTCTGAAG
CCAGAGGACACTGCCATGTACTACTGCGCTGCCGATTCTAGGGTGTACGGCGGC
AGCTGGTATGAGAGGCTCTGCGGCCCTTACACATACGAGTACAACTACTGGGGC
CAAGGCACACAAGTGACTGTCTCGTCTGCTAGCCACCATCACCATCACCAC
>SEQ ID NO: 211; DR422(DR233-DR238)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGCGGAGGAAGCGTGCAAGCC
GGAGGATCTCTGAGACTGAGCTGCGCTGTGAGCGGCTACGCCTACTCCACATACT
GCATGGGCTGGTTTAGGCAAGCCCCCGGCAAAGAGAGAGAGGGCGTGGCTGCTA
TCGATAGCGGCGGCAGCACAAGCTACGCCGATAGCGTGAAGGGAAGGTTCACAA
TCAGCAAGGACAACGCCAAGAACACACTGTATCTGAGGATGAACTCTCTGAAGC
CAGAGGACACAGCCATGTACTACTGTGCTGCTGTGCCTCCTCCTCCAGATGGCGG
CAGCTGTCTGTTTCTGGGACCAGAGATCAAGGTCAGCAAGGCCGATTTTAGGTAC
TGGGGCCAAGGCACACAAGTGACAGTCTCGTCTGCTAGCCACCATCACCATCAC
CAC
>SEQ ID NO: 212; DR423(DR233-DR239)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGAGGAGGAAGCGTGCAAGCC
GGAGGCTCTCTGAGGCTGAGCTGTACAGTGTCCGGCTACACTTACAGCTCCAATT
GCATGGGCTGGTTTAGGCAAGCCCCCGGCAAGGAAAGAGAGGGCGTGGCCACTA
TCTACACTGGCGGCGGCAACACATACTACGCCGATAGCGTGAAGGGAAGGTTCA
CTATCAGCCAAGATAACGCCAAGAACACAGTGTATCTGCAGATGAACAATCTGA
AGCCAGAGGACACTGCCATGTACTACTGTGCTGCTGAGCCACTGTCTAGGGTGTA
CGGCGGCAGCTGCCCAACTCCTACATTCGACTACTGGGGCCAAGGCACACAAGT
GACTGTCTCGTCTGCTAGCCACCATCACCATCACCAC
>SEQ ID NO: 213; DR424(DR233-DR240)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGAGGAGGAAGCGTCCAAGCC
GGAGGCTCTCTGAGGCTGAGCTGTGGAGCCAGCGGCTACACTTACAGCAGCTAC
TGTATGGGCTGGTTTAGGCAAGTGCCCGGCAAGGAGAGAGAGGGCGTGGCCGTG
ATCGATTCCGATGGCAGCACAAGCTACGCTGACAGCGTGAAGGGAAGGTTCACA
ATCAGCAAGGACAACGGCAAGAACACACTCTATCTGCAGATGAACAGCCTCAAG
CCAGAGGACACAGCCATGTACTACTGCGCCGCTGATCTGGGCCACTATAGGCCTC
CTTGTGGCGTGCTGTATCTGGGCATGGATTACTGGGGCAAGGGCACACAAGTGA
CAGTCTCGTCTGCTAGCCACCATCACCATCACCAC
>SEQ ID NO: 214; DR425(DR233-DR241)
CAAGTGCAGCTGCAAGAGAGCGGAGGCGGAAGCGTGCAAGCTGGAGGATCTCT
GAGGCTGAGCTGCACAGCCAGCGGCTTCACTTTCGATGACAGCGACATGGGCTG
GTATAGGCAAGCCCCCGGCAATGAGTGTGAGCTGGTGAGCACAATCAGCAGCGA
CGGCTCCACTTACTACGCCGACAGCGTCAAGGGAAGGTTCACAATCAGCCAAGA
TAACGCCAAGAACACTGTGTATCTGCAGATGAACTCTCTGAAGCCAGAGGACAC
AGCCGTGTACTACTGTGCTGCCGAGCCTAGGGGCTACTATAGCAACTACGGCGG
AAGGAGGGAGTGCAATTACTGGGGCCAAGGCACACAAGTGACAGTCTCGAGCG
GCGGAGGATCCCAAGTGCAGCTGCAAGAGAGCGGAGGAGGAAGCGTCCAAGCC
GGAGGCTCTCTGAGACTGAGCTGTGCCGCCAGCGGCTACTCCAACTGCAGCTAC
GACATGACTTGGTATAGGCAAGCCCCCGGCAAGGAGAGGGAGTTCGTGTCCGCC
ATCCACAGCGACGGCAGCACTAGATACGCCGACAGCGTGAAGGGAAGGTTCTTC
ATCAGCCAAGATAACGCCAAGAACACAGTGTATCTGCAGATGAACTCCCTCAAG
CCAGAGGACACTGCCATGTACTACTGCAAGACAGACCCACTGCACTGCAGAGCC
CATGGCGGCAGCTGGTATAGCGTGAGGGCCAACTACTGGGGCCAAGGCACACAA
GTGACAGTCTCGTCTGCTAGCCACCATCACCATCACCAC
>SEQ ID NO: 215; DR426(DR234-DR235)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGAGGAGGAAGCGTGCA
AGCCGGAGGCTCTCTGAGACTGAGCTGTGCCGCCTCTAGGTATCTGTACAGCATC
GACTACATGGCTTGGTTCAGACAGAGCCCCGGCAAGGAGAGGGAGCCAGTGGCT
GTCATCTACACTGCCTCCGGCGCCACATTCTATCCAGATAGCGTGAAGGGAAGGT
TCACTATCAGCCAAGATAACGCCAAGATGACAGTGTATCTGCAGATGAACTCTCT
GAAGAGCGAGGACACTGCCATGTACTACTGTGCCGCCGTGAGGAAGACAGATAG
CTACCTCTTCGACGCCCAGAGCTTCACATACTGGGGCCAAGGCACACAAGTGAC
AGTCTCGTCTGCTAGCCACCATCACCATCACCAC
>SEQ ID NO: 216; DR427(DR234-DR236)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGAGGAGGAAGCGTGCA
AGCCGGAGGCTCTCTGAGGCTGAGCTGTGCCGCCTCTAGGTTCACATACAGCAGC
TACTGCATGGGCTGGTTCAGACAAGCCCCCGGCAAAGAGAGAGAAGGCGTGGCC
AGCATCGATAGCGATGGCTCCACTAGCTACACTGACAGCGTGAAGGGAAGGTTC
ACTATCAGCAAGGACAACGCCAAGAACACTCTGTATCTGCAGATGAACTCTCTG
AAGCCAGAGGACACAGCCATGTACTACTGTGCCCTCGATCTGATGAGCACAGTG
GTGCCCGGCTTCTGTGGCTTTCTGCTGAGCGCTGGCATGGATTACTGGGGCAAGG
GCACTCAAGTGACTGTCTCGTCTGCTAGCCACCATCACCATCACCAC
>SEQ ID NO: 217; DR428(DR234-DR237)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGTCCGGAGGAGGCAGCGTCCA
AGCCGGAGGCTCTCTGAGGCTGAGCTGTGCTGCCAGCGGCTACACTTACAGCAT
GTACTGCATGGGCTGGTTCAGACAAGCCCCCGGCAAGGAAAGAGAGGGCGTGGC
CCAGATCAATAGCGATGGCAGCACAAGCTACGCCGACAGCGTGAAGGGAAGGTT
CACTATCTCCAAGGACAACGCCAAGAACACTCTGTATCTGCAGATGAACTCTCTG
AAGCCAGAGGACACTGCCATGTACTACTGCGCTGCCGATTCTAGGGTGTACGGC
GGCAGCTGGTATGAGAGGCTCTGCGGCCCTTACACATACGAGTACAACTACTGG
GGCCAAGGCACACAAGTGACTGTCTCGTCTGCTAGCCACCATCACCATCACCAC
>SEQ ID NO: 218; DR429(DR234-DR238)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGCGGAGGAAGCGTGCA
AGCCGGAGGATCTCTGAGACTGAGCTGCGCTGTGAGCGGCTACGCCTACTCCAC
ATACTGCATGGGCTGGTTTAGGCAAGCCCCCGGCAAAGAGAGAGAGGGCGTGGC
TGCTATCGATAGCGGCGGCAGCACAAGCTACGCCGATAGCGTGAAGGGAAGGTT
CACAATCAGCAAGGACAACGCCAAGAACACACTGTATCTGAGGATGAACTCTCT
GAAGCCAGAGGACACAGCCATGTACTACTGTGCTGCTGTGCCTCCTCCTCCAGAT
GGCGGCAGCTGTCTGTTTCTGGGACCAGAGATCAAGGTCAGCAAGGCCGATTTT
AGGTACTGGGGCCAAGGCACACAAGTGACAGTCTCGTCTGCTAGCCACCATCAC
CATCACCAC
>SEQ ID NO: 219; DR430(DR234-DR239)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGAGGAGGAAGCGTGCA
AGCCGGAGGCTCTCTGAGGCTGAGCTGTACAGTGTCCGGCTACACTTACAGCTCC
AATTGCATGGGCTGGTTTAGGCAAGCCCCCGGCAAGGAAAGAGAGGGCGTGGCC
ACTATCTACACTGGCGGCGGCAACACATACTACGCCGATAGCGTGAAGGGAAGG
TTCACTATCAGCCAAGATAACGCCAAGAACACAGTGTATCTGCAGATGAACAAT
CTGAAGCCAGAGGACACTGCCATGTACTACTGTGCTGCTGAGCCACTGTCTAGGG
TGTACGGCGGCAGCTGCCCAACTCCTACATTCGACTACTGGGGCCAAGGCACAC
AAGTGACTGTCTCGTCTGCTAGCCACCATCACCATCACCAC
>SEQ ID NO: 220; DR431(DR234-DR240)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGAGGAGGAAGCGTCCA
AGCCGGAGGCTCTCTGAGGCTGAGCTGTGGAGCCAGCGGCTACACTTACAGCAG
CTACTGTATGGGCTGGTTTAGGCAAGTGCCCGGCAAGGAGAGAGAGGGCGTGGC
CGTGATCGATTCCGATGGCAGCACAAGCTACGCTGACAGCGTGAAGGGAAGGTT
CACAATCAGCAAGGACAACGGCAAGAACACACTCTATCTGCAGATGAACAGCCT
CAAGCCAGAGGACACAGCCATGTACTACTGCGCCGCTGATCTGGGCCACTATAG
GCCTCCTTGTGGCGTGCTGTATCTGGGCATGGATTACTGGGGCAAGGGCACACAA
GTGACAGTCTCGTCTGCTAGCCACCATCACCATCACCAC
>SEQ ID NO: 221; DR432(DR234-DR241)
CAAGTGCAGCTGCAAGAGAGCGGAGGAGGAAGCGTGCAAGCCGGAGGCTCTCT
GAGGCTGAGCTGTGTGGCTAGTGGCTACACTTTCAGCAGCTACTGCATGGGCTGG
TTCAGACAAGCCCCCGGCAAGGAAAGGGAAGGAGTGGCCGCTCTGGGCGGAGG
AAGCACATACTACGCTGACAGCGTGAAGGGAAGGTTCACAATCAGCCAAGATAA
CGCCAAGAACACACTGTATCTGCAGATGAACTCTCTGAAGCCAGAGGACACAGC
CATGTACTACTGTGCCGCTGCTTGGGTCGCTTGTCTGGAGTTCGGCGGCAGCTGG
TACGATCTGGCTAGGTACAAGCACTGGGGCCAAGGCACACAAGTGACAGTCTCG
AGCGGCGGAGGATCCCAAGTGCAGCTGCAAGAGAGCGGAGGAGGAAGCGTCCA
AGCCGGAGGCTCTCTGAGACTGAGCTGTGCCGCCAGCGGCTACTCCAACTGCAG
CTACGACATGACTTGGTATAGGCAAGCCCCCGGCAAGGAGAGGGAGTTCGTGTC
CGCCATCCACAGCGACGGCAGCACTAGATACGCCGACAGCGTGAAGGGAAGGTT
CTTCATCAGCCAAGATAACGCCAAGAACACAGTGTATCTGCAGATGAACTCCCTC
AAGCCAGAGGACACTGCCATGTACTACTGCAAGACAGACCCACTGCACTGCAGA
GCCCATGGCGGCAGCTGGTATAGCGTGAGGGCCAACTACTGGGGCCAAGGCACA
CAAGTGACAGTCTCGTCTGCTAGCCACCATCACCATCACCAC
>SEQ ID NO: 222; DR433(DR235-DR229)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGACTGGTCCAGCCCGGC
GGCTCTCTGAGGCTGAGCTGCACTGCTTCCGGCTTCAGCTTCAGCAGCTACCCTA
TGACATGGGCTAGGCAAGCCCCCGGCAAAGGACTGGAATGGGTGAGCACTATTG
CCAGCGATGGAGGCAGCACAGCCTACGCTGCCAGCGTGGAGGGAAGGTTCACAA
TCTCTAGGGACAATGCCAAGAGCACACTGTATCTGCAGCTGAACTCTCTGAAGAC
AGAGGACACTGCCATGTACTACTGCACTAAGGGCTACGGCGATGGCACACCAGC
TCCCGGCCAAGGCACACAAGTGACTGTCTCGTCTGCTAGCCACCATCACCATCAC
CAC
>SEQ ID NO: 223; DR434(DR235-DR230)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGACTGGTGCAGCCCGGC
GGCTCTCTGAGGCTGAGCTGTGCTGCCAGCGGCTTCACTTTCAGCAGCGCTCACA
TGAGCTGGGTGAGGCAAGCCCCCGGCAAAGGAAGGGAGTGGATCGCCTCCATCT
ACAGCGGCGGCGGAACATTCTACGCCGACAGCGTGAAGGGAAGGTTCACAATCT
CTAGGGACAACGCCAAGAACACACTGTATCTGCAGCTGAACTCTCTGAAGGCCG
AGGACACTGCCATGTACTACTGCGCCACTAATAGGCTGCACTACTACAGCGACG
ATGATTCTCTGAGGGGCCAAGGCACACAAGTGACAGTCTCGTCTGCTAGCCACC
ATCACCATCACCAC
>SEQ ID NO: 224; DR435(DR235-DR231)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGAAGCGTGCAAGCCGGC
GGATCTCTGAGACTCAGCTGTACTGCCTCCGGCTTCACATTCGACGATAGGGAGA
TGAACTGGTATAGGCAAGCCCCCGGCAATGAGTGCGAGCTGGTGAGCACAATCT
CCAGCGATGGCAGCACTTACTACGCCGATAGCGTGAAGGGAAGGTTCACTATCT
CCCAAGATAACGCCAAGAACACAGTCTATCTGCAGATGGACTCCGTCAAGCCAG
AGGATACTGCCGTGTACTACTGCGCCGCCGACTTCATGATCGCCATCCAAGCCCC
CGGCGCTGGCTGTTGGGGACAAGGCACTCAAGTGACAGTCTCGTCTGCTAGCCA
CCATCACCATCACCAC
>SEQ ID NO: 225; DR436(DR235-DR232)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGCAGCGTGCAAGCCGGA
GGCTCTCTGAGACTGAGCTGTGTGGCTAGTGGCTACACAAGCTGCATGGGCTGGT
TTAGGCAAGCCCCCGGCAAGGAGAGAGAGGCCGTGGCCACAATCTACACTAGGG
GAAGGAGCATCTACTACGCCGACAGCGTGAAAGGAAGGTTCACAATCAGCCAAG
ATAACGCCAAGAACACTCTGTATCTGCAGATGAACAGCCTCAAGCCAGAGGACA
TCGCCATGTATAGCTGTGCTGCTGGCGGCTATAGCTGGAGCGCTGGCTGCGAGTT
CAATTACTGGGGCCAAGGCACACAAGTGACTGTCTCGTCTGCTAGCCACCATCAC
CATCACCAC
>SEQ ID NO: 226; DR437(DR235-DR233)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGCGGAAGCGTGCAAGCTGGA
GGATCTCTGAGGCTGAGCTGCACAGCCAGCGGCTTCACTTTCGATGACAGCGAC
ATGGGCTGGTATAGGCAAGCCCCCGGCAATGAGTGTGAGCTGGTGAGCACAATC
AGCAGCGACGGCTCCACTTACTACGCCGACAGCGTCAAGGGAAGGTTCACAATC
AGCCAAGATAACGCCAAGAACACTGTGTATCTGCAGATGAACTCTCTGAAGCCA
GAGGACACAGCCGTGTACTACTGTGCTGCCGAGCCTAGGGGCTACTATAGCAAC
TACGGCGGAAGGAGGGAGTGCAATTACTGGGGCCAAGGCACACAAGTGACAGT
CTCGTCTGCTAGCCACCATCACCATCACCAC
>SEQ ID NO: 227; DR438(DR235-DR234)
CAAGTGCAGCTGCAAGAGAGCGGAGGAGGAAGCGTGCAAGCCGGAGGCTCTCT
GAGACTGAGCTGTGCCGCCTCTAGGTATCTGTACAGCATCGACTACATGGCTTGG
TTCAGACAGAGCCCCGGCAAGGAGAGGGAGCCAGTGGCTGTCATCTACACTGCC
TCCGGCGCCACATTCTATCCAGATAGCGTGAAGGGAAGGTTCACTATCAGCCAA
GATAACGCCAAGATGACAGTGTATCTGCAGATGAACTCTCTGAAGAGCGAGGAC
ACTGCCATGTACTACTGTGCCGCCGTGAGGAAGACAGATAGCTACCTCTTCGACG
CCCAGAGCTTCACATACTGGGGCCAAGGCACACAAGTGACAGTCTCGAGCGGCG
GAGGATCCCAAGTGCAGCTGCAAGAGAGCGGAGGAGGAAGCGTGCAAGCCGGA
GGCTCTCTGAGGCTGAGCTGTGTGGCTAGTGGCTACACTTTCAGCAGCTACTGCA
TGGGCTGGTTCAGACAAGCCCCCGGCAAGGAAAGGGAAGGAGTGGCCGCTCTGG
GCGGAGGAAGCACATACTACGCTGACAGCGTGAAGGGAAGGTTCACAATCAGCC
AAGATAACGCCAAGAACACACTGTATCTGCAGATGAACTCTCTGAAGCCAGAGG
ACACAGCCATGTACTACTGTGCCGCTGCTTGGGTCGCTTGTCTGGAGTTCGGCGG
CAGCTGGTACGATCTGGCTAGGTACAAGCACTGGGGCCAAGGCACACAAGTGAC
AGTCTCGTCTGCTAGCCACCATCACCATCACCAC
>SEQ ID NO: 228; DR439(DR236-DR229)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGACTGG
TCCAGCCCGGCGGCTCTCTGAGGCTGAGCTGCACTGCTTCCGGCTTCAGCTTCAG
CAGCTACCCTATGACATGGGCTAGGCAAGCCCCCGGCAAAGGACTGGAATGGGT
GAGCACTATTGCCAGCGATGGAGGCAGCACAGCCTACGCTGCCAGCGTGGAGGG
AAGGTTCACAATCTCTAGGGACAATGCCAAGAGCACACTGTATCTGCAGCTGAA
CTCTCTGAAGACAGAGGACACTGCCATGTACTACTGCACTAAGGGCTACGGCGA
TGGCACACCAGCTCCCGGCCAAGGCACACAAGTGACTGTCTCGTCTGCTAGCCAC
CATCACCATCACCAC
>SEQ ID NO: 229; DR440(DR236-DR230)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGACTGG
TGCAGCCCGGCGGCTCTCTGAGGCTGAGCTGTGCTGCCAGCGGCTTCACTTTCAG
CAGCGCTCACATGAGCTGGGTGAGGCAAGCCCCCGGCAAAGGAAGGGAGTGGA
TCGCCTCCATCTACAGCGGCGGCGGAACATTCTACGCCGACAGCGTGAAGGGAA
GGTTCACAATCTCTAGGGACAACGCCAAGAACACACTGTATCTGCAGCTGAACT
CTCTGAAGGCCGAGGACACTGCCATGTACTACTGCGCCACTAATAGGCTGCACTA
CTACAGCGACGATGATTCTCTGAGGGGCCAAGGCACACAAGTGACAGTCTCGTC
TGCTAGCCACCATCACCATCACCAC
>SEQ ID NO: 230; DR441(DR236-DR231)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGAAGCG
TGCAAGCCGGCGGATCTCTGAGACTCAGCTGTACTGCCTCCGGCTTCACATTCGA
CGATAGGGAGATGAACTGGTATAGGCAAGCCCCCGGCAATGAGTGCGAGCTGGT
GAGCACAATCTCCAGCGATGGCAGCACTTACTACGCCGATAGCGTGAAGGGAAG
GTTCACTATCTCCCAAGATAACGCCAAGAACACAGTCTATCTGCAGATGGACTCC
GTCAAGCCAGAGGATACTGCCGTGTACTACTGCGCCGCCGACTTCATGATCGCCA
TCCAAGCCCCCGGCGCTGGCTGTTGGGGACAAGGCACTCAAGTGACAGTCTCGT
CTGCTAGCCACCATCACCATCACCAC
>SEQ ID NO: 231; DR442(DR236-DR232)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGCAGCG
TGCAAGCCGGAGGCTCTCTGAGACTGAGCTGTGTGGCTAGTGGCTACACAAGCT
GCATGGGCTGGTTTAGGCAAGCCCCCGGCAAGGAGAGAGAGGCCGTGGCCACAA
TCTACACTAGGGGAAGGAGCATCTACTACGCCGACAGCGTGAAAGGAAGGTTCA
CAATCAGCCAAGATAACGCCAAGAACACTCTGTATCTGCAGATGAACAGCCTCA
AGCCAGAGGACATCGCCATGTATAGCTGTGCTGCTGGCGGCTATAGCTGGAGCG
CTGGCTGCGAGTTCAATTACTGGGGCCAAGGCACACAAGTGACTGTCTCGTCTGC
TAGCCACCATCACCATCACCAC
>SEQ ID NO: 232; DR443(DR236-DR233)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGCGGAAGCG
TGCAAGCTGGAGGATCTCTGAGGCTGAGCTGCACAGCCAGCGGCTTCACTTTCGA
TGACAGCGACATGGGCTGGTATAGGCAAGCCCCCGGCAATGAGTGTGAGCTGGT
GAGCACAATCAGCAGCGACGGCTCCACTTACTACGCCGACAGCGTCAAGGGAAG
GTTCACAATCAGCCAAGATAACGCCAAGAACACTGTGTATCTGCAGATGAACTC
TCTGAAGCCAGAGGACACAGCCGTGTACTACTGTGCTGCCGAGCCTAGGGGCTA
CTATAGCAACTACGGCGGAAGGAGGGAGTGCAATTACTGGGGCCAAGGCACACA
AGTGACAGTCTCGTCTGCTAGCCACCATCACCATCACCAC
>SEQ ID NO: 233; DR444(DR236-DR234)
CAAGTGCAGCTGCAAGAGAGCGGAGGAGGAAGCGTGCAAGCCGGAGGCTCTCT
GAGGCTGAGCTGTGCCGCCTCTAGGTTCACATACAGCAGCTACTGCATGGGCTGG
TTCAGACAAGCCCCCGGCAAAGAGAGAGAAGGCGTGGCCAGCATCGATAGCGAT
GGCTCCACTAGCTACACTGACAGCGTGAAGGGAAGGTTCACTATCAGCAAGGAC
AACGCCAAGAACACTCTGTATCTGCAGATGAACTCTCTGAAGCCAGAGGACACA
GCCATGTACTACTGTGCCCTCGATCTGATGAGCACAGTGGTGCCCGGCTTCTGTG
GCTTTCTGCTGAGCGCTGGCATGGATTACTGGGGCAAGGGCACTCAAGTGACTGT
CTCGAGCGGCGGAGGATCCCAAGTGCAGCTGCAAGAGAGCGGAGGAGGAAGCG
TGCAAGCCGGAGGCTCTCTGAGGCTGAGCTGTGTGGCTAGTGGCTACACTTTCAG
CAGCTACTGCATGGGCTGGTTCAGACAAGCCCCCGGCAAGGAAAGGGAAGGAGT
GGCCGCTCTGGGCGGAGGAAGCACATACTACGCTGACAGCGTGAAGGGAAGGTT
CACAATCAGCCAAGATAACGCCAAGAACACACTGTATCTGCAGATGAACTCTCT
GAAGCCAGAGGACACAGCCATGTACTACTGTGCCGCTGCTTGGGTCGCTTGTCTG
GAGTTCGGCGGCAGCTGGTACGATCTGGCTAGGTACAAGCACTGGGGCCAAGGC
ACACAAGTGACAGTCTCGTCTGCTAGCCACCATCACCATCACCAC
>SEQ ID NO: 234; DR445(DR237-DR229)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGAG
GACTGGTCCAGCCCGGCGGCTCTCTGAGGCTGAGCTGCACTGCTTCCGGCTTCAG
CTTCAGCAGCTACCCTATGACATGGGCTAGGCAAGCCCCCGGCAAAGGACTGGA
ATGGGTGAGCACTATTGCCAGCGATGGAGGCAGCACAGCCTACGCTGCCAGCGT
GGAGGGAAGGTTCACAATCTCTAGGGACAATGCCAAGAGCACACTGTATCTGCA
GCTGAACTCTCTGAAGACAGAGGACACTGCCATGTACTACTGCACTAAGGGCTA
CGGCGATGGCACACCAGCTCCCGGCCAAGGCACACAAGTGACTGTCTCGTCTGC
TAGCCACCATCACCATCACCAC
>SEQ ID NO: 235; DR446(DR237-DR230)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGAG
GACTGGTGCAGCCCGGCGGCTCTCTGAGGCTGAGCTGTGCTGCCAGCGGCTTCAC
TTTCAGCAGCGCTCACATGAGCTGGGTGAGGCAAGCCCCCGGCAAAGGAAGGGA
GTGGATCGCCTCCATCTACAGCGGCGGCGGAACATTCTACGCCGACAGCGTGAA
GGGAAGGTTCACAATCTCTAGGGACAACGCCAAGAACACACTGTATCTGCAGCT
GAACTCTCTGAAGGCCGAGGACACTGCCATGTACTACTGCGCCACTAATAGGCT
GCACTACTACAGCGACGATGATTCTCTGAGGGGCCAAGGCACACAAGTGACAGT
CTCGTCTGCTAGCCACCATCACCATCACCAC
>SEQ ID NO: 236; DR447(DR237-DR231)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGAG
GAAGCGTGCAAGCCGGCGGATCTCTGAGACTCAGCTGTACTGCCTCCGGCTTCAC
ATTCGACGATAGGGAGATGAACTGGTATAGGCAAGCCCCCGGCAATGAGTGCGA
GCTGGTGAGCACAATCTCCAGCGATGGCAGCACTTACTACGCCGATAGCGTGAA
GGGAAGGTTCACTATCTCCCAAGATAACGCCAAGAACACAGTCTATCTGCAGAT
GGACTCCGTCAAGCCAGAGGATACTGCCGTGTACTACTGCGCCGCCGACTTCATG
ATCGCCATCCAAGCCCCCGGCGCTGGCTGTTGGGGACAAGGCACTCAAGTGACA
GTCTCGTCTGCTAGCCACCATCACCATCACCAC
>SEQ ID NO: 237; DR448(DR237-DR232)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGAG
GCAGCGTGCAAGCCGGAGGCTCTCTGAGACTGAGCTGTGTGGCTAGTGGCTACA
CAAGCTGCATGGGCTGGTTTAGGCAAGCCCCCGGCAAGGAGAGAGAGGCCGTGG
CCACAATCTACACTAGGGGAAGGAGCATCTACTACGCCGACAGCGTGAAAGGAA
GGTTCACAATCAGCCAAGATAACGCCAAGAACACTCTGTATCTGCAGATGAACA
GCCTCAAGCCAGAGGACATCGCCATGTATAGCTGTGCTGCTGGCGGCTATAGCTG
GAGCGCTGGCTGCGAGTTCAATTACTGGGGCCAAGGCACACAAGTGACTGTCTC
GTCTGCTAGCCACCATCACCATCACCAC
>SEQ ID NO: 238; DR449(DR237-DR233)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGCG
GAAGCGTGCAAGCTGGAGGATCTCTGAGGCTGAGCTGCACAGCCAGCGGCTTCA
CTTTCGATGACAGCGACATGGGCTGGTATAGGCAAGCCCCCGGCAATGAGTGTG
AGCTGGTGAGCACAATCAGCAGCGACGGCTCCACTTACTACGCCGACAGCGTCA
AGGGAAGGTTCACAATCAGCCAAGATAACGCCAAGAACACTGTGTATCTGCAGA
TGAACTCTCTGAAGCCAGAGGACACAGCCGTGTACTACTGTGCTGCCGAGCCTA
GGGGCTACTATAGCAACTACGGCGGAAGGAGGGAGTGCAATTACTGGGGCCAAG
GCACACAAGTGACAGTCTCGTCTGCTAGCCACCATCACCATCACCAC
>SEQ ID NO: 239; DR450(DR237-DR234)
CAAGTGCAGCTGCAAGAGTCCGGAGGAGGCAGCGTCCAAGCCGGAGGCTCTCTG
AGGCTGAGCTGTGCTGCCAGCGGCTACACTTACAGCATGTACTGCATGGGCTGGT
TCAGACAAGCCCCCGGCAAGGAAAGAGAGGGCGTGGCCCAGATCAATAGCGAT
GGCAGCACAAGCTACGCCGACAGCGTGAAGGGAAGGTTCACTATCTCCAAGGAC
AACGCCAAGAACACTCTGTATCTGCAGATGAACTCTCTGAAGCCAGAGGACACT
GCCATGTACTACTGCGCTGCCGATTCTAGGGTGTACGGCGGCAGCTGGTATGAGA
GGCTCTGCGGCCCTTACACATACGAGTACAACTACTGGGGCCAAGGCACACAAG
TGACTGTCTCGAGCGGCGGAGGATCCCAAGTGCAGCTGCAAGAGAGCGGAGGAG
GAAGCGTGCAAGCCGGAGGCTCTCTGAGGCTGAGCTGTGTGGCTAGTGGCTACA
CTTTCAGCAGCTACTGCATGGGCTGGTTCAGACAAGCCCCCGGCAAGGAAAGGG
AAGGAGTGGCCGCTCTGGGCGGAGGAAGCACATACTACGCTGACAGCGTGAAGG
GAAGGTTCACAATCAGCCAAGATAACGCCAAGAACACACTGTATCTGCAGATGA
ACTCTCTGAAGCCAGAGGACACAGCCATGTACTACTGTGCCGCTGCTTGGGTCGC
TTGTCTGGAGTTCGGCGGCAGCTGGTACGATCTGGCTAGGTACAAGCACTGGGG
CCAAGGCACACAAGTGACAGTCTCGTCTGCTAGCCACCATCACCATCACCAC
>SEQ ID NO: 240; DR451(DR238-DR229)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGAGGACTGGTCCAGCCCGGCGGCTCTCTGAGGCTGAGCTGCACTGC
TTCCGGCTTCAGCTTCAGCAGCTACCCTATGACATGGGCTAGGCAAGCCCCCGGC
AAAGGACTGGAATGGGTGAGCACTATTGCCAGCGATGGAGGCAGCACAGCCTAC
GCTGCCAGCGTGGAGGGAAGGTTCACAATCTCTAGGGACAATGCCAAGAGCACA
CTGTATCTGCAGCTGAACTCTCTGAAGACAGAGGACACTGCCATGTACTACTGCA
CTAAGGGCTACGGCGATGGCACACCAGCTCCCGGCCAAGGCACACAAGTGACTG
TCTCGTCTGCTAGCCACCATCACCATCACCAC
>SEQ ID NO: 241; DR452(DR238-DR230)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGAGGACTGGTGCAGCCCGGCGGCTCTCTGAGGCTGAGCTGTGCTGC
CAGCGGCTTCACTTTCAGCAGCGCTCACATGAGCTGGGTGAGGCAAGCCCCCGG
CAAAGGAAGGGAGTGGATCGCCTCCATCTACAGCGGCGGCGGAACATTCTACGC
CGACAGCGTGAAGGGAAGGTTCACAATCTCTAGGGACAACGCCAAGAACACACT
GTATCTGCAGCTGAACTCTCTGAAGGCCGAGGACACTGCCATGTACTACTGCGCC
ACTAATAGGCTGCACTACTACAGCGACGATGATTCTCTGAGGGGCCAAGGCACA
CAAGTGACAGTCTCGTCTGCTAGCCACCATCACCATCACCAC
>SEQ ID NO: 242; DR453(DR238-DR231)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGAGGAAGCGTGCAAGCCGGCGGATCTCTGAGACTCAGCTGTACTGC
CTCCGGCTTCACATTCGACGATAGGGAGATGAACTGGTATAGGCAAGCCCCCGG
CAATGAGTGCGAGCTGGTGAGCACAATCTCCAGCGATGGCAGCACTTACTACGC
CGATAGCGTGAAGGGAAGGTTCACTATCTCCCAAGATAACGCCAAGAACACAGT
CTATCTGCAGATGGACTCCGTCAAGCCAGAGGATACTGCCGTGTACTACTGCGCC
GCCGACTTCATGATCGCCATCCAAGCCCCCGGCGCTGGCTGTTGGGGACAAGGC
ACTCAAGTGACAGTCTCGTCTGCTAGCCACCATCACCATCACCAC
>SEQ ID NO: 243; DR454(DR238-DR232)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGAGGCAGCGTGCAAGCCGGAGGCTCTCTGAGACTGAGCTGTGTGGC
TAGTGGCTACACAAGCTGCATGGGCTGGTTTAGGCAAGCCCCCGGCAAGGAGAG
AGAGGCCGTGGCCACAATCTACACTAGGGGAAGGAGCATCTACTACGCCGACAG
CGTGAAAGGAAGGTTCACAATCAGCCAAGATAACGCCAAGAACACTCTGTATCT
GCAGATGAACAGCCTCAAGCCAGAGGACATCGCCATGTATAGCTGTGCTGCTGG
CGGCTATAGCTGGAGCGCTGGCTGCGAGTTCAATTACTGGGGCCAAGGCACACA
AGTGACTGTCTCGTCTGCTAGCCACCATCACCATCACCAC
>SEQ ID NO: 244; DR455(DR238-DR233)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGCGGAAGCGTGCAAGCTGGAGGATCTCTGAGGCTGAGCTGCACAGC
CAGCGGCTTCACTTTCGATGACAGCGACATGGGCTGGTATAGGCAAGCCCCCGG
CAATGAGTGTGAGCTGGTGAGCACAATCAGCAGCGACGGCTCCACTTACTACGC
CGACAGCGTCAAGGGAAGGTTCACAATCAGCCAAGATAACGCCAAGAACACTGT
GTATCTGCAGATGAACTCTCTGAAGCCAGAGGACACAGCCGTGTACTACTGTGCT
GCCGAGCCTAGGGGCTACTATAGCAACTACGGCGGAAGGAGGGAGTGCAATTAC
TGGGGCCAAGGCACACAAGTGACAGTCTCGTCTGCTAGCCACCATCACCATCAC
CAC
>SEQ ID NO: 245; DR456(DR238-DR234)
CAAGTGCAGCTGCAAGAGAGCGGCGGAGGAAGCGTGCAAGCCGGAGGATCTCT
GAGACTGAGCTGCGCTGTGAGCGGCTACGCCTACTCCACATACTGCATGGGCTG
GTTTAGGCAAGCCCCCGGCAAAGAGAGAGAGGGCGTGGCTGCTATCGATAGCGG
CGGCAGCACAAGCTACGCCGATAGCGTGAAGGGAAGGTTCACAATCAGCAAGG
ACAACGCCAAGAACACACTGTATCTGAGGATGAACTCTCTGAAGCCAGAGGACA
CAGCCATGTACTACTGTGCTGCTGTGCCTCCTCCTCCAGATGGCGGCAGCTGTCT
GTTTCTGGGACCAGAGATCAAGGTCAGCAAGGCCGATTTTAGGTACTGGGGCCA
AGGCACACAAGTGACAGTCTCGAGCGGCGGAGGATCCCAAGTGCAGCTGCAAGA
GAGCGGAGGAGGAAGCGTGCAAGCCGGAGGCTCTCTGAGGCTGAGCTGTGTGGC
TAGTGGCTACACTTTCAGCAGCTACTGCATGGGCTGGTTCAGACAAGCCCCCGGC
AAGGAAAGGGAAGGAGTGGCCGCTCTGGGCGGAGGAAGCACATACTACGCTGA
CAGCGTGAAGGGAAGGTTCACAATCAGCCAAGATAACGCCAAGAACACACTGTA
TCTGCAGATGAACTCTCTGAAGCCAGAGGACACAGCCATGTACTACTGTGCCGCT
GCTTGGGTCGCTTGTCTGGAGTTCGGCGGCAGCTGGTACGATCTGGCTAGGTACA
AGCACTGGGGCCAAGGCACACAAGTGACAGTCTCGTCTGCTAGCCACCATCACC
ATCACCAC
>SEQ ID NO: 246; DR457(DR239-DR229)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGAGGACTGGTCCAG
CCCGGCGGCTCTCTGAGGCTGAGCTGCACTGCTTCCGGCTTCAGCTTCAGCAGCT
ACCCTATGACATGGGCTAGGCAAGCCCCCGGCAAAGGACTGGAATGGGTGAGCA
CTATTGCCAGCGATGGAGGCAGCACAGCCTACGCTGCCAGCGTGGAGGGAAGGT
TCACAATCTCTAGGGACAATGCCAAGAGCACACTGTATCTGCAGCTGAACTCTCT
GAAGACAGAGGACACTGCCATGTACTACTGCACTAAGGGCTACGGCGATGGCAC
ACCAGCTCCCGGCCAAGGCACACAAGTGACTGTCTCGTCTGCTAGCCACCATCAC
CATCACCAC
>SEQ ID NO: 247; DR458(DR239-DR230)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGAGGACTGGTGCAG
CCCGGCGGCTCTCTGAGGCTGAGCTGTGCTGCCAGCGGCTTCACTTTCAGCAGCG
CTCACATGAGCTGGGTGAGGCAAGCCCCCGGCAAAGGAAGGGAGTGGATCGCCT
CCATCTACAGCGGCGGCGGAACATTCTACGCCGACAGCGTGAAGGGAAGGTTCA
CAATCTCTAGGGACAACGCCAAGAACACACTGTATCTGCAGCTGAACTCTCTGA
AGGCCGAGGACACTGCCATGTACTACTGCGCCACTAATAGGCTGCACTACTACA
GCGACGATGATTCTCTGAGGGGCCAAGGCACACAAGTGACAGTCTCGTCTGCTA
GCCACCATCACCATCACCAC
>SEQ ID NO: 248; DR459(DR239-DR231)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGAGGAAGCGTGCAA
GCCGGCGGATCTCTGAGACTCAGCTGTACTGCCTCCGGCTTCACATTCGACGATA
GGGAGATGAACTGGTATAGGCAAGCCCCCGGCAATGAGTGCGAGCTGGTGAGCA
CAATCTCCAGCGATGGCAGCACTTACTACGCCGATAGCGTGAAGGGAAGGTTCA
CTATCTCCCAAGATAACGCCAAGAACACAGTCTATCTGCAGATGGACTCCGTCAA
GCCAGAGGATACTGCCGTGTACTACTGCGCCGCCGACTTCATGATCGCCATCCAA
GCCCCCGGCGCTGGCTGTTGGGGACAAGGCACTCAAGTGACAGTCTCGTCTGCTA
GCCACCATCACCATCACCAC
>SEQ ID NO: 249; DR460(DR239-DR232)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGAGGCAGCGTGCAA
GCCGGAGGCTCTCTGAGACTGAGCTGTGTGGCTAGTGGCTACACAAGCTGCATG
GGCTGGTTTAGGCAAGCCCCCGGCAAGGAGAGAGAGGCCGTGGCCACAATCTAC
ACTAGGGGAAGGAGCATCTACTACGCCGACAGCGTGAAAGGAAGGTTCACAATC
AGCCAAGATAACGCCAAGAACACTCTGTATCTGCAGATGAACAGCCTCAAGCCA
GAGGACATCGCCATGTATAGCTGTGCTGCTGGCGGCTATAGCTGGAGCGCTGGCT
GCGAGTTCAATTACTGGGGCCAAGGCACACAAGTGACTGTCTCGTCTGCTAGCCA
CCATCACCATCACCAC
>SEQ ID NO: 250; DR461(DR239-DR233)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGCGGAAGCGTGCAA
GCTGGAGGATCTCTGAGGCTGAGCTGCACAGCCAGCGGCTTCACTTTCGATGACA
GCGACATGGGCTGGTATAGGCAAGCCCCCGGCAATGAGTGTGAGCTGGTGAGCA
CAATCAGCAGCGACGGCTCCACTTACTACGCCGACAGCGTCAAGGGAAGGTTCA
CAATCAGCCAAGATAACGCCAAGAACACTGTGTATCTGCAGATGAACTCTCTGA
AGCCAGAGGACACAGCCGTGTACTACTGTGCTGCCGAGCCTAGGGGCTACTATA
GCAACTACGGCGGAAGGAGGGAGTGCAATTACTGGGGCCAAGGCACACAAGTG
ACAGTCTCGTCTGCTAGCCACCATCACCATCACCAC
>SEQ ID NO: 251; DR462(DR239-DR234)
CAAGTGCAGCTGCAAGAGAGCGGAGGAGGAAGCGTGCAAGCCGGAGGCTCTCT
GAGGCTGAGCTGTACAGTGTCCGGCTACACTTACAGCTCCAATTGCATGGGCTGG
TTTAGGCAAGCCCCCGGCAAGGAAAGAGAGGGCGTGGCCACTATCTACACTGGC
GGCGGCAACACATACTACGCCGATAGCGTGAAGGGAAGGTTCACTATCAGCCAA
GATAACGCCAAGAACACAGTGTATCTGCAGATGAACAATCTGAAGCCAGAGGAC
ACTGCCATGTACTACTGTGCTGCTGAGCCACTGTCTAGGGTGTACGGCGGCAGCT
GCCCAACTCCTACATTCGACTACTGGGGCCAAGGCACACAAGTGACTGTCTCGA
GCGGCGGAGGATCCCAAGTGCAGCTGCAAGAGAGCGGAGGAGGAAGCGTGCAA
GCCGGAGGCTCTCTGAGGCTGAGCTGTGTGGCTAGTGGCTACACTTTCAGCAGCT
ACTGCATGGGCTGGTTCAGACAAGCCCCCGGCAAGGAAAGGGAAGGAGTGGCC
GCTCTGGGCGGAGGAAGCACATACTACGCTGACAGCGTGAAGGGAAGGTTCACA
ATCAGCCAAGATAACGCCAAGAACACACTGTATCTGCAGATGAACTCTCTGAAG
CCAGAGGACACAGCCATGTACTACTGTGCCGCTGCTTGGGTCGCTTGTCTGGAGT
TCGGCGGCAGCTGGTACGATCTGGCTAGGTACAAGCACTGGGGCCAAGGCACAC
AAGTGACAGTCTCGTCTGCTAGCCACCATCACCATCACCAC
>SEQ ID NO: 252; DR463(DR240-DR229)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGACTGGTCCAGCC
CGGCGGCTCTCTGAGGCTGAGCTGCACTGCTTCCGGCTTCAGCTTCAGCAGCTAC
CCTATGACATGGGCTAGGCAAGCCCCCGGCAAAGGACTGGAATGGGTGAGCACT
ATTGCCAGCGATGGAGGCAGCACAGCCTACGCTGCCAGCGTGGAGGGAAGGTTC
ACAATCTCTAGGGACAATGCCAAGAGCACACTGTATCTGCAGCTGAACTCTCTGA
AGACAGAGGACACTGCCATGTACTACTGCACTAAGGGCTACGGCGATGGCACAC
CAGCTCCCGGCCAAGGCACACAAGTGACTGTCTCGTCTGCTAGCCACCATCACCA
TCACCAC
>SEQ ID NO: 253; DR464(DR240-DR230)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGACTGGTGCAGCC
CGGCGGCTCTCTGAGGCTGAGCTGTGCTGCCAGCGGCTTCACTTTCAGCAGCGCT
CACATGAGCTGGGTGAGGCAAGCCCCCGGCAAAGGAAGGGAGTGGATCGCCTCC
ATCTACAGCGGCGGCGGAACATTCTACGCCGACAGCGTGAAGGGAAGGTTCACA
ATCTCTAGGGACAACGCCAAGAACACACTGTATCTGCAGCTGAACTCTCTGAAG
GCCGAGGACACTGCCATGTACTACTGCGCCACTAATAGGCTGCACTACTACAGC
GACGATGATTCTCTGAGGGGCCAAGGCACACAAGTGACAGTCTCGTCTGCTAGC
CACCATCACCATCACCAC
>SEQ ID NO: 254; DR465(DR240-DR231)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGAAGCGTGCAAGC
CGGCGGATCTCTGAGACTCAGCTGTACTGCCTCCGGCTTCACATTCGACGATAGG
GAGATGAACTGGTATAGGCAAGCCCCCGGCAATGAGTGCGAGCTGGTGAGCACA
ATCTCCAGCGATGGCAGCACTTACTACGCCGATAGCGTGAAGGGAAGGTTCACT
ATCTCCCAAGATAACGCCAAGAACACAGTCTATCTGCAGATGGACTCCGTCAAG
CCAGAGGATACTGCCGTGTACTACTGCGCCGCCGACTTCATGATCGCCATCCAAG
CCCCCGGCGCTGGCTGTTGGGGACAAGGCACTCAAGTGACAGTCTCGTCTGCTAG
CCACCATCACCATCACCAC
>SEQ ID NO: 255; DR466(DR240-DR232)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGCAGCGTGCAAGC
CGGAGGCTCTCTGAGACTGAGCTGTGTGGCTAGTGGCTACACAAGCTGCATGGG
CTGGTTTAGGCAAGCCCCCGGCAAGGAGAGAGAGGCCGTGGCCACAATCTACAC
TAGGGGAAGGAGCATCTACTACGCCGACAGCGTGAAAGGAAGGTTCACAATCAG
CCAAGATAACGCCAAGAACACTCTGTATCTGCAGATGAACAGCCTCAAGCCAGA
GGACATCGCCATGTATAGCTGTGCTGCTGGCGGCTATAGCTGGAGCGCTGGCTGC
GAGTTCAATTACTGGGGCCAAGGCACACAAGTGACTGTCTCGTCTGCTAGCCACC
ATCACCATCACCAC
>SEQ ID NO: 256; DR467(DR240-DR233)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGCGGAAGCGTGCAAGC
TGGAGGATCTCTGAGGCTGAGCTGCACAGCCAGCGGCTTCACTTTCGATGACAGC
GACATGGGCTGGTATAGGCAAGCCCCCGGCAATGAGTGTGAGCTGGTGAGCACA
ATCAGCAGCGACGGCTCCACTTACTACGCCGACAGCGTCAAGGGAAGGTTCACA
ATCAGCCAAGATAACGCCAAGAACACTGTGTATCTGCAGATGAACTCTCTGAAG
CCAGAGGACACAGCCGTGTACTACTGTGCTGCCGAGCCTAGGGGCTACTATAGC
AACTACGGCGGAAGGAGGGAGTGCAATTACTGGGGCCAAGGCACACAAGTGAC
AGTCTCGTCTGCTAGCCACCATCACCATCACCAC
>SEQ ID NO: 257; DR468(DR240-DR234)
CAAGTGCAGCTGCAAGAGAGCGGAGGAGGAAGCGTCCAAGCCGGAGGCTCTCT
GAGGCTGAGCTGTGGAGCCAGCGGCTACACTTACAGCAGCTACTGTATGGGCTG
GTTTAGGCAAGTGCCCGGCAAGGAGAGAGAGGGCGTGGCCGTGATCGATTCCGA
TGGCAGCACAAGCTACGCTGACAGCGTGAAGGGAAGGTTCACAATCAGCAAGGA
CAACGGCAAGAACACACTCTATCTGCAGATGAACAGCCTCAAGCCAGAGGACAC
AGCCATGTACTACTGCGCCGCTGATCTGGGCCACTATAGGCCTCCTTGTGGCGTG
CTGTATCTGGGCATGGATTACTGGGGCAAGGGCACACAAGTGACAGTCTCGAGC
GGCGGAGGATCCCAAGTGCAGCTGCAAGAGAGCGGAGGAGGAAGCGTGCAAGC
CGGAGGCTCTCTGAGGCTGAGCTGTGTGGCTAGTGGCTACACTTTCAGCAGCTAC
TGCATGGGCTGGTTCAGACAAGCCCCCGGCAAGGAAAGGGAAGGAGTGGCCGCT
CTGGGCGGAGGAAGCACATACTACGCTGACAGCGTGAAGGGAAGGTTCACAATC
AGCCAAGATAACGCCAAGAACACACTGTATCTGCAGATGAACTCTCTGAAGCCA
GAGGACACAGCCATGTACTACTGTGCCGCTGCTTGGGTCGCTTGTCTGGAGTTCG
GCGGCAGCTGGTACGATCTGGCTAGGTACAAGCACTGGGGCCAAGGCACACAAG
TGACAGTCTCGTCTGCTAGCCACCATCACCATCACCAC
>SEQ ID NO: 258; DR469(DR241-DR229)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGACTGGTCC
AGCCCGGCGGCTCTCTGAGGCTGAGCTGCACTGCTTCCGGCTTCAGCTTCAGCAG
CTACCCTATGACATGGGCTAGGCAAGCCCCCGGCAAAGGACTGGAATGGGTGAG
CACTATTGCCAGCGATGGAGGCAGCACAGCCTACGCTGCCAGCGTGGAGGGAAG
GTTCACAATCTCTAGGGACAATGCCAAGAGCACACTGTATCTGCAGCTGAACTCT
CTGAAGACAGAGGACACTGCCATGTACTACTGCACTAAGGGCTACGGCGATGGC
ACACCAGCTCCCGGCCAAGGCACACAAGTGACTGTCTCGTCTGCTAGCCACCATC
ACCATCACCAC
>SEQ ID NO: 259; DR470(DR241-DR230)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGACTGGTGC
AGCCCGGCGGCTCTCTGAGGCTGAGCTGTGCTGCCAGCGGCTTCACTTTCAGCAG
CGCTCACATGAGCTGGGTGAGGCAAGCCCCCGGCAAAGGAAGGGAGTGGATCGC
CTCCATCTACAGCGGCGGCGGAACATTCTACGCCGACAGCGTGAAGGGAAGGTT
CACAATCTCTAGGGACAACGCCAAGAACACACTGTATCTGCAGCTGAACTCTCTG
AAGGCCGAGGACACTGCCATGTACTACTGCGCCACTAATAGGCTGCACTACTAC
AGCGACGATGATTCTCTGAGGGGCCAAGGCACACAAGTGACAGTCTCGTCTGCT
AGCCACCATCACCATCACCAC
>SEQ ID NO: 260; DR471(DR241-DR231)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGAAGCGTGC
AAGCCGGCGGATCTCTGAGACTCAGCTGTACTGCCTCCGGCTTCACATTCGACGA
TAGGGAGATGAACTGGTATAGGCAAGCCCCCGGCAATGAGTGCGAGCTGGTGAG
CACAATCTCCAGCGATGGCAGCACTTACTACGCCGATAGCGTGAAGGGAAGGTT
CACTATCTCCCAAGATAACGCCAAGAACACAGTCTATCTGCAGATGGACTCCGTC
AAGCCAGAGGATACTGCCGTGTACTACTGCGCCGCCGACTTCATGATCGCCATCC
AAGCCCCCGGCGCTGGCTGTTGGGGACAAGGCACTCAAGTGACAGTCTCGTCTG
CTAGCCACCATCACCATCACCAC
>SEQ ID NO: 261; DR472(DR241-DR232)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGCAGCGTGC
AAGCCGGAGGCTCTCTGAGACTGAGCTGTGTGGCTAGTGGCTACACAAGCTGCA
TGGGCTGGTTTAGGCAAGCCCCCGGCAAGGAGAGAGAGGCCGTGGCCACAATCT
ACACTAGGGGAAGGAGCATCTACTACGCCGACAGCGTGAAAGGAAGGTTCACAA
TCAGCCAAGATAACGCCAAGAACACTCTGTATCTGCAGATGAACAGCCTCAAGC
CAGAGGACATCGCCATGTATAGCTGTGCTGCTGGCGGCTATAGCTGGAGCGCTG
GCTGCGAGTTCAATTACTGGGGCCAAGGCACACAAGTGACTGTCTCGTCTGCTAG
CCACCATCACCATCACCAC
>SEQ ID NO: 262; DR473(DR241-DR233)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGCGGAAGCGTGC
AAGCTGGAGGATCTCTGAGGCTGAGCTGCACAGCCAGCGGCTTCACTTTCGATG
ACAGCGACATGGGCTGGTATAGGCAAGCCCCCGGCAATGAGTGTGAGCTGGTGA
GCACAATCAGCAGCGACGGCTCCACTTACTACGCCGACAGCGTCAAGGGAAGGT
TCACAATCAGCCAAGATAACGCCAAGAACACTGTGTATCTGCAGATGAACTCTCT
GAAGCCAGAGGACACAGCCGTGTACTACTGTGCTGCCGAGCCTAGGGGCTACTA
TAGCAACTACGGCGGAAGGAGGGAGTGCAATTACTGGGGCCAAGGCACACAAG
TGACAGTCTCGTCTGCTAGCCACCATCACCATCACCAC
>SEQ ID NO: 263; DR474(DR241-DR234)
CAAGTGCAGCTGCAAGAGAGCGGAGGAGGAAGCGTCCAAGCCGGAGGCTCTCT
GAGACTGAGCTGTGCCGCCAGCGGCTACTCCAACTGCAGCTACGACATGACTTG
GTATAGGCAAGCCCCCGGCAAGGAGAGGGAGTTCGTGTCCGCCATCCACAGCGA
CGGCAGCACTAGATACGCCGACAGCGTGAAGGGAAGGTTCTTCATCAGCCAAGA
TAACGCCAAGAACACAGTGTATCTGCAGATGAACTCCCTCAAGCCAGAGGACAC
TGCCATGTACTACTGCAAGACAGACCCACTGCACTGCAGAGCCCATGGCGGCAG
CTGGTATAGCGTGAGGGCCAACTACTGGGGCCAAGGCACACAAGTGACAGTCTC
GAGCGGCGGAGGATCCCAAGTGCAGCTGCAAGAGAGCGGAGGAGGAAGCGTGC
AAGCCGGAGGCTCTCTGAGGCTGAGCTGTGTGGCTAGTGGCTACACTTTCAGCAG
CTACTGCATGGGCTGGTTCAGACAAGCCCCCGGCAAGGAAAGGGAAGGAGTGGC
CGCTCTGGGCGGAGGAAGCACATACTACGCTGACAGCGTGAAGGGAAGGTTCAC
AATCAGCCAAGATAACGCCAAGAACACACTGTATCTGCAGATGAACTCTCTGAA
GCCAGAGGACACAGCCATGTACTACTGTGCCGCTGCTTGGGTCGCTTGTCTGGAG
TTCGGCGGCAGCTGGTACGATCTGGCTAGGTACAAGCACTGGGGCCAAGGCACA
CAAGTGACAGTCTCGTCTGCTAGCCACCATCACCATCACCAC

It is understood that the embodiments described herein are for illustrative purposes only and that various modifications or changes in light thereof will be suggested to persons skilled in the art and are to be included within the spirit and purview of this application and scope of the appended claims. The sequences of the sequence accession numbers cited herein are hereby incorporated by reference.

1. An IL10Rα/IL2Rγ binding protein that specifically binds to IL10Rα and IL2Rγ, comprising an anti-IL10Rα V_{H}H antibody and an anti-IL2Rγ V_{H}H antibody, wherein,
(A) the anti-IL100Rα V_{H}H antibody comprises a complementarity determining region 1 (CDR1) comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(B) the anti-IL10Rα V_{H}H antibody comprises a complementarity determining region 1 (CDR1) comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(C) the anti-IL10Rα V_{H}H antibody comprises a complementarity determining region 1 (CDR1) comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO:11; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(D) the anti-IL10Rα V_{H}H antibody comprises a complementarity determining region 1 (CDR1) comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(E) the anti-IL10Rα V_{H}H antibody comprises a complementarity determining region 1 (CDR1) comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(F) the anti-IL10Rα V_{H}H antibody comprises a complementarity determining region 1 (CDR1) comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47.

2. (canceled)

3. (canceled)

4. The IL10Rα/IL2Rγ binding protein of claim 1, wherein the anti-L10Rα V_{H}H antibody comprises a sequence having at least 90% identity to a sequence of any one of DR235 (SEQ ID NO:4), DR236 (SEQ ID NO:8), DR237 (SEQ ID NO:12), DR239 (SEQ ID NO:16), DR240 (SEQ ID NO:20), and DR241 (SEQ ID NO:24).

5. (canceled)

6. (canceled)

7. The IL10Rα/IL2Rγ binding protein of claim 1, wherein the anti-IL10Rα V_{H}H antibody comprises a sequence having at least 90% identity to a sequence of any one of DR229 (SEQ ID NO:28), DR230 (SEQ ID NO:32), DR231 (SEQ ID NO:36), DR232 (SEQ ID NO:40), DR233 (SEQ ID NO:44), and DR234 (SEQ ID NO:48).

8. The IL10Rα/IL2Rγ binding protein of claim 1, wherein the anti-IL10Rα V_{H}H antibody is at the N-terminus and the anti-IL2Rγ V_{H}H antibody is at the C-terminus.

9. The IL10Rα/IL2Rγ binding protein of claim 8, wherein the binding protein comprises a sequence having at least 90% identity to a sequence of any one of SEQ ID NOS:49-59.

10. The IL10Rα/IL2Rγ binding protein of claim 1, wherein the anti-IL2Rγ V_{H}H antibody is at the N-terminus and the anti-IL10Rα V_{H}H antibody is at the C-terminus.

11. The IL10Rα/IL2Rγ binding protein of claim 10, wherein the binding protein comprises at least 90% identity to a sequence of any one of SEQ ID NOS:60 and 61.

12. The IL10Rα/IL2Rγ binding protein of claim 1, wherein the anti-IL10Rα V_{H}H antibody and the anti-IL2Rγ V_{H}H antibody are joined by a peptide linker.

13. The IL10Rα/IL2Rγ binding protein of claim 12, wherein the peptide linker comprises between 1 and 50 amino acids.

14. The IL10Rα/IL2Rγ binding protein of claim 1, wherein the binding protein comprises a sequence with at least 90% (e.g., 90%, 91%, 92%, 93%, 91%, 95%, 96%, 97%, 98%, 99%, 100%) identity to a sequence of any one of SEQ ID NOS:49-61 or 96-179, optionally without a HHHHHH sequence.

15. The IL10Rα/IL2Rγ binding protein of claim 1, wherein the binding protein is conjugated to an Fc polypeptide or an Fc domain.

16. The IL10Rα/IL2Rγ binding protein of claim 15, wherein the Fc polypeptide or the Fc domain is from an IgG1, IgG2, IgG3 or IgG4.

17. The IL10Rα/IL2Rγ binding protein of claim 16, wherein the IL10Rα/IL2Rγ binding protein comprises SEQ ID NO: 556 or SEQ ID NO:558.

18. The IL10Rα/IL2Rγ binding protein of claim 1, wherein the binding protein is PEGylated.

19. An IL10Rα/IL2Rγ binding protein that specifically binds to IL10Rα and IL2Rγ, comprising an anti-IL10Rα V_{H}H antibody and an anti-IL2Rγ V_{H}H antibody, wherein the IL10Rα/IL2Rγ binding protein is linked to a Fc polypeptide or a Fc domain from an IgG1, IgG2, IgG3 or IgG4.

20. A heterodimeric IL10Rα binding protein/IL2Rγ binding protein pair, the heterodimeric IL10Rα binding protein/IL2Rγ binding protein pair comprising a first polypeptide of the formula #1:
anti-IL10Rα V_{H}H antibody-L1ₐ-UH1-Fc1 [1]
and a second polypeptide of the formula #2:
anti-IL2Rγ V_{H}H antibody-L2_{b}-UH2-Fc2 [2]
wherein:
L1 and L2 are GSA linkers and a and b are independently selected from 0 (absent) or 1 (present);
UH1 and UH2 are each an upper hinge domain of human immunoglobulin independently selected from the group consisting of the IgG1, IgG2, IgG3 and IgG4 upper hinge, optionally comprising the amino acid substitution C220S (EU numbering);
Fc1 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc2, and
FC2 is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc1, and
wherein the polypeptide of formula 1 and the polypeptide of formula 2 are linked by at least one interchain disulfide bond, and wherein
(A) the anti-IL100Rα V_{H}H antibody comprises a complementarity determining region 1 (CDR1) comprising an amino acid sequence of SEQ ID NO:1 or SEQ ID NO:264, a CDR2 comprising an amino acid sequence of SEQ ID NO:2, and a CDR3 comprising an amino acid sequence of SEQ ID NO:3; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(B) the anti-IL10Rα V_{H}H antibody comprises a complementarity determining region 1 (CDR1) comprising an amino acid sequence of SEQ ID NO:5 or SEQ ID NO:265, a CDR2 comprising an amino acid sequence of SEQ ID NO:6, and a CDR3 comprising an amino acid sequence of SEQ ID NO:7; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(C) the anti-IL10Rα V_{H}H antibody comprises a complementarity determining region 1 (CDR1) comprising an amino acid sequence of SEQ ID NO:9 or SEQ ID NO:266, a CDR2 comprising an amino acid sequence of SEQ ID NO:10, and a CDR3 comprising an amino acid sequence of SEQ ID NO:11; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(D) the anti-IL10Rα V_{H}H antibody comprises a complementarity determining region 1 (CDR1) comprising an amino acid sequence of SEQ ID NO:13 or SEQ ID NO:267, a CDR2 comprising an amino acid sequence of SEQ ID NO:14, and a CDR3 comprising an amino acid sequence of SEQ ID NO:15; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(E) the anti-IL10Rα V_{H}H antibody comprises a complementarity determining region 1 (CDR1) comprising an amino acid sequence of SEQ ID NO:17 or SEQ ID NO:268, a CDR2 comprising an amino acid sequence of SEQ ID NO:18, and a CDR3 comprising an amino acid sequence of SEQ ID NO:19; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(F) the anti-IL10Rα V_{H}H antibody comprises a complementarity determining region 1 (CDR1) comprising an amino acid sequence of SEQ ID NO:21 or SEQ ID NO:269, a CDR2 comprising an amino acid sequence of SEQ ID NO:22, and a CDR3 comprising an amino acid sequence of SEQ ID NO:23; and
wherein the anti-IL2Rγ V_{H}H antibody comprises:
i) a CDR1 comprising an amino acid sequence of SEQ ID NO:25 or SEQ ID NO:270, a CDR2 comprising an amino acid sequence of SEQ ID NO:26, and a CDR3 comprising an amino acid sequence of SEQ ID NO:27;
ii) a CDR1 comprising an amino acid sequence of SEQ ID NO:29 or SEQ ID NO:271, a CDR2 comprising an amino acid sequence of SEQ ID NO:30, and a CDR3 comprising an amino acid sequence of SEQ ID NO:31;
iii) a CDR1 comprising an amino acid sequence of SEQ ID NO:33 or SEQ ID NO:272, a CDR2 comprising an amino acid sequence of SEQ ID NO:34, and a CDR3 comprising an amino acid sequence of SEQ ID NO:35;
iv) a CDR1 comprising an amino acid sequence of SEQ ID NO:37 or SEQ ID NO:273, a CDR2 comprising an amino acid sequence of SEQ ID NO:38, and a CDR3 comprising an amino acid sequence of SEQ ID NO:39;
v) a CDR1 comprising an amino acid sequence of SEQ ID NO:41 or SEQ ID NO:274, a CDR2 comprising an amino acid sequence of SEQ ID NO:42, and a CDR3 comprising an amino acid sequence of SEQ ID NO:43; or
vi) a CDR1 comprising an amino acid sequence of SEQ ID NO:45 or SEQ ID NO:275, a CDR2 comprising an amino acid sequence of SEQ ID NO:46, and a CDR3 comprising an amino acid sequence of SEQ ID NO:47; or
(G) the anti-IL10Rα V_{H}H antibody comprises:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 in a row of Table 10;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 in a row of Table 10; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 10; and
the anti-IL2Rγ V_{H}H antibody comprises:
a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR1 listed in Table 11 or Table 12;
a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR2 listed in Table 11 or Table 12; and
a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any CDR3 listed in Table 11 or Table 12.

21. An isolated nucleic acid encoding the IL10Rα/IL2Rγ binding protein of claim 1.

22. An expression vector comprising the nucleic acid of claim 21.

23. An isolated host cell comprising the vector of claim 22.

24. A pharmaceutical composition comprising the IL10Rα/IL2Rγ binding protein of claim 1 and a pharmaceutically acceptable carrier.

25. A method of treating a neoplastic disease in a subject in need thereof, comprising administering to the subject a therapeutically effective amount of an IL10Rα/IL2Rγ binding protein of claim 1.

26-28. (canceled)

29. A method of treating an autoimmune or inflammatory disease, disorder, or condition in a subject in need thereof, comprising administering to the subject a therapeutically effective amount of an IL10Rα/IL2Rγ binding protein of claim 1.

30-33. (canceled)

34. A method to selectively induce activity in one or more of a first cell type over one or more of a second cell type, comprising contacting a population of cells comprising both the first and second cell types with an IL10Ra/IL2Rγ binding protein of claim 1, thereby selectively inducing activity in one or more of the first cell type over one or more of the second cell type.

35-42. (canceled)
